(19) United States
(12) Patent Application Publication
(10) Pub. No.: US 2023/0365641 A1
(43) Pub. Date: Nov. 16, 2023

(54) TARGETED CYTOKINES AND METHODS OF USE THEREOF

(71) Applicant: Xilio Development, Inc., Waltham, MA (US)

(72) Inventors: Parker Johnson, Waltham, MA (US); Ertan Eryilmaz, Waltham, MA (US); Dheeraj Tomar, Waltham, MA (US); Benjamin Nicholson, Waltham, MA (US); Kurt Jenkins, Waltham, MA (US); Carl Uli Bialucha, Waltham, MA (US)

(21) Appl. No.: 18/115,527

(22) Filed: Feb. 28, 2023

Related U.S. Application Data
(60) Provisional application No. 63/314,928, filed on Feb. 28, 2022.
(60) Provisional application No. 63/314,926, filed on Feb. 28, 2022.

(51) Int. Cl. C07K 14/52; C07K 14/705; C07K 16/28; A61P 37/02

(52) U.S. Cl. CPC C07K 14/52; C07K 14/70596; C07K 16/2818; A61P 37/02; C07K 2319/30; C07K 2317/565

(57) ABSTRACT

The present invention relates to targeted, masked cytokines, comprising a cytokine or functional fragment thereof, a masking moiety, a targeting moiety and a proteolytically cleavable linker. The targeting moiety comprises an antigen-binding moiety that specifically binds to an antigen expressed on the surface of a target cell The masking moiety masks the cytokine or functional fragment thereof thereby reducing or preventing binding of the cytokine or functional fragment thereof to its cognate receptor, but upon proteolytic cleavage of the cleavable linker at a target site, the cytokine or functional fragment thereof becomes activated, which renders it capable or more capable of binding to its cognate receptor.

### CROSS-REFERENCE TO RELATED APPLICATIONS

This application claims priority to, and the benefit of, U.S. provisional application No. 63/314,928, filed on Feb. 28, 2022, and U.S. provisional application No. 63/314,926, filed on Feb. 28, 2022, the contents of each of which is hereby incorporated by reference in its entirety.

### SEQUENCE LISTING

The instant application is being filed with an electronically filed Sequence Listing in XML format. The sequence listing file entitled XTX_4GC_P0114US1_SL.xml, was created on Jul. 19, 2023, and is 254,390 bytes in size; the information in electronic format of the Sequence Listing is incorporated herein by reference in its entirety.

### BACKGROUND

Cancer is the second leading cause of death in the United States, accounting for more deaths than the next five leading causes (chronic respiratory disease, stroke, accidents, Alzheimer's disease, and diabetes). While great strides have been made especially with targeted therapies, there remains a great deal of work to do in this space. Immunotherapy and a branch of this field, immuno-oncology, is creating viable and exciting therapeutic options for treating malignancies. Specifically, it is now recognized that one hallmark of cancer is immune evasion and significant efforts have identified targets and developed therapies to these targets to reactivate the immune system to recognize and treat cancer.

Cytokine therapy is an effective strategy for stimulating the immune system to induce anti-tumor cytotoxicity. In particular, Proleukin (aldesleukin), a recombinant form of interleukin-2 (IL-2), has been approved by the FDA for the treatment of metastatic renal cell carcinoma and melanoma. Unfortunately, cytokines that are administered to patients generally have a very short half-life, thereby requiring frequent dosing. For instance, the product label of aldesleukin, marketed under the brand name Proleukin, states that the drug was shown to have a half-life of 85 minutes in patients who received a 5-minute intravenous (IV) infusion. In addition, administration of high doses of cytokine can cause adverse health outcomes, such as vascular leakage, through systemic immune activation. These findings illustrate the need for developing IL-2 cytokine therapeutics that effectively target tumors without the side effects associated with systemic immune activation. Provided herein are targeted, masked IL-2 cytokines, cleavage products of said targeted IL-2 cytokines, and compositions thereof and methods of use thereof for addressing this need.

### SUMMARY OF THE INVENTION

The present invention provides, among other things, methods and compositions for use in the treatment of cancer. The present invention is based, in part, on the surprising discovery that a targeting masked cytokines of the present invention specifically target a cell of interest for effective treatment of cancer without causing undesired side effects. The targeting cytokines of the present invention specifically bind the target cells of interest, accumulate in tumor, and be activated by tumor proteases. Furthermore, treatment with the targeting masked cytokines of the present invention increases the percentage of CD8 T cells in the tumor microenvironment and significantly inhibits tumor growth with minimal toxicity in vivo.

In one aspect, the present invention provides, among other things, a targeted cytokine comprising a targeting moiety, a cytokine or fragment thereof, a masking moiety, and an Fc domain comprising a first Fc polypeptide linked to the cytokine or a fragment thereof through a first linker and a second Fc polypeptide linked to the masking moiety through a second linker.

In one aspect, the present invention provides, among other things, a targeted cytokine comprising a targeting moiety, a cytokine or a fragment thereof, a masking moiety, and an Fc domain comprising a first Fc polypeptide linked to the IL-2 cytokine or a fragment thereof through a first linker and a second Fc polypeptide linked to the masking moiety through a second linker, wherein the wherein the masking moiety binds to the IL-2 cytokine or a fragment thereof, wherein the first or the second linker is a cleavable linker such that the masking moiety releases the IL-2 cytokine or a fragment thereof upon cleavage, wherein the cleavable linker comprises a sequence selected from Table 1 and wherein the targeting moiety is linked to the Fc domain through one or both of the first and second Fc polypeptides.

In one aspect, the present invention provides, among other things, a targeted cytokine comprising a targeting moiety, a cytokine or a fragment thereof, a masking moiety, and an Fc domain comprising a first Fc polypeptide linked to the IL-2 cytokine or a fragment thereof through a first linker and a second Fc polypeptide linked to the masking moiety through a second linker, wherein the wherein the masking moiety binds to the IL-2 cytokine or a fragment thereof, wherein the first or the second linker is a cleavable linker such that the masking moiety releases the IL-2 cytokine or a fragment thereof upon cleavage, wherein the cleavable linker comprises MPYDLYHP (SEQ ID NO: 34) or VPLSLYSG (SEQ ID NO: 42) and wherein the targeting moiety is linked to the Fc domain through one or both of the first and second Fc polypeptides.

In some embodiments, the cleavable linker comprises PVSLRSGS (SEQ ID NO: 196), or GMPKDLYHAS (SEQ ID NO. 197), or RPLALWRS (SEQ ID NO: 193), or TQKPLGLS (SEQ ID NO:194), or APAGLIVPYN (SEQ ID NO:195), or PANLVAPDP (SEQ ID NO: 183), or IVGRPRHQGV (SEQ ID NO:199), or RSKYLATA (SEQ ID NO:198).

In some embodiments, the cleavable linker comprises MPYDLYHP (SEQ ID NO: 34), RAAAVKSP, or VPLSLYSG (SEQ ID NO: 42).

In some embodiments, a masking moiety binds to the cytokine or a fragment thereof. In some embodiments, a masking moiety comprises an agent that binds to a cytokine or a fragment thereof. In some embodiments, a masking moiety comprises a peptide that binds to a cytokine or a fragment thereof. In some embodiments, a masking moiety comprises a polypeptide that binds to a cytokine or a fragment thereof. In some embodiments, a masking moiety comprises a protein that binds to a cytokine or a fragment thereof. In some embodiments, a masking moiety comprises a nucleic acid that binds to a cytokine or a fragment thereof.

In some embodiments, a masking moiety comprises a Fab. In some embodiments, a masking moiety comprises a single chain Fv (scFv). In some embodiments, a masking moiety comprises a single domain antibody (VHH). In some embodiments, a masking moiety comprises one or more CDRs. In some embodiments, a masking moiety comprises a variable heavy chain (VH). In some embodiments, a masking moiety comprises a variable light chain (VL). In some embodiments, a masking moiety comprises a Fab-like bispecific antibodies (bsFab). In some embodiments, a masking moiety comprises a single-domain antibody-linked Fab (s-Fab). In some embodiments, a masking moiety comprises an antibody. In some embodiments, a masking moiety comprises a combination thereof. In some embodiments, a masking moiety comprises a Fab that binds to a cytokine. In some embodiments, a masking moiety comprises a single chain Fv (scFv) that binds to a cytokine. In some embodiments, a masking moiety comprises a single domain antibody (VHH) that binds to a cytokine. In some embodiments, a masking moiety comprises one or more CDRs that bind to a cytokine. In some embodiments, a masking moiety comprises a variable heavy chain (VH) that binds to a cytokine. In some embodiments, a masking moiety comprises a variable light chain (VL) that binds to a cytokine. In some embodiments, a masking moiety comprises a Fab-like bispecific antibodies (bsFab) that binds to a cytokine. In some embodiments, a masking moiety comprises a single-domain antibody-linked Fab (s-Fab) that binds to a cytokine. In some embodiments, a masking moiety comprises an antibody or a fragment thereof that binds to a cytokine. In some embodiments, the masking moiety comprises an antibody against the cytokine or a binding fragment of the antibody.

In some embodiments, the masking moiety is a receptor of the cytokine. In some embodiments, the masking moiety is a fragment of a receptor of the cytokine. In some embodiments, the masking moiety is an extracellular domain (ECD) of a receptor of the cytokine.

In some embodiments, the masking moiety is CD121. In some embodiments, the masking moiety is IL-18Rα. In some embodiments, the masking moiety is IL-18Rβ. In some embodiments, the masking moiety is CD25. In some embodiments, the masking moiety is CD132. In some embodiments, the masking moiety is CD124. In some embodiments, the masking moiety is CD213a13. In some embodiments, the masking moiety is CD132. In some embodiments, the masking moiety is CD127. In some embodiments, the masking moiety is IL-9R. In some embodiments, the masking moiety is CD213a1. In some embodiments, the masking moiety is CD213a2. In some embodiments, the masking moiety is CD1243. In some embodiments, the masking moiety is CD132. In some embodiments, the masking moiety is IL-15Ra. In some embodiments, the masking moiety is CDw131. In some embodiments, the masking moiety is CDw125. In some embodiments, the masking moiety is CD131. In some embodiments, the masking moiety is CD116. In some embodiments, the masking moiety is CD126. In some embodiments, the masking moiety is CD130. In some embodiments, the masking moiety is IL-11Ra. In some embodiments, the masking moiety is CD114. In some embodiments, the masking moiety CD212. In some embodiments, the masking moiety is LIFR. In some embodiments, the masking moiety is OSMR. In some embodiments, the masking moiety is IL-20Rα. In some embodiments, the masking moiety is IL-20Rβ. In some embodiments, the masking moiety is IL-14R. In some embodiments, the masking moiety is CD4. In some embodiments, the masking moiety is CDw127. In some embodiments, the masking moiety is CD118. In some embodiments, the masking moiety is CDw119. In some embodiments, the masking moiety is CD40. In some embodiments, the masking moiety is LTβR. In some embodiments, the masking moiety is CD120a. In some embodiments, the masking moiety is CD120b. In some embodiments, the masking moiety is CDw137. In some embodiments, the masking moiety is BCMA. In some embodiments, the masking moiety is TACI. In some embodiments, the masking moiety is CD27. In some embodiments, the masking moiety is CD30. In some embodiments, the masking moiety is CD95. In some embodiments, the masking moiety is GITR. In some embodiments, the masking moiety is LTbR. In some embodiments, the masking moiety is HVEM. In some embodiments, the masking moiety is OX40. In some embodiments, the masking moiety is TRAILR1-4. In some embodiments, the masking moiety is Apo3. In some embodiments, the masking moiety is RANK. In some embodiments, the masking moiety is OPG. In some embodiments, the masking moiety is TGF-βR1. In some embodiments, the masking moiety is TGF-βR2. In some embodiments, the masking moiety is TGF-βR3. In some embodiments, the masking moiety is CD115. In some embodiments, the masking moiety is CDw136.

In some embodiments, the masking moiety is CD122. In some embodiments, the CD122 is an engineered CD122 polypeptide or a fragment thereof comprising one or more mutations relative to a wild-type CD122 amino acid sequence.

In some embodiments, the engineered CD122 comprises F8C mutation. In some embodiments, the engineered CD122 comprises A94C mutation. In some embodiments, the engineered CD122 comprises L106C mutation. In some embodiments, the engineered CD122 comprises C122S mutation. In some embodiments, the engineered CD122 comprises C122V mutation. In some embodiments, the engineered CD122 comprises C122A mutation. In some embodiments, the engineered CD122 comprises N123C mutation. In some embodiments, the engineered CD122 comprises N123Q mutation. In some embodiments, the engineered CD122 comprises C168V mutation. In some embodiments, the engineered CD122 comprises C168A mutation. In some embodiments, the engineered CD122 comprises C168S mutation. In some embodiments, the engineered CD122 comprises L169C mutation. In some embodiments, the engineered CD122 comprises Q177C mutation. In some embodiments, the engineered CD122 comprises V184C mutation. In some embodiments, the engineered CD122 comprises S195C mutation. In some embodiments, the engineered CD122 comprises R204C mutation.

In some embodiments, the engineered CD122 comprises C122S and C168S mutations. In some embodiments, the engineered CD122 comprises C122A and N123C mutations.

In some embodiments, the engineered CD122 comprises C122V and C168V mutations. In some embodiments, the engineered CD122 comprises C122A and C168V mutations. In some embodiments, the engineered CD122 comprises C122V and C168A mutations. In some embodiments, the engineered CD122 comprises C122V and N123C mutations. In some embodiments, the engineered CD122 comprises C122A and C168A mutations. In some embodiments, the engineered CD122 comprises V117C, N123Q and C168A mutations. In some embodiments, the engineered CD122 comprises N123Q, C168A, and L169C mutations. In some embodiments, the engineered CD122 comprises L106C, C122A, C168A and S195C mutations. In some embodiments, the engineered CD122 comprises L106C, C122A, C168A and V184C mutations. In some embodiments, the engineered CD122 comprises C122A, C168A, V184C, and S195C mutations. In some embodiments, the engineered CD122 comprises C122A, C168A, Q177C, and R204C mutations. In some embodiments, the engineered CD122 comprises L106C, C122V, C168V, and S195C mutations. In some embodiments, the engineered CD122 comprises F8C, A94C, C122V, and C168V mutations.

In some embodiments, the engineered CD122 polypeptide or a fragment thereof comprises a mutation at amino acid 122 and 168 relative to SEQ ID NO: 15, wherein the engineered CD122 has improved stability compared to a wild-type CD122. In some embodiments, the engineered CD122 polypeptide or a fragment thereof comprises a mutation to facilitate disulfide bond formation. In some embodiments, the engineered CD122 polypeptide or a fragment thereof comprises a mutation that creates a salt bridge.

In some embodiments, the masking moiety comprises the extracellular domain of human IL-12Rβ1 or a fragment, portion, or variant thereof that retains or otherwise demonstrates an affinity to IL-12. In some embodiments, the masking moiety comprises residues 24 to 237 of human IL-12Rβ1. In some embodiments, the masking moiety comprises a sequence having SEQ ID NO: 126. In some embodiments, the masking moiety comprises residues 24 to 545 of human IL-12Rβ1. In some embodiments, the masking moiety comprises a sequence having SEQ ID NO: 127.

In some embodiments, the masking moiety comprises the extracellular domain of human IL-12Rβ2 or a fragment, portion, or variant thereof that retains or otherwise demonstrates an affinity to IL-12. In some embodiments, the masking moiety comprises residues 24 to 212 of human IL-12Rβ1. In some embodiments, the masking moiety comprises a sequence having SEQ ID NO: 128. In some embodiments, the masking moiety comprises residues 24 to 222 of human IL-12Rβ2. In some embodiments, the masking moiety comprises a sequence having SEQ ID NO: 129. In some embodiments, the masking moiety comprises residues 24 to 227 of human IL-12Rβ2. In some embodiments, the masking moiety comprises a sequence having SEQ ID NO: 133. In some embodiments, the masking moiety comprises residues 24 to 319 of human IL-12Rβ2. In some embodiments, the masking moiety comprises a sequence having SEQ ID NO: 130.

In some embodiments, the masking moiety comprises at least one amino acid modification as compared to the sequence of SEQ ID NO: 130. In some embodiments, the masking moiety comprises at least one amino acid modification as compared to the sequence of SEQ ID NO: 130, optionally wherein said modifications are cysteine substitution mutations. In some embodiments, the masking moiety comprises SEQ ID NO: 131. In some embodiments, the masking moiety comprises residues 24 to 622 of human IL-12Rβ2. In some embodiments, the masking moiety comprises a sequence having SEQ ID NO: 132.

In some embodiments, a cytokine is IL-1 or a variant thereof. In some embodiments, a cytokine is IL-2 or a variant thereof. In some embodiments, a cytokine is IL-3 or a variant thereof. In some embodiments, a cytokine is IL-4 or a variant thereof. In some embodiments, a cytokine is IL-5 or a variant thereof. In some embodiments, a cytokine is IL-6 or a variant thereof. In some embodiments, a cytokine is IL-7 or a variant thereof. In some embodiments, a cytokine is IL-9 or a variant thereof. In some embodiments, a cytokine is IL-10 or a variant thereof. In some embodiments, a cytokine is IL-11 or a variant thereof. In some embodiments, a cytokine is IL-12 or a variant thereof. In some embodiments, a cytokine is IL-13 or a variant thereof. In some embodiments, a cytokine is IL-14 or a variant thereof. In some embodiments, a cytokine is IL-15 or a variant thereof. In some embodiments, a cytokine is IL-16 or a variant thereof. In some embodiments, a cytokine is IL-17 or a variant thereof. In some embodiments, a cytokine is IL-20 or a variant thereof. In some embodiments, a cytokine is TNF-α or a variant thereof. In some embodiments, a cytokine TNF-β or a variant thereof. In some embodiments, a cytokine is CXCL8 (IL-18) or a variant thereof. In some embodiments, a cytokine is G-CSF or a variant thereof. In some embodiments, a cytokine is CXCL8 (IL-18) or a variant thereof. In some embodiments, a cytokine is GM-CSF or a variant thereof. In some embodiments, a cytokine is LIF or a variant thereof. In some embodiments, a cytokine is OSM or a variant thereof. In some embodiments, a cytokine is IFN-α or a variant thereof. In some embodiments, a cytokine is IFN-β or a variant thereof. In some embodiments, a cytokine is IFN-γ or a variant thereof. In some embodiments, a cytokine is CD154 or a variant thereof. In some embodiments, a cytokine is LT-β or a variant thereof. In some embodiments, a cytokine is 4-1BBL or a variant thereof. In some embodiments, a cytokine is APRIL or a variant thereof. In some embodiments, a cytokine is CD153 or a variant thereof. In some embodiments, a cytokine is CD70 or a variant thereof. In some embodiments, a cytokine is CD178 or a variant thereof. In some embodiments, a cytokine is GITRL or a variant thereof. In some embodiments, a cytokine is LIGHT or a variant thereof. In some embodiments, a cytokine is OX40L or a variant thereof. In some embodiments, a cytokine is TALL-1 or a variant thereof. In some embodiments, a cytokine is TRAIL or a variant thereof. In some embodiments, a cytokine is TWEAK or a variant thereof. In some embodiments, a cytokine is TRANCE or a variant thereof. In some embodiments, a cytokine is TGF-β or a variant thereof. In some embodiments, a cytokine is M-CSF or a variant thereof. In some embodiments, a cytokine is MSP or a variant thereof.

In some embodiments, the IL-2 is a modified IL-2 cytokine or functional fragment thereof compared to the sequence of a mature IL-2 having SEQ ID NO: 13. In some embodiments, the modified IL-2 cytokine or functional fragment thereof comprises modifications R38A relative to the sequence of a mature IL-2 having SEQ ID NO: 13. In some embodiments, the modified IL-2 cytokine or functional fragment thereof comprises modifications F42A relative to the sequence of a mature IL-2 having SEQ ID NO: 13. In some embodiments, the modified IL-2 cytokine or functional fragment thereof comprises modifications Y45A relative to the sequence of a mature IL-2 having SEQ ID NO: 13. In some embodiments, the modified IL-2 cytokine or functional fragment thereof comprises modifications E62A relative to the sequence of a mature IL-2 having SEQ ID NO: 13. In some embodiments, the modified IL-2 cytokine or functional fragment thereof comprises modifications R38A, F42A, Y45A, and E62A relative to the sequence of a mature IL-2 having SEQ ID NO: 13. In some embodiments, the modified IL-2 cytokine or functional fragment thereof comprises the modification C125A relative to the sequence of a mature IL-2 having SEQ ID NO: 13.

In some embodiments, the modified IL-2 cytokine or functional fragment thereof comprises modifications R38A, F42A, Y45A, and E62A relative to the sequence of a mature IL-2 having SEQ ID NO: 13. In some embodiments, the modified IL-2 cytokine or functional fragment thereof comprises an amino acid sequence of SEQ ID NO: 14.

In some embodiments, the modified IL-2 cytokine or functional fragment thereof comprises modifications that reduce or eliminate binding to CD25.

In some embodiments, the modified IL-2 cytokine or functional fragment thereof comprises modifications that enhance binding to CD25.

In some embodiments, the modified IL-2 cytokine or functional fragment thereof comprises modifications that reduce or eliminate binding to CD122.

In some embodiments, the modified IL-2 cytokine or functional fragment thereof comprises modifications that enhance binding to CD122.

In some embodiments, the IL-15 polypeptide comprises an amino acid sequence of SEQ ID NO: 93 or an amino acid sequence having at least one amino acid modification as compared to the amino acid sequence of SEQ ID NO: 93. In some embodiments, the IL-15 cytokine or functional fragment thereof comprises an amino acid sequence having one or more amino acid substitutions at positions D22 as compared to the amino acid sequence of SEQ ID NO: 93. In some embodiments, the IL-15 cytokine or functional fragment thereof comprises an amino acid sequence having one or more amino acid substitutions at positions E46 as compared to the amino acid sequence of SEQ ID NO: 93. In some embodiments, the IL-15 cytokine or functional fragment thereof comprises an amino acid sequence having one or more amino acid substitutions at positions E53 as compared to the amino acid sequence of SEQ ID NO: 93. In some embodiments, the IL-15 cytokine or functional fragment thereof comprises an amino acid sequence having one or more amino acid substitutions at positions N71 as compared to the amino acid sequence of SEQ ID NO: 93. In some embodiments, the IL-15 cytokine or functional fragment thereof comprises an amino acid sequence having one or more amino acid substitutions at positions N79 as compared to the amino acid sequence of SEQ ID NO: 93. In some embodiments, the IL-15 cytokine or functional fragment thereof comprises an amino acid sequence having one or more amino acid substitutions at positions N112 as compared to the amino acid sequence of SEQ ID NO: 93.

In some embodiments, the IL-15 cytokine or functional fragment thereof comprises an amino acid sequence having one or more amino acid substitutions at positions D22, E46, or E53 as compared to the amino acid sequence of SEQ ID NO: 93. In some embodiments, the IL-15 cytokine or functional fragment thereof comprises an amino acid sequence having an amino acid substitution at position N71 and N79 as compared to the amino acid sequence of SEQ ID NO: 93. In some embodiments, the IL-15 cytokine or functional fragment thereof comprises an amino acid sequence having an amino acid substitution at position N71 and N112 as compared to the amino acid sequence of SEQ ID NO: 93. In some embodiments, the IL-15 cytokine or functional fragment thereof comprises an amino acid sequence having an amino acid substitution at position N79 and N112 as compared to the amino acid sequence of SEQ ID NO: 93. In some embodiments, the IL-15 cytokine or functional fragment thereof comprises an amino acid sequence having one or more amino acid substitutions at positions D22, E46, E53, N71, N79, or N112 as compared to the amino acid sequence of SEQ ID NO: 93. In some embodiments, the IL-15 cytokine or functional fragment thereof comprises an amino acid sequence of SEQ ID NO: 94.

In some embodiments, the IL-12 polypeptide or functional fragment thereof comprises an IL-12p40 polypeptide or functional fragment thereof covalently linked to an IL-12p35 polypeptide or functional fragment thereof. In some embodiments, the IL-12p40-IL-12p35 linker is between 2 and 40 amino acids in length. In some embodiments, the IL-12p40-IL-12p35 linker is between 4 and 30 amino acids in length. In some embodiments, the IL-12p40-IL-12p35 linker is between 5 and 20 amino acids in length. In some embodiments, the IL-12p40-IL-12p35 linker is between 7 and 15 amino acids in length. In some embodiments, the IL-12p40-IL-12p35 linker is between 10 and 12 amino acids in length. In some embodiments, the IL-12p40-IL-12p35 linker is 2 amino acids in length. In some embodiments, the IL-12p40-IL-12p35 linker is 2 amino acids in length. In some embodiments, the IL-12p40-IL-12p35 linker is 2 amino acids in length. In some embodiments, the IL-12p40-IL-12p35 linker is 2 amino acids in length. In some embodiments, the IL-12p40-IL-12p35 linker is 2 amino acids in length. In some embodiments, the IL-12p40-IL-12p35 linker is 2 amino acids in length. In some embodiments, the IL-12p40-IL-12p35 linker is 3 amino acids in length. In some embodiments, the IL-12p40-IL-12p35 linker is 4 amino acids in length. In some embodiments, the IL-12p40-IL-12p35 linker is 5 amino acids in length. In some embodiments, the IL-12p40-IL-12p35 linker is 6 amino acids in length. In some embodiments, the IL-12p40-IL-12p35 linker is 7 amino acids in length. In some embodiments, the IL-12p40-IL-12p35 linker is 8 amino acids in length. In some embodiments, the IL-12p40-IL-12p35 linker is 9 amino acids in length. In some embodiments, the IL-12p40-IL-12p35 linker is 10 amino acids in length. In some embodiments, the IL-12p40-IL-12p35 linker is 11 amino acids in length. In some embodiments, the IL-12p40-IL-12p35 linker is 12 amino acids in length. In some embodiments, the IL-12p40-IL-12p35 linker is 13 amino acids in length. In some embodiments, the IL-12p40-IL-12p35 linker is 14 amino acids in length. In some embodiments, the IL-12p40-IL-12p35 linker is 15 amino acids in length. In some embodiments, the IL-12p40-IL-12p35 linker is 16 amino acids in length. In some embodiments, the IL-12p40-IL-12p35 linker is 17 amino acids in length. In some embodiments, the IL-12p40-IL-12p35 linker is 18 amino acids in length. In some embodiments, the IL-12p40-IL-12p35 linker is 19 amino acids in length. In some embodiments, the IL-12p40-IL-12p35 linker is 20 amino acids in length. In some embodiments, the IL-12p40-IL-12p35 linker is 21 amino acids in length. In some embodiments, the IL-12p40-IL-12p35 linker is 22 amino acids in length. In some embodiments, the IL-12p40-IL-12p35 linker is 23 amino acids in length. In some embodiments, the IL-12p40-IL-12p35 linker is 24 amino acids in length. In some embodiments, the IL-12p40-IL-12p35 linker is 25 amino acids in length.

In some embodiments, the IL-12p40-IL-12p35 linker comprises amino acid residues A, P, G and S. In some embodiments, the IL-12p40-IL-12p35 linker is rich in amino acid residues G and S. In some embodiments, the IL-12p40-IL-12p35 linker comprises SEQ ID NO: 119 (GGGGSGGGGSGGGGS). In some embodiments the IL-12p40 polypeptide comprises SEQ ID NO: 111. In some embodiments the IL-12p40 polypeptide comprises an amino acid sequence having at least one amino acid modification as compared to the amino acid sequence of SEQ ID NO: 111. In some embodiments the IL-12p40 polypeptide comprises an amino acid sequence having one amino acid modification as compared to the amino acid sequence of SEQ ID NO: 111. In some embodiments the IL-12p40 polypeptide comprises an amino acid sequence having two amino acid modifications as compared to the amino acid sequence of SEQ ID NO: 111. In some embodiments the IL-12p40 polypeptide comprises an amino acid sequence having three amino acid modifications as compared to the amino acid sequence of SEQ ID NO: 111. In some embodiments the IL-12p40 polypeptide comprises an amino acid sequence having four amino acid modifications as compared to the amino acid sequence of SEQ ID NO: 111. In some embodiments the IL-12p40 polypeptide comprises an amino acid sequence having five amino acid modifications as compared to the amino acid sequence of SEQ ID NO: 111.

In some embodiments, IL-12p40 polypeptide comprises at least one amino acid modification to the GAG-binding domain (KSKREKKDRV) as compared to the amino acid sequence of SEQ ID NO: 111.

In some embodiments, the IL-12p40 polypeptide comprises SEQ ID NO: 115. In some embodiments, the IL-12p40 polypeptide comprises SEQ ID NO: 116.

In some embodiments, IL-12p40 polypeptide comprises an amino acid sequence having one or more cysteine substitution mutations as compared to the amino acid sequence of SEQ ID NO: 111. In some embodiments, the IL-12p40 polypeptide comprises SEQ ID NO: 117. In some embodiments, the IL-12p40 polypeptide comprises SEQ ID NO: 118.

In some embodiments, the IL-12p35 polypeptide comprises SEQ ID NO: 112. In some embodiments, the IL-12p35 polypeptide comprises an amino acid sequence having at least one amino acid modification as compared to the amino acid sequence of SEQ ID NO: 112. In some embodiments, the IL-12p35 polypeptide comprises an amino acid sequence having at least two amino acid modifications as compared to the amino acid sequence of SEQ ID NO: 112. In some embodiments, the IL-12p35 polypeptide comprises an amino acid sequence having at least three amino acid modifications as compared to the amino acid sequence of SEQ ID NO: 112. In some embodiments, the IL-12p35 polypeptide comprises an amino acid sequence having at least four amino acid modifications as compared to the amino acid sequence of SEQ ID NO: 112. In some embodiments, the IL-12p35 polypeptide comprises an amino acid sequence having at least five amino acid modifications as compared to the amino acid sequence of SEQ ID NO: 112. In some embodiments, the IL-12p35 polypeptide comprises an amino acid sequence having at least six amino acid modifications as compared to the amino acid sequence of SEQ ID NO: 112. In some embodiments, the IL-12p35 polypeptide comprises an amino acid sequence having at least seven amino acid modifications as compared to the amino acid sequence of SEQ ID NO: 112. In some embodiments, the IL-12p35 polypeptide comprises an amino acid sequence having at least eight amino acid modifications as compared to the amino acid sequence of SEQ ID NO: 112. In some embodiments, the IL-12p35 polypeptide comprises an amino acid sequence having at least nine amino acid modifications as compared to the amino acid sequence of SEQ ID NO: 112. In some embodiments, the IL-12p35 polypeptide comprises an amino acid sequence having at least ten amino acid modifications as compared to the amino acid sequence of SEQ ID NO: 112.

In some embodiments, the IL-12p35 polypeptide comprises an amino acid sequence having one amino acid modification as compared to the amino acid sequence of SEQ ID NO: 112. In some embodiments, the IL-12p35 polypeptide comprises an amino acid sequence having two amino acid modifications as compared to the amino acid sequence of SEQ ID NO: 112. In some embodiments, the IL-12p35 polypeptide comprises an amino acid sequence having three amino acid modifications as compared to the amino acid sequence of SEQ ID NO: 112. In some embodiments, the IL-12p35 polypeptide comprises an amino acid sequence having four amino acid modifications as compared to the amino acid sequence of SEQ ID NO: 112. In some embodiments, the IL-12p35 polypeptide comprises an amino acid sequence having five amino acid modifications as compared to the amino acid sequence of SEQ ID NO: 112. In some embodiments, the IL-12p35 polypeptide comprises an amino acid sequence having six amino acid modifications as compared to the amino acid sequence of SEQ ID NO: 112. In some embodiments, the IL-12p35 polypeptide comprises an amino acid sequence having seven amino acid modifications as compared to the amino acid sequence of SEQ ID NO: 112. In some embodiments, the IL-12p35 polypeptide comprises an amino acid sequence having eight amino acid modifications as compared to the amino acid sequence of SEQ ID NO: 112. In some embodiments, the IL-12p35 polypeptide comprises an amino acid sequence having nine amino acid modifications as compared to the amino acid sequence of SEQ ID NO: 112. In some embodiments, the IL-12p35 polypeptide comprises an amino acid sequence having ten amino acid modifications as compared to the amino acid sequence of SEQ ID NO: 112.

(SEQ ID NO: 150)
IWELKKDVYVVELDWYPDAPGEMVVLTCDTPEEDGITWTLDQSSEVLGS
GKTLTIQVKEFGDAGQYTCHKGGEVLSHSLLLLHKKEDGIWSTDILKDQ
KEPKNKTFLRCEAKNYSGRFTCWWLTTISTDLTFSVKSSRGSSDPQGVT
CGAATLSAERVRGDNKEYEYSVECQEDSACPAAEESLPIEVMVDAVHKL
KYENYTSSFFIRDIIKPDPPKNLQLKPLKNSRQVEVSWEYPDTWSTPHS
YFSLTFCVQVQGKSKREKKDRVFTDKTSATVICRKNASISVRAQDRYYS
SSWSEWASVPCSGGGGSGGGGSGGGGSRNLPVATPDPGMFPCLHHSQNL
LRAVSNMLQKARQTLEFYPCTSEEIDHEDITKDKTSTVEACLPLELTKN
ESCLNSRETSFITNGSCLASRKTSFMMALCLSSIYEDLKMYQVEFKTMN
AKLLMDPKRQIFLDQNMLAVIDELMQALNFNSETVPQKSSLEEPDFYKT
KIKLCILLHAFRIRAVTIDRVMSYLNAS
(SEQ ID NO: 151)
IWELKKDVYVVELDWYPDAPGEMVVLTCDTPEEDGITWTLDQSSEVLGS
GKTLTIQVKEFGDAGQYTCHKGGEVLSHSLLLLHKKEDGIWSTDILKDQ
KEPKNKTFLRCEAKNYSGRFTCWWLTTISTDLTFSVKSSRGSSDPQGVT
CGAATLSAERVRGDNKEYEYSVECQEDSACPAAEESLPIEVMVDAVHKL
KYENYTSSFFIRDIIKPDPPKNLQLKPLKNSRQVEVSWEYPDTWSTPHS
YFSLTFCVQVQGKDNTERVFTDKTSATVICRKNASISVRAQDRYYSSSW
SEWASVPCSGGGGSGGGGSGGGGSRNLPVATPDPGMFPCLHHSQNLLRA
VSNMLQKARQTLEFYPCTSEEIDHEDITKDKTSTVEACLPLELTKNESC
LNSRETSFITNGSCLASRKTSFMMALCLSSIYEDLKMYQVEFKTMNAKL
LMDPKRQIFLDQNMLAVIDELMQALNFNSETVPQKSSLEEPDFYKTKIK
LCILLHAFRIRAVTIDRVMSYLNAS
(SEQ ID NO: 152)
IWELKKDVYVVELDWYPDAPGEMVVLTCDTPEEDGITWTLDQSSEVLGS
GKTLTIQVKEFGDAGQYTCHKGGEVLSHSLLLLHKKEDGIWSTDILKDQ
KEPKNKTFLRCEAKNYSGRFTCWWLTTISTDLTFSVKSSRGSSDPQGVT
CGAATLSAERVRGDNKEYEYSVECQEDSACPAAEESLPIEVMVDAVHKL
KYENYTSSFFIRDIIKPDPPKNLQLKPLKNSRQVEVSWEYPDTWSTPHS
YFSLTFCVQVQGKDNTEGRVFTDKTSATVICRKNASISVRAQDRYYSSS
WSEWASVPCSGGGGSGGGGSGGGGSRNLPVATPDPGMFPCLHHSQNLLR
AVSNMLQKARQTLEFYPCTSEEIDHEDITKDKTSTVEACLPLELTKNES
CLNSRETSFITNGSCLASRKTSFMMALCLSSIYEDLKMYQVEFKTMNAK
LLMDPKRQIFLDQNMLAVIDELMQALNFNSETVPQKSSLEEPDFYKTKI
KLCILLHAFRIRAVTIDRVMSYLNAS
(SEQ ID NO: 153)
IWELKKDVYVVELDWYPDAPGEMVVLTCDTPEEDGITWTLDQSSEVLGS
GKTLTIQVKEFGDAGQYTCHKGGEVLSHSLLLLHKKEDGIWSTDILKDQ
KEPKNKTFLRCEAKNYSGRFTCWWLTTISTDLTFSVKSSRGSSDPQGVT
CGAATLSAERVRGDNKEYEYSVECQEDSACPAAEESLPIEVMVDAVHKL
KYENYTSSFFIRDIIKPDPPKNLQLKPLKNSRQVEVSWEYPDTWSTPHS
YFSLTFSVQVQGKSKREKKDRVFTDKTSATVICRKNASISVRAQDRYYS
SSWSEWASVPCSGGGGSGGGGSGGGGSRNLPVATPDPGMFPCLHHSQNL
LRAVSNMLQKARQTLEFYPCTSEEIDHEDITKDKTSTVEACLPLELTKN
ESCLNSRETSFITNGSCLASRKTSFMMALCLSSIYEDLKMYQVEFKTMN
AKLLMDPKRQIFLDQNMLAVIDELMQALNFNSETVPQKSSLEEPDFYKT
KIKLCILLHAFRIRAVTIDRVMSYLNAS
(SEQ ID NO: 154)
IWELKKDVYVVELDWYPDAPGEMVVLTCDTPEEDGITWTLDQSSEVLGS
GKTLTIQVKEFGDAGQYTCHKGGEVLSHSLLLLHKKEDGIWSTDILKDQ
KEPKNKTFLRCEAKNYSGRFTCWWLTTISTDLTFSVKSSRGSSDPQGVT
CGAATLSAERVRGDNKEYEYSVECQEDSACPAAEESLPIEVMVDAVHKL
KYENYTSSFFIRDIIKPDPPKNLQLKPLKNSRQVEVSWEYPDTWSTPHS
YFSLTFSVQVQGKDNTEGRVFTDKTSATVICRKNASISVRAQDRYYSSS
WSEWASVPCSGGGGSGGGGSGGGGSRNLPVATPDPGMFPCLHHSQNLLR
AVSNMLQKARQTLEFYPCTSEEIDHEDITKDKTSTVEACLPLELTKNES
CLNSRETSFITNGSCLASRKTSFMMALCLSSIYEDLKMYQVEFKTMNAK
LLMDPKRQIFLDQNMLAVIDELMQALNFNSETVPQKSSLEEPDFYKTKI
KLCILLHAFRIRAVTIDRVMSYLNAS

In some embodiments, the IL-12 cytokine or functional fragment thereof comprises SEQ ID NO: 150. In some embodiments, the IL-12 cytokine or functional fragment thereof comprises SEQ ID NO: 151. In some embodiments, the IL-12 cytokine or functional fragment thereof comprises SEQ ID NO: 152. In some embodiments, the IL-12 cytokine or functional fragment thereof comprises SEQ ID NO: 153. In some embodiments, the IL-12 cytokine or functional fragment thereof comprises SEQ ID NO: 154.

In some embodiments the first linker or the second linker is a cleavable linker such that the masking moiety releases the cytokine or a fragment thereof upon cleavage.

In some embodiments, the cleavable linker comprises MPYDLYHP. In some embodiments, the cleavable linker comprises VPLSLYSG (SEQ ID NO: 42). In some embodiments, the cleavable linker comprises MPYDLYHP (SEQ ID NO: 34) and VPLSLYSG (SEQ ID NO: 42). In some embodiments, the cleavable linker comprises DSGGFMLT (SEQ ID NO: 35). In some embodiments, the cleavable linker comprises HEQLTV (SEQ ID NO: 36). In some embodiments, the cleavable linker comprises RAAAVKSP (SEQ ID NO: 37). In some embodiments, the cleavable linker comprises VPLSLY (SEQ ID NO: 38). In some embodiments, the cleavable linker comprises DLLAVVAAS (SEQ ID NO: 39). In some embodiments, the cleavable linker comprises ISSGLLSGRS (SEQ ID NO: 40).

In some embodiments, the cleavable linker comprises between 6-60 amino acid residues. In some embodiments, the cleavable linker comprises between 8-50 amino acid residues. In some embodiments, the cleavable linker comprises between 8-30 amino acid residues. In some embodiments, the cleavable linker comprises between 8-25 amino acid residues. In some embodiments, the cleavable linker comprises 8 amino acid residues. In some embodiments, the cleavable linker comprises 9 amino acid residues. In some embodiments, the cleavable linker comprises 10 amino acid residues. In some embodiments, the cleavable linker comprises 11 amino acid residues. In some embodiments, the cleavable linker comprises 12 amino acid residues. In some embodiments, the cleavable linker comprises 13 amino acid residues. In some embodiments, the cleavable linker comprises 14 amino acid residues. In some embodiments, the cleavable linker comprises 15 amino acid residues. In some embodiments, the cleavable linker comprises 16 amino acid residues. In some embodiments, the cleavable linker comprises 17 amino acid residues. In some embodiments, the cleavable linker comprises 18 amino acid residues. In some embodiments, the cleavable linker comprises 19 amino acid residues. In some embodiments, the cleavable linker comprises 20 amino acid residues. In some embodiments, the cleavable linker comprises 21 amino acid residues. In some embodiments, the cleavable linker comprises 22 amino acid residues. In some embodiments, the cleavable linker comprises 23 amino acid residues. In some embodiments, the cleavable linker comprises 24 amino acid residues. In some embodiments, the cleavable linker comprises 25 amino acid residues.

In some embodiments, the cleavable linker comprises a proteolytically cleavable peptide. In some embodiments, the cleavable linker comprises a proteolytically cleavable peptide flanked on the N-terminus by a spacer domain. In some embodiments, the cleavable linker comprises a proteolytically cleavable peptide flanked on the C-terminus by a spacer domain. In some embodiments, the cleavable linker comprises a proteolytically cleavable peptide flanked on both sides by a spacer domain. In some embodiments, the spacer domain is between 2 and 20 amino acids in length. In some embodiments, the spacer domain is between 3 and 10 amino acids in length. In some embodiments, the spacer domain is between 3 and 6 amino acids in length. In some embodiments, the spacer domain is 2 amino acids in length. In some embodiments, the spacer domain is 3 amino acids in length. In some embodiments, the spacer domain is 4 amino acids in length. In some embodiments, the spacer domain is 5 amino acids in length. In some embodiments, the spacer domain is 6 amino acids in length. In some embodiments, the spacer domain is 7 amino acids in length. In some embodiments, the spacer domain is 8 amino acids in length. In some embodiments, the spacer domain is 9 amino acids in length. In some embodiments, the spacer domain is 10 amino acids in length. In some embodiments, the spacer domain is 11 amino acids in length. In some embodiments, the spacer domain is 12 amino acids in length. In some embodiments, the spacer domain is 13 amino acids in length. In some embodiments, the spacer domain is 14 amino acids in length. In some embodiments, the spacer domain is 15 amino acids in length.

In some embodiments, the targeting moiety comprises at least one antigen binding domain. In some embodiments, the targeting moiety comprises a first antigen binding domain and a second antigen binding domain. In some embodiments, the targeting moiety comprises a first antigen binding domain, a second antigen binding domain, and a third antigen binding domain. In some embodiments, the targeting moiety comprises a first antigen binding domain, a second antigen binding domain, a third antigen binding domain, and a fourth antigen binding domain. In some embodiments the first and second antigen binding domains specifically bind to the same target. In some embodiments the first, second, and a third antigen binding domains specifically bind to the same target. In some embodiments the first, second, third, and fourth antigen binding domains specifically bind to the same target. In some embodiments the first and second antigen binding domains specifically bind to different targets. In some embodiments the first, second, and third antigen binding domains each specifically bind to different targets. In some embodiments the first, second, third, and fourth antigen binding domains each specifically bind to different targets.

In some embodiments, the first and second antigen binding domains comprise different amino acid sequences. In some embodiments, the first and second antigen binding domains comprise same amino acid sequences. In some embodiments, the first, second, and third antigen binding domains comprise different amino acid sequences. In some embodiments, the first, second, and third antigen binding domains comprise same amino acid sequences. In some embodiments, the first, second, third, and fourth, antigen binding domains comprise different amino acid sequences. In some embodiments, the first, second, third, and fourth antigen binding domains comprise same amino acid sequences.

In some embodiments, the first Fc polypeptide comprises a first IgG1 Fc domain or a fragment thereof. In some embodiments, the first Fc polypeptide comprises a first IgG2 Fc domain or a fragment thereof. In some embodiments, the first Fc polypeptide comprises a first IgG3 Fc domain or a fragment thereof. In some embodiments, the first Fc polypeptide comprises a first IgG4 Fc domain or a fragment thereof. In some embodiments, the second Fc polypeptide comprises a second IgG1 Fc domain or a fragment thereof. In some embodiments, the second Fc polypeptide comprises a second IgG2 Fc domain or a fragment thereof. In some embodiments, the second Fc polypeptide comprises a second IgG3 Fc domain or a fragment thereof. In some embodiments, the second Fc polypeptide comprises a second IgG4 Fc domain or a fragment thereof.

In some embodiments, the first Fc domain contain one or more modifications that promote the non-covalent association of the first and the second Fc polypeptide. In some embodiments, the second Fc domain contain one or more modifications that promote the non-covalent association of the first and the second Fc polypeptide. In some embodiments, the first and or the second Fc domains each contain one or more modifications that promote the non-covalent association of the first and the second Fc polypeptide. In some embodiments, the first and the second Fc domains each contain one or more modifications that promote the non-covalent association of the first and the second Fc polypeptide.

In some embodiments, the first Fc domain comprises Y349C mutation. In some embodiments, the first Fc domain comprises T366S mutation. In some embodiments, the first Fc domain comprises L368A mutation. In some embodiments, the first Fc domain comprises Y407V mutation. In some embodiments, the first Fc domain comprises N297A mutation. In some embodiments, the first Fc domain comprises S354C mutation. In some embodiments, the first Fc domain comprises T366W mutation. In some embodiments, the first Fc domain comprises I253A mutation. In some embodiments, the second Fc domain comprises Y349C mutation. In some embodiments, the second Fc domain comprises T366S mutation. In some embodiments, the second Fc domain comprises L368A mutation. In some embodiments, the second Fc domain comprises Y407V mutation. In some embodiments, the second Fc domain comprises N297A mutation. In some embodiments, the second Fc domain comprises S354C mutation. In some embodiments, the second Fc domain comprises T366W mutation. In some embodiments, the second Fc domain comprises I253A mutation.

In some embodiments, the first Fc domain comprises Y349C and T366S mutations. In some embodiments, the first Fc domain comprises Y349C, T366S, and L368A mutations. In some embodiments, the first Fc domain comprises Y349C and L368A mutations. In some embodiments, the first Fc domain comprises Y349C, T366S, L368A, and Y407V mutations. In some embodiments, the first Fc domain comprises Y349C, T366S, and Y407V mutations. In some embodiments, the first Fc domain comprises T366S, L368A, and Y407V mutations. In some embodiments, the first Fc domain comprises Y349C, L368A, and Y407V mutations. In some embodiments, the first Fc domain comprises Y349C, T366S, L368A, Y407V, and N297A mutations. In some embodiments, the first Fc domain comprises S354C and T366W mutations. In some embodiments, the first Fc domain comprises S354C and N297A mutations. In some embodiments, the first Fc domain comprises T366W and N297A mutations. In some embodiments, the first Fc domain comprises S354C, T366W and N297A mutations. In some embodiments, the second Fc domain comprises Y349C and T366S mutations. In some embodiments, the second Fc domain comprises Y349C, T366S, and L368A mutations. In some embodiments, the second Fc domain comprises Y349C and L368A mutations. In some embodiments, the second Fc domain comprises Y349C, T366S, L368A, and Y407V mutations. In some embodiments, the second Fc domain comprises Y349C, T366S, and Y407V mutations. In some embodiments, the second Fc domain comprises T366S, L368A, and Y407V mutations. In some embodiments, the second Fc domain comprises Y349C, L368A, and Y407V mutations. In some embodiments, the second Fc domain comprises Y349C, T366S, L368A, Y407V, and N297A mutations. In some embodiments, the second Fc domain comprises S354C and T366W mutations. In some embodiments, the second Fc domain comprises S354C and N297A mutations. In some embodiments, the second Fc domain comprises T366W and N297A mutations. In some embodiments, the second Fc domain comprises S354C, T366W and N297A mutations.

In some embodiments, the first Fc domain comprises Y349C, T366S, L368A, Y407V, and N297A mutations and the second Fc domain comprises S354C, T366W and N297A mutations. In some embodiments, the second Fc domain comprises Y349C, T366S, L368A, Y407V, and N297A mutations and the first Fc domain comprises S354C, T366W and N297A mutations.

In some embodiments, the first Fc domain comprises Y349C, T366S, L368A, Y407V, and I253A mutations and the second Fc domain comprises S354C, T366W and I253A mutations. In some embodiments, the second Fc domain comprises Y349C, T366S, L368A, Y407V, and I253A mutations and the first Fc domain comprises S354C, T366W and I253A mutations.

In some embodiments, the first Fc domain comprises Y349C, T366S, L368A, Y407V, N297A, and I253A mutations and the second Fc domain comprises S354C, T366W, N297A and I253A mutations. In some embodiments, the second Fc domain comprises Y349C, T366S, L368A, Y407V, N297A, I253A mutations and the first Fc domain comprises S354C, T366W, N297A and I253A mutations.

In some embodiments, the first Fc polypeptide comprises a first CH3 domain comprising a modification that reduces or eliminates binding to Protein A. In some embodiments, the second Fc polypeptide comprises a second CH3 domain comprising a modification that reduces or eliminates binding to Protein A. In some embodiments, the first Fc polypeptide comprises a first CH3 domain that binds to Protein A. In some embodiments, the second Fc polypeptide comprises a second CH3 domain that binds to Protein A.

In some embodiments, the first Fc polypeptide comprises a first CH3 domain comprising a modification that reduces or eliminates binding to Protein A and the second Fc polypeptide comprises a second CH3 domain that binds to Protein A. In some embodiments, the second Fc polypeptide comprises a second CH3 domain comprising a modification that reduces or eliminates binding to Protein A and the first Fc polypeptide comprises a first CH3 domain that binds to Protein A.

In some embodiments, the CH3 domain that binds to Protein A is a human IgG1 sequence. In some embodiments, the CH3 domain that binds to Protein A is a human IgG2 sequence. In some embodiments, the CH3 domain that binds to Protein A is a human IgG4 sequence.

In some embodiments, the first CH3 domain is a human IgG1 sequence comprising a modification at position H435 according to Kabat numbering. In some embodiments, the first CH3 domain is a human IgG1 sequence comprising a modification at position Y436 according to Kabat numbering. In some embodiments, the first CH3 domain is a human IgG1 sequence comprising modifications at positions H435 and Y436 according to Kabat numbering. In some embodiments, the first CH3 domain is a human IgG1 sequence comprising a H435R modification according to Kabat numbering. In some embodiments, the first CH3 domain is a human IgG1 sequence comprising an Y436F modification. In some embodiments, the first CH3 domain is a human IgG1 sequence comprising modifications H435R and Y436F according to Kabat numbering.

In some embodiments, the first CH3 domain is a human IgG2 sequence comprising a modification at position H435 according to Kabat numbering. In some embodiments, the first CH3 domain is a human IgG2 sequence comprising a modification at position Y436 according to Kabat numbering. In some embodiments, the first CH3 domain is a human IgG2 sequence comprising modifications at positions H435 and Y436 according to Kabat numbering. In some embodiments, the first CH3 domain is a human IgG2 sequence comprising a H435R modification according to Kabat numbering. In some embodiments, the first CH3 domain is a human IgG2 sequence comprising an Y436F modification. In some embodiments, the first CH3 domain is a human IgG2 sequence comprising modifications H435R and Y436F according to Kabat numbering.

In some embodiments, the first CH3 domain is a human IgG3 sequence comprising a modification at position H435 according to Kabat numbering. In some embodiments, the first CH3 domain is a human IgG3 sequence comprising a modification at position Y436 according to Kabat numbering. In some embodiments, the first CH3 domain is a human IgG3 sequence comprising modifications at positions H435 and Y436 according to Kabat numbering. In some embodiments, the first CH3 domain is a human IgG3 sequence comprising a H435R modification according to Kabat numbering. In some embodiments, the first CH3 domain is a human IgG3 sequence comprising an Y436F modification. In some embodiments, the first CH3 domain is a human IgG3 sequence comprising modifications H435R and Y436F according to Kabat numbering.

In some embodiments, the second CH3 domain is a human IgG1 sequence comprising a modification at position H435 according to Kabat numbering. In some embodiments, the second CH3 domain is a human IgG1 sequence comprising a modification at position Y436 according to Kabat numbering. In some embodiments, the second CH3 domain is a human IgG1 sequence comprising modifications at positions H435 and Y436 according to Kabat numbering. In some embodiments, the second CH3 domain is a human IgG1 sequence comprising a H435R modification according to Kabat numbering. In some embodiments, the second CH3 domain is a human IgG1 sequence comprising an Y436F modification. In some embodiments, the second CH3 domain is a human IgG1 sequence comprising modifications H435R and Y436F according to Kabat numbering.

In some embodiments, the second CH3 domain is a human IgG2 sequence comprising a modification at position H435 according to Kabat numbering. In some embodiments, the second CH3 domain is a human IgG2 sequence comprising a modification at position Y436 according to Kabat numbering. In some embodiments, the second CH3 domain is a human IgG2 sequence comprising modifications at positions H435 and Y436 according to Kabat numbering. In some embodiments, the second CH3 domain is a human IgG2 sequence comprising a H435R modification according to Kabat numbering. In some embodiments, the second CH3 domain is a human IgG2 sequence comprising an Y436F modification. In some embodiments, the second CH3 domain is a human IgG2 sequence comprising modifications H435R and Y436F according to Kabat numbering.

In some embodiments, the second CH3 domain is a human IgG3 sequence comprising a modification at position H435 according to Kabat numbering. In some embodiments, the second CH3 domain is a human IgG3 sequence comprising a modification at position Y436 according to Kabat numbering. In some embodiments, the second CH3 domain is a human IgG3 sequence comprising modifications at positions H435 and Y436 according to Kabat numbering. In some embodiments, the second CH3 domain is a human IgG3 sequence comprising a H435R modification according to Kabat numbering. In some embodiments, the second CH3 domain is a human IgG3 sequence comprising an Y436F modification. In some embodiments, the second CH3 domain is a human IgG3 sequence comprising modifications H435R and Y436F according to Kabat numbering.

In some embodiments, the first Fc domain comprises SEQ ID NO: 85. In some embodiments, the first Fc domain comprises SEQ ID NO: 80. In some embodiments, the second Fc domain comprises SEQ ID NO: 85. In some embodiments, the second Fc domain comprises SEQ ID NO: 80. In some embodiments, the first Fc domain comprises SEQ ID NO: 85 and the second Fc domain comprises SEQ ID NO: 80. In some embodiments, the second Fc domain comprises SEQ ID NO: 85 and the first Fc domain comprises SEQ ID NO: 80.

In some embodiments, the first CH3 domain comprises a human IgG3 sequence. In some embodiments, the second CH3 domain comprises a human IgG3 sequence.

In some embodiments, the first Fc domain comprises SEQ ID NO: 84. In some embodiments, the first Fc domain comprises SEQ ID NO: 80. In some embodiments, the second Fc domain comprises SEQ ID NO: 84. In some embodiments, the second Fc domain comprises SEQ ID NO: 80. In some embodiments, the first Fc domain comprises SEQ ID NO: 84 and the second Fc domain comprises SEQ ID NO: 80. In some embodiments, the second Fc domain comprises SEQ ID NO: 84 and the first Fc domain comprises SEQ ID NO: 80.

In some embodiments, the first antigen binding domain comprises VH, and the second antigen binding domain comprises a VL. In some embodiments, the first antigen binding domain comprises three HCDRs and the second antigen binding domain comprises three LCDRs.

In some embodiments, the targeting moiety is linked to the Fc domain through one or both of the first and second Fc polypeptide. In some embodiments, the targeting moiety is linked to the Fc domain through the first Fc polypeptide. In some embodiments, the targeting moiety is linked to the Fc domain through the second Fc polypeptide. In some embodiments, the targeting moiety is linked to the Fc domain through both the first and the second Fc polypeptide. In some embodiments, the targeting moiety is linked to N-terminus of the Fc domain through one or both of the first and second Fc polypeptide. In some embodiments, the targeting moiety is linked to N-terminus of the Fc domain through the first Fc polypeptide. In some embodiments, the targeting moiety is linked to N-terminus of the Fc domain through the second Fc polypeptide. In some embodiments, the targeting moiety is linked to N-terminus of the Fc domain through both of the first and the second Fc polypeptide. In some embodiments, the targeting moiety is linked to C-terminus of the Fc domain through one or both of the first and second Fc polypeptide. In some embodiments, the targeting moiety is linked to C-terminus of the Fc domain through the first Fc polypeptide. In some embodiments, the targeting moiety is linked to C-terminus of the Fc domain through the second Fc polypeptide. In some embodiments, the targeting moiety is linked to C-terminus of the Fc domain through both of the first and the second Fc polypeptide.

In some embodiments, the targeting moiety comprises a variable heavy region (VH) and is linked to the Fc domain through one or both of the first and second Fc polypeptide. In some embodiments, the targeting moiety comprises a variable heavy region (VH) and is linked to the Fc domain through the first Fc polypeptide. In some embodiments, the targeting moiety comprises a variable heavy region (VH) and is linked to the Fc domain through the second Fc polypeptide. In some embodiments, the targeting moiety comprises a variable heavy region (VH) and is linked to the Fc domain through both of the first and the second Fc polypeptide. In some embodiments, the targeting moiety comprises a variable heavy region (VH) and is linked to N-terminus of the Fc domain through one or both of the first and second Fc polypeptide. In some embodiments, the targeting moiety comprises a variable heavy region (VH) and is linked to N-terminus of the Fc domain through the first Fc polypeptide. In some embodiments, the targeting moiety comprises a variable heavy region (VH) and is linked to N-terminus of the Fc domain through the second Fc polypeptide. In some embodiments, the targeting moiety comprises a variable heavy region (VH) and is linked to N-terminus of the Fc domain through both of the first and the second Fc polypeptide. In some embodiments, the targeting moiety comprises a variable heavy region (VH) and is linked to C-terminus of the Fc domain through one or both of the first and second Fc polypeptide. In some embodiments, the targeting moiety comprises a variable heavy region (VH) and is linked to C-terminus of the Fc domain through the first Fc polypeptide. In some embodiments, the targeting moiety comprises a variable heavy region (VH) and is linked to C-terminus of the Fc domain through the second Fc polypeptide. In some embodiments, the targeting moiety comprises a variable heavy region (VH) and is linked to C-terminus of the Fc domain through both of the first and the second Fc polypeptide.

In some embodiments, the targeting moiety comprises a Fab. In some embodiments, the targeting moiety comprises a Fab having HCDR1 of SEQ ID NO: 4 (GYTFTNYY), HCDR2 of SEQ ID NO: 5 (INPSNGGT), HCDR3 SEQ ID NO: 6 (ARRDYRFDMGFDY), LCDR1 of SEQ ID NO: 9 (KGVSTSGYSY), LCDR2 of SEQ ID NO: 10 (LAS), LCDR3 of SEQ ID NO: 11 (QHSRDLPLT).

In some embodiment, the targeting moiety comprises a variable heavy chain (VH) of SEQ ID NO: 1 and a variable light chain (VL) of SEQ ID NO: 7. In some embodiments, the targeting moiety comprises a heavy chain of SEQ ID NO: 2 and a light chain of SEQ ID NO: 8.

In some embodiments, a cytokine is linked to a first Fc polypeptide through a non-cleavable linker. In some embodiments, the N-terminus of a cytokine is linked to the C-terminus of a first Fc polypeptide through a non-cleavable linker. In some embodiments, the C-terminus of a cytokine is linked to the N-terminus of a first Fc polypeptide through a non-cleavable linker. In some embodiments, a cytokine is linked to a first Fc polypeptide through a cleavable linker. In some embodiments, the N-terminus of a cytokine is linked to the C-terminus of a first Fc polypeptide through a cleavable linker. In some embodiments, the C-terminus of a cytokine is linked to the N-terminus of a first Fc polypeptide through a cleavable linker.

In some embodiments, a masking moiety is linked to a second Fc polypeptide through a non-cleavable linker. In some embodiments, the N-terminus a masking moiety is linked to the C-terminus of a second Fc polypeptide through a non-cleavable linker. In some embodiments, the C-terminus a masking moiety is linked to the N-terminus of a second Fc polypeptide through a non-cleavable linker. In some embodiments, a masking moiety is linked to a second Fc polypeptide through a cleavable linker. In some embodiments, the N-terminus a masking moiety is linked to the C-terminus of a second Fc polypeptide through a cleavable linker. In some embodiments, the C-terminus a masking moiety is linked to the N-terminus of a second Fc polypeptide through a cleavable linker.

In some embodiments, the C-terminus of the first antigen binding domain is linked to the N-terminus of the first Fc polypeptide. In some embodiments, the C-terminus of the first Fc polypeptide is linked to the N-terminus of the cytokine or a fragment thereof. In some embodiments, the C-terminus of the second antigen binding domain is linked to the N-terminus of the second Fc polypeptide. In some embodiments, the C-terminus of the second Fc polypeptide is linked to the N-terminus of the masking moiety.

In some embodiments, a targeting moiety comprises an agent that specifically binds to a target. In some embodiments, a targeting moiety comprises a peptide that specifically binds to a target. In some embodiments, a targeting moiety comprises a polypeptide that specifically binds to a target. In some embodiments, a targeting moiety comprises a polypeptide that specifically binds to a target. In some embodiments, a targeting moiety comprises a protein that specifically binds to a target. In some embodiments, a targeting moiety comprises a nucleic acid that specifically binds to a target.

In some embodiments, a targeting moiety comprises a Fab, a single chain Fv (scFv), a single domain antibody (VHH), one or more CDRs, a variable heavy chain (VH), a variable light chain (VL), a Fab-like bispecific antibodies (bsFab), a single-domain antibody-linked Fab (s-Fab), an antibody, or a combination thereof. In some embodiments, a targeting moiety comprises a Fab. In some embodiments, a targeting moiety comprises a single chain Fv (scFv). In some embodiments, a targeting moiety comprises a single domain antibody (VHH). In some embodiments, a targeting moiety comprises one or more CDRs. In some embodiments, a targeting moiety comprises a variable heavy chain (VH). In some embodiments, a targeting moiety comprises a variable light chain (VL). In some embodiments, a targeting moiety comprises a Fab-like bispecific antibodies (bsFab). In some embodiments, a targeting moiety comprises a single-domain antibody-linked Fab (s-Fab). In some embodiments, a targeting moiety comprises an antibody or a fragment thereof.

In some embodiments, the targeting moiety comprises an antigen-binding moiety, wherein the antigen is expressed on an immune cell. In some embodiments, the targeting moiety specifically binds PD-1. In some embodiments, the targeting moiety specifically binds PD-L1. In some embodiments, the targeting moiety specifically binds PD-L2. In some embodiments, the targeting moiety specifically binds CTLA-4. In some embodiments, the targeting moiety specifically binds TIGIT. In some embodiments, the targeting moiety specifically binds TIM-3. In some embodiments, the targeting moiety specifically binds LAG-3. In some embodiments, the targeting moiety specifically binds CD25. In some embodiments, the targeting moiety specifically binds CD16a. In some embodiments, the targeting moiety specifically binds CD16b. In some embodiments, the targeting moiety specifically binds NKG2D. In some embodiments, the targeting moiety specifically binds NKP44. In some embodiments, the targeting moiety specifically binds NKP3. In some embodiments, the targeting moiety specifically binds CD19. In some embodiments, the targeting moiety specifically binds CD20. In some embodiments, the targeting moiety specifically binds CD30. In some embodiments, the targeting moiety specifically binds CD38. In some embodiments, the targeting moiety specifically binds BMCA. In some embodiments, the targeting moiety specifically binds HER2. In some embodiments, the targeting moiety specifically binds HER3. In some embodiments, the targeting moiety specifically binds DLL3. In some embodiments, the targeting moiety specifically binds DLL4. In some embodiments, the targeting moiety specifically binds EGFR. In some embodiments, the targeting moiety specifically binds GPC3. In some embodiments, the targeting moiety specifically binds c-MET. In some embodiments, the targeting moiety specifically binds VEGF-R1. In some embodiments, the targeting moiety specifically binds VEGF-R2. In some embodiments, the targeting moiety specifically binds OX40. In some embodiments, the targeting moiety specifically binds DRS. In some embodiments, the targeting moiety specifically binds ICOS. In some embodiments, the targeting moiety specifically binds GITR. In some embodiments, the targeting moiety specifically binds CD73. In some embodiments, the targeting moiety specifically binds CD39. In some embodiments, the targeting moiety specifically binds CD25. In some embodiments, the targeting moiety specifically binds CD16a. In some embodiments, the targeting moiety specifically binds CD8. In some embodiments, the targeting moiety specifically binds KLRC1. In some embodiments, the targeting moiety specifically binds KLRD1. In some embodiments, the targeting moiety specifically binds KLRB1. In some embodiments, the targeting moiety specifically binds CD40. In some embodiments, the targeting moiety specifically binds CD137. In some embodiments, the targeting moiety specifically binds CD28.

In some embodiments, the cleavable linker has an in vitro cleavage efficiency yielding about 1-99% active cytokine. In some embodiments, the cleavable linker has an in vitro cleavage efficiency yielding about 5-80% active cytokine. In some embodiments, the cleavable linker has an in vitro cleavage efficiency yielding about 8-60% active cytokine. In some embodiments, the cleavable linker has an in vitro cleavage efficiency yielding about 10-40% active cytokine. In some embodiments, the cleavable linker has an in vitro cleavage efficiency yielding about 10-20% active cytokine. In some embodiments, the cleavable linker has an in vitro cleavage efficiency yielding at least 3% active cytokine. In some embodiments, the cleavable linker has an in vitro cleavage efficiency yielding at least 5% active cytokine. In some embodiments, the cleavable linker has an in vitro cleavage efficiency yielding at least 8% active cytokine. In some embodiments, the cleavable linker has an in vitro cleavage efficiency yielding at least 10% active cytokine. In some embodiments, the cleavable linker has an in vitro cleavage efficiency yielding at least 12% active cytokine. In some embodiments, the cleavable linker has an in vitro cleavage efficiency yielding at least 15% active cytokine. In some embodiments, the cleavable linker has an in vitro cleavage efficiency yielding at least 20% active cytokine. In some embodiments, the cleavable linker has an in vitro cleavage efficiency yielding at least 25% active cytokine. In some embodiments, the cleavable linker has an in vitro cleavage efficiency yielding at least 30% active cytokine. In some embodiments, the cleavable linker has an in vitro cleavage efficiency yielding at least 35% active cytokine. In some embodiments, the cleavable linker has an in vitro cleavage efficiency yielding at least 40% active cytokine. In some embodiments, the cleavable linker has an in vitro cleavage efficiency yielding at least 45% active cytokine. In some embodiments, the cleavable linker has an in vitro cleavage efficiency yielding at least 50% active cytokine. In some embodiments, the cleavable linker has an in vitro cleavage efficiency yielding at least 60% active cytokine. In some embodiments, the cleavable linker has an in vitro cleavage efficiency yielding at least 70% active cytokine. In some embodiments, the cleavable linker has an in vitro cleavage efficiency yielding at least 80% active cytokine.

In one aspect, the present invention provides, among other things, a targeted cytokine comprising a targeting moiety, a cytokine or a fragment thereof, a masking moiety, and an Fc domain comprising a first Fc polypeptide linked to the cytokine or a fragment thereof through a first linker and a second Fc polypeptide linked to the masking moiety through a second linker, wherein the masking moiety binds to the cytokine or a fragment thereof, wherein the first or the second linker is a cleavable linker such that the masking moiety releases the cytokine or a fragment thereof upon cleavage, wherein the cleavable linker comprises MPYDLYHP (SEQ ID NO: 34) or VPLSLYSG (SEQ ID NO: 42) and wherein the targeting moiety is linked to the Fc domain through one or both of the first and second Fc polypeptides.

In one aspect, the present invention provides, among other things, a targeted cytokine comprising a targeting moiety, a cytokine or a fragment thereof, a masking moiety, and an Fc domain comprising a first Fc polypeptide linked to the cytokine or a fragment thereof through a first linker and a second Fc polypeptide linked to the masking moiety through a second linker, wherein the masking moiety binds to the cytokine or a fragment thereof, wherein the first or the second linker is a tumor specific cleavable linker such that the masking moiety releases the cytokine or a fragment thereof upon cleavage by a tumor specific protease, wherein the tumor specific cleavable linker comprises between 8-50 amino acid residues, and wherein the targeting moiety is linked to the Fc domain through one or both of the first and second Fc polypeptides.

In one aspect, the present invention provides, among other things, a targeted cytokine comprising a targeting moiety, a cytokine or a fragment thereof, a masking moiety, and an Fc domain comprising a first Fc polypeptide linked to the cytokine or a fragment thereof through a first linker and a second Fc polypeptide linked to the masking moiety through a second linker, wherein the masking moiety binds to the cytokine or a fragment thereof, wherein the first or the second linker is a cleavable linker such that the masking moiety releases the cytokine or a fragment thereof upon cleavage to yield active cytokine, wherein the cleavable linker has an in vitro cleavage efficiency yielding at least 10% active cytokine and wherein the targeting moiety is linked to the Fc domain through one or both of the first and second Fc polypeptides.

In one aspect, the present invention provides, among other things, a targeted cytokine comprising a targeting moiety, a cytokine or a fragment thereof, a masking moiety comprising CD122 or a fragment thereof comprising one or more mutations selected from the group consisting of F8C, A94C, L106C, C122S, C122V, C122A, N123C, N123Q, C168V, C168A, C168S, L169C, Q177C, V184C, S195C, R204C, and an Fc domain comprising a first Fc polypeptide linked to the cytokine or a fragment thereof through a first linker and a second Fc polypeptide linked to the masking moiety through a second linker, wherein the masking moiety binds to the cytokine or a fragment thereof, wherein the first or the second linker is a cleavable linker such that the masking moiety releases the cytokine or a fragment thereof upon cleavage, and wherein the targeting moiety is linked to the Fc domain through one or both of the first and second Fc polypeptides.

In one aspect, the present invention provides, among other things, a targeted cytokine comprising a targeting moiety, a masking moiety, a cytokine or fragment thereof, and a Fc domain comprising a first Fc polypeptide comprising a CH3 domain comprising a modification that reduces or eliminates binding to Protein A, and a second Fc polypeptide comprising a CH3 domain that binds to Protein A.

In one aspect, the present invention provides, among other things, a targeted cytokine comprising a targeting moiety, a cytokine or a fragment thereof, a masking moiety, and an Fc domain comprising a first Fc polypeptide linked to the cytokine or a fragment thereof through a first linker and a second Fc polypeptide linked to the masking moiety through a second linker, wherein the masking moiety binds to the cytokine or a fragment thereof, wherein the first or the second linker is a cleavable linker such that the masking moiety releases the cytokine or a fragment thereof upon cleavage, wherein the cleavable linker comprises MPYDLYHP (SEQ ID NO: 34) or VPLSLYSG (SEQ ID NO: 42) and wherein the targeting moiety is linked to the Fc domain through one or both of the first and second Fc polypeptides.

In one aspect, the present invention provides, among other things, a targeted cytokine comprising a targeting moiety that specifically binds to PD-1 comprising HCDR1 of SEQ ID NO: 4 (GYTFTNYY), HCDR2 of SEQ ID NO: 5 (INPSNGGT), HCDR3 SEQ ID NO: 6 (ARRDYRFDMGFDY), LCDR1 of SEQ ID NO: 9 (KGVSTSGYSY), LCDR2 of SEQ ID NO: 10 (LAS), LCDR3 of SEQ ID NO: 11 (QHSRDLPLT), a cytokine or fragment thereof, and an Fc domain.

In one aspect, the present invention provides, among other things, a targeted IL-2 cytokine comprising SEQ ID NO: 87.

In some embodiments, the targeted IL-2 cytokine comprises SEQ ID NO: 88.

In some embodiments, the targeted IL-2 cytokine comprises SEQ ID NO: 89.

In some embodiments, the targeted IL-2 cytokine comprises SEQ ID NO: 90.

In some embodiments, the targeted IL-2 cytokine comprises SEQ ID NO: 91.

In one aspect, the present invention provides, among other things, a targeted IL-2 cytokine comprising SEQ ID NO: 87 and SEQ ID NO: 88, and a light chain comprising SEQ ID NO: 8.

In one aspect, the present invention provides, among other things, a targeted IL-2 cytokine comprising SEQ ID NO: 87 and SEQ ID NO: 89, and a light chain comprising SEQ ID NO: 8.

In one aspect, the present invention provides, among other things, a targeted IL-2 cytokine comprising SEQ ID NO: 87 and SEQ ID NO: 90, and a light chain comprising SEQ ID NO: 8.

In one aspect, the present invention provides, among other things, a targeted IL-2 cytokine comprising SEQ ID NO: 87 and SEQ ID NO: 91, and a light chain comprising SEQ ID NO: 8.

In one aspect, the present invention provides, among other things, a nucleic acid encoding a target cytokine of the present invention. In one aspect, the present invention provides, among other things, a vector comprising a nucleic acid encoding a target cytokine of the present invention. In one aspect, the present invention provides, among other things, a host cell comprising a nucleic acid encoding a target cytokine of the present invention.

In one aspect, the present invention provides, among other things, a method of producing a targeted cytokine comprising culturing the host cell comprising a nucleic acid encoding a target cytokine of the present invention under a condition that produces the targeted cytokine.

In one aspect, the present invention provides, among other things, a composition comprising the targeted cytokine of the present invention.

In one aspect, the present invention provides, among other things, a pharmaceutical composition comprising the targeted cytokine of the present invention and a pharmaceutically acceptable carrier.

In one aspect, the present invention provides, among other things, a kit comprising the targeted cytokine of the present invention.

In one aspect, the present invention provides, among other things, a method of treating or preventing a neoplastic disease in a subject, the method comprising administering to the subject an effective amount of the targeted cytokine of the present invention.

In one aspect, the present invention provides, among other things, a method of treating or preventing a neoplastic disease in a subject, the method comprising administering to the subject an effective amount of a composition comprising the targeted cytokine of the present invention.

In one aspect, the present invention provides, among other things, a method of treating or preventing a neoplastic disease in a subject, the method comprising administering to the subject an effective amount of a pharmaceutical composition comprising the targeted cytokine of the present invention.

In one aspect, the present invention provides, among other things, a method of treating or preventing an inflammatory or autoimmune disease in a subject, the method comprising administering to the subject an effective amount of the targeted cytokine of the present invention.

In one aspect, the present invention provides, among other things, a method of treating or preventing an inflammatory or autoimmune disease in a subject, the method comprising administering to the subject an effective amount of a composition comprising the targeted cytokine of the present invention.

In one aspect, the present invention provides, among other things, a method of treating or preventing an inflammatory or autoimmune disease in a subject, the method comprising administering to the subject an effective amount of a pharmaceutical composition comprising the targeted cytokine of the present invention.

### BRIEF DESCRIPTION OF THE DRAWINGS

The drawings are for illustration purposes only, not for limitation.

FIG. 1 illustrates exemplary schematics of the Fc domains used in the targeted IL-2 cytokines of the present invention. The first Fc polypeptide comprises the CH2 and CH3 domains derived from IgG1. The second chain of the “chimeric Fc” comprise a CH2 domain derived from the IgG1, and a CH3 domain derived from IgG3. The second chain of the “CH3 mutant” comprises CH2 and CH3 domains derived from IgG1 with “RF mutations” (H435R and Y436F) in the CH3 domain.

FIG. 2A is series of exemplary SDS-PAGE analysis of purified masked IL-2 cytokines comprising either the native Fc, CH3 mutant, or chimeric Fc domain. Each construct was purified by Protein A chromatography and size exclusion chromatography (SEC) at different ratios of the first and second Fc polypeptides. FIG. 2B is a series of exemplary EC50 graphs of purified masked IL-2 cytokines comprising either the native Fc, CH3 mutant, or chimeric Fc domain. EC50 values were determined in a cell-based reporter assay (HEK Blue IL-2 assay) which measures % calculated active cytokine.

FIG. 3 illustrates exemplary schematics of the targeted IL-2 cytokine (TC) molecules of the present invention, and their purities as determined by SEC, non-reducing capillary electrophoresis SDS (CE-SDS), and reducing CE-SDS). The TC molecules tested in this experiment all comprise bivalent Fabs that bind to PD-1, a first Fc polypeptide with “hole mutations” (Y349C; T366S; L368A; and Y407V), a second Fc polypeptide with “knob mutations” (S354C and T366W) fused to an IL-2 cytokine. TC1 is “unmasked,” without a masking moiety that binds to IL-2. TC2, TC3, and TC4 comprise CD122 as a masking moiety that is fused to the first Fc polypeptide. TC3 and TC4 have a cleavable linker comprising MPYDLYHP (SEQ ID NO: 34) and VPLSLYSG (SEQ ID NO: 42) cleavage peptides, respectively, between the first Fc polypeptide and the masking moiety.

FIG. 4A illustrates exemplary schematics of different targeted IL-2 cytokines of the present invention. The targeted cytokines can be in a bivalent targeting format (Format A), comprising a bivalent Fabs that bind to a specific antigen or in a monovalent targeting format (Formats B1, B2, and C), comprising a monovalent Fab. FIG. 4B is a series of exemplary EC50 graphs of purified targeted IL-2 cytokines shown in FIG. 4A. EC50 values were determined in a cell-based reporter assay (HEK Blue IL-2 assay) which measures % calculated active cytokine.

FIG. 5A is a series of exemplary binding graphs illustrating binding of PD-1 multifunctional constructs on Raji cells expressing PD-1 and CD8+ T cells within CD3/CD28 activated PBMCs. FIG. 5B is a series of exemplary graphs illustrating surface dwell time of PD-1 multifunctional constructs on CD8+ T cells within CD3/CD28 activated PBMCs at 37° C. and 4° C. FIG. 5C is an exemplary graph and a table showing proliferation of CD3 and CD28 primed human PBMCs in response to stimulation with the indicated constructs up to 1000 nM.

FIG. 6A is a series of exemplary graphs illustrating changes in tumor weight and tumor volume over time after treatment with targeted IL-2 cytokines of the present invention. FIG. 6B is a series of exemplary graphs illustrating changes in amount of CD8+ T cells and CD8 proliferation in tumor, spleen, and peripheral blood post treatment with the targeted IL-2 cytokines of the present invention. FIG. 6C is a series of exemplary graphs illustrating fold-change in CD8+ T cell expansion in tumor microenvironment compared to periphery post treatment with the targeted IL-2 cytokines. FIG. 6D is a series of exemplary graphs illustrating a change in the number of antigen-specific CD8+ T cell post treatment with targeted IL-2 cytokines. FIG. 6E is a series of exemplary graphs illustrating a potential increase of drug accumulation in tumor post treatment with targeted IL-2 cytokines. FIG. 6F is a series of exemplary graphs illustrating half-life, maximum plasma concentration, AUC, volume of distribution, and clearance data of targeted IL-2 cytokines post treatment.

FIG. 7A is an exemplary table illustrating binding affinity data and statistics of the targeted IL-2 cytokines shown in FIG. 3. Pembrolizumab (anti-PD-1 antibody; “pembro”) and anti-human PD-L1-hFc fusion were used as controls. FIG. 7B is a series of exemplary EC50 graphs of purified targeted IL-2 cytokines as performed in a cell-based reporter assay (HEK Blue IL-2 assay) which determines % calculated active cytokine. Each graph shows the binding curves the absence and in the presence of various matrix metalloproteases (MMPs). Recombinant human IL-2 (rhIL-2) and TC0 (unmasked, Fc domain fused to IL-2) were used as a positive control. FIG. 7C is an exemplary graph and a table showing proliferation of CD3 and CD28 primed human PBMCs in response to stimulation with the indicated constructs up to 3000 nM. FIG. 7D is an exemplary graph and a table illustrating STAT5 phosphorylation on resting PBMCs using uncleaved targeted cytokines. FIG. 7E is an exemplary graph and a table illustrating STAT5 phosphorylation on resting PBMCs using cleaved targeted cytokines.

FIG. 8A is a series of bar graphs illustrating cleavage of the targeted IL-2 cytokines in tumor in vivo. FIG. 8B illustrates that cleavage of the targeted IL-2 cytokines in tumor as compared to in plasma, demonstrating tumor-specific cleavage in vivo.

FIG. 9A is an exemplary graph showing tumor volume post treatment with the targeted IL-2 cytokines of the present invention. Pembrolizumab (anti-PD-1 antibody; “pembro”) was used as a negative control. The table illustrates percentage of tumor growth inhibition for each targeted cytokine. FIG. 9B is a series of exemplary graphs illustrating percentage of CD8+ T cells in peripheral blood and splenic cells as compared to tumor cells, illustrating that the targeted cytokines of the present invention increased the percentage of CD8+ T cells in the tumor microenvironment. FIG. 9C illustrates the % of CD8+ T cells post treatment with targeted IL-2 cytokines. FIG. 9D is a series of exemplary graphs illustrating body weight % change and pulmonary edema post treatment with targeted cytokines, demonstrating minimal toxicity caused by the targeted IL-2 cytokines of the present invention.

FIG. 10A is an exemplary table illustrating the engineered cleavable linker sequences incorporated in the targeted cytokines of the present invention and the yield and purity post purification with Protein A chromatography and size exclusion chromatography (SEC). FIG. 10B is a series of exemplary tables illustrating cleavage efficiency of the targeted molecules with various engineered cleavable linker sequences by different matrix metalloproteases (MMPs). FIG. 10C is an exemplary graph illustrating frequency of cleavage of targeted cytokines by secreted proteases from human tumor tissue conditioned media. FIG. 10D is an exemplary graph illustrating mean percentage of cleaved targeted cytokines in human plasma from lung tumor patients (n=8), as determined by Western Blot.

FIG. 11A is an exemplary graph and a table illustrating EC50 values as determined in a cell-based reporter assay (HEK Blue IL-2 assay) which measures % calculated active cytokine of targeted cytokines comprising MPY sequence as cleavable substrate. FIG. 11B is an exemplary graph and a table illustrating EC50 values as determined in a cell-based reporter assay (HEK Blue IL-2 assay) which measures % calculated active cytokine of targeted cytokines comprising VPL sequence as cleavable substrate. FIG. 11C is an exemplary graph and a table illustrating EC50 values of targeted cytokines comprising MPY sequence as cleavable substrate after full cleavage with MMP protease. FIG. 11D is an exemplary graph and a table illustrating EC50 values of targeted cytokines comprising VPL sequence as cleavable substrate after full cleavage with MMP protease. FIG. 11E is an exemplary graph and a table illustrating EC50 values of targeted cytokines comprising MPY sequence as cleavable substrate after minimal cleavage with MMP protease. FIG. 11F is a series of exemplary spider plots illustrating cleavage efficiency of various targeted cytokines with different MMP proteases.

FIG. 12 is an exemplary graph illustrating binding of PD-1 targeting cytokines on CD8+ T cells within CD3 activated PBMCs by frequency anti-hIgG+.

FIG. 13A is a series of exemplary graphs illustrating mean tumor volume and mean body weight change curves of unmasked PD-1 targeted TC29 versus Pembrolizumab. FIG. 13B is a series of exemplary graphs illustrating mean tumor volume and mean body weight change curves of PD-1 targeted TC3 versus Pembrolizumab.

FIG. 14A is a series of exemplary graphs illustrating mean tumor volume and mean body weight change curves of various targeted cytokines and pembrolizumab. FIG. 14B is a series of exemplary graphs illustrating amount of peripheral, splenic tumor infiltrating CD8+ T cells. FIG. 14C is an exemplary graph illustrating amount of tumor antigen specific CD8 T cells post administration with targeted cytokines of the present invention. FIG. 14D is a series of exemplary graphs illustrating construct concentration in plasma and in tumor lysates.

FIG. 15 is an exemplary graph illustrating results of an RGA assay of various targeted masked cytokines prior to protease cleavage.

FIG. 16A-16D is a series of exemplary graphs illustrating RGA assay results of various targeted masked cytokines cleaved with MMP1 (FIG. 14A), MMP7 (FIG. 16B), MMP9 (FIG. 16C) and MMP10 (FIG. 16D).

FIG. 17 is an exemplary graph demonstrating the average cleavage efficiency of each cleavage site based on kinetic analysis for various constructs.

FIG. 18A-18F shows exemplary ELISA results demonstrating binding affinity of various constructs to PD-1. Exemplary constructs tested comprise pembrolizumab scFv in VHVL format (FIG. 18A), pembrolizumab scFv in VLVH format (FIG. 18B), nivolumab (nivo) scFv (FIG. 18C), pembrolizumab Fab (FIG. 18D) or different masking moieties (FIG. 18F). FIG. 18E shows exemplary Kd values for each construct.

FIG. 19A-19F show exemplary results of PD1/PD-L1 checkpoint blockade (fold induction) demonstrated by PD-1 RGA assay. Exemplary constructs tested comprise pembrolizumab scFv in VHVL format (FIG. 19A), pembrolizumab scFv in VLVH format (FIG. 19B), nivolumab (nivo) scFv (FIG. 19C), pembrolizumab Fab (FIG. 19D) or different masking moieties (FIG. 19F). FIG. 19E shows exemplary Kd values for each construct.

FIG. 20A-20E show exemplary results of on-cell binding of various constructs to PD1 expressing Raji cells. Exemplary constructs tested comprise pembrolizumab Fab (FIG. 20A) pembrolizumab scFv in VHVL format (FIG. 20B), pembrolizumab scFv in VLVH format (FIG. 2C), or nivolumab (nivo) scFv (FIG. 20D). FIG. 20E shows exemplary EC50 for each construct.

FIG. 21A-21D show exemplary results of peripheral T cell expansion (FIG. 21A), peripheral T cell activation (FIG. 21B), peripheral NK cell expansion (FIG. 21C) and peripheral NK cell activation (FIG. 21D) upon administration of various targeted cytokine constructs.

FIG. 22A-22B show exemplary tumor growth curves of subcutaneous MC38 tumors in human hPD1 transgenic mice (FIG. 22A) and overall survival (FIG. 22B) following treatment with exemplary masked cytokines.

FIG. 23A-23F show exemplary tumor growth curves of subcutaneous MC38 tumors in human hPD1 transgenic mice (FIG. 23A, FIG. 23D), overall survival (FIG. 23B, FIG. 23E) and body weight change (FIG. 23C, FIG. 23E) following treatment with exemplary masked cytokines at a dose of administered 8 mg/kg (FIG. 23A-23C) or 2 mg/kg (FIG. 23D-23F) of each construct.

FIG. 24A-24E show exemplary tumor growth curves of subcutaneous MC38 tumors in human hPD1 transgenic mice (FIG. 24A), overall survival (FIG. 24B) and body weight change (FIG. 24C) following treatment with exemplary masked cytokines. Schematics of the exemplary tested constructs are shown in FIG. 24D and FIG. 24E.

FIG. 25A shows exemplary results of construct exposure in plasma over time following administration to NHPs. FIG. 25B shows changes in lymphocytes following administration of exemplary constructs to NHPs.

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

It is to be understood that this invention is not limited to particular compositions or biological systems, which can, of course, vary. It is also to be understood that the terminology used herein is for the purpose of describing particular embodiments only, and is not intended to be limiting.

As used in this specification and the appended claims, the singular forms “a,” “an,” and “the” include plural referents unless the content clearly dictates otherwise. Thus, for example, reference to “an IL-2 polypeptide” optionally includes a combination of two or more such polypeptides, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

As used herein, the term “and/or” refers to any one of the items, any combination of the items, or all of the items with which the term is associated. For instance, the phrase “A, B, and/or C” is intended to encompass each of the following embodiments: A, B, and C; A, B, or C; A or B; A or C; B or C; A and B; A and C; B and C; A and B or C; B and A or C; C and A or B; A (alone); B (alone); and C (alone).

The term “antibody” includes polyclonal antibodies, monoclonal antibodies (including full length antibodies which have an immunoglobulin Fc region), antibody compositions with polyepitopic specificity, multispecific antibodies (e.g., bispecific antibodies, diabodies, and single-chain molecules, as well as antibody fragments (e.g., Fab, F(ab′)2, and Fv). The term “immunoglobulin” (Ig) is used interchangeably with “antibody” herein.

The term “diabodies” refers to small antibody fragments with two antigen-binding sites, which comprise a heavy chain variable (VH) domain connected to a light chain variable (VL) domain in the same polypeptide chain (VH-VL).

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the a and y chains and four CH domains for p and s isotypes. Each L chain has at the N-terminus, a variable domain (VL) followed by a constant domain at its other end. The VL is aligned with the VH, and the CL is aligned with the first constant domain of the heavy chain (CHI). Particular amino acid residues are believed to form an interface between the light chain and heavy chain variable domains. The pairing of a VH and VL together forms a single antigen-binding site. For the structure and properties of the different classes of antibodies, see e.g., Basic and Clinical Immunology, 8th Edition, Daniel P. Stites, Abba I. Ten and Tristram G. Parslow (eds), Appleton & Lange, Norwalk, C T, 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated a, 8, e, y and p, respectively. They and a classes are further divided into subclasses on the basis of relatively minor differences in the CH sequence and function, e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable for use in the invention. Common allotypic variants in human populations are those designated by the letters a,f,n,z.

An “isolated” antibody is one that has been identified, separated and/or recovered from a component of its production environment (e.g., naturally or recombinantly). In some embodiments, the isolated polypeptide is free of association with all other components from its production environment. Contaminant components of its production environment, such as that resulting from recombinant transfected cells, are materials that would typically interfere with research, diagnostic or therapeutic uses for the antibody, and may include enzymes, hormones, and other proteinaceous or non-proteinaceous solutes. In some embodiments, the polypeptide is purified: (1) to greater than 95% by weight of antibody as determined by, for example, the Lowry method, and in some embodiments, to greater than 99% by weight; (1) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or silver stain. Isolated antibody includes the antibody in situ within recombinant cells since at least one component of the antibody's natural environment will not be present. Ordinarily, however, an isolated polypeptide or antibody is prepared by at least one purification step.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. In some embodiments, monoclonal antibodies have a C-terminal cleavage at the heavy chain and/or light chain. For example, 1, 2, 3, 4, or 5 amino acid residues are cleaved at the C-terminus of heavy chain and/or light chain. In some embodiments, the C-terminal cleavage removes a C-terminal lysine from the heavy chain. In some embodiments, monoclonal antibodies have an N-terminal cleavage at the heavy chain and/or light chain. For example, 1, 2, 3, 4, or 5 amino acid residues are cleaved at the N-terminus of heavy chain and/or light chain. In some embodiments truncated forms of monoclonal antibodies can be made by recombinant techniques. In some embodiments, monoclonal antibodies are highly specific, being directed against a single antigenic site. In some embodiments, monoclonal antibodies are highly specific, being directed against multiple antigenic sites (such as a bispecific antibody or a multispecific antibody). The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method, recombinant DNA methods, phage-display technologies, and technologies for producing human or human-like antibodies in animals that have parts or all of the human immunoglobulin loci or genes encoding human immunoglobulin sequences.

The terms “full-length antibody,” “intact antibody” or “whole antibody” are used interchangeably to refer to an antibody in its substantially intact form, as opposed to an antibody fragment. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, such as the antigen binding region and/or the variable region of the intact antibody, and/or the constant region of the intact antibody. Examples of an antibody fragment include the Fc region of the antibody, a portion of the Fc region, or a portion of the antibody comprising the Fc region. Examples of antigen-binding antibody fragments include domain antibodies (dAbs), Fab, Fab′, F(ab′)2 and Fv fragments; diabodies; linear antibodies (see U.S. Pat. No. 5,641,870, Example 2; Zapata et ah, Protein Eng. 8(10): 1057-1062 [1995]); single-chain antibody molecules, and multispecific antibodies formed from antibody fragments, a single chain Fv (scFv), a single domain antibody (VHH), one or more CDRs, a variable heavy chain (VH), a variable light chain (VL), a Fab-like bispecific antibodies (bsFab), a single-domain antibody-linked Fab (s-Fab), and a combination thereof. Single heavy chain antibodies or single light chain antibodies can be engineered, or in the case of the heavy chain, can be isolated from camelids, shark, libraries or mice engineered to produce single heavy chain molecules.

Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CHI). Each Fab fragment is monovalent with respect to antigen binding, i.e., it has a single antigen-binding site. Pepsin treatment of an antibody yields a single large F(ab′)2 fragment which roughly corresponds to two disulfide linked Fab fragments having different antigen-binding activity and is still capable of cross-linking antigen. Fab′ fragments differ from Fab fragments by having a few additional residues at the carboxy terminus of the CHI domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known. The Fc fragment comprises the carboxy-terminal portions of both H chains held together by disulfide bonds. The effector functions of antibodies are determined by sequences and glycan in the Fc region, the region which is also recognized by Fc receptors (FcR) found on certain types of cells.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For example, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

Antibody “effector functions” refer to those biological activities attributable to the Fc region (a native sequence Fc region or amino acid sequence variant Fc region) of an antibody and vary with the antibody isotype. Examples of antibody effector functions include: Clq binding and complement dependent cytotoxicity; Fc receptor binding; antibody-dependent cell-mediated cytotoxicity (ADCC); phagocytosis; down regulation of cell surface receptors (e.g., B cell receptors); and B cell activation.

“Binding affinity” as used herein refers to the strength of the non-covalent interactions between a single binding site of a molecule (e.g., a cytokine) and its binding partner (e.g., a cytokine receptor). In some embodiments, the affinity of a binding protein (e.g., a cytokine) can generally be represented by a dissociation constant (Kd). Affinity can be measured by common methods known in the art, including those described herein.

An “isolated” nucleic acid molecule encoding the cytokine polypeptides described herein is a nucleic acid molecule that is identified and separated from at least one contaminant nucleic acid molecule with which it is ordinarily associated in the environment in which it was produced. In some embodiments, the isolated nucleic acid is free of association with all components associated with the production environment. The isolated nucleic acid molecules encoding the polypeptides and cytokine polypeptides herein is in a form other than in the form or setting in which it is found in nature. Isolated nucleic acid molecules therefore are distinguished from nucleic acid encoding the polypeptides and cytokine polypeptides herein existing naturally in cells.

The term “pharmaceutical formulation” refers to a preparation that is in such form as to permit the biological activity of the active ingredient to be effective, and that contains no additional components that are unacceptably toxic to a subject to which the formulation would be administered.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

As used herein, the term “treatment” refers to clinical intervention designed to alter the natural course of the individual or cell being treated during the course of clinical pathology. Desirable effects of treatment include decreasing the rate of disease progression, ameliorating, or palliating the disease state, and remission or improved prognosis. An individual is successfully “treated”, for example, if one or more symptoms associated with a disorder (e.g., a neoplastic disease) are mitigated or eliminated. For example, an individual is successfully “treated” if treatment results in increasing the quality of life of those suffering from a disease, decreasing the dose of other medications required for treating the disease, reducing the frequency of recurrence of the disease, lessening severity of the disease, delaying the development or progression of the disease, and/or prolonging survival of individuals.

As used herein, “in conjunction with” or “in combination with” refers to administration of one treatment modality in addition to another treatment modality. As such, “in conjunction with” or “in combination with” refers to administration of one treatment modality before, during or after administration of the other treatment modality to the individual.

As used herein, the term “prevention” includes providing prophylaxis with respect to occurrence or recurrence of a disease in an individual. An individual may be predisposed to, susceptible to a disorder, or at risk of developing a disorder, but has not yet been diagnosed with the disorder. In some embodiments, targeted cytokines described herein are used to delay development of a disorder.

As used herein, an individual “at risk” of developing a disorder may or may not have detectable disease or symptoms of disease and may or may not have displayed detectable disease or symptoms of disease prior to the treatment methods described herein. “At risk” denotes that an individual has one or more risk factors, which are measurable parameters that correlate with development of the disease, as known in the art. An individual having one or more of these risk factors has a higher probability of developing the disorder than an individual without one or more of these risk factors.

An “effective amount” refers to at least an amount effective, at dosages and for periods of time necessary, to achieve the desired or indicated effect, including a therapeutic or prophylactic result.

An effective amount can be provided in one or more administrations. A “therapeutically effective amount” is at least the minimum concentration required to effect a measurable improvement of a particular disorder. A therapeutically effective amount herein may vary according to factors such as the disease state, age, sex, and weight of the patient, and the ability of the antibody to elicit a desired response in the individual. A therapeutically effective amount may also be one in which any toxic or detrimental effects of the targeted cytokine are outweighed by the therapeutically beneficial effects. A “prophylactically effective amount” refers to an amount effective, at the dosages and for periods of time necessary, to achieve the desired prophylactic result. Typically, but not necessarily, since a prophylactic dose is used in subjects prior to or at the earlier stage of disease, the prophylactically effective amount can be less than the therapeutically effective amount.

“Chronic” administration refers to administration of the medicament(s) in a continuous as opposed to acute mode, so as to main the initial therapeutic effect (activity) for an extended period of time. “Intermittent” administration is treatment that is not consecutively done without interruption, but rather is cyclic in nature.

As used herein, an “individual” or a “subject” is a mammal. A “mammal” for purposes of treatment includes humans, domestic and farm animals, and zoo, sports, or pet animals, such as dogs, horses, rabbits, cattle, pigs, hamsters, gerbils, mice, ferrets, rats, cats, etc. In some embodiments, the individual or subject is human.

Amino acid: As used herein, term “amino acid,” in its broadest sense, refers to any compound and/or substance that can be incorporated into a polypeptide chain. In some embodiments, an amino acid has the general structure H2N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an 1-amino acid. “Standard amino acid” refers to any of the twenty standard 1-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically engineered animal, and/or a clone.

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active.

Delivery: As used herein, the term “delivery” encompasses both local and systemic delivery. For example, delivery of mRNA encompasses situations in which an mRNA is delivered to a target tissue and the encoded protein is expressed and retained within the target tissue (also referred to as “local distribution” or “local delivery”), and situations in which an mRNA is delivered to a target tissue and the encoded protein is expressed and secreted into patient's circulation system (e.g., serum) and systematically distributed and taken up by other tissues (also referred to as “systemic distribution” or “systemic delivery).

Dosing interval: As used herein dosing interval in the context of a method for treating a disease is the frequency of administering a therapeutic composition in a subject (mammal) in need thereof, for example an mRNA composition, at an effective dose of the mRNA, such that one or more symptoms associated with the disease is reduced; or one or more biomarkers associated with the disease is reduced, at least for the period of the dosing interval. Dosing frequency and dosing interval may be used interchangeably in the current disclosure.

Expression: As used herein, “expression” of a nucleic acid sequence refers to translation of an mRNA into a polypeptide, assemble multiple polypeptides into an intact protein (e.g., enzyme) and/or post-translational modification of a polypeptide or fully assembled protein (e.g., enzyme). In this application, the terms “expression” and “production,” and grammatical equivalent, are used inter-changeably.

Effective dose: As used herein, an effective dose is a dose of the mRNA in the pharmaceutical composition which when administered to the subject in need thereof, hereby a mammalian subject, according to the methods of the invention, is effective to bring about an expected outcome in the subject, for example reduce a symptom associated with the disease.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Pharmaceutically acceptable salt: Pharmaceutically acceptable salts are well known in the art. For example, S. M. Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C1-4 alkyl)4 salts. Representative alkali or alkaline earth metal salts include sodium, lithium, potassium, calcium, magnesium, and the like. Further pharmaceutically acceptable salts include, when appropriate, nontoxic ammonium. quaternary ammonium, and amine cations formed using counterions such as halide, hydroxide, carboxylate, sulfate, phosphate, nitrate, sulfonate and aryl sulfonate. Further pharmaceutically acceptable salts include salts formed from the quarternization of an amine using an appropriate electrophile, e.g., an alkyl halide, to form a quarternized alkylated amino salt.

Subject: As used herein, the term “subject” refers to a human or any non-human animal (e.g., mouse, rat, rabbit, dog, cat, cattle, swine, sheep, horse, or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Target tissues: As used herein, the term “target tissues” refers to any tissue that is affected by a disease to be treated. In some embodiments, target tissues include those tissues that display disease-associated pathology, symptom, or feature.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Various aspects of the invention are described in detail in the following sections. The use of sections is not meant to limit the invention. Each section can apply to any aspect of the invention. In this application, the use of “or” means “and/or” unless stated otherwise.

### DETAILED DESCRIPTION

### Targeted Cytokine

The present invention provides, among other things, a targeted cytokine which comprises a targeting moiety, a cytokine or a variant thereof, a masking moiety, and an Fc domain. Targeted cytokines of the present invention become active at the site of disease, and is able to specifically target a cell of interest for effective treatment of cancer without causing undesired side effects.

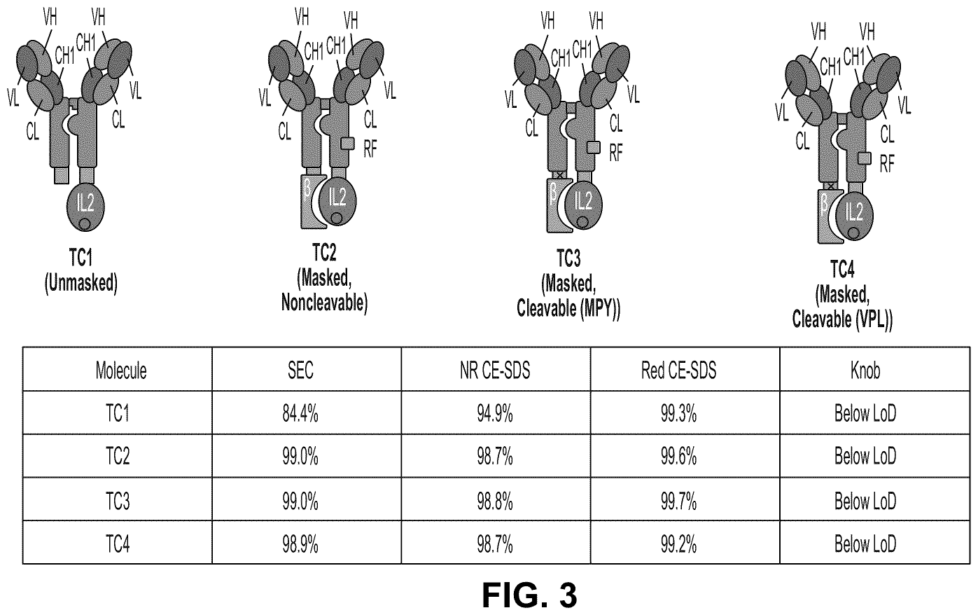

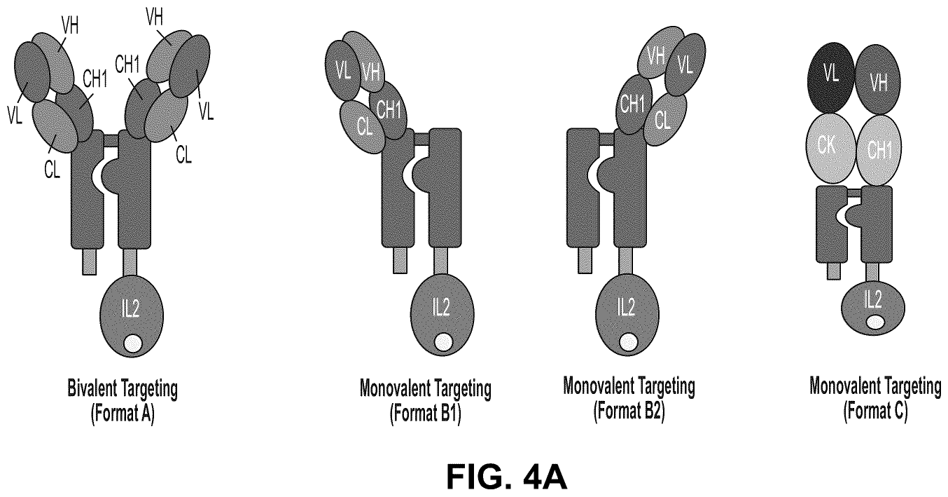

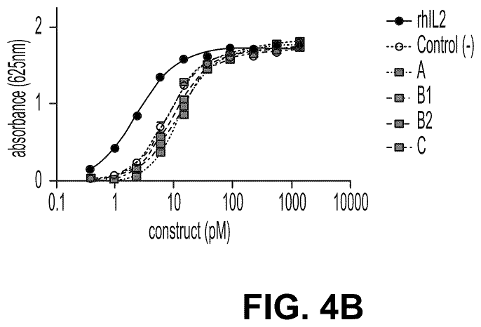

In some embodiments, a cytokine is linked to an Fc domain via a cleavable or a non-cleavable linker. In some embodiments, a masking moiety is linked to an Fc domain via a cleavable or a non-cleavable linker. In some embodiments, a targeting moiety is linked to an Fc domain with or without cleavable or a non-cleavable linker. Exemplary targeted cytokines of the present invention are shown in FIG. 3 and FIG. 4A. In some embodiments, a masking moiety is linked to an Fc polypeptide via a cleavable linker. In some embodiments, a masking moiety is linked to an Fc polypeptide via a non-cleavable linker. In some embodiments, a cytokine or a variant thereof is linked to an Fc polypeptide via a cleavable linker. In some embodiments, a cytokine or a variant thereof is linked to an Fc polypeptide via a non-cleavable linker.

Targeted IL-2 cytokines according to the disclosure can combine an IL-2 cytokine or functional fragment thereof as described anywhere herein; a masking moiety as described anywhere herein; first and second Fc domains as described anywhere herein; cleavable and non-cleavable linkers as described anywhere herein; and targeting moieties as described anywhere herein.

### Targeted Cytokine Formats

In some embodiments, a targeted cytokine is a bivalent targeting format, comprising 1) a first chain comprising a variable heavy region and an IgG1 or IgG4 heavy constant region with “hole mutations” (Y349C; T366S; L368A; and Y407V); 2) a second chain comprising a variable heavy region, a heavy constant region with “knob mutations” (S354C and T366W) fused to a cytokine or a variant thereof, wherein the CH1 and CH2 domains are from IgG1 or IgG4, and the CH3 domain is from IgG3; and 3) a third chain comprising a variable light region and immunoglobulin kappa or lambda constant region.

In some embodiments, a targeted cytokine is a bivalent targeting format, comprising 1) a first chain comprising a variable heavy region and an IgG1 or IgG4 heavy constant region with “hole mutations” (Y349C; T366S; L368A; and Y407V); 2) a second chain comprising a variable heavy region, a heavy constant region from IgG1 or IgG4 with “knob mutations” (S354C and T366W) and “RF mutations” (H435R and Y436F) fused to a cytokine or a variant thereof; and 3) a third chain comprising a variable light region and immunoglobulin kappa or lambda constant region.

In some embodiments, a targeted cytokine is a bivalent targeting format, comprising 1) a first chain comprising a variable heavy region and an IgG1 or IgG4 heavy constant region with “hole mutations” (Y349C; T366S; L368A; and Y407V) fused to a cytokine or a variant thereof; 2) a second chain comprising a variable heavy region, a heavy constant region with “knob mutations” (S354C and T366W) fused to a masking moiety, wherein the CH1 and CH2 domains are from IgG1 or IgG4, and the CH3 domain is from IgG3; and 3) a third chain comprising a variable light region and immunoglobulin kappa or lambda constant region.

In some embodiments, a targeted cytokine is a bivalent targeting format, comprising 1) a first chain comprising a variable heavy region and an IgG1 or IgG4 heavy constant region with “hole mutations” (Y349C; T366S; L368A; and Y407V) fused to a cytokine or a variant thereof; 2) a second chain comprising a variable heavy region, a heavy constant region from IgG1 or IgG4 with “knob mutations” (S354C and T366W) and “RF mutations” (H435R and Y436F) fused to a masking moiety; and 3) a third chain comprising a variable light region and immunoglobulin kappa or lambda constant region.

In some embodiments, a targeted cytokine is a bivalent targeting format, comprising 1) a first chain comprising a variable heavy region and an IgG1 or IgG4 heavy constant region with “hole mutations” (Y349C; T366S; L368A; and Y407V) fused to a masking moiety; 2) a second chain comprising a variable heavy region, a heavy constant region with “knob mutations” (S354C and T366W) fused to a cytokine or a variant thereof, wherein the CH1 and CH2 domains are from IgG1 or IgG4, and the CH3 domain is from IgG3; and 3) a third chain comprising a variable light region and immunoglobulin kappa or lambda constant region.

In some embodiments, a targeted cytokine is a bivalent targeting format, comprising 1) a first chain comprising a variable heavy region and an IgG1 or IgG4 heavy constant region with “hole mutations” (Y349C; T366S; L368A; and Y407V) fused to a masking moiety; 2) a second chain comprising a variable heavy region, a heavy constant region from IgG1 or IgG4 with “knob mutations” (S354C and T366W) and “RF mutations” (H435R and Y436F) fused to a cytokine or a variant thereof; and 3) a third chain comprising a variable light region and immunoglobulin kappa or lambda constant region.

In some embodiments, a targeted cytokine is a monovalent targeting format, comprising 1) a first chain comprising a Fab fused to a first Fc polypeptide chain from an IgG1 or IgG4 with “hole mutations” (Y349C; T366S; L368A; and Y407V); and 2) a second chain comprising a second Fc polypeptide chain from IgG1 or IgG4 with “knob mutations” (S354C and T366W) and “RF mutations” (H435R and Y436F).

In some embodiments, a targeted cytokine is a monovalent targeting format, comprising 1) a first chain comprising a Fab fused to a first Fc polypeptide chain from an IgG1 or IgG4 with “hole mutations” (Y349C; T366S; L368A; and Y407V); and 2) a second chain comprising a second Fc polypeptide chain with “knob mutations” (S354C and T366W) wherein the CH1 and CH2 domains are from IgG1 or IgG4, and the CH3 domain is from IgG3.

In some embodiments, a targeted cytokine is a monovalent targeting format, comprising 1) a first chain comprising a first Fc polypeptide chain from an IgG1 or IgG4 with “hole mutations” (Y349C; T366S; L368A; and Y407V); and 2) a second chain comprising a Fab fused to a second Fc polypeptide chain from IgG1 or IgG4 with “knob mutations” (S354C and T366W) and “RF mutations” (H435R and Y436F). In some embodiments, a cytokine is fused to a first Fc polypeptide chain. In some embodiments, a cytokine is fused to a second Fc polypeptide chain. In some embodiments, a masking moiety is fused to a first Fc polypeptide chain. In some embodiments, a masking moiety is fused to a second Fc polypeptide chain.

In some embodiments, a targeted cytokine is a monovalent targeting format, comprising 1) a first chain comprising a first Fc polypeptide chain from an IgG1 or IgG4 with “hole mutations” (Y349C; T366S; L368A; and Y407V); and 2) a second chain comprising a Fab fused to a second Fc polypeptide chain with “knob mutations” (S354C and T366W) wherein the CH1 and CH2 domains are from IgG1 or IgG4, and the CH3 domain is from IgG3. In some embodiments, a cytokine is fused to a second Fc polypeptide chain. In some embodiments, a masking moiety is fused to a first Fc polypeptide chain. In some embodiments, a masking moiety is fused to a second Fc polypeptide chain.

In some embodiments, a targeted cytokine is a monovalent targeting format, comprising 1) a first chain comprising a variable light region and IgG kappa or lambda constant region fused to an IgG1 or IgG4 heavy constant region with “hole mutations” (Y349C; T366S; L368A; and Y407V; and 2) a second chain comprising a variable heavy region fused to an IgG1 or IgG4 heavy constant region with “knob mutations” (S354C and T366W) and “RF mutations” (H435R and Y436F), wherein the C-terminal of the CH3 domain is fused to a cytokine or a variant thereof. In some embodiments, a cytokine is fused to a second Fc polypeptide chain. In some embodiments, a masking moiety is fused to a first Fc polypeptide chain. In some embodiments, a masking moiety is fused to a second Fc polypeptide chain.

In some embodiments, a targeted cytokine is a monovalent targeting format, comprising 1) a first chain comprising a variable light region and IgG kappa or lambda constant region fused to an IgG1 or IgG4 heavy constant region with “hole mutations” (Y349C; T366S; L368A; and Y407V; and 2) a second chain comprising a variable heavy region fused to a heavy constant region with “knob mutations” (S354C and T366W) fused to a cytokine or a variant thereof, wherein the CH1 and CH2 domains are from IgG1 or IgG4, and the CH3 domain is from IgG3.

In some embodiments, a targeted cytokine is a monovalent targeting format, comprising 1) a first chain comprising a variable heavy region fused to an IgG1 or IgG4 heavy constant region with “hole mutations” (Y349C; T366S; L368A; and Y407V; and 2) a second chain comprising a variable light region and IgG kappa or lambda constant region fused to an IgG1 or IgG4 heavy constant region with “knob mutations” (S354C and T366W) and “RF mutations” (H435R and Y436F), wherein the C-terminal of the CH3 domain is fused to a cytokine or a variant thereof.

In some embodiments, a targeted cytokine is a monovalent targeting format, comprising 1) a first chain comprising a variable heavy region fused to an IgG1 or IgG4 heavy constant region with “hole mutations” (Y349C; T366S; L638A; and Y407V; and 2) a second chain comprising a light variable region and IgG kappa or lambda constant region fused to a heavy constant region with “knob mutations” (S354C and T366W) fused to a cytokine or a variant thereof, wherein the CH1 and CH2 domains are from IgG1 or IgG4, and the CH3 domain is from IgG3.

### Targeting Moiety

Provided herein is a targeted cytokine that comprises a targeting moiety. In some embodiments, a targeting moiety comprises an antigen-binding moiety that binds to an antigen expressed on the surface of a target cell.

In some embodiments, the targeting moiety comprises an antigen-binding moiety, wherein the antigen is expressed on an immune cell. In some embodiments, the targeting moiety comprises an antigen-binding moiety, wherein the antigen is selected from PD-1, PD-L1, CTLA-4, TIGIT, TIM-3, LAG-3, OX40, DR5, ICOS, GITR, CD73, CD39, CD25, CD16a, CD8, KLRC1, KLRD1, KLRB1, CD40, CD137, CD28 and CD16b.

In some embodiments, a targeting moiety specifically binds PD-1, PD-L1, PD-L2, CTLA-4, TIGIT, TIM-3, LAG-3, CD25, CD16a, CD16b, OX40, DR5, ICOS, GITR, NKG2D, KLRC1, KLRD1, KLRB1, NKP44, NKP30, BCMA, human epidermal growth factor receptor 2 (HER2), MICA, DLK1, human epidermal growth factor receptor 3 (HER3), delta-like protein 3 (DLL3), delta-like protein 4 (DLL4), epidermal growth factor receptor (EGFR), glypican-3 (GPC3), c-MET, vascular endothelial growth factor receptor 1 (VEGF RD, vascular endothelial growth factor receptor 2 (VEG FR2), Nectin-4, Liv-1, glycoprotein NMB (GPNMB), prostate specific membrane antigen (PSMA), Trop-2, carbonic anhydrase IX (CA9), endothelin B receptor (ETBR), six transmembrane epithelial antigen of the prostate 1 (STEAP1), NAPI2B, folate receptor alpha (FR-a), SLIT and NTRK-like protein 6 (SLITRK6), carbonic anhydrase VI (CA6), ectonucleotide pyrophosphatase/phosphodiesterase family member 3 (ENPP3), mesothelin, trophoblast glycoprotein (TPBG), CD19, CD8, CD20, CD22, CD28, CD33, CD39, CD40, CD56, CD66e, CD70, CD73, CD74, CD79b, CD98, CD123, CD137, CD138, CD352, CD47, signal-regulatory protein alpha (SIRPα), Claudin 18.2, Claudin 6, 5T4, fibroblast activation protein alpha (FAPα), fibronectin, the melanoma-associated chondroitin sulfate proteoglycan (MCSP), epithelial cellular adhesion molecule (EPCAM), or combinations thereof.

In some embodiments, a targeting moiety binds a tumor-associated antigen. In some embodiments, a targeting moiety is an antibody or an antigen binding fragment that binds a tumor-associated antigen. In some embodiments, a targeting moiety is a bispecific antibody or an antigen binding fragment that binds a tumor-associated antigen. In some embodiments, a targeting moiety is an anti-alpha-fetoprotein (AFP) antibody or a fragment thereof. In some embodiments, a targeting moiety is an anti-B2M antibody or a fragment thereof. In some embodiments, a targeting moiety is an anti-beta-human chorionic gonadotropin (beta-hCG) antibody or a fragment thereof. In some embodiments, a targeting moiety is an anti-CD117 antibody or a fragment thereof. In some embodiments, a targeting moiety is an anti-CD19 antibody or a fragment thereof. In some embodiments, a targeting moiety is an anti-CD20 antibody or a fragment thereof. In some embodiments, a targeting moiety is an anti-CD22 antibody or a fragment thereof. In some embodiments, a targeting moiety is an anti-CD25 antibody or a fragment thereof. In some embodiments, a targeting moiety is an anti-CD30 antibody or a fragment thereof. In some embodiments, a targeting moiety is an anti-CD33 antibody or a fragment thereof. In some embodiments, a targeting moiety is an anti-CD151 antibody or a fragment thereof. In some embodiments, a targeting moiety is an anti-MUC-1 antibody or a fragment thereof.

In some embodiments, a targeting moiety specifically binds PD-1. In some embodiments, a targeting moiety is an anti-PD1 Fab. In some embodiments, a targeting moiety is an anti-PD1 scFv.

In some embodiments, the targeting moiety is derived from an anti-PD1 antibody. In some embodiments, the targeting moiety is derived from pembrolizumab or nivolumab.

In some embodiments, a targeting moiety binds PD-L1.

In some embodiments, a targeting moiety comprises an agent, a peptide, or a polypeptide that specifically binds to a target.

In some embodiments, a targeting moiety comprises a Fab, a single chain Fv (scFv), a single domain antibody (VHH), one or more CDRs, a variable heavy chain (VH), a variable light chain (VL), a Fab-like bispecific antibodies (bsFab), a single-domain antibody-linked Fab (s-Fab), an antibody, or a combination thereof. In some embodiments, a targeting moiety comprises a Fab. In some embodiments, a targeting moiety comprises a single chain Fv (scFv). In some embodiments, a targeting moiety comprises a single domain antibody (VHH). In some embodiments, a targeting moiety comprises one or more CDRs. In some embodiments, a targeting moiety comprises a variable heavy chain (VH). In some embodiments, a targeting moiety comprises a variable light chain (VL). In some embodiments, a targeting moiety comprises a Fab-like bispecific antibodies (bsFab). In some embodiments, a targeting moiety comprises a single-domain antibody-linked Fab (s-Fab). In some embodiments, a targeting moiety comprises an antibody or a fragment thereof.

In some embodiments, a targeting moiety comprises a heavy chain variable region of

(SEQ ID NO: 1)
QVQLVQSGVEVKKPGASVKVSCKASGYTFTNYYMYWVRQAPGQGLEWMG
GINPSNGGTNFNEKFKNRVTLTTDSSTTTAYMELKSLQFDDTAVYYCAR
RDYRFDMGFDYWGQGTTVTVSS.

In some embodiments, a targeting moiety comprises an amino acid sequence having at least about 80% sequence identity to SEQ ID NO: 1. In some embodiments, a targeting moiety comprises an amino acid sequence having at least about 85% sequence identity to SEQ ID NO: 1. In some embodiments, a targeting moiety comprises an amino acid sequence having at least about 90% sequence identity to SEQ ID NO: 1. In some embodiments, a targeting moiety comprises an amino acid sequence having at least about 92% sequence identity to SEQ ID NO: 1. In some embodiments, a targeting moiety comprises an amino acid sequence having at least about 95% sequence identity to SEQ ID NO: 1. In some embodiments, a targeting moiety comprises an amino acid sequence having at least about 96% sequence identity to SEQ ID NO: 1. In some embodiments, a targeting moiety comprises an amino acid sequence having at least about 97% sequence identity to SEQ ID NO: 1. In some embodiments, a targeting moiety comprises an amino acid sequence having at least about 98% sequence identity to SEQ ID NO: 1. In some embodiments, a targeting moiety comprises an amino acid sequence having at least about 99% sequence identity to SEQ ID NO: 1.

In some embodiments, a targeting moiety comprises a heavy chain of

(SEQ ID NO: 2)
QVQLVQSGVEVKKPGASVKVSCKASGYTFTNYYMYWVRQAPGQGLEWMG
GINPSNGGTNFNEKFKNRVTLTTDSSTTTAYMELKSLQFDDTAVYYCAR
RDYRFDMGFDYWGQGTTVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSC.

In some embodiments, a targeting moiety comprises an amino acid sequence having at least about 80% sequence identity to SEQ ID NO: 2. In some embodiments, a targeting moiety comprises an amino acid sequence having at least about 85% sequence identity to SEQ ID NO: 2. In some embodiments, a targeting moiety comprises an amino acid sequence having at least about 90% sequence identity to SEQ ID NO: 2. In some embodiments, a targeting moiety comprises an amino acid sequence having at least about 92% sequence identity to SEQ ID NO: 2. In some embodiments, a targeting moiety comprises an amino acid sequence having at least about 95% sequence identity to SEQ ID NO: 2. In some embodiments, a targeting moiety comprises an amino acid sequence having at least about 96% sequence identity to SEQ ID NO: 2. In some embodiments, a targeting moiety comprises an amino acid sequence having at least about 97% sequence identity to SEQ ID NO: 2. In some embodiments, a targeting moiety comprises an amino acid sequence having at least about 98% sequence identity to SEQ ID NO: 2. In some embodiments, a targeting moiety comprises an amino acid sequence having at least about 99% sequence identity to SEQ ID NO: 2.

In some embodiments, a targeting moiety comprises a heavy chain of

(SEQ ID NO: 3)
QVQLVQSGVEVKKPGASVKVSCKASGYTFTNYYMYWVRQAPGQGLEWMG
GINPSNGGTNFNEKFKNRVTLTTDSSTTTAYMELKSLQFDDTAVYYCAR
RDYRFDMGFDYWGQGTTVTVSSASTKGPSVFPLAPCSRSTSESTAALGC
LVKDYFPEPVTVSWNSGALTSGVHTFPAVLQSSGLYSLSSVVTVPSSSL
GTKTYTCNVDHKPSNTKVDKRVESKYGPPCPPCPAPEFLGGPSVFLFPP
KPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSL
SLSLGK

In some embodiments, a targeting moiety comprises an amino acid sequence having at least about 80% sequence identity to SEQ ID NO: 3. In some embodiments, a targeting moiety comprises an amino acid sequence having at least about 85% sequence identity to SEQ ID NO: 3. In some embodiments, a targeting moiety comprises an amino acid sequence having at least about 90% sequence identity to SEQ ID NO: 3. In some embodiments, a targeting moiety comprises an amino acid sequence having at least about 92% sequence identity to SEQ ID NO: 3. In some embodiments, a targeting moiety comprises an amino acid sequence having at least about 95% sequence identity to SEQ ID NO: 3. In some embodiments, a targeting moiety comprises an amino acid sequence having at least about 96% sequence identity to SEQ ID NO: 3. In some embodiments, a targeting moiety comprises an amino acid sequence having at least about 97% sequence identity to SEQ ID NO: 3 In some embodiments, a targeting moiety comprises an amino acid sequence having at least about 98% sequence identity to SEQ ID NO: 3. In some embodiments, a targeting moiety comprises an amino acid sequence having at least about 99% sequence identity to SEQ ID NO: 3.

In some embodiments, a targeting moiety comprises a heavy chain CDR1 sequence of GYTFTNYY (SEQ ID NO: 4). In some embodiments, a targeting moiety comprises a heavy chain CDR2 sequence of INPSNGGT (SEQ ID NO: 5). In some embodiments, a targeting moiety comprises a heavy chain CDR3 sequence of ARRDYRFDMGFDY (SEQ ID NO: 6). In some embodiments, a targeting moiety comprises a HCDR1 of SEQ ID NO: 4, a HCDR2 of SEQ ID NO: 5, and a HCDR3 of SEQ ID NO: 6.

In some embodiments, a targeting moiety comprises a light chain variable region of

(SEQ ID NO: 7)
EIVLTQSPATLSLSPGERATLSCRASKGVSTSGYSYLHWYQQKPGQAPR
LLIYLASYLESGVPARFSGSGSGTDFTLTISSLEPEDFAVYYCQHSRDL
PLTFGGGTKVEIKTSENLYFQ.

In some embodiments, a targeting moiety comprises an amino acid sequence having at least about 80% sequence identity to SEQ ID NO: 7. In some embodiments, a targeting moiety comprises an amino acid sequence having at least about 85% sequence identity to SEQ ID NO: 7. In some embodiments, a targeting moiety comprises an amino acid sequence having at least about 90% sequence identity to SEQ ID NO: 7. In some embodiments, a targeting moiety comprises an amino acid sequence having at least about 92% sequence identity to SEQ ID NO: 7. In some embodiments, a targeting moiety comprises an amino acid sequence having at least about 95% sequence identity to SEQ ID NO: 7. In some embodiments, a targeting moiety comprises an amino acid sequence having at least about 96% sequence identity to SEQ ID NO: 7. In some embodiments, a targeting moiety comprises an amino acid sequence having at least about 97% sequence identity to SEQ ID NO: 7. In some embodiments, a targeting moiety comprises an amino acid sequence having at least about 98% sequence identity to SEQ ID NO: 7. In some embodiments, a targeting moiety comprises an amino acid sequence having at least about 99% sequence identity to SEQ ID NO: 7.

In some embodiments, a targeting moiety comprises a light chain of

(SEQ ID NO: 8)
EIVLTQSPATLSLSPGERATLSCRASKGVSTSGYSYLHWYQQKPGQAPR
LLIYLASYLESGVPARFSGSGSGTDFTLTISSLEPEDFAVYYCQHSRDL
PLTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC.

In some embodiments, a targeting moiety comprises an amino acid sequence having at least about 80% sequence identity to SEQ ID NO: 8. In some embodiments, a targeting moiety comprises an amino acid sequence having at least about 85% sequence identity to SEQ ID NO: 8. In some embodiments, a targeting moiety comprises an amino acid sequence having at least about 90% sequence identity SEQ ID NO: 8. In some embodiments, a targeting moiety comprises an amino acid sequence having at least about 92% sequence identity to SEQ ID NO: 8. In some embodiments, a targeting moiety comprises an amino acid sequence having at least about 95% sequence identity to SEQ ID NO: 8. In some embodiments, a targeting moiety comprises an amino acid sequence having at least about 96% sequence identity to SEQ ID NO: 8. In some embodiments, a targeting moiety comprises an amino acid sequence having at least about 97% sequence identity to SEQ ID NO: 8. In some embodiments, a targeting moiety comprises an amino acid sequence having at least about 98% sequence identity to SEQ ID NO: 8. In some embodiments, a targeting moiety comprises an amino acid sequence having at least about 99% sequence identity to SEQ ID NO: 8.

In some embodiments, a targeting moiety comprises a light chain CDR1 sequence of KGVSTSGYSY (SEQ ID NO: 9). In some embodiments, a targeting moiety comprises a light chain CDR2 sequence of LAS (SEQ ID NO: 10). In some embodiments, a targeting moiety comprises a light chain CDR3 sequence of QHSRDLPLT (SEQ ID NO: 11). In some embodiments, a targeting moiety comprises a LCDR1 of SEQ ID NO: 9, a LCDR2 of SEQ ID NO: 10, and a LCDR3 of SEQ ID NO: 11.

In some embodiments, a targeting moiety comprises a HCDR1 of SEQ ID NO: 4, a HCDR2 of SEQ ID NO: 5, a HCDR3 of SEQ ID NO: 6, a LCDR1 of SEQ ID NO: 9, a LCDR2 of SEQ ID NO: 10, and a LCDR3 of SEQ ID NO: 11.

In some embodiments, a targeting moiety comprises a heavy chain variable region or a light chain variable region of nivolumab.

In some embodiments, a targeting moiety comprises a heavy chain variable region of

(SEQ ID NO: 159)
QVQLVESGGGVVQPGRSLRLDCKASGITFSNSGMHWVRQAPGKGLEWVA
VIWYDGSKRYYADSVKGRFTISRDNSKNTLFLQMNSLRAEDTAVYYCAT
NDDYWGQGTLVTVSS.

In some embodiments, a targeting moiety comprises an amino acid sequence having at least about 80% sequence identity to SEQ ID NO: 159. In some embodiments, a targeting moiety comprises an amino acid sequence having at least about 85% sequence identity to SEQ ID NO: 159. In some embodiments, a targeting moiety comprises an amino acid sequence having at least about 90% sequence identity to SEQ ID NO: 159. In some embodiments, a targeting moiety comprises an amino acid sequence having at least about 92% sequence identity to SEQ ID NO: 159. In some embodiments, a targeting moiety comprises an amino acid sequence having at least about 95% sequence identity to SEQ ID NO: 159. In some embodiments, a targeting moiety comprises an amino acid sequence having at least about 96% sequence identity to SEQ ID NO: 159. In some embodiments, a targeting moiety comprises an amino acid sequence having at least about 97% sequence identity to SEQ ID NO: 159. In some embodiments, a targeting moiety comprises an amino acid sequence having at least about 98% sequence identity to SEQ ID NO: 159. In some embodiments, a targeting moiety comprises an amino acid sequence having at least about 99% sequence identity to SEQ ID NO: 159.

In some embodiments, a targeting moiety comprises a heavy chain of

(SEQ ID NO: 157)
QVQLVESGGG VVQPGRSLRL DCKASGITFS NSGMHWVRQA PGKGLEWVAV
IWYDGSKRYY ADSVKGRFTI SRDNSKNTLF LQMNSLRAED TAVYYCATND
DYWGQGTLVT VSSASTKGPS VFPLAPCSRS TSESTAALGC LVKDYFPEPV
TVSWNSGALT SGVHTFPAVL QSSGLYSLSS VVTVPSSSLG TKTYTCNVDH
KPSNTKVDKR VESKYGPPCP PCPAPEFLGG PSVFLFPPKPKDTLMISRTP EVTCVVVDVS
QEDPEVQFNW YVDGVEVHNA KTKPREEQFN STYRVVSVLT VLHQDWLNGK
EYKCKVSNKG LPSSIEKTIS KAKGQPREPQ VYTLPPSQEE MTKNQVSLTC LVKGFYPSDI
AVEWESNGOP ENNYKTTPPV LDSDGSFFLY SRLTVDKSRW QEGNVFSCSV
MHEALHNHYT QKSLSLSLGK.

In some embodiments, a targeting moiety comprises an amino acid sequence having at least about 80% sequence identity to SEQ ID NO: 157. In some embodiments, a targeting moiety comprises an amino acid sequence having at least about 85% sequence identity to SEQ ID NO: 157. In some embodiments, a targeting moiety comprises an amino acid sequence having at least about 90% sequence identity to SEQ ID NO: 157. In some embodiments, a targeting moiety comprises an amino acid sequence having at least about 92% sequence identity to SEQ ID NO: 157. In some embodiments, a targeting moiety comprises an amino acid sequence having at least about 95% sequence identity to SEQ ID NO: 157. In some embodiments, a targeting moiety comprises an amino acid sequence having at least about 96% sequence identity to SEQ ID NO: 157. In some embodiments, a targeting moiety comprises an amino acid sequence having at least about 97% sequence identity to SEQ ID NO: 157. In some embodiments, a targeting moiety comprises an amino acid sequence having at least about 98% sequence identity to SEQ ID NO: 157. In some embodiments, a targeting moiety comprises an amino acid sequence having at least about 99% sequence identity to SEQ ID NO: 157.

In some embodiments, a targeting moiety comprises a heavy chain CDR1 sequence of GITFSNSG (SEQ ID NO: 161). In some embodiments, a targeting moiety comprises a heavy chain CDR2 sequence of VIWYDGSKRYYADSVKG (SEQ ID NO: 162). In some embodiments, a targeting moiety comprises a heavy chain CDR3 sequence of ATNDDY(SEQ ID NO: 163)). In some embodiments, a targeting moiety comprises a HCDR1 of GITFSNSG (SEQ ID NO: 161), a HCDR2 of VIWYDGSKRYYADSVKG (SEQ ID NO: 162) and a HCDR3 of ATNDDY (SEQ ID NO: 163).

In some embodiments, the targeting moiety comprises a heavy chain variable region and a CH1 domain comprising SEQ ID NO: 264.

(SEQ ID NO: 264)
QVQLVESGGGVVQPGRSLRLDCKASGITFSNSGMHWVRQAPGKGLEWVA
VIWYDGSKRYYADSVKGRFTISRDNSKNTLFLQMNSLRAEDTAVYYCAT
NDDYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYIC
NVNHKPSNTKVDKKVEPKSC.

In some embodiments, a targeting moiety comprises a light chain variable region of

(SEQ ID NO: 160)
EIVLTQSPATLSLSPGERATLSCRASQSVSSYLAWYQQKPGQAPRLLIY
DASNRATGIPARFSGSGSGTDFTLTISSLEPEDFAVYYCQQSSNWPRTF
GQGTKVEIK

In some embodiments, a targeting moiety comprises an amino acid sequence having at least about 80% sequence identity to SEQ ID NO: 160. In some embodiments, a targeting moiety comprises an amino acid sequence having at least about 85% sequence identity to SEQ ID NO: 160. In some embodiments, a targeting moiety comprises an amino acid sequence having at least about 90% sequence identity to SEQ ID NO: 160. In some embodiments, a targeting moiety comprises an amino acid sequence having at least about 92% sequence identity to SEQ ID NO: 160. In some embodiments, a targeting moiety comprises an amino acid sequence having at least about 95% sequence identity to SEQ ID NO: 160. In some embodiments, a targeting moiety comprises an amino acid sequence having at least about 96% sequence identity to SEQ ID NO: 160. In some embodiments, a targeting moiety comprises an amino acid sequence having at least about 97% sequence identity to SEQ ID NO: 160. In some embodiments, a targeting moiety comprises an amino acid sequence having at least about 98% sequence identity to SEQ ID NO: 160. In some embodiments, a targeting moiety comprises an amino acid sequence having at least about 99% sequence identity to SEQ ID NO: 160.

In some embodiments, a targeting moiety comprises a light chain of EIVLTQSPAT LSLSPGERAT LSCRASQSVS SYLAWYQQKP GQAPRLLIYD ASNRATGIPA RFSGSGSGTD FTLTISSLEP EDFAVYYCQQ SSNWPRTFGQ GTKVEIKRTV AAPSVFIFPP SDEQLKSGTA SVVCLLNNFY PREAKVQWKV DNALQSGNSQ ESVTEQDSKD STYSLSSTLT LSKADYEKHK VYACEVTHQG LSSPVTKSFN RGE (SEQ ID NO: 158).

In some embodiments, a targeting moiety comprises an amino acid sequence having at least about 80% sequence identity to SEQ ID NO: 158. In some embodiments, a targeting moiety comprises an amino acid sequence having at least about 85% sequence identity to SEQ ID NO: 158. In some embodiments, a targeting moiety comprises an amino acid sequence having at least about 90% sequence identity SEQ ID NO: 158. In some embodiments, a targeting moiety comprises an amino acid sequence having at least about 92% sequence identity to SEQ ID NO: 158. In some embodiments, a targeting moiety comprises an amino acid sequence having at least about 95% sequence identity to SEQ ID NO: 158. In some embodiments, a targeting moiety comprises an amino acid sequence having at least about 96% sequence identity to SEQ ID NO: 158. In some embodiments, a targeting moiety comprises an amino acid sequence having at least about 97% sequence identity to SEQ ID NO: 158. In some embodiments, a targeting moiety comprises an amino acid sequence having at least about 98% sequence identity to SEQ ID NO: 158. In some embodiments, a targeting moiety comprises an amino acid sequence having at least about 99% sequence identity to SEQ ID NO: 158.

In some embodiments, a targeting moiety comprises a light chain CDR1 sequence of QSVSSY (SEQ ID NO: 164). In some embodiments, a targeting moiety comprises a light chain CDR2 sequence of DAS (SEQ ID NO: 165). In some embodiments, a targeting moiety comprises a light chain CDR3 sequence of QQSSNWPRT (SEQ ID NO: 166). In some embodiments, a targeting moiety comprises a LCDR1 of SEQ ID NO: 164, a LCDR2 of SEQ ID NO: 165, and a LCDR3 of SEQ ID NO: 166.

In some embodiments, a targeting moiety comprises a HCDR1 of SEQ ID NO: 161, a HCDR2 of SEQ ID NO: 162, a HCDR3 of SEQ ID NO: 163, a LCDR1 of SEQ ID NO: 164, a LCDR2 of SEQ ID NO: 165, and a LCDR3 of SEQ ID NO: 166.

In some embodiments, the targeting moiety comprises a heavy chain variable region, and a CH1 domain. In some embodiments, the heavy chain variable region and the CH1 domain comprises the amino acid sequence (SEQ ID NO: 264)

(SEQ ID NO: 264)
QVQLVESGGGVVQPGRSLRLDCKASGITFSNSGMHWVRQAPGKGLEWVA
VIWYDGSKRYYADSVKGRFTISRDNSKNTLFLQMNSLRAEDTAVYYCAT
NDDYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYIC
NVNHKPSNTKVDKKVEPKSC.

In some embodiments, a targeting domain is fused to an Fc polypeptide. In some embodiments, the C-terminus of a targeting domain is fused to the N-terminus of an Fc polypeptide. In some embodiments, the heavy chain of a Fab is fused to an Fc polypeptide. In some embodiments, the C-terminus of the heavy chain of a Fab is fused to the N-terminus of an Fc polypeptide. In some embodiments, an Fc polypeptide comprises a cleavage site.

In some embodiments, the targeting moiety comprises a heavy chain variable region, a light chain variable region and a CH1 domain. In some embodiments, the CH1 domain is derived from IgG1, IgG2, IgG3 or IgG4. In some embodiments, the CH1 domain is derived from IgG1. In some embodiments, the CH1 domain is derived from IgG4.

In some embodiments, the targeting moiety comprises a CH1 domain comprising a sequence that is at least 90%, at least 91%, at least 92%, at least 93%, at least 94%, at least 95%, at least 96%, at least 97%, at least 98%, at least 99% or identical to SEQ ID NO: 156.

(SEQ ID NO: 156)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSC.

### Cytokines

The immune system is skilled in communication and designed to respond quickly, specifically and globally to protect an organism against foreign invaders and disease. The cytokine superfamily of proteins is an integral part of the signalling network between cells and is essential in generating and regulating the immune system. These interacting biological signals have remarkable capabilities, such as influencing growth and development, hematopoiesis, lymphocyte recruitment, T cell subset differentiation and inflammation.

Cytokines can be part of a bigger immune program, e.g., T cell subset differentiation. Mature CD4 and CD8 T cells leave the thymus with a naive phenotype and produce a variety of cytokines. In the periphery, these T cells encounter antigen presenting cells (APCs) displaying either major histocompatibility complex (MHC) class I molecules (present peptides generated in the cytosol to CD8 T cells) or MHC class II molecules (present peptides degraded in intracellular vesicles to CD4 T cells). Following activation, characteristic cytokine and chemokine secretion profiles allow the classification of CD4 T helper (Th) cells into two major subpopulations in mice and humans. 3-7Th1 cells secrete mainly IL-2, interferon-γ (IFN-γ) and tumor necrosis factor-β (TNF-β), whereas Th2 cells secrete mainly IL-4, IL-5, IL-6, IL-10 and IL-13. Th1 cells support cell-mediated immunity and as a consequence promote inflammation, cytotoxicity and delayed-type hypersensitivity (DTH). Th2 cells support humoral immunity and serve to downregulate the inflammatory actions of Th1 cells. This paradigm is a great example of an integrated biological network and is very useful in simplifying our understanding of typical immune responses and those that turn pathogenic. For example, the failure to communicate “self” can lead to a loss of tolerance to our own antigens and prompt destructive immune responses to self-tissues and autoimmune disease. Autoimmunity, the major focus of this book, is the underlying mechanism of a set of conditions, such as type 1 diabetes mellitus, multiple sclerosis and rheumatoid arthritis. Autoimmune diseases may be caused in part by cytokine- and chemokine-mediated dysregulation of Th cell subset differentiation. The main factors affecting the development of Th subsets, aside from the context in which the antigen and costimulatory signals are presented, are the cytokines and chemokines in the stimulatory milieu. A better understanding of the properties and interactions of the individual cytokines and chemokines that play a role in Th cell activation may lead to more advanced treatments for autoimmune disease.

The targeted cytokine of the present invention can comprise any cytokine or a variant thereof that is known in the art. See, e.g., Cameron M J, Kelvin D J. Cytokines, Chemokines and Their Receptors. In: Madame Curie Bioscience Database. Austin (TX): Landes Bioscience; 2000-2013, the contents of which is incorporated in its entirety. For example, a cytokine incorporated in the targeted cytokine can be IL-1, IL-2, IL-3, IL-4, IL-5, IL-6, IL-7, IL-9, IL-10, IL-11, IL-12, IL-13, IL-14, IL-15, IL-16, IL-17, IL-20, TNF-α, TNF-β, CXCL8 (IL-18), G-CSF, GM-CSF, LIF, OSM, IFN-α, IFN-β, IFN-γ, CD154, LT-β, 4-1BBL, APRIL, CD70, CD153, CD178, GITRL, LIGHT, OX40L, TALL-1, TRAIL, TWEAK, TRANCE, TGF-β, M-CSF, or MSP or a fragment thereof.

In some embodiments, a cytokine is IL-1 or a variant thereof. In some embodiments, a cytokine is IL-2 or a variant thereof. In some embodiments, a cytokine is IL-3 or a variant thereof. In some embodiments, a cytokine is IL-4 or a variant thereof. In some embodiments, a cytokine is IL-5 or a variant thereof. In some embodiments, a cytokine is IL-6 or a variant thereof. In some embodiments, a cytokine is IL-7 or a variant thereof. In some embodiments, a cytokine is IL-9 or a variant thereof. In some embodiments, a cytokine is IL-10 or a variant thereof. In some embodiments, a cytokine is IL-11 or a variant thereof. In some embodiments, a cytokine is IL-12 or a variant thereof. In some embodiments, a cytokine is IL-13 or a variant thereof. In some embodiments, a cytokine is IL-14 or a variant thereof. In some embodiments, a cytokine is IL-15 or a variant thereof. In some embodiments, a cytokine is IL-16 or a variant thereof. In some embodiments, a cytokine is IL-17 or a variant thereof. In some embodiments, a cytokine is IL-20 or a variant thereof. In some embodiments, a cytokine is TNF-α or a variant thereof. In some embodiments, a cytokine TNF-β or a variant thereof. In some embodiments, a cytokine is CXCL8 (IL-18) or a variant thereof. In some embodiments, a cytokine is G-CSF or a variant thereof. In some embodiments, a cytokine is CXCL8 (IL-18) or a variant thereof. In some embodiments, a cytokine is GM-CSF or a variant thereof. In some embodiments, a cytokine is LIF or a variant thereof. In some embodiments, a cytokine is OSM or a variant thereof. In some embodiments, a cytokine is IFN-α or a variant thereof. In some embodiments, a cytokine is IFN-β or a variant thereof. In some embodiments, a cytokine is IFN-γ or a variant thereof. In some embodiments, a cytokine is CD154 or a variant thereof. In some embodiments, a cytokine is LT-β or a variant thereof. In some embodiments, a cytokine is 4-1BBL or a variant thereof. In some embodiments, a cytokine is APRIL or a variant thereof. In some embodiments, a cytokine is CD153 or a variant thereof. In some embodiments, a cytokine is CD70 or a variant thereof. In some embodiments, a cytokine is CD178 or a variant thereof. In some embodiments, a cytokine is GITRL or a variant thereof. In some embodiments, a cytokine is LIGHT or a variant thereof. In some embodiments, a cytokine is OX40L or a variant thereof. In some embodiments, a cytokine is TALL-1 or a variant thereof. In some embodiments, a cytokine is TRAIL or a variant thereof. In some embodiments, a cytokine is TWEAK or a variant thereof. In some embodiments, a cytokine is TRANCE or a variant thereof. In some embodiments, a cytokine is TGF-β or a variant thereof. In some embodiments, a cytokine is M-CSF or a variant thereof. In some embodiments, a cytokine is MSP or a variant thereof.

Interleukin 2 (IL-2)

Provided herein is an IL-2 cytokine or functional fragment thereof for use in a targeted cytokine or cleavage product thereof. A cytokine plays a role in cellular signalling, particularly in cells of the immune system. IL-2 is an interleukin, which is a type of cytokine signalling molecule in the immune system that regulates activities of white blood cells. Suitable IL-2 cytokines for use in the present invention can be any IL-2 or functional fragment thereof. In some embodiments, the IL-2 is naturally occurring IL-2, an IL-2 comprising one or more substitutions (e.g., an IL-2 mutein, or IL-2 variant), or truncated IL-2. In some embodiments, the IL-2 is polypeptide that retains at least one property of IL-2 biological activity.

In some embodiments, the IL-2 is naturally occurring IL-2. In some embodiments, the IL-2 comprises C125A substitution of mature IL-2 (SEQ ID NO: 13).

In some embodiments, the amino acid substitutions reduce the affinity of the IL-2 polypeptide or functional fragment thereof for CD25 (IL-2Rα).

In some embodiments, the IL-2 polypeptide or functional fragment thereof comprises an amino acid sequence produced by introducing one or more amino acid substitutions into the amino acid sequence of the IL-2 polypeptide or functional fragment thereof that increases the affinity of the IL-2 polypeptide or functional fragment thereof for IL-2Rb or IL-2Rγ. In some embodiments, the IL-2 polypeptide or functional fragment thereof comprises an amino acid sequence having one or more amino acid substitutions compared to the amino acid sequence of wildtype IL-2 that enhances the affinity of the IL-2 polypeptide or functional fragment thereof for IL-2Rb (CD 122). In some embodiments, the IL-2 polypeptide or functional fragment thereof comprises an amino acid sequence having one or more amino acid substitutions compared to the amino acid sequence of wildtype IL-2 that reduces the affinity of the IL-2 peptide or functional fragment thereof for IL-2Rα (CD25), and one or more amino acid substitutions compared to the amino acid sequence of wildtype IL-2 that enhances the affinity of the IL-2 polypeptide or functional fragment thereof for IL-2R (CD122).

In some embodiments, the IL-2 binds to IL-2Rα with an affinity similar to or higher than wildtype IL-2. In some embodiments, the IL-2 preferentially binds to CD25 (e.g., alpha-biased). In some embodiments, the IL-2 has reduced affinity for CD122 and/or CD132. In some embodiments, the IL-2 comprises N88D and C125A relative to SEQ ID NO: 13. In some embodiments, the IL-2 comprises SEQ ID NO: 167.

(SEQ ID NO: 167)
APTSSSTKKTQLQLEHLLLDLQMILNGINNYKNPKLTRMLTFKFYMPKK
ATELKHLQCLEEELKPLEEVLNLAQSKNFHLRPRDLISDINVIVLELKG
SETTFMCEYADETATIVEFLNRWITFAQSIISTLT

In eukaryotic cells, naturally occurring IL-2 is synthesized as a precursor polypeptide of 153 amino acids, which has SEQ ID NO: 12.

(SEQ ID NO: 12)
MYRMQLLSCIALSLALVTNSAPTSSSTKKTQLQLEHLLLDLQMILNGIN
NYKNPKLTRMLTFKFYMPKKATELKHLQCLEEELKPLEEVLNLAQSKNF
HLRPRDLISNINVIVLELKGSETTFMCEYADETATIVEFLNRWITFCQS
IISTLT

This is then processed into mature IL-2 by the removal of amino acid residues 1-20. This results in a mature form of IL-2 consisting of 133 amino acids (amino acid residues 21-153), which has SEQ ID NO: 13.

(SEQ ID NO: 13)
APTSSSTKKTQLQLEHLLLDLQMILNGINNYKNPKLTRMLTFKFYMPKK
ATELKHLQCLEEELKPLEEVLNLAQSKNFHLRPRDLISNINVIVLELKG
SETTFMCEYADETATIVEFLNRWITFAQSIISTLT

“Functional fragments” of an IL-2 cytokine comprise a portion of a full-length cytokine protein which retains or has modified cytokine receptor binding capability (e.g., within at least 50%, 80%, 90%, 95%, 96%, 97%, 98%, 99% or 100% activity compared to the full length cytokine protein). Cytokine receptor binding capability can be shown, for example, by the capability of a cytokine to bind to the cytokine's cognate receptor or a component thereof (e.g., one or more chain(s) of a heterotrimeric receptor complex).

In some embodiments, the IL-2 cytokine or functional fragment thereof is any naturally occurring interleukin-2 (IL-2) protein or modified variant thereof capable of binding to an interleukin-2 receptor, particularly the IL-2Rα chain. In the context of IL-2 cytokine binding, the target protein could be IL-2R (comprising the IL-2Rα, IL-2Rβ, and IL-2Rγ chains), the IL-2Rα chain, the IL-2Rβ chain, or the IL-2Rα/β dimeric complex. In some embodiments, the IL-2 cytokine or functional fragment thereof comprises the amino acid sequence of amino acid residues 21-153 of SEQ ID NO: 13. In some embodiments, the IL-2 polypeptide or functional fragment thereof comprises the amino acid sequence of mature IL-2, SEQ ID NO: 12.

In some embodiments, the IL-2 cytokine or functional fragment thereof comprises an amino acid sequence having at least one amino acid modification as compared to the amino acid sequence of SEQ ID NO: 13. Each of the at least one amino acid modifications can be any amino acid modification, such as a substitution, insertion, or deletion. In some embodiments, the IL-2 cytokine or functional fragment thereof comprises an amino acid sequence having at least 1, at least 2, at least 3, at least 4, at least 5, at least 6, at least 7, at least 8, at least 9, or at least 10 amino acid substitutions as compared to the amino acid sequence of SEQ ID NO: 13. In some embodiments, the IL-2 cytokine or functional fragment thereof comprises an amino acid sequence having at least 5 amino acid substitutions as compared to the amino acid sequence of SEQ ID NO: 13.

In some embodiments, the IL-2 cytokine or functional fragment thereof comprises an amino acid sequence having at least 80% sequence identity to SEQ ID NO: 13. In some embodiments, the IL-2 cytokine or functional fragment thereof comprises an amino acid sequence having at least 85% sequence identity to SEQ ID NO: 13. In some embodiments, the IL-2 cytokine or functional fragment thereof comprises an amino acid sequence having at least 90% sequence identity to SEQ ID NO: 13. In some embodiments, the IL-2 cytokine or functional fragment thereof comprises an amino acid sequence having at least 91% sequence identity to SEQ ID NO: 13. In some embodiments, the IL-2 cytokine or functional fragment thereof comprises an amino acid sequence having at least 92% sequence identity to SEQ ID NO: 13. In some embodiments, the IL-2 cytokine or functional fragment thereof comprises an amino acid sequence having at least 93% sequence identity to SEQ ID NO: 13. In some embodiments, the IL-2 cytokine or functional fragment thereof comprises an amino acid sequence having at least 94% sequence identity to SEQ ID NO: 13. In some embodiments, the IL-2 cytokine or functional fragment thereof comprises an amino acid sequence having at least 95% sequence identity to SEQ ID NO: 13. In some embodiments, the IL-2 cytokine or functional fragment thereof comprises an amino acid sequence having at least 96% sequence identity to SEQ ID NO: 13. In some embodiments, the IL-2 cytokine or functional fragment thereof comprises an amino acid sequence having at least 97% sequence identity to SEQ ID NO: 13. In some embodiments, the IL-2 cytokine or functional fragment thereof comprises an amino acid sequence having at least 98% sequence identity to SEQ ID NO: 13. In some embodiments, the IL-2 cytokine or functional fragment thereof comprises an amino acid sequence having at least 99% sequence identity to SEQ ID NO: 13.

In some embodiments, the IL-2 cytokine or functional fragment thereof comprises an amino acid sequence having one or more amino acid substitutions as compared to the amino acid sequence of wild-type IL-2 of SEQ ID NO: 13 that reduces the affinity of the IL-2 peptide or functional fragment thereof for IL-2Rα (CD25). In some embodiments, the IL-2 cytokine or functional fragment thereof comprises an amino acid sequence having one or more amino acid substitutions as compared to the amino acid sequence of SEQ ID NO: 13, such that one or more of amino acid residues 38, 42, 45, and 62 is an alanine (A). In some embodiments, the IL-2 cytokine or functional fragment thereof comprises an amino acid sequence having one or more amino acid substitutions as compared to the amino acid sequence of SEQ ID NO: 13, such that amino acid residues 38, 42, 45, and 62 are an alanine (A).

In some embodiments, the IL-2 cytokine or functional fragment thereof comprises amino acid sequence substitution C125A as compared to the amino acid sequence of SEQ ID NO: 13.

In some embodiments, the IL-2 cytokine or functional fragment thereof comprises an amino acid sequence having one or more amino acid substitutions as compared to the amino acid sequence of SEQ ID NO: 13, such that amino acid residues 38, 42, 45, and 62 are an alanine (A) and amino acid residue 125 is an alanine (A). In some embodiments, the IL-2 cytokine or functional fragment thereof comprises an amino acid sequence having amino acid residues R38, F42, Y45, and E62 substituted for alanine in the amino acid sequence of SEQ ID NO: 13. In some embodiments, the IL-2 cytokine or functional fragment thereof comprises an amino acid sequence having amino acid residues R38, F42, Y45, and E62 substituted for alanine (A) and amino acid residue C125 substituted for alanine (A) in the amino acid sequence of SEQ ID NO: 13.

In some embodiments, the IL-2 cytokine or functional fragment thereof comprises the amino acid sequence of SEQ ID NO: 14.

(SEQ ID NO: 14)
APTSSSTKKTQLQLEHLLLDLQMILNGINNYKNPKLTAMLTAKFAMPKK
ATELKHLQCLEEALKPLEEVLNLAQSKNFHLRPRDLISNINVIVLELKG
SETTFMCEYADETATIVEFLNRWITFAQSIISTLT

In some embodiments, the IL-2 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 14. In some embodiments, the IL-2 cytokine or functional fragment thereof comprises an amino acid sequence having at least about 80% identity to SEQ ID NO: 14. In some embodiments, the IL-2 cytokine or functional fragment thereof comprises an amino acid sequence having at least about 85% identity to SEQ ID NO: 14. In some embodiments, the IL-2 cytokine or functional fragment thereof comprises an amino acid sequence having at least about 90% identity to SEQ ID NO: 14. In some embodiments, the IL-2 cytokine or functional fragment thereof comprises an amino acid sequence having at least about 92% identity to SEQ ID NO: 14. In some embodiments, the IL-2 cytokine or functional fragment thereof comprises an amino acid sequence having at least about 95% identity to SEQ ID NO: 14. In some embodiments, the IL-2 cytokine or functional fragment thereof comprises an amino acid sequence having at least about 97% identity to SEQ ID NO: 14. In some embodiments, the IL-2 cytokine or functional fragment thereof comprises an amino acid sequence having at least about 99% identity to SEQ ID NO: 14.

In some embodiments, the IL-2 cytokine or functional fragment thereof has one or more amino acid residues e.g., residues 1-3, removed as compared to the amino acid sequence of the mature IL-2 of SEQ ID NO: 12, for the purpose of removing an O-glycosylation site. In some embodiments, the IL-2 cytokine or functional fragment thereof has one or more amino acid residues substituted as compared to the amino acid sequence of the mature IL-2 of SEQ ID NO: 12, for the purpose of removing an O-glycosylation site. In some embodiments, the IL-2 cytokine or functional fragment thereof has one or more amino acid residues inserted, e.g., in the region of residues 1-3, as compared to the amino acid sequence of the mature IL-2 of SEQ ID NO: 12, for the purpose of removing an O-glycosylation site. In some embodiments, the IL-2 cytokine or functional fragment thereof does not have an O-glycosylation site within residues 1-3.

Interleukin 15 (IL-15)

Provided herein is an IL-15 cytokine or functional fragment thereof for use in a targeted cytokine or cleavage product thereof. A cytokine plays a role in cellular signalling, particularly in cells of the immune system. IL-15 is an interleukin, which is a type of cytokine signalling molecule in the immune system that regulates activities of white blood cells.

In eukaryotic cells, IL-15 is synthesized as a precursor polypeptide of 162 amino acids (SEQ ID NO: 92), which is then processed into mature IL-15 by the removal of amino acid residues 1-48. This results in a mature form of IL-15 consisting of 114 amino acids (amino acid residues 49-162) that is secreted in a mature, active form (see SEQ ID NO: 93).

IL-15 precursor polypeptide (SEQ ID NO: 92):
MRISKPHLRSISIQCYLCLLLNSHFLTEAGIHVFILGCFSAGLPKTEAN
WVNVISDLKKIEDLIQSMHIDATLYTESDVHPSCKVTAMKCFLLELQVI
SLESGDASIHDTVENLIILANNSLSSNGNVTESGCKECEELEEKNIKEF
LQSFVHIVQMFINTS
IL-15 mature polypeptide (SEQ ID NO: 93):
NWVNVISDLKKIEDLIQSMHIDATLYTESDVHPSCKVTAMKCFLLELQV
ISLESGDASIHDTVENLIILANNSLSSNGNVTESGCKECEELEEKNIKE
FLQSFVHIVQMFINTS

The term “IL-15” or “IL-15 polypeptide” as used herein refers to any interleukin-15 (IL-15) protein, or a functional fragment or variant thereof. The term encompasses any native IL-15 from any vertebrate source, including mammals such as primates (e.g., humans) and rodents (e.g., rats and mice). The term encompasses unprocessed IL-15 (e.g., a full length, precursor form of IL-15 that consists of amino acid residues 1-162) as well as any form of IL-15 that results from processing in the cell (e.g., a mature form of IL-15 that consists of amino acid residues 49-162). As such, the term encompasses a protein encoded by the amino acid sequence of SEQ ID NO: 93, as well as sequence variants thereof. The term also encompasses naturally occurring variants of IL-15. The term also encompasses non-naturally occurring variants of IL-15, such as truncations, deletions, forms where IL-15 is linked to another molecule, and variants caused by at least one amino acid change to the amino acid sequence (e.g., by substitution, addition, or deletion). In some aspects, the variants or homologs have at least 90%, 95%, 96%, 97%, 98%, 99% or 100% amino acid sequence identity across the whole sequence or a portion of the sequence (e.g., a 50, 100, or 114 continuous amino acid portion) compared to a naturally occurring IL-15 polypeptide, such as an IL-15 polypeptide encoded by the amino acid sequence of SEQ ID NO: 92 or 93. As such, the term “IL-15” or “IL-15 polypeptide” includes an IL-15 protein comprising the amino acid sequence of SEQ ID NO: 92 or 93, including variants thereof, such as variants created by one or more amino acid substitutions to the amino acid sequence of SEQ ID NO: 92 or 93.

“Functional fragments” of an IL-15 cytokine comprise a portion of a full length cytokine protein which retains or has modified cytokine receptor binding capability (e.g., within at least 50%, 80%, 90%, 95%, 96%, 97%, 98%, 99% or 100% activity compared to the full length cytokine protein). Cytokine receptor binding capability can be shown, for example, by the capability of a cytokine to bind to the cytokine's cognate receptor or a component thereof (e.g., one or more chain(s) of a heterotrimeric receptor complex).

In some embodiments, the IL-15 cytokine or functional fragment thereof is any naturally occurring interleukin-2 (IL-15) protein or modified variant thereof capable of binding to an interleukin-2 receptor, particularly the IL-15Rα chain.

In some embodiments, the IL-15 cytokine or fragment thereof comprises SEQ ID NO: 93 or a functional fragment thereof.

In some embodiments, the IL-15 cytokine or functional fragment thereof comprises an amino acid sequence of SEQ ID NO: 93.

In some embodiments, the IL-15 cytokine or functional fragment thereof comprises an amino acid sequence having at least one amino acid modification as compared to the amino acid sequence of SEQ ID NO: 93. Each of the at least one amino acid modifications can be any amino acid modification, such as a substitution, insertion, or deletion. In some embodiments, the IL-15 cytokine or functional fragment thereof comprises an amino acid sequence having at least 1, at least 2, at least 3, at least 4, at least 5, at least 6, at least 7, at least 8, at least 9, or at least 10 amino acid substitutions as compared to the amino acid sequence of SEQ ID NO: 93. In some embodiments, the IL-15 cytokine or functional fragment thereof comprises an amino acid sequence having at least 5 amino acid substitutions as compared to the amino acid sequence of SEQ ID NO: 93. In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 93.

In some embodiments, the IL-15 cytokine or functional fragment thereof comprises an amino acid sequence having at least about 85% sequence identity to SEQ ID NO: 93. In some embodiments, the IL-15 cytokine or functional fragment thereof comprises an amino acid sequence having at least about 90% sequence identity to SEQ ID NO: 93. In some embodiments, the IL-15 cytokine or functional fragment thereof comprises an amino acid sequence having at least about 92% sequence identity to SEQ ID NO: 93. In some embodiments, the IL-15 cytokine or functional fragment thereof comprises an amino acid sequence having at least about 93% sequence identity to SEQ ID NO: 93. In some embodiments, the IL-15 cytokine or functional fragment thereof comprises an amino acid sequence having at least about 94% sequence identity to SEQ ID NO: 93. In some embodiments, the IL-15 cytokine or functional fragment thereof comprises an amino acid sequence having at least about 95% sequence identity to SEQ ID NO: 93. In some embodiments, the IL-15 cytokine or functional fragment thereof comprises an amino acid sequence having at least about 96% sequence identity to SEQ ID NO: 93. In some embodiments, the IL-15 cytokine or functional fragment thereof comprises an amino acid sequence having at least about 97% sequence identity to SEQ ID NO: 93. In some embodiments, the IL-15 cytokine or functional fragment thereof comprises an amino acid sequence having at least about 98% sequence identity to SEQ ID NO: 93. In some embodiments, the IL-15 cytokine or functional fragment thereof comprises an amino acid sequence having at least about 99% sequence identity to SEQ ID NO: 93.

In some embodiments, the IL-15 cytokine or functional fragment thereof comprises an amino acid sequence having one or more amino acid substitutions as compared to the amino acid sequence of SEQ ID NO: 93.

In some embodiments, the IL-15 cytokine or functional fragment thereof comprises an amino acid sequence having one or more amino acid substitutions at positions D22, E46, E53 as compared to the amino acid sequence of SEQ ID NO: 93. In some embodiments, the IL-15 cytokine or functional fragment thereof comprises an amino acid sequence having one or more amino acid substitutions at positions D22, E46, E53, N71, N79, and N112 as compared to the amino acid sequence of SEQ ID NO: 93.

In some embodiments, the IL-15 cytokine or functional fragment thereof comprises an amino acid sequence having an amino acid substitution at position D22 as compared to the amino acid sequence of SEQ ID NO: 93.

In some embodiments, the IL-15 cytokine or functional fragment thereof comprises an amino acid sequence having an amino acid substitution at position E46 as compared to the amino acid sequence of SEQ ID NO: 93.

In some embodiments, the IL-15 cytokine or functional fragment thereof comprises an amino acid sequence having an amino acid substitution at position E53 as compared to the amino acid sequence of SEQ ID NO: 93.

In some embodiments, the IL-15 cytokine or functional fragment thereof comprises an amino acid sequence having an amino acid substitution at position N71 as compared to the amino acid sequence of SEQ ID NO: 93.

In some embodiments, the IL-15 cytokine or functional fragment thereof comprises an amino acid sequence having an amino acid substitution at position N79 as compared to the amino acid sequence of SEQ ID NO: 93.

In some embodiments, the IL-15 cytokine or functional fragment thereof comprises an amino acid sequence having an amino acid substitution at position N112 as compared to the amino acid sequence of SEQ ID NO: 93.

In some embodiments, the IL-15 cytokine or functional fragment thereof comprises an amino acid sequence having amino acid substitutions at positions E46 and E53 as compared to the amino acid sequence of SEQ ID NO: 93.

In some embodiments, the IL-15 cytokine or functional fragment thereof comprises an amino acid sequence having an amino acid substitution at position N71 and N79 as compared to the amino acid sequence of SEQ ID NO: 93.

In some embodiments, the IL-15 cytokine or functional fragment thereof comprises an amino acid sequence having an amino acid substitution at position N71 and N112 as compared to the amino acid sequence of SEQ ID NO: 93.

In some embodiments, the IL-15 cytokine or functional fragment thereof comprises an amino acid sequence having an amino acid substitution at position N79 and N112 as compared to the amino acid sequence of SEQ ID NO: 93.

In some embodiments, the IL-15 cytokine or functional fragment thereof comprises an amino acid sequence having an amino acid substitution at position N71, N79 and N112 as compared to the amino acid sequence of SEQ ID NO: 93.

In some embodiments, the amino acid substitution at position D22 is D22A.

In some embodiments, the amino acid substitution at position E46 is E46A.

In some embodiments, the amino acid substitution at position E46 is E46R.

In some embodiments, the amino acid substitution at position E46 is E46S.

In some embodiments, the amino acid substitution at position E53 is E53A.

In some embodiments, the amino acid substitution at position E53 is E53R.

In some embodiments, the amino acid substitution at position E53 is E53S.

In some embodiments, the amino acid substitution at position N71 is N71Q.

In some embodiments, the amino acid substitution at position N79 is N79Q.

In some embodiments, the amino acid substitution at position N112 is N112Q.

In some embodiments, the IL-15 cytokine or functional fragment thereof comprises an amino acid sequence having an amino acid substitution D22A as compared to the amino acid sequence of SEQ ID NO: 93.

In some embodiments, the IL-15 cytokine or functional fragment thereof comprises an amino acid sequence of SEQ ID NO: 94.

(SEQ ID NO: 94)
NWVNVISDLKKIEDLIQSMHIAATLYTESDVHPSCKVTAMKCFLLELQV
ISLESGDASIHDTVENLIILANNSLSSNGNVTESGCKECEELEEKNIKE
FLQSFVHIVQMFINTS

In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 94.

In some embodiments, the IL-15 cytokine or functional fragment thereof comprises an amino acid sequence having an amino acid substitution E46A as compared to the amino acid sequence of SEQ ID NO: 93. In some embodiments, the IL-15 cytokine or functional fragment thereof comprises an amino acid sequence of SEQ ID NO: 95.

(SEQ ID NO: 95)
NWVNVISDLKKIEDLIQSMHIDATLYTESDVHPSCKVTAMKCFLLALQV
ISLESGDASIHDTVENLIILANNSLSSNGNVTESGCKECEELEEKNIKE
FLQSFVHIVQMFINTS

In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 95.

In some embodiments, the IL-15 cytokine or functional fragment thereof comprises an amino acid sequence having amino acid substitutions E46A and E53A as compared to the amino acid sequence of SEQ ID NO: 93. In some embodiments, the IL-15 cytokine or functional fragment thereof comprises an amino acid sequence of SEQ ID NO: 96. In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 96.

(SEQ ID NO: 96)
NWVNVISDLKKIEDLIQSMHIDATLYTESDVHPSCKVTAMKCFLLALQV
ISLASGDASIHDTVENLIILANNSLSSNGNVTESGCKECEELEEKNIKE
FLQSFVHIVQMFINTS

In some embodiments, the IL-15 cytokine or functional fragment thereof comprises an amino acid sequence having amino acid substitutions E46R and E53R as compared to the amino acid sequence of SEQ ID NO: 93. In some embodiments, the IL-15 cytokine or functional fragment thereof comprises an amino acid sequence of SEQ ID NO: 97. In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 6.

(SEQ ID NO: 97)
NWVNVISDLKKIEDLIQSMHIDATLYTESDVHPSCKVTAMKCFLLRLQV
ISLRSGDASIHDTVENLIILANNSLSSNGNVTESGCKECEELEEKNIKE
FLQSFVHIVQMFINTS

In some embodiments, the IL-15 cytokine or functional fragment thereof comprises an amino acid sequence having amino acid substitutions E46S and E53S as compared to the amino acid sequence of SEQ ID NO: 93. In some embodiments, the IL-15 cytokine or functional fragment thereof comprises an amino acid sequence of SEQ ID NO: 98. In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 98.

(SEQ ID NO: 98)
NWVNVISDLKKIEDLIQSMHIDATLYTESDVHPSCKVTAMKCFLLSLQV
ISLSSGDASIHDTVENLIILANNSLSSNGNVTESGCKECEELEEKNIKE
FLQSFVHIVQMFINTS

In some embodiments, the IL-15 cytokine or functional fragment thereof comprises an amino acid sequence having an amino acid substitution E53A as compared to the amino acid sequence of SEQ ID NO: 93. In some embodiments, the IL-15 cytokine or functional fragment thereof comprises an amino acid sequence of SEQ ID NO: 99. In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 99.

(SEQ ID NO: 99)
NWVNVISDLKKIEDLIQSMHIDATLYTESDVHPSCKVTAMKCFLLELQV
ISLASGDASIHDTVENLIILANNSLSSNGNVTESGCKECEELEEKNIKE
FLQSFVHIVQMFINTS

In some embodiments, the IL-15 cytokine or functional fragment thereof comprises an amino acid sequence of SEQ ID NO: 100 and an amino acid sequence of SEQ ID NO: 101. In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 100 and an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 101.

(SEQ ID NO: 100)
NWVNVISDLKKIEDLIQS
(SEQ ID NO: 101)
KVTAMKCFLLELQVISLESGDASIHDTVENLIILANNSLSSNGNVTESG
CKECEELEEKNIKEFLQSFVHIVQMFINTS

In some embodiments, the IL-15 cytokine or functional fragment thereof comprises an amino acid sequence having an amino acid substitution N71Q as compared to the amino acid sequence of SEQ ID NO: 93. In some embodiments, the IL-15 cytokine or functional fragment thereof comprises an amino acid sequence of SEQ ID NO: 102. In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 102.

(SEQ ID NO: 102)
NWVNVISDLKKIEDLIQSMHIDATLYTESDVHPSCKVTAMKCFLLELQV
ISLESGDASIHDTVENLIILAQNSLSSNGNVTESGCKECEELEEKNIKE
FLQSFVHIVQMFINTS

In some embodiments, the IL-15 cytokine or functional fragment thereof comprises an amino acid sequence having an amino acid substitution N79Q as compared to the amino acid sequence of SEQ ID NO: 93. In some embodiments, the IL-15 cytokine or functional fragment thereof comprises an amino acid sequence of SEQ ID NO: 103. In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 103.

(SEQ ID NO: 103)
NWVNVISDLKKIEDLIQSMHIDATLYTESDVHPSCKVTAMKCFLLELQV
ISLESGDASIHDTVENLIILANNSLSSNGQVTESGCKECEELEEKNIKE
FLQSFVHIVQMFINTS

In some embodiments, the IL-15 cytokine or functional fragment thereof comprises an amino acid sequence having an amino acid substitution N112Q as compared to the amino acid sequence of SEQ ID NO: 93. In some embodiments, the IL-15 cytokine or functional fragment thereof comprises an amino acid sequence of SEQ ID NO: 104. In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 104.

(SEQ ID NO: 104)
NWVNVISDLKKIEDLIQSMHIDATLYTESDVHPSCKVTAMKCFLLELQV
ISLESGDASIHDTVENLIILANNSLSSNGNVTESGCKECEELEEKNIKE
FLQSFVHIVQMFIQTS

In some embodiments, the IL-15 cytokine or functional fragment thereof comprises an amino acid sequence having an amino acid substitution N71Q and N79Q as compared to the amino acid sequence of SEQ ID NO: 93. In some embodiments, the IL-15 cytokine or functional fragment thereof comprises an amino acid sequence of SEQ ID NO: 105. In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 105.

(SEQ ID NO: 105)
NWVNVISDLKKIEDLIQSMHIDATLYTESDVHPSCKVTAMKCFLLELQV
ISLESGDASIHDTVENLIILAQNSLSSNGQVTESGCKECEELEEKNIKE
FLQSFVHIVQMFINTS

In some embodiments, the IL-15 cytokine or functional fragment thereof comprises an amino acid sequence having an amino acid substitution N71Q and N112Q as compared to the amino acid sequence of SEQ ID NO: 93. In some embodiments, the IL-15 cytokine or functional fragment thereof comprises an amino acid sequence of SEQ ID NO: 106. In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 106.

(SEQ ID NO: 106)
NWVNVISDLKKIEDLIQSMHIDATLYTESDVHPSCKVTAMKCFLLELQV
ISLESGDASIHDTVENLIILAQNSLSSNGNVTESGCKECEELEEKNIKE
FLQSFVHIVQMFIQTS

In some embodiments, the IL-15 cytokine or functional fragment thereof comprises an amino acid sequence having an amino acid substitution N79Q and N112Q as compared to the amino acid sequence of SEQ ID NO: 93. In some embodiments, the IL-15 cytokine or functional fragment thereof comprises an amino acid sequence of SEQ ID NO: 107. In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 107.

(SEQ ID NO: 107)
NWVNVISDLKKIEDLIQSMHIDATLYTESDVHPSCKVTAMKCFLLELQV
ISLESGDASIHDTVENLIILANNSLSSNGQVTESGCKECEELEEKNIKE
FLQSFVHIVQMFIQTS

In some embodiments, the IL-15 cytokine or functional fragment thereof comprises an amino acid sequence having an amino acid substitution N71Q, N79Q and N112Q as compared to the amino acid sequence of SEQ ID NO: 93. In some embodiments, the IL-15 cytokine or functional fragment thereof comprises an amino acid sequence of SEQ ID NO: 108. In some embodiments, the IL-15 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 108.

(SEQ ID NO: 108)
NWVNVISDLKKIEDLIQSMHIDATLYTESDVHPSCKVTAMKCFLLELQV
ISLESGDASIHDTVENLIILAQNSLSSNGQVTESGCKECEELEEKNIKE
FLQSFVHIVQMFIQTS

In some embodiments, an additional mutation may be included in any of the sequences above at position N71. In some embodiments, the mutation is N71A, N71R, N71W, N71F, N71P, N71M, N71L, N71T, N71S, or N71Y.

In some embodiments, an additional mutation may be included in any of the sequences above at position S73. In some embodiments, the mutation is S73A, S73W, S73V, or S73M.

In some embodiments, an additional mutation may be included in any of the sequences above at one or more of amino acid positions N72, N79, V80, T81, and N112. In some embodiments, one or more additional mutations selected from N72A, N79A, V80A, T81A and N112R may be included in any of the sequences above.

In some embodiments, an additional mutation may be included in any of the sequences above at one or more of amino acid positions N72, S73, N79, V80, T81, and N112. In some embodiments, one or more additional mutations N72A, S73A, N79A, V80A, T81A, and N112 may be included in any of the sequences above.

In some embodiments, the IL-15 cytokine or functional fragment thereof has one or more amino acid residues e.g., residues 1-3 s removed as compared to the amino acid sequence of the mature IL-15 of SEQ ID NO: 93, for the purpose of removing an O-glycosylation site. In some embodiments, the IL-15 cytokine or functional fragment thereof has one or more amino acid residues substituted as compared to the amino acid sequence of the mature IL-15 of SEQ ID NO: 93, for the purpose of removing an O-glycosylation site. In some embodiments, the IL-15 cytokine or functional fragment thereof has one or more amino acid residues inserted, e.g., in the region of residues 1-3, as compared to the amino acid sequence of the mature IL-15 of SEQ ID NO: 93, for the purpose of removing an O-glycosylation site. In some embodiments, the IL-15 cytokine or functional fragment thereof does not have an O-glycosylation site within residues 1-3.

Interleukin 12 (IL-12)

Provided herein is an IL-12 cytokine or functional fragment thereof for use in a targeted cytokine or cleavage product thereof. A cytokine plays a role in cellular signalling, particularly in cells of the immune system. IL-12 is an interleukin, which is a type of cytokine signalling molecule in the immune system that regulates activities of white blood cells.

Endogenous IL-12 exists as two distinct molecules IL-12 p40 and IL-12 p35 that dimerize in the cell during biosynthesis.

The full sequences of IL-12 p40 and IL-12 p35 are (pro-peptides cleaved off during biosynthesis are shown) in bold):

IL-12 p40 subunit:
(SEQ ID NO: 109)
MCHQQLVISWFSLVFLASPLVAIWELKKDVYVVELDWYPDAPGEMVVLT
CDTPEEDGITWTLDQSSEVLGSGKTLTIQVKEFGDAGQYTCHKGGEVLS
HSLLLLHKKEDGIWSTDILKDQKEPKNKTFLRCEAKNYSGRFTCWWLTT
ISTDLTFSVKSSRGSSDPQGVTCGAATLSAERVRGDNKEYEYSVECQED
SACPAAEESLPIEVMVDAVHKLKYENYTSSFFIRDIIKPDPPKNLQLKP
LKNSRQVEVSWEYPDTWSTPHSYFSLTFCVQVQGKSKREKKDRVFTDKT
SATVICRKNASISVRAQDRYYSSSWSEWASVPCS
IL-12 p35 subunit:
(SEQ ID NO: 110)
MCPARSLLLVATLVLLDHLSLARNLPVATPDPGMFPCLHHSQNLLRAVS
NMLQKARQTLEFYPCTSEEIDHEDITKDKTSTVEACLPLELTKNESCLN
SRETSFITNGSCLASRKTSFMMALCLSSIYEDLKMYQVEFKTMNAKLLM
DPKRQIFLDQNMLAVIDELMQALNFNSETVPQKSSLEEPDFYKTKIKLC
ILLHAFRIRAVTIDRVMSYLNAS

The mature forms are as follows:

IL-12 p40 subunit:
(SEQ ID NO: 111)
IWELKKDVYVVELDWYPDAPGEMVVLTCDTPEEDGITWTLDQSSEVLGS
GKTLTIQVKEFGDAGQYTCHKGGEVLSHSLLLLHKKEDGIWSTDILKDQ
KEPKNKTFLRCEAKNYSGRFTCWWLTTISTDLTFSVKSSRGSSDPQGVT
CGAATLSAERVRGDNKEYEYSVECQEDSACPAAEESLPIEVMVDAVHKL
KYENYTSSFFIRDIIKPDPPKNLQLKPLKNSRQVEVSWEYPDTWSTPHS
YFSLTFCVQVQGKSKREKKDRVFTDKTSATVICRKNASISVRAQDRYYS
SSWSEWASVPCS
IL-12 p35 subunit:
(SEQ ID NO: 112)
RNLPVATPDPGMFPCLHHSQNLLRAVSNMLQKARQTLEFYPCTSEEIDH
EDITKDKTSTVEACLPLELTKNESCLNSRETSFITNGSCLASRKTSFMM
ALCLSSIYEDLKMYQVEFKTMNAKLLMDPKRQIFLDQNMLAVIDELMQA
LNFNSETVPQKSSLEEPDFYKTKIKLCILLHAFRIRAVTIDRVMSYLNA
S

They are expressed as two chains that covalently dimerize during biosynthesis through a disulfide bound between the two subunits: Cysteine C199 of the p40 subunit associates with Cysteine C96 of the p35 subunit.

“Functional fragments” of an IL-12 cytokine comprise a portion of a full-length cytokine protein which retains or has modified cytokine receptor binding capability (e.g., within at least 50%, 80%, 90%, 95%, 96%, 97%, 98%, 99% or 100% activity compared to the full length cytokine protein). Cytokine receptor binding capability can be shown, for example, by the capability of a cytokine to bind to the cytokine's cognate receptor or a component thereof.

In some embodiments, the IL-12 cytokine or functional fragment thereof is any naturally occurring interleukin-2 (IL-12) protein or modified variant thereof capable of binding to an interleukin-12 receptor.

In some embodiments, the IL-12 polypeptide or functional fragment thereof comprises an IL-12p40 polypeptide or functional fragment thereof covalently linked to an IL-12p35 polypeptide or functional fragment thereof.

The IL-12p40 polypeptide or functional fragment thereof may be attached to the first half life extension domain such that the first polypeptide chain comprises:

N′HL1-L1-MM C′

and the second polypeptide chain comprises:

N′HL2-L2-[IL-12p40-linker-IL-12p35]C′

where ‘IL-12p40’ is the IL-12p40 polypeptide or functional fragment thereof and ‘IL-12p35’ is the IL-12p35 polypeptide or functional fragment thereof.

In some embodiments, the IL-12p40 polypeptide comprises SEQ ID NO: 111. In some embodiments, the IL-12p40 polypeptide comprises an amino acid sequence having at least one amino acid modification as compared to the amino acid sequence of SEQ ID NO: 111. Each of the at least one amino acid modifications can be any amino acid modification, such as a substitution, insertion, or deletion. In some embodiments, the IL-12 cytokine or functional fragment thereof comprises an amino acid sequence having at least 1, at least 2, at least 3, at least 4, at least 5, at least 6, at least 7, at least 8, at least 9, or at least 10 amino acid substitutions as compared to the amino acid sequence of SEQ ID NO: 111. In some embodiments, the IL-12 cytokine or functional fragment thereof comprises an amino acid sequence having at least 5 amino acid substitutions as compared to the amino acid sequence of SEQ ID NO: 111.

The IL-12p40 polypeptide comprises a glycosaminoglycan (GAG)-binding domain. GAGs, such as heparin and heparan sulphate, have been shown to bind numerous growth factors and cytokines, including IL-12. The physiological significance of this binding is two-fold. First, GAGs can serve as co-receptors on cell surfaces to maintain high, local concentrations of cytokines. Second, GAGs can regulate bioactivities of growth factors and cytokines through multiple mechanisms including dimerization and protection from proteolytic degradation.

The GAG-binding domain in the mature form of the IL-12 p40 subunit is shown below in bold:

(SEQ ID NO: 113)
IWELKKDVYVVELDWYPDAPGEMVVLTCDTPEEDGITWTLDQSSEVLGSG
KTLTIQVKEFGDAGQYTCHKGGEVLSHSLLLLHKKEDGIWSTDILKDQKE
PKNKTFLRCEAKNYSGRFTCWWLTTISTDLTFSVKSSRGSSDPQGVTCGA
ATLSAERVRGDNKEYEYSVECQEDSACPAAEESLPIEVMVDAVHKLKYEN
YTSSFFIRDIIKPDPPKNLQLKPLKNSRQVEVSWEYPDTWSTPHSYFSLT
FCVQVQGKSKREKKDRVFTDKTSATVICRKNASISVRAQDRYYSSSWSEW
ASVPCS

Modifications to the GAG-binding domain KSKREKKDRV (SEQ ID NO: 114) has been shown herein to increase the PK profile of constructs comprising an IL-12 cytokine with a mutated GAG-binding domain, without any decrease in cytokine activity. Thus, in some embodiments, the IL-12p40 polypeptide comprises at least one amino acid modification to the GAG-binding domain. In some embodiments, the modification to the GAG-binding domain is a deletion mutation. In some embodiments, the modification to the GAG-binding domain is a deletion mutation and at least one substitution mutation.

In some embodiments, the GAG-binding domain comprises the amino acid sequence KDNTERV. In some embodiments, the IL-12p40 polypeptide comprises the amino acid sequence SEQ ID NO: 115.

(SEQ ID NO: 115)
IWELKKDVYVVELDWYPDAPGEMVVLTCDTPEEDGITWTLDQSSEVLGSG
KTLTIQVKEFGDAGQYTCHKGGEVLSHSLLLLHKKEDGIWSTDILKDQKE
PKNKTFLRCEAKNYSGRFTCWWLTTISTDLTFSVKSSRGSSDPQGVTCGA
ATLSAERVRGDNKEYEYSVECQEDSACPAAEESLPIEVMVDAVHKLKYEN
YTSSFFIRDIIKPDPPKNLQLKPLKNSRQVEVSWEYPDTWSTPHSYFSLT
FCVQVQGKDNTERVFTDKTSATVICRKNASISVRAQDRYYSSSWSEWASV
PCS

In some embodiments, the GAG-binding domain comprises the amino acid sequence KDNTEGRV. In some embodiments, the IL-12p40 polypeptide comprises the amino acid sequence SEQ ID NO: 116.

(SEQ ID NO: 116)
IWELKKDVYVVELDWYPDAPGEMVVLTCDTPEEDGITWTLDQSSEVLGSG
KTLTIQVKEFGDAGQYTCHKGGEVLSHSLLLLHKKEDGIWSTDILKDQKE
PKNKTFLRCEAKNYSGRFTCWWLTTISTDLTFSVKSSRGSSDPQGVTCGA
ATLSAERVRGDNKEYEYSVECQEDSACPAAEESLPIEVMVDAVHKLKYEN
YTSSFFIRDIIKPDPPKNLQLKPLKNSRQVEVSWEYPDTWSTPHSYFSLT
FCVQVQGKDNTEGRVFTDKTSATVICRKNASISVRAQDRYYSSSWSEWAS
VPCS

In some embodiments, the GAG-binding domain consists of the amino acid sequence KDNTERV. In some embodiments, the IL-12p40 polypeptide comprises the amino acid sequence SEQ ID NO: 115. In some embodiments, the GAG-binding domain consists of the amino acid sequence KDNTEGRV. In some embodiments, the IL-12p40 polypeptide comprises the amino acid sequence SEQ ID NO: 116.

In some embodiments, the IL-12p40 polypeptide comprises an amino acid sequence having one or more cysteine substitutions as compared to the amino acid sequence of SEQ ID NO: 111. In some embodiments, the IL-12p40 polypeptide comprises an amino acid sequence having an amino acid substitution at position C252 as compared to the amino acid sequence of SEQ ID NO: 111. In some embodiments, the amino acid substitution at position C252 is C252S. In some embodiments, the IL-12p40 polypeptide comprises an amino acid sequence of SEQ ID NO: 117. In some embodiments, the IL-12p40 polypeptide comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 117. In some embodiments, the IL-12p40 polypeptide consists of an amino acid sequence of SEQ ID NO: 117.

(SEQ ID NO: 117)
IWELKKDVYVVELDWYPDAPGEMVVLTCDTPEEDGITWTLDQSSEVLGSG
KTLTIQVKEFGDAGQYTCHKGGEVLSHSLLLLHKKEDGIWSTDILKDQKE
PKNKTFLRCEAKNYSGRFTCWWLTTISTDLTFSVKSSRGSSDPQGVTCGA
ATLSAERVRGDNKEYEYSVECQEDSACPAAEESLPIEVMVDAVHKLKYEN
YTSSFFIRDIIKPDPPKNLQLKPLKNSRQVEVSWEYPDTWSTPHSYFSLT
FSVQVQGKSKREKKDRVFTDKTSATVICRKNASISVRAQDRYYSSSWSEW
ASVPCS

In some embodiments, the IL-12p40 polypeptide comprises an amino acid sequence having one or more cysteine substitutions as compared to the amino acid sequence of SEQ ID NO: 111, and at least one amino acid modification to the GAG-binding domain. In some embodiments, the IL-12p40 polypeptide comprises an amino acid substitution at position C252S as compared to the amino acid sequence of SEQ ID NO: 111, and the GAG-binding domain comprises the amino acid sequence KDNTERV. In some embodiments, the IL-12p40 polypeptide comprises an amino acid substitution at position C252S as compared to the amino acid sequence of SEQ ID NO: 111, and the GAG-binding domain comprises the amino acid sequence KDNTEGRV. In some embodiments, the IL-12p40 polypeptide comprises an amino acid sequence of SEQ ID NO: 118. In some embodiments, the IL-12p40 polypeptide comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 118. In some embodiments, the IL-12p40 polypeptide consists of an amino acid sequence of SEQ ID NO: 118.

(SEQ ID NO: 118)
IWELKKDVYVVELDWYPDAPGEMVVLTCDTPEEDGITWTLDQSSEVLGSG
KTLTIQVKEFGDAGQYTCHKGGEVLSHSLLLLHKKEDGIWSTDILKDQKE
PKNKTFLRCEAKNYSGRFTCWWLTTISTDLTFSVKSSRGSSDPQGVTCGA
ATLSAERVRGDNKEYEYSVECQEDSACPAAEESLPIEVMVDAVHKLKYEN
YTSSFFIRDIIKPDPPKNLQLKPLKNSRQVEVSWEYPDTWSTPHSYFSLT
FSVQVQGKDNTEGRVFTDKTSATVICRKNASISVRAQDRYYSSSWSEWAS
VPCS

In some embodiments, the IL-12p35 polypeptide comprises SEQ ID NO: 112. In some embodiments, the IL-12p35 polypeptide comprises an amino acid sequence having at least one amino acid modification as compared to the amino acid sequence of SEQ ID NO: 112. Each of the at least one amino acid modifications can be any amino acid modification, such as a substitution, insertion, or deletion. In some embodiments, the IL-12 cytokine or functional fragment thereof comprises an amino acid sequence having at least 1, at least 2, at least 3, at least 4, at least 5, at least 6, at least 7, at least 8, at least 9, or at least 10 amino acid substitutions as compared to the amino acid sequence of SEQ ID NO: 112. In some embodiments, the IL-12 cytokine or functional fragment thereof comprises an amino acid sequence having at least 5 amino acid substitutions as compared to the amino acid sequence of SEQ ID NO: 112.

In some embodiments, the IL-12p40-IL-12p35 linker is between 5 and 20 amino acids in length.

In some embodiments, the IL-12p40-IL-12p35 linker is rich in amino acid residues G and S.

In some embodiments, the IL-12p40-IL-12p35 linker only includes amino acid residue types selected from the group consisting of G and S.

In some embodiments, the IL-12p40-IL-12p35 linker includes [(G)nS], where n=4 or 5.

In some embodiments, the IL-12p40-IL-12p35 linker includes a (GGGGS) repeat.

In some embodiments, IL-12p40-IL-12p35 linker comprises SEQ ID NO: 119. (GGGGSGGGGSGGGGS)

In some embodiments, the IL-12 cytokine or functional fragment thereof comprises SEQ ID NO: 120. In some embodiments, the IL-12 cytokine or functional fragment thereof comprises an amino acid sequence having at least one amino acid modification as compared to the amino acid sequences of SEQ ID NO: 111 and 112. Each of the at least one amino acid modifications can be any amino acid modification, such as a substitution, insertion, or deletion. In some embodiments, the IL-12 cytokine or functional fragment thereof comprises an amino acid sequence having at least 1, at least 2, at least 3, at least 4, at least 5, at least 6, at least 7, at least 8, at least 9, or at least 10 amino acid substitutions as compared to the amino acid sequences of SEQ ID NO: 111 and 112. In some embodiments, the IL-12 cytokine or functional fragment thereof comprises an amino acid sequence having at least 5 amino acid substitutions as compared to the amino acid sequences of SEQ ID NO: 111 and 112.

(SEQ ID NO: 120)
IWELKKDVYVVELDWYPDAPGEMVVLTCDTPEEDGITWTLDQSSEVLGSG
KTLTIQVKEFGDAGQYTCHKGGEVLSHSLLLLHKKEDGIWSTDILKDQKE
PKNKTFLRCEAKNYSGRFTCWWLTTISTDLTFSVKSSRGSSDPQGVTCGA
ATLSAERVRGDNKEYEYSVECQEDSACPAAEESLPIEVMVDAVHKLKYEN
YTSSFFIRDIIKPDPPKNLQLKPLKNSRQVEVSWEYPDTWSTPHSYFSLT
FCVQVQGKSKREKKDRVFTDKTSATVICRKNASISVRAQDRYYSSSWSEW
ASVPCSGGGGSGGGGSGGGGSRNLPVATPDPGMFPCLHHSQNLLRAVSNM
LQKARQTLEFYPCTSEEIDHEDITKDKTSTVEACLPLELTKNESCLNSRE
TSFITNGSCLASRKTSFMMA

In some embodiments, the IL-12 cytokine or functional fragment thereof comprises the amino acid sequence of SEQ ID NO: 120. In some embodiments, the IL-12 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 120.

In some embodiments, the IL-12 cytokine or functional fragment thereof comprises the amino acid sequence of SEQ ID NO: 121. In some embodiments, the IL-12 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 121.

(SEQ ID NO: 121)
IWELKKDVYVVELDWYPDAPGEMVVLTCDTPEEDGITWTLDQSSEVLGSG
KTLTIQVKEFGDAGQYTCHKGGEVLSHSLLLLHKKEDGIWSTDILKDQKE
PKNKTFLRCEAKNYSGRFTCWWLTTISTDLTFSVKSSRGSSDPQGVTCGA
ATLSAERVRGDNKEYEYSVECQEDSACPAAEESLPIEVMVDAVHKLKYEN
YTSSFFIRDIIKPDPPKNLQLKPLKNSRQVEVSWEYPDTWSTPHSYFSLT
FCVQVQGKDNTERVFTDKTSATVICRKNASISVRAQDRYYSSSWSEWASV
PCSGGGGSGGGGSGGGGSRNLPVATPDPGMFPCLHHSQNLLRAVSNMLQK
ARQTLEFYPCTSEEIDHEDITKDKTSTVEACLPLELTKNESCLNSRETSF
ITNGSCLASRKTSFMMALCLSSIYEDLKMYQVEFKTMNAKLLMDPKRQIF
LDQNMLAVIDELMQALNFNSETVPQKSSLEEPDFYKTKIKLCILLHAFRI
RAVTIDRVMSYLNAS

In some embodiments, the IL-12 cytokine or functional fragment thereof comprises the amino acid sequence of SEQ ID NO: 122. In some embodiments, the IL-12 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 122.

(SEQ ID NO: 122)
IWELKKDVYVVELDWYPDAPGEMVVLTCDTPEEDGITWTLDQSSEVLGSG
KTLTIQVKEFGDAGQYTCHKGGEVLSHSLLLLHKKEDGIWSTDILKDQKE
PKNKTFLRCEAKNYSGRFTCWWLTTISTDLTFSVKSSRGSSDPQGVTCGA
ATLSAERVRGDNKEYEYSVECQEDSACPAAEESLPIEVMVDAVHKLKYEN
YTSSFFIRDIIKPDPPKNLQLKPLKNSRQVEVSWEYPDTWSTPHSYFSLT
FCVQVQGKDNTEGRVFTDKTSATVICRKNASISVRAQDRYYSSSWSEWAS
VPCSGGGGSGGGGSGGGGSRNLPVATPDPGMFPCLHHSQNLLRAVSNMLQ
KARQTLEFYPCTSEEIDHEDITKDKTSTVEACLPLELTKNESCLNSRETS
FITNGSCLASRKTSFMMALCLSSIYEDLKMYQVEFKTMNAKLLMDPKRQI
FLDQNMLAVIDELMQALNFNSETVPQKSSLEEPDFYKTKIKLCILLHAFR
IRAVTIDRVMSYLNAS

In some embodiments, the IL-12 cytokine or functional fragment thereof comprises the amino acid sequence of SEQ ID NO: 123. In some embodiments, the IL-12 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 123.

(SEQ ID NO: 123)
IWELKKDVYVVELDWYPDAPGEMVVLTCDTPEEDGITWTLDQSSEVLGSG
KTLTIQVKEFGDAGQYTCHKGGEVLSHSLLLLHKKEDGIWSTDILKDQKE
PKNKTFLRCEAKNYSGRFTCWWLTTISTDLTFSVKSSRGSSDPQGVTCGA
ATLSAERVRGDNKEYEYSVECQEDSACPAAEESLPIEVMVDAVHKLKYEN
YTSSFFIRDIIKPDPPKNLQLKPLKNSRQVEVSWEYPDTWSTPHSYFSLT
FSVQVQGKSKREKKDRVFTDKTSATVICRKNASISVRAQDRYYSSSWSEW
ASVPCSGGGGSGGGGSGGGGSRNLPVATPDPGMFPCLHHSQNLLRAVSNM
LQKARQTLEFYPCTSEEIDHEDITKDKTSTVEACLPLELTKNESCLNSRE
TSFITNGSCLASRKTSFMMALCLSSIYEDLKMYQVEFKTMNAKLLMDPKR
QIFLDQNMLAVIDELMQALNFNSETVPQKSSLEEPDFYKTKIKLCILLHA
FRIRAVTIDRVMSYLNAS

In some embodiments, the IL-12 cytokine or functional fragment thereof comprises the amino acid sequence of SEQ ID NO: 124. In some embodiments, the IL-12 cytokine or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 124.

(SEQ ID NO: 124)
IWELKKDVYVVELDWYPDAPGEMVVLTCDTPEEDGITWTLDQSSEVLGSG
KTLTIQVKEFGDAGQYTCHKGGEVLSHSLLLLHKKEDGIWSTDILKDQKE
PKNKTFLRCEAKNYSGRFTCWWLTTISTDLTFSVKSSRGSSDPQGVTCGA
ATLSAERVRGDNKEYEYSVECQEDSACPAAEESLPIEVMVDAVHKLKYEN
YTSSFFIRDIIKPDPPKNLQLKPLKNSRQVEVSWEYPDTWSTPHSYFSLT
FSVQVQGKDNTEGRVFTDKTSATVICRKNASISVRAQDRYYSSSWSEWAS
VPCSGGGGSGGGGSGGGGSRNLPVATPDPGMFPCLHHSQNLLRAVSNMLQ
KARQTLEFYPCTSEEIDHEDITKDKTSTVEACLPLELTKNESCLNSRETS
FITNGSCLASRKTSFMMALC

### Masking Moiety

Provided herein is a masking moiety for use in a targeted cytokine. It will be understood that the masking moiety is cleaved from the targeted cytokine to form the cleavage product thereof. A masking moiety binds to the cytokine moiety and inhibits a biological activity of the cytokine. Upon cleavage, a masking moiety is released from a cytokine, activating the function of cytokine in a target of interest.

In some embodiments, a masking moiety comprises an agent, a peptide, or a polypeptide that binds to a cytokine.

In some embodiments, a masking moiety comprises a Fab, a single chain Fv (scFv), a single domain antibody (VHH), one or more CDRs, a variable heavy chain (VH), a variable light chain (VL), a Fab-like bispecific antibodies (bsFab), a single-domain antibody-linked Fab (s-Fab), an antibody, or a combination thereof. In some embodiments, a masking moiety comprises a Fab that binds to a cytokine. In some embodiments, a masking moiety comprises a single chain Fv (scFv) that binds to a cytokine. In some embodiments, a masking moiety comprises a single domain antibody (VHH) that binds to a cytokine. In some embodiments, a masking moiety comprises one or more CDRs that bind to a cytokine. In some embodiments, a masking moiety comprises a variable heavy chain (VH) that binds to a cytokine. In some embodiments, a masking moiety comprises a variable light chain (VL) that binds to a cytokine. In some embodiments, a masking moiety comprises a Fab-like bispecific antibodies (bsFab) that binds to a cytokine. In some embodiments, a masking moiety comprises a single-domain antibody-linked Fab (s-Fab) that binds to a cytokine. In some embodiments, a masking moiety comprises an antibody or a fragment thereof that binds to a cytokine. In some embodiments, the masking moiety comprises an antibody against the cytokine or a binding fragment of the antibody.

In some embodiments, the masking moiety is a receptor of the cytokine. In some embodiments, the masking moiety is a fragment of a receptor of the cytokine. In some embodiments, the masking moiety is an extracellular domain (ECD) of a receptor of the cytokine.

In some embodiments, the cytokine is an IL-1α or an IL-1β; and wherein the masking moiety is a Fab, a single chain Fv (scFv), or a single domain antibody against IL-1α or an IL-1β. In some embodiments, the cytokine is an IL-1α or an IL-1β; and wherein the masking moiety is CD121a, CDw121b, or a fragment thereof.

In some embodiments, the cytokine is an IL-2; and wherein the masking moiety is a Fab, a single chain Fv (scFv), or a single domain antibody against IL-2. In some embodiments, the cytokine is an IL-2; and wherein the masking moiety is IL-2Rα, IL-2Rβ, CD25, CD122, CD132, or a fragment thereof.

In some embodiments, the cytokine is an IL-18; and wherein the masking moiety is a Fab, a single chain Fv (scFv), or a single domain antibody against IL-18. In some embodiments, the cytokine is an IL-18; and wherein the masking moiety is IL-18Rα, IL-18Rβ, or a fragment thereof.

In some embodiments, the cytokine is an IL-4; and wherein the masking moiety is a Fab, a single chain Fv (scFv), or a single domain antibody against IL-4. In some embodiments, the cytokine is an IL-4; and wherein the masking moiety is CD124, CD213a13, CD132, or a fragment thereof.

In some embodiments, the cytokine is an IL-7; and wherein the masking moiety is a Fab, a single chain Fv (scFv), or a single domain antibody against IL-7. In some embodiments, the cytokine is an IL-7; and wherein the masking moiety is CD127, CD132, or a fragment thereof.

In some embodiments, the cytokine is an IL-9; and wherein the masking moiety is a Fab, a single chain Fv (scFv), or a single domain antibody against IL-9. In some embodiments, the cytokine is an IL-9, and wherein the masking moiety is IL-9R, CD132, or a fragment thereof.

In some embodiments, the cytokine is an IL-13; and wherein the masking moiety is a Fab, a single chain Fv (scFv), or a single domain antibody against IL-13. In some embodiments, the cytokine is an IL-13; and wherein the masking moiety is CD213a1, CD213a2, or a fragment thereof.

In some embodiments, the cytokine is an IL-15; and wherein the masking moiety is a Fab, a single chain Fv (scFv), or a single domain antibody against IL-15. In some embodiments, the cytokine is an IL-15; and wherein the masking moiety is IL-15Ra, CD122, CD132, or a fragment thereof.

In some embodiments, the cytokine is an IL-3; and wherein the masking moiety is a Fab, a single chain Fv (scFv), or a single domain antibody against IL-3. In some embodiments, the cytokine is an IL-3; and wherein the masking moiety is CD123, CDw131, or a fragment thereof.

In some embodiments, the cytokine is an IL-5; and wherein the masking moiety is a Fab, a single chain Fv (scFv), or a single domain antibody against IL-5. In some embodiments, the cytokine is an IL-5; and wherein the masking moiety is CDw125, CD131, or a fragment thereof.

In some embodiments, the cytokine is a GM-CSF; and wherein the masking moiety is a Fab, a single chain Fv (scFv), or a single domain antibody against GM-CSF. In some embodiments, the cytokine is a GM-CSF; and wherein the masking moiety is CD116, CDw131, or a fragment thereof.

In some embodiments, the cytokine is an IL-6; and wherein the masking moiety is a Fab, a single chain Fv (scFv), or a single domain antibody against IL-6. In some embodiments, the cytokine is an IL-6; and wherein the masking moiety is CD126, CD130, or a fragment thereof.

In some embodiments, the cytokine is an IL-11; and wherein the masking moiety is a Fab, a single chain Fv (scFv), or a single domain antibody against IL-11. In some embodiments, the cytokine is an IL-11; and wherein the masking moiety is IL-11Ra, CD130, or a fragment thereof.

In some embodiments, the cytokine is a G-CSF; and wherein the masking moiety is a Fab, a single chain Fv (scFv), or a single domain antibody against G-CSF. In some embodiments, the cytokine is a G-CSF; and wherein the masking moiety is CD114, or a fragment thereof.

In some embodiments, the cytokine is an IL-12; and wherein the masking moiety is a Fab, a single chain Fv (scFv), or a single domain antibody against IL-12. In some embodiments, the cytokine is an IL-12; and wherein the masking moiety is CD212, or a fragment thereof.

In some embodiments, the cytokine is an LIF; and wherein the masking moiety is a Fab, a single chain Fv (scFv), or a single domain antibody against LIF. In some embodiments, the cytokine is an LIF; and wherein the masking moiety is LIFR, CD130 or a fragment thereof.

In some embodiments, the cytokine is an OSM; and wherein the masking moiety is a Fab, a single chain Fv (scFv), or a single domain antibody against OSM. In some embodiments, the cytokine is an OSM; and wherein the masking moiety is OSMR, CD130, or a fragment thereof.

In some embodiments, the cytokine is an IL-10; and wherein the masking moiety is a Fab, a single chain Fv (scFv), or a single domain antibody against IL-10. In some embodiments, the cytokine is an IL-10; and wherein the masking moiety is CDw210 or a fragment thereof.

In some embodiments, the cytokine is an IL-20; and wherein the masking moiety is a Fab, a single chain Fv (scFv), or a single domain antibody against IL-20. In some embodiments, the cytokine is an IL-20; and wherein the masking moiety is IL-20Rα, IL-20Rβ, or a fragment thereof.

In some embodiments, the cytokine is an IL-14; and wherein the masking moiety is a Fab, a single chain Fv (scFv), or a single domain antibody against IL-14. In some embodiments, the cytokine is an IL-14; and wherein the masking moiety is IL-14R, or a fragment thereof.

In some embodiments, the cytokine is an IL-16; and wherein the masking moiety is a Fab, a single chain Fv (scFv), or a single domain antibody against IL-16. In some embodiments, the cytokine is an IL-16; and wherein the masking moiety is CD4, or a fragment thereof.

In some embodiments, the cytokine is an IL-17; and wherein the masking moiety is a Fab, a single chain Fv (scFv), or a single domain antibody against IL-17. In some embodiments, the cytokine is an IL-17; and wherein the masking moiety is CDw217, or a fragment thereof.

In some embodiments, the cytokine is an IFN-α; and wherein the masking moiety is a Fab, a single chain Fv (scFv), or a single domain antibody against IFN-α. In some embodiments, the cytokine is an IFN-α; and wherein the masking moiety is CD118, or a fragment thereof.

In some embodiments, the cytokine is an IFN-β; and wherein the masking moiety is a Fab, a single chain Fv (scFv), or a single domain antibody against IFN-β. In some embodiments, the cytokine is an IFN-β; and wherein the masking moiety is CD118, or a fragment thereof.

In some embodiments, the cytokine is an IFN-γ; and wherein the masking moiety is a Fab, a single chain Fv (scFv), or a single domain antibody against IFN-γ. In some embodiments, the cytokine is an IFN-γ; and wherein the masking moiety is CDw119, or a fragment thereof.

In some embodiments, the cytokine is a CD154; and wherein the masking moiety is a Fab, a single chain Fv (scFv), or a single domain antibody against CD154. In some embodiments, the cytokine is a CD154; and wherein the masking moiety is CD40, or a fragment thereof.

In some embodiments, the cytokine is an LT-β; and wherein the masking moiety is a Fab, a single chain Fv (scFv), or a single domain antibody against LT-β. In some embodiments, the cytokine is an LT-β; and wherein the masking moiety is LT-βR, or a fragment thereof.

In some embodiments, the cytokine is a TNF-α; and wherein the masking moiety is a Fab, a single chain Fv (scFv), or a single domain antibody against TNF-α. In some embodiments, the cytokine is a TNF-α; and wherein the masking moiety is CD120a, CD120b, or a fragment thereof.

In some embodiments, the cytokine is a TNF-β; and wherein the masking moiety is a Fab, a single chain Fv (scFv), or a single domain antibody against TMF-β. In some embodiments, the cytokine is a TNF-β; and wherein the masking moiety is CD120a, CD120b, or a fragment thereof.

In some embodiments, the cytokine is a 4-1BBL; and wherein the masking moiety is a Fab, a single chain Fv (scFv), or a single domain antibody against 4-1BBL. In some embodiments, the cytokine is a 4-1BBL; and wherein the masking moiety is CDw137, 4-1BB, or a fragment thereof.

In some embodiments, the cytokine is an APRIL; and wherein the masking moiety is a Fab, a single chain Fv (scFv), or a single domain antibody against APRIL. In some embodiments, the cytokine is an APRIL; and wherein the masking moiety is BCMA, TACI, or a fragment thereof.

In some embodiments, the cytokine is a CD70; and wherein the masking moiety is a Fab, a single chain Fv (scFv), or a single domain antibody against CD70. In some embodiments, the cytokine is a CD70; and wherein the masking moiety is CD27, or a fragment thereof.

In some embodiments, the cytokine is a CD153; and wherein the masking moiety is a Fab, a single chain Fv (scFv), or a single domain antibody against CD153. In some embodiments, the cytokine is a CD153; and wherein the masking moiety is CD30, or a fragment thereof.

In some embodiments, the cytokine is a CD178; and wherein the masking moiety is a Fab, a single chain Fv (scFv), or a single domain antibody against CD178. In some embodiments, the cytokine is a CD178; and wherein the masking moiety is CD95, or a fragment thereof.

In some embodiments, the cytokine is a GITRL; and wherein the masking moiety is a Fab, a single chain Fv (scFv), or a single domain antibody against GITRL. In some embodiments, the cytokine is a GITRL; and wherein the masking moiety is GITR, or a fragment thereof.

In some embodiments, the cytokine is a LIGHT; and wherein the masking moiety is a Fab, a single chain Fv (scFv), or a single domain antibody against LIGHT. In some embodiments, the cytokine is a LIGHT; and wherein the masking moiety is LTβR, HVEM, or a fragment thereof.

In some embodiments, the cytokine is an OX40; and wherein the masking moiety is a Fab, a single chain Fv (scFv), or a single domain antibody against OX40. In some embodiments, the cytokine is an OX40; and wherein the masking moiety is OX40, or a fragment thereof.

In some embodiments, the cytokine is a TALL-1; and wherein the masking moiety is a Fab, a single chain Fv (scFv), or a single domain antibody against TALL-1. In some embodiments, the cytokine is a TALL-1; and wherein the masking moiety is BCMA, TACI, or a fragment thereof.

In some embodiments, the cytokine is a TRAIL; and wherein the masking moiety is a Fab, a single chain Fv (scFv), or a single domain antibody against TRAIL. In some embodiments, the cytokine is a TRAIL; and wherein the masking moiety is TRAILR1-4, or a fragment thereof.

In some embodiments, the cytokine is a TWEAK; and wherein the masking moiety is a Fab, a single chain Fv (scFv), or a single domain antibody against TWEAK. In some embodiments, the cytokine is a TWEAK; and wherein the masking moiety is Apo3, or a fragment thereof.

In some embodiments, the cytokine is a TRANCE; and wherein the masking moiety is a Fab, a single chain Fv (scFv), or a single domain antibody against TRANCE. In some embodiments, the cytokine is a TRANCE; and wherein the masking moiety is RANK, OPG, or a fragment thereof.

In some embodiments, the cytokine is a TGF-β1; and wherein the masking moiety is a Fab, a single chain Fv (scFv), or a single domain antibody against TGF-β1. In some embodiments, the cytokine is a TGF-β1; and wherein the masking moiety is TGF-βR1, or a fragment thereof.

In some embodiments, the cytokine is a TGF-β2; and wherein the masking moiety is a Fab, a single chain Fv (scFv), or a single domain antibody against TGF-β2. In some embodiments, the cytokine is a TGF-β2; and wherein the masking moiety is TGF-βR2, or a fragment thereof.

In some embodiments, the cytokine is a TGF-β3; and wherein the masking moiety is a Fab, a single chain Fv (scFv), or a single domain antibody against TGF-β3. In some embodiments, the cytokine is a TGF-β3; and wherein the masking moiety is TGF-βR3, or a fragment thereof.

In some embodiments, the cytokine is an Epo; and wherein the masking moiety is a Fab, a single chain Fv (scFv), or a single domain antibody against Epo. In some embodiments, the cytokine is an Epo; and wherein the masking moiety is EpoR, or a fragment thereof.

In some embodiments, the cytokine is a Tpo; and wherein the masking moiety is a Fab, a single chain Fv (scFv), or a single domain antibody against Tpo. In some embodiments, the cytokine is a Tpo; and wherein the masking moiety is TpoR, or a fragment thereof.

In some embodiments, the cytokine is an Flt-3L; and wherein the masking moiety is a Fab, a single chain Fv (scFv), or a single domain antibody against Flt-3L. In some embodiments, the cytokine is an Flt-3L; and wherein the masking moiety is Flt-3, or a fragment thereof.

In some embodiments, the cytokine is an SCF; and wherein the masking moiety is a Fab, a single chain Fv (scFv), or a single domain antibody against SCF. In some embodiments, the cytokine is an SCF; and wherein the masking moiety is CD117, or a fragment thereof.

In some embodiments, the cytokine is an M-CSF; and wherein the masking moiety is a Fab, a single chain Fv (scFv), or a single domain antibody against M-CSF. In some embodiments, the cytokine is an M-CSF; and wherein the masking moiety is CD115, or a fragment thereof.

In some embodiments, the cytokine is an MSP; and wherein the masking moiety is a Fab, a single chain Fv (scFv), or a single domain antibody against MSP. In some embodiments, the cytokine is an MSP; and wherein the masking moiety is CDw136, or a fragment thereof.

In some embodiments, the cytokine is an IL-15 agonist polypeptide; and wherein the masking moiety is a Fab, a single chain Fv (scFv), or a single domain antibody against IL-15. In some embodiments, the cytokine moiety comprises an IL-15 agonist polypeptide, wherein the chimeric molecule further comprises the sushi domain of IL-15 receptor a (IL-15Ra sushi domain).

In some embodiments, a cytokine is an IL-2 agonist polypeptide; and wherein the masking moiety is the extracellular domain of IL-21 receptor a (IL-21Ra ECD) or a functional analog thereof. In some embodiments, the cytokine is an IL-2 agonist polypeptide or an IL-15 agonist polypeptide; and wherein the masking moiety is the extracellular domain of IL-2 receptor b (IL-2R (3 ECD). In some embodiments, the cytokine is an IL-21 agonist polypeptide; and wherein the masking moiety is a Fab, a single chain Fv (scFv) or a single domain antibody against IL-21.

In some embodiments, a cytokine is an IL-2 agonist polypeptide; and wherein the masking moiety is the extracellular domain of IL-21 receptor a (IL-21Ra ECD) or a functional analog thereof. In some embodiments, the cytokine is an IL-2 agonist polypeptide or an IL-15 agonist polypeptide; and wherein the masking moiety is the extracellular domain of IL-2 receptor b (IL-2R (3 ECD). In some embodiments, the cytokine is an IL-21 agonist polypeptide; and wherein the masking moiety is a Fab, a single chain Fv (scFv) or a single domain antibody against IL-21.

CD122 (Masking Moiety for IL-2 and IL-15)

The masking moiety masks the cytokine or functional fragment thereof in the targeted cytokine thereby reducing or preventing binding of the cytokine or functional fragment thereof to its cognate receptor. In some embodiments, the masking moiety reduces or prevents binding of the IL-2 cytokine or functional fragment thereof to IL-2Rα (CD25). In some embodiments, the masking moiety as provided herein refers to a moiety capable of binding to, or otherwise exhibiting an affinity for the IL-2 cytokine or functional fragment thereof, such as an anti-IL-2 antibody or IL-2 cognate receptor protein. In some embodiments, the masking moiety reduces or prevents binding of the IL-15 cytokine or functional fragment thereof to IL-15Rα. In some embodiments, the masking moiety as provided herein refers to a moiety capable of binding to, or otherwise exhibiting an affinity for the IL-15 cytokine or functional fragment thereof, such as an anti-IL-15 antibody or IL-15 cognate receptor protein. Methods for determining the extent of binding of a protein (e.g., cytokine) to a cognate protein (e.g., cytokine receptor) are well known in the art.

In some embodiments, the masking moiety comprises an IL-2 cytokine receptor, or a subunit or functional fragment thereof.

In some embodiments, the masking moiety comprises CD122 (also referred to as IL-2Rβ) or a fragment, portion, or variant thereof that retains or otherwise demonstrates an affinity to IL-2 and IL-15.

In some embodiments, the masking moiety comprises the amino acid sequence of SEQ ID NO: 15.

(SEQ ID NO: 15)
AVNGTSQFTCFYNSRANISCVWSQDGALQDTSCQVHAWPDRRRWNQTCEL
LPVSQASWACNLILGAPDSQKLTTVDIVTLRVLCREGVRWRVMAIQDFKP
FENLRLMAPISLQVVHVETHRCNISWEISQASHYFERHLEFEARTLSPGH
TWEEAPLLTLKQKQEWICLETLTPDTQYEFQVRVKPLQGEFTTWSPWSQP
LAFRTKPAALGKD

In some embodiments, the masking moiety comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 15. In some embodiments, the masking moiety comprises an amino acid sequence having the amino acid sequence of SEQ ID NO: 15 with one to four amino acid substitutions. In some embodiments, the masking moiety comprises an amino acid sequence having the amino acid sequence of SEQ ID NO: 15 with one or two amino acid substitutions.

In some embodiments, the CD122 or a fragment, portion or variant thereof has mutation at amino acid position C122 as compared to CD122 of SEQ ID NO: 15.

In some embodiments, the CD122 or a fragment, portion or variant thereof has mutation C122S at amino acid position 122 as compared to CD122 of SEQ ID NO: 15.

In some embodiments, the masking moiety comprises an amino acid sequence of SEQ ID NO: 15 with a C122 mutation.

In some embodiments, the masking moiety comprises an amino acid sequence of SEQ ID NO: 15 with a C122S mutation.

In some embodiments, the CD122 or a fragment, portion or variant thereof has mutation at amino acid position C168 as compared to CD122 of SEQ ID NO: 15.

In some embodiments, the CD122 or a fragment, portion or variant thereof has mutation C168S at amino acid position 168 as compared to CD122 of SEQ ID NO: 15.

In some embodiments, the masking moiety comprises an amino acid sequence of SEQ ID NO: 15 with a C168 mutation.

In some embodiments, the masking moiety comprises an amino acid sequence of SEQ ID NO: 15 with a C168S mutation.

In some embodiments, the CD122 or a fragment, portion or variant thereof has mutation at amino acid positions C122 and C168 as compared to CD122 of SEQ ID NO: 15.

In some embodiments, the CD122 or a fragment, portion or variant thereof has mutation C122S and C168S as compared to CD122 of SEQ ID NO: 15.

In some embodiments, the masking moiety comprises an amino acid sequence of SEQ ID NO: 16.

(SEQ ID NO: 16)
AVNGTSQFTCFYNSRANISCVWSQDGALQDTSCQVHAWPDRRRWNQTCEL
LPVSQASWACNLILGAPDSQKLTTVDIVTLRVLCREGVRWRVMAIQDFKP
FENLRLMAPISLQVVHVETHRSNISWEISQASHYFERHLEFEARTLSPGH
TWEEAPLLTLKQKQEWISLETLTPDTQYEFQVRVKPLQGEFTTWSPWSQP
LAFRTKPAALGKD

In some embodiments, the masking moiety comprises an amino acid sequence having at least about 80% identity to SEQ ID NO: 16. In some embodiments, the masking moiety comprises an amino acid sequence having at least about 85% identity to SEQ ID NO: 16. In some embodiments, the masking moiety comprises an amino acid sequence having at least about 90% identity to SEQ ID NO: 16. In some embodiments, the masking moiety comprises an amino acid sequence having at least about 91% identity to SEQ ID NO: 16. In some embodiments, the masking moiety comprises an amino acid sequence having at least about 92% identity to SEQ ID NO: 16. In some embodiments, the masking moiety comprises an amino acid sequence having at least about 93% identity to SEQ ID NO: 16. In some embodiments, the masking moiety comprises an amino acid sequence having at least about 94% identity to SEQ ID NO: 16. In some embodiments, the masking moiety comprises an amino acid sequence having at least about 95% identity to SEQ ID NO: 16. In some embodiments, the masking moiety comprises an amino acid sequence having at least about 96% identity to SEQ ID NO: 16. In some embodiments, the masking moiety comprises an amino acid sequence having at least about 97% identity to SEQ ID NO: 16. In some embodiments, the masking moiety comprises an amino acid sequence having at least about 98% identity to SEQ ID NO: 16. In some embodiments, the masking moiety comprises an amino acid sequence having at least about 99% identity to SEQ ID NO: 16.

In some embodiments, a masking moiety has a mutation at amino acid positions F8 as compared to CD122 of SEQ ID NO: 15. In some embodiments, a masking moiety has a mutation F8C as compared to CD122 of SEQ ID NO: 15.

In some embodiments, a masking moiety has a mutation at amino acid positions A94 as compared to CD122 of SEQ ID NO: 15. In some embodiments, a masking moiety has a mutation A94C as compared to CD122 of SEQ ID NO: 15.

In some embodiments, a masking moiety has a mutation at amino acid positions L106 as compared to CD122 of SEQ ID NO: 15. In some embodiments, a masking moiety has a mutation L106C as compared to CD122 of SEQ ID NO: 15.

In some embodiments, a masking moiety has a mutation at amino acid positions V117 as compared to CD122 of SEQ ID NO: 15. In some embodiments, a masking moiety has a mutation V117C as compared to CD122 of SEQ ID NO: 15.

In some embodiments, a masking moiety has a mutation at amino acid positions C122 as compared to CD122 of SEQ ID NO: 15. In some embodiments, a masking moiety has a mutation C122S as compared to CD122 of SEQ ID NO: 15. In some embodiments, a masking moiety has a mutation C122V as compared to CD122 of SEQ ID NO: 15. In some embodiments, a masking moiety has a mutation C122A as compared to CD122 of SEQ ID NO: 15.

In some embodiments, a masking moiety has a mutation at amino acid positions N123 as compared to CD122 of SEQ ID NO: 15. In some embodiments, a masking moiety has a mutation N123C as compared to CD122 of SEQ ID NO: 15. In some embodiments, a masking moiety has a mutation N123Q as compared to CD122 of SEQ ID NO: 15.

In some embodiments, a masking moiety has a mutation at amino acid positions C168 as compared to CD122 of SEQ ID NO: 15. In some embodiments, a masking moiety has a mutation C168S as compared to CD122 of SEQ ID NO: 15. In some embodiments, a masking moiety has a mutation C168V as compared to CD122 of SEQ ID NO: 15. In some embodiments, a masking moiety has a mutation C168A as compared to CD122 of SEQ ID NO: 15.

In some embodiments, a masking moiety has a mutation at amino acid positions L169 as compared to CD122 of SEQ ID NO: 15. In some embodiments, a masking moiety has a mutation L169C as compared to CD122 of SEQ ID NO: 15.

In some embodiments, a masking moiety has a mutation at amino acid positions Q177 as compared to CD122 of SEQ ID NO: 15. In some embodiments, a masking moiety has a mutation Q177C as compared to CD122 of SEQ ID NO: 15.

In some embodiments, a masking moiety has a mutation at amino acid positions V184 as compared to CD122 of SEQ ID NO: 15. In some embodiments, a masking moiety has a mutation V184C as compared to CD122 of SEQ ID NO: 15.

In some embodiments, a masking moiety has a mutation at amino acid positions S195 as compared to CD122 of SEQ ID NO: 15. In some embodiments, a masking moiety has a mutation S195C as compared to CD122 of SEQ ID NO: 15.

In some embodiments, a masking moiety has a mutation at amino acid positions R204 as compared to CD122 of SEQ ID NO: 15. In some embodiments, a masking moiety has a mutation R204C as compared to CD122 of SEQ ID NO: 15.

In some embodiments, a masking moiety has mutations C122V/C168V as compared to CD122 of SEQ ID NO: 15. In some embodiments, a masking moiety comprises an amino acid sequence of SEQ ID NO: 17.

(SEQ ID NO: 17)
AVNGTSQFTCFYNSRANISCVWSQDGALQDTSCQVHAWPDRRRWNQTCEL
LPVSQASWACNLILGAPDSQKLTTVDIVTLRVLCREGVRWRVMAIQDFKP
FENLRLMAPISLQVVHVETHRVNISWEISQASHYFERHLEFEARTLSPGH
TWEEAPLLTLKQKQEWIVLETLTPDTQYEFQVRVKPLQGEFTTWSPWSQP
LAFRTKPAALGKD

In some embodiments, a masking moiety has mutations C122A/C168V as compared to CD122 of SEQ ID NO: 15. In some embodiments, a masking moiety comprises an amino acid sequence of SEQ ID NO: 18.

(SEQ ID NO: 18)
AVNGTSQFTCFYNSRANISCVWSQDGALQDTSCQVHAWPDRRRWNQTCEL
LPVSQASWACNLILGAPDSQKLTTVDIVTLRVLCREGVRWRVMAIQDFKP
FENLRLMAPISLQVVHVETHRANISWEISQASHYFERHLEFEARTLSPGH
TWEEAPLLTLKQKQEWIVLETLTPDTQYEFQVRVKPLQGEFTTWSPWSQP
LAFRTKPAALGKD

In some embodiments, a masking moiety has a mutations C168V as compared to CD122 of SEQ ID NO: 15. In some embodiments, a masking moiety comprises an amino acid sequence of SEQ ID NO: 19.

(SEQ ID NO: 19)
AVNGTSQFTCFYNSRANISCVWSQDGALQDTSCQVHAWPDRRRWNQTCEL
LPVSQASWACNLILGAPDSQKLTTVDIVTLRVLCREGVRWRVMAIQDFKP
FENLRLMAPISLQVVHVETHRCNISWEISQASHYFERHLEFEARTLSPGH
TWEEAPLLTLKQKQEWIVLETLTPDTQYEFQVRVKPLQGEFTTWSPWSQP
LAFRTKPAALGKD

In some embodiments, a masking moiety has mutations C122V/C168A as compared to CD122 of SEQ ID NO: 15. In some embodiments, a masking moiety comprises an amino acid sequence of SEQ ID NO: 20.

(SEQ ID NO: 20)
AVNGTSQFTCFYNSRANISCVWSQDGALQDTSCQVHAWPDRRRWNQTCE
LLPVSQASWACNLILGAPDSQKLTTVDIVTLRVLCREGVRWRVMAIQDF
KPFENLRLMAPISLQVVHVETHRVNISWEISQASHYFERHLEFEARTLS
PGHTWEEAPLLTLKQKQEWIALETLTPDTQYEFQVRVKPLQGEFTTWSP
WSQPLAFRTKPAALGKD

In some embodiments, a masking moiety has mutations C122A/N123C as compared to CD122 of SEQ ID NO: 15. In some embodiments, a masking moiety comprises an amino acid sequence of SEQ ID NO: 21.

(SEQ ID NO: 21)
AVNGTSQFTCFYNSRANISCVWSQDGALQDTSCQVHAWPDRRRWNQTCE
LLPVSQASWACNLILGAPDSQKLTTVDIVTLRVLCREGVRWRVMAIQDF
KPFENLRLMAPISLQVVHVETHRACISWEISQASHYFERHLEFEARTLS
PGHTWEEAPLLTLKQKQEWICLETLTPDTQYEFQVRVKPLQGEFTTWSP
WSQPLAFRTKPAALGKD

In some embodiments, the masking moiety comprises an amino acid sequence having at least about 80% identity to SEQ ID NO: 21. In some embodiments, the masking moiety comprises an amino acid sequence having at least about 85% identity to SEQ ID NO: 21. In some embodiments, the masking moiety comprises an amino acid sequence having at least about 90% identity to SEQ ID NO: 21. In some embodiments, the masking moiety comprises an amino acid sequence having at least about 91% identity to SEQ ID NO: 21. In some embodiments, the masking moiety comprises an amino acid sequence having at least about 92% identity to SEQ ID NO: 21. In some embodiments, the masking moiety comprises an amino acid sequence having at least about 93% identity to SEQ ID NO: 21. In some embodiments, the masking moiety comprises an amino acid sequence having at least about 94% identity to SEQ ID NO: 21. In some embodiments, the masking moiety comprises an amino acid sequence having at least about 95% identity to SEQ ID NO: 21. In some embodiments, the masking moiety comprises an amino acid sequence having at least about 96% identity to SEQ ID NO: 21. In some embodiments, the masking moiety comprises an amino acid sequence having at least about 97% identity to SEQ ID NO: 21. In some embodiments, the masking moiety comprises an amino acid sequence having at least about 98% identity to SEQ ID NO: 21. In some embodiments, the masking moiety comprises an amino acid sequence having at least about 99% identity to SEQ ID NO: 21.

In some embodiments, a masking moiety has mutations C122V/N123C as compared to CD122 of SEQ ID NO: 15. In some embodiments, a masking moiety comprises an amino acid sequence of SEQ ID NO: 22.

(SEQ ID NO: 22)
AVNGTSQFTCFYNSRANISCVWSQDGALQDTSCQVHAWPDRRRWNQTCE
LLPVSQASWACNLILGAPDSQKLTTVDIVTLRVLCREGVRWRVMAIQDF
KPFENLRLMAPISLQVVHVETHRVCISWEISQASHYFERHLEFEARTLS
PGHTWEEAPLLTLKQKQEWICLETLTPDTQYEFQVRVKPLQGEFTTWSP
WSQPLAFRTKPAALGKD

In some embodiments, a masking moiety has mutations C122A/C168A as compared to CD122 of SEQ ID NO: 15. In some embodiments, a masking moiety comprises an amino acid sequence of SEQ ID NO: 23.

(SEQ ID NO: 23)
AVNGTSQFTCFYNSRANISCVWSQDGALQDTSCQVHAWPDRRRWNQTCE
LLPVSQASWACNLILGAPDSQKLTTVDIVTLRVLCREGVRWRVMAIQDF
KPFENLRLMAPISLQVVHVETHRANISWEISQASHYFERHLEFEARTLS
PGHTWEEAPLLTLKQKQEWIALETLTPDTQYEFQVRVKPLQGEFTTWSP
WSQPLAFRTKPAALGKD

In some embodiments, a masking moiety has mutations V117C/N123Q/C168A as compared to CD122 of SEQ ID NO: 15. In some embodiments, a masking moiety comprises an amino acid sequence of SEQ ID NO: 24.

(SEQ ID NO: 24)
AVNGTSQFTCFYNSRANISCVWSQDGALQDTSCQVHAWPDRRRWNQTCE
LLPVSQASWACNLILGAPDSQKLTTVDIVTLRVLCREGVRWRVMAIQDF
KPFENLRLMAPISLQVVHCETHRCQISWEISQASHYFERHLEFEARTLS
PGHTWEEAPLLTLKQKQEWIALETLTPDTQYEFQVRVKPLQGEFTTWSP
WSQPLAFRTKPAALGKD

In some embodiments, a masking moiety has mutations N123Q/C168A/L169C as compared to CD122 of SEQ ID NO: 15. In some embodiments, a masking moiety comprises an amino acid sequence of SEQ ID NO: 25.

(SEQ ID NO: 25)
AVNGTSQFTCFYNSRANISCVWSQDGALQDTSCQVHAWPDRRRWNQTCE
LLPVSQASWACNLILGAPDSQKLTTVDIVTLRVLCREGVRWRVMAIQDF
KPFENLRLMAPISLQVVHVETHRCQISWEISQASHYFERHLEFEARTLS
PGHTWEEAPLLTLKQKQEWIACETLTPDTQYEFQVRVKPLQGEFTTWSP
WSQPLAFRTKPAALGKD

In some embodiments, a masking moiety has mutations L106C/C122A/C168A/S195C as compared to CD122 of SEQ ID NO: 15. In some embodiments, a masking moiety comprises an amino acid sequence of SEQ ID NO: 26.

(SEQ ID NO: 26)
AVNGTSQFTCFYNSRANISCVWSQDGALQDTSCQVHAWPDRRRWNQTCE
LLPVSQASWACNLILGAPDSQKLTTVDIVTLRVLCREGVRWRVMAIQDF
KPFENLRCMAPISLQVVHVETHRANISWEISQASHYFERHLEFEARTLS
PGHTWEEAPLLTLKQKQEWIALETLTPDTQYEFQVRVKPLQGEFTTWCP
WSQPLAFRTKPAALGKD

In some embodiments, a masking moiety has mutations L106C/C122A/C168A/V184C as compared to CD122 of SEQ ID NO: 15. In some embodiments, a masking moiety comprises an amino acid sequence of SEQ ID NO: 27.

(SEQ ID NO: 27)
AVNGTSQFTCFYNSRANISCVWSQDGALQDTSCQVHAWPDRRRWNQTCE
LLPVSQASWACNLILGAPDSQKLTTVDIVTLRVLCREGVRWRVMAIQDF
KPFENLRCMAPISLQVVHVETHRANISWEISQASHYFERHLEFEARTLS
PGHTWEEAPLLTLKQKQEWIALETLTPDTQYEFQVRCKPLQGEFTTWSP
WSQPLAFRTKPAALGKD

In some embodiments, a masking moiety has mutations C122A/C168A/V184C/S195C as compared to CD122 of SEQ ID NO: 15. In some embodiments, a masking moiety comprises an amino acid sequence of SEQ ID NO: 28.

(SEQ ID NO: 28)
AVNGTSQFTCFYNSRANISCVWSQDGALQDTSCQVHAWPDRRRWNQTCE
LLPVSQASWACNLILGAPDSQKLTTVDIVTLRVLCREGVRWRVMAIQDF
KPFENLRLMAPISLQVVHVETHRANISWEISQASHYFERHLEFEARTLS
PGHTWEEAPLLTLKQKQEWIALETLTPDTQYEFQVRCKPLQGEFTTWCP
WSQPLAFRTKPAALGKD

In some embodiments, a masking moiety has mutations C122A/C168A/Q177C/R204C as compared to CD122 of SEQ ID NO: 15. In some embodiments, a masking moiety comprises an amino acid sequence of SEQ ID NO: 29.

(SEQ ID NO: 29)
AVNGTSQFTCFYNSRANISCVWSQDGALQDTSCQVHAWPDRRRWNQTCE
LLPVSQASWACNLILGAPDSQKLTTVDIVTLRVLCREGVRWRVMAIQDF
KPFENLRLMAPISLQVVHVETHRANISWEISQASHYFERHLEFEARTLS
PGHTWEEAPLLTLKQKQEWIALETLTPDTCYEFQVRVKPLQGEFTTWSP
WSQPLAFCTKPAALGKD

In some embodiments, a masking moiety has mutations L106C/C122V/C168V/S195C as compared to CD122 of SEQ ID NO: 15. In some embodiments, a masking moiety comprises an amino acid sequence of SEQ ID NO: 30.

(SEQ ID NO: 30)
AVNGTSQFTCFYNSRANISCVWSQDGALQDTSCQVHAWPDRRRWNQTCE
LLPVSQASWACNLILGAPDSQKLTTVDIVTLRVLCREGVRWRVMAIQDF
KPFENLRCMAPISLQVVHVETHRVNISWEISQASHYFERHLEFEARTLS
PGHTWEEAPLLTLKQKQEWIVLETLTPDTQYEFQVRVKPLQGEFTTWCP
WSQPLAFRTKPAALGKD

In some embodiments, a masking moiety has mutations F8C/A94C/C122V/C168V as compared to CD122 of SEQ ID NO: 15. In some embodiments, a masking moiety comprises an amino acid sequence of SEQ ID NO: 31.

(SEQ ID NO: 31)
AVNGTSQCTCFYNSRANISCVWSQDGALQDTSCQVHAWPDRRRWNQTCE
LLPVSQASWACNLILGAPDSQKLTTVDIVTLRVLCREGVRWRVMCIQDF
KPFENLRLMAPISLQVVHVETHRVNISWEISQASHYFERHLEFEARTLS
PGHTWEEAPLLTLKQKQEWIVLETLTPDTQYEFQVRVKPLQGEFTTWSP
WSQPLAFRTKPAALGKD

IL-12 Receptor

The masking moiety masks the IL-12 cytokine or functional fragment thereof in the targeted cytokine thereby reducing or preventing binding of the IL-cytokine or functional fragment thereof to its cognate receptor.

The IL-12 receptor, beta 1, or IL-12Rβ1 is a subunit of the IL-12 receptor complex. IL-12Rβ1 is also known as CD212. This protein binds to interleukin-12 (IL-12) with a low affinity. This protein forms a disulfide-linked oligomer, which is required for its IL-12 binding activity. The IL-12 receptor, beta 2, or IL-12Rβ2 is a subunit of the IL-12 receptor complex. The coexpression of IL-12Rβ1 and IL-12Rβ2 protein has been shown to lead to the formation of high-affinity IL-12 binding sites.

In some embodiments, the masking moiety comprises an extracellular domain of an IL-12 cytokine receptor, or a subunit or functional fragment thereof.

Interleukin-12 receptor subunit beta-1, also
called CD212 has the sequence:
(SEQ ID NO: 124)
MEPLVTWVVPLLFLFLLSRQGAACRTSECCFQDPPYPDADSGSASGPRD
LRCYRISSDRYECSWQYEGPTAGVSHFLRCCLSSGRCCYFAAGSATRLQ
FSDQAGVSVLYTVTLWVESWARNQTEKSPEVTLQLYNSVKYEPPLGDIK
VSKLAGQLRMEWETPDNQVGAEVQFRHRTPSSPWKLGDCGPQDDDTESC
LCPLEMNVAQEFQLRRRQLGSQGSSWSKWSSPVCVPPENPPQPQVRFSV
EQLGQDGRRRLTLKEQPTQLELPEGCQGLAPGTEVTYRLQLHMLSCPCK
AKATRTLHLGKMPYLSGAAYNVAVISSNQFGPGLNQTWHIPADTHTEPV
ALNISVGTNGTTMYWPARAQSMTYCIEWQPVGQDGGLATCSLTAPQDPD
PAGMATYSWSRESGAMGQEKCYYITIFASAHPEKLTLWSTVLSTYHFGG
NASAAGTPHHVSVKNHSLDSVSVDWAPSLLSTCPGVLKEYVVRCRDEDS
KQVSEHPVQPTETQVTLSGLRAGVAYTVQVRADTAWLRGVWSQPQRFSI
EVQVSDWLIFFASLGSFLSILLVGVLGYLGLNRAARHLCPPLPTPCASS
AIEFPGGKETWQWINPVDFQEEASLQEALVVEMSWDKGERTEPLEKTEL
PEGAPELALDTELSLEDGDRCKAKM
Interleukin-12 receptor subunit beta-2 has the
sequence:
(SEQ ID NO: 125)
MAHTFRGCSLAFMFIITWLLIKAKIDACKRGDVTVKPSHVILLGSTVNI
TCSLKPRQGCFHYSRRNKLILYKFDRRINFHHGHSLNSQVTGLPLGTTL
FVCKLACINSDEIQICGAEIFVGVAPEQPQNLSCIQKGEQGTVACTWER
GRDTHLYTEYTLQLSGPKNLTWQKQCKDIYCDYLDFGINLTPESPESNF
TAKVTAVNSLGSSSSLPSTFTFLDIVRPLPPWDIRIKFQKASVSRCTLY
WRDEGLVLLNRLRYRPSNSRLWNMVNVTKAKGRHDLLDLKPFTEYEFQI
SSKLHLYKGSWSDWSESLRAQTPEEEPTGMLDVWYMKRHIDYSRQQISL
FWKNLSVSEARGKILHYQVTLQELTGGKAMTQNITGHTSWTTVIPRTGN
WAVAVSAANSKGSSLPTRINIMNLCEAGLLAPRQVSANSEGMDNILVTW
QPPRKDPSAVQEYVVEWRELHPGGDTQVPLNWLRSRPYNVSALISENIK
SYICYEIRVYALSGDQGGCSSILGNSKHKAPLSGPHINAITEEKGSILI
SWNSIPVQEQMGCLLHYRIYWKERDSNSQPQLCEIPYRVSQNSHPINSL
QPRVTYVLWMTALTAAGESSHGNEREFCLQGKANWMAFVAPSICIAIIM
VGIFSTHYFQQKVFVLLAALRPQWCSREIPDPANSTCAKKYPIAEEKTQ
LPLDRLLIDWPTPEDPEPLVISEVLHQVTPVFRHPPCSNWPQREKGIQG
HQASEKDMMHSASSPPPPRALQAESRQLVDLYKVLESRGSDPKPENPAC
PWTVLPAGDLPTHDGYLPSNIDDLPSHEAPLADSLEELEPQHISLSVFP
SSSLHPLTFSCGDKLTLDQLKMRCDSLML

The bold indicates the pro-peptide, the italics with underline indicates the extracellular domain, the italics indicates the transmembrane domain and the bold with underline indicates the cytoplasmic domain.

In some embodiments, the masking moiety comprises the extracellular domain of human IL-12Rβ1 or a fragment, portion, or variant thereof that retains or otherwise demonstrates an affinity to IL-12.

In some embodiments, the masking moiety comprises an amino acid sequence having an amino acid sequence of human IL-12Rβ1 (SEQ ID NO: 125) with one to four amino acid substitutions. In some embodiments, the masking moiety comprises an amino acid sequence having an amino acid sequence of human IL-12Rβ1 with one or two amino acid substitutions.

In some embodiments, the masking moiety comprises residues 24 to 237 of human IL-12Rβ1, namely a sequence having SEQ ID NO: 126 or a fragment, portion, or variant thereof that retains or otherwise demonstrates an affinity to IL-12.

(SEQ ID NO: 126)
CRTSECCFQDPPYPDADSGSASGPRDLRCYRISSDRYECSWQYEGPTAG
VSHFLRCCLSSGRCCYFAAGSATRLQFSDQAGVSVLYTVTLWVESWARN
QTEKSPEVTLQLYNSVKYEPPLGDIKVSKLAGQLRMEWETPDNQVGAEV
QFRHRTPSSPWKLGDCGPQDDDTESCLCPLEMNVAQEFQLRRRQLGSQG
SSWSKWSSPVCVPPENP

In some embodiments, the masking moiety comprises IL-12Rβ1 having SEQ ID NO: 126. In some embodiments, the masking moiety comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 126. In some embodiments, the masking moiety comprises an amino acid sequence having at least about 85% sequence identity to SEQ ID NO: 126. In some embodiments, the masking moiety comprises an amino acid sequence having at least about 90% sequence identity to SEQ ID NO: 126. In some embodiments, the masking moiety comprises an amino acid sequence having at least about 92% sequence identity to SEQ ID NO: 126. In some embodiments, the masking moiety comprises an amino acid sequence having at least about 93% sequence identity to SEQ ID NO: 126. In some embodiments, the masking moiety comprises an amino acid sequence having at least about 94% sequence identity to SEQ ID NO: 126. In some embodiments, the masking moiety comprises an amino acid sequence having at least about 95% sequence identity to SEQ ID NO: 126. In some embodiments, the masking moiety comprises an amino acid sequence having at least about 96% sequence identity to SEQ ID NO: 126. In some embodiments, the masking moiety comprises an amino acid sequence having at least about 97% sequence identity to SEQ ID NO: 126. In some embodiments, the masking moiety comprises an amino acid sequence having at least about 98% sequence identity to SEQ ID NO: 126. In some embodiments, the masking moiety comprises an amino acid sequence having at least about 99% sequence identity to SEQ ID NO: 126.

In some embodiments, the masking moiety comprises an amino acid sequence having the amino acid sequence of SEQ ID NO: 126 with one to four amino acid substitutions. In some embodiments, the masking moiety comprises an amino acid sequence having the amino acid sequence of SEQ ID NO: 126 with one or two amino acid substitutions.

In some embodiments, the masking moiety comprises residues 24 to 545 of human IL-12Rβ1, namely a sequence having SEQ ID NO: 127 or a fragment, portion, or variant thereof that retains or otherwise demonstrates an affinity to IL-12.

(SEQ ID NO: 127)
CRTSECCFQDPPYPDADSGSASGPRDLRCYRISSDRYECSWQYEGPTAG
VSHFLRCCLSSGRCCYFAAGSATRLQFSDQAGVSVLYTVTLWVESWARN
QTEKSPEVTLQLYNSVKYEPPLGDIKVSKLAGQLRMEWETPDNQVGAEV
QFRHRTPSSPWKLGDCGPQDDDTESCLCPLEMNVAQEFQLRRRQLGSQG
SSWSKWSSPVCVPPENPPQPQVRFSVEQLGQDGRRRLTLKEQPTQLELP
EGCQGLAPGTEVTYRLQLHMLSCPCKAKATRTLHLGKMPYLSGAAYNVA
VISSNQFGPGLNQTWHIPADTHTEPVALNISVGTNGTTMYWPARAQSMT
YCIEWQPVGQDGGLATCSLTAPQDPDPAGMATYSWSRESGAMGQEKCYY
ITIFASAHPEKLTLWSTVLSTYHFGGNASAAGTPHHVSVKNHSLDSVSV
DWAPSLLSTCPGVLKEYVVRCRDEDSKQVSEHPVQPTETQVTLSGLRAG
VAYTVQVRADTAWLRGVWSQPQRFSIEVQVSD

In some embodiments, the masking moiety comprises IL-12Rβ1 having SEQ ID NO: 127. In some embodiments, the masking moiety comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 127. In some embodiments, the masking moiety comprises an amino acid sequence having at least about 85% sequence identity to SEQ ID NO: 127. In some embodiments, the masking moiety comprises an amino acid sequence having at least about 90% sequence identity to SEQ ID NO: 127. In some embodiments, the masking moiety comprises an amino acid sequence having at least about 92% sequence identity to SEQ ID NO: 127. In some embodiments, the masking moiety comprises an amino acid sequence having at least about 93% sequence identity to SEQ ID NO: 127. In some embodiments, the masking moiety comprises an amino acid sequence having at least about 94% sequence identity to SEQ ID NO: 127. In some embodiments, the masking moiety comprises an amino acid sequence having at least about 95% sequence identity to SEQ ID NO: 127. In some embodiments, the masking moiety comprises an amino acid sequence having at least about 96% sequence identity to SEQ ID NO: 127. In some embodiments, the masking moiety comprises an amino acid sequence having at least about 97% sequence identity to SEQ ID NO: 127. In some embodiments, the masking moiety comprises an amino acid sequence having at least about 98% sequence identity to SEQ ID NO: 127. In some embodiments, the masking moiety comprises an amino acid sequence having at least about 99% sequence identity to SEQ ID NO: 127.

In some embodiments, the masking moiety comprises an amino acid sequence having the amino acid sequence of SEQ ID NO: 127 with one to four amino acid substitutions. In some embodiments, the masking moiety comprises an amino acid sequence having the amino acid sequence of SEQ ID NO: 127 with one or two amino acid substitutions.

In some embodiments, the masking moiety comprises the extracellular domain of human IL-12Rβ2 or a fragment, portion, or variant thereof that retains or otherwise demonstrates an affinity to IL-12. In some embodiments, the masking moiety comprises an amino acid sequence having an amino acid sequence of human IL-12Rβ2 (SEQ ID NO: 155) with one to four amino acid substitutions. In some embodiments, the masking moiety comprises an amino acid sequence having an amino acid sequence of human IL-12Rβ2 with one or two amino acid substitutions.

(SEQ ID NO: 155)
MAHTFRGCSLAFMFIITWLLIKAKIDACKRGDVTVKPSHVILLGSTVNI
TCSLKPRQGCFHYSRRNKLILYKFDRRINFHHGHSLNSQVTGLPLGTTL
FVCKLACINSDEIQICGAEIFVGVAPEQPQNLSCIQKGEQGTVACTWER
GRDTHLYTEYTLQLSGPKNLTWQKQCKDIYCDYLDFGINLTPESPESNF
TAKVTAVNSLGSSSSLPSTFTFLDIVRPLPPWDIRIKFQKASVSRCTLY
WRDEGLVLLNRLRYRPSNSRLWNMVNVTKAKGRHDLLDLKPFTEYEFQI
SSKLHLYKGSWSDWSESLRAQTPEEEPTGMLDVWYMKRHIDYSRQQISL
FWKNLSVSEARGKILHYQVTLQELTGGKAMTQNITGHTSWTTVIPRTGN
WAVAVSAANSKGSSLPTRINIMNLCEAGLLAPRQVSANSEGMDNILVTW
QPPRKDPSAVQEYVVEWRELHPGGDTQVPLNWLRSRPYNVSALISENIK
SYICYEIRVYALSGDQGGCSSILGNSKHKAPLSGPHINAITEEKGSILI
SWNSIPVQEQMGCLLHYRIYWKERDSNSQPQLCEIPYRVSQNSHPINSL
QPRVTYVLWMTALTAAGESSHGNEREFCLQGKANWMAFVAPSICIAIIM
VGIFSTHYFQQKVFVLLAALRPQWCSREIPDPANSTCAKKYPIAEEKTQ
LPLDRLLIDWPTPEDPEPLVISEVLHQVTPVFRHPPCSNWPQREKGIQG
HQASEKDMMHSASSPPPPRALQAESRQLVDLYKVLESRGSDPKPENPAC
PWTVLPAGDLPTHDGYLPSNIDDLPSHEAPLADSLEELEPQHISLSVFP
SSSLHPLTFSCGDKLTLDQLKMRCDSLML

In some embodiments, the masking moiety comprises residues 24 to 212 of human IL-12Rβ2, namely a sequence having SEQ ID NO: 128.

(SEQ ID NO: 128)
KIDACKRGDVTVKPSHVILLGSTVNITCSLKPRQGCFHYSRRNKLILYK
FDRRINFHHGHSLNSQVTGLPLGTTLFVCKLACINSDEIQICGAEIFVG
VAPEQPQNLSCIQKGEQGTVACTWERGRDTHLYTEYTLQLSGPKNLTWQ
KQCKDIYCDYLDFGINLTPESPESNFTAKVTAVNSLGSSSSL

In some embodiments, the masking moiety comprises IL-12Rβ1 having SEQ ID NO: 128. In some embodiments, the masking moiety comprises an amino acid sequence having about or at least about 85%, 86%, 87%, 88%, 89%, 90%, 91%, 92%, 93%, 94%, 95%, 96%, 97%, 98%, or 99% sequence identity to any one of the amino acid sequence of SEQ ID NO: 128. In some embodiments, the masking moiety comprises an amino acid sequence having at least about 85% sequence identity to SEQ ID NO: 128. In some embodiments, the masking moiety comprises an amino acid sequence having at least about 90% sequence identity to SEQ ID NO: 128. In some embodiments, the masking moiety comprises an amino acid sequence having at least about 92% sequence identity to SEQ ID NO: 128. In some embodiments, the masking moiety comprises an amino acid sequence having at least about 93% sequence identity to SEQ ID NO: 128. In some embodiments, the masking moiety comprises an amino acid sequence having at least about 94% sequence identity to SEQ ID NO: 128. In some embodiments, the masking moiety comprises an amino acid sequence having at least about 95% sequence identity to SEQ ID NO: 128. In some embodiments, the masking moiety comprises an amino acid sequence having at least about 96% sequence identity to SEQ ID NO: 128. In some embodiments, the masking moiety comprises an amino acid sequence having at least about 97% sequence identity to SEQ ID NO: 128. In some embodiments, the masking moiety comprises an amino acid sequence having at least about 98% sequence identity to SEQ ID NO: 128. In some embodiments, the masking moiety comprises an amino acid sequence having at least about 99% sequence identity to SEQ ID NO: 128.

In some embodiments, the masking moiety comprises an amino acid sequence having the amino acid sequence of SEQ ID NO: 128 with one to four amino acid substitutions. In some embodiments, the masking moiety comprises an amino acid sequence having the amino acid sequence of SEQ ID NO: 128 with one or two amino acid substitutions.

In some embodiments, the masking moiety comprises residues 24 to 222 of human IL-12Rβ2, namely a sequence having SEQ ID NO: 129.

(SEQ ID NO: 129)
KIDACKRGDVTVKPSHVILLGSTVNITCSLKPRQGCFHYSRENKLILYK
FDRRINFHHGHSLNSQVTGLPLGTTLFVCKLACINSDEIQICGAEIFVG
VAPEQPQNLSCIQKGEQGTVACTWERGRDTHLYTEYTLQLSGPKNLTWQ
KQCKDIYCDYLDFGINLTPESPESNFTAKVTAVNSLGSSSSLPSTFTFL
DIV

In some embodiments, the masking moiety comprises IL-12Rβ1 having SEQ ID NO: 129. In some embodiments, the masking moiety comprises an amino acid sequence having about or at least about 85%, 86%, 87%, 88%, 89%, 90%, 91%, 92%, 93%, 94%, 95%, 96%, 97%, 98%, or 99% sequence identity to any one of the amino acid sequence of SEQ ID NO: 129. In some embodiments, the masking moiety comprises an amino acid sequence having at least about 85% sequence identity to SEQ ID NO: 129. In some embodiments, the masking moiety comprises an amino acid sequence having at least about 90% sequence identity to SEQ ID NO: 129. In some embodiments, the masking moiety comprises an amino acid sequence having at least about 92% sequence identity to SEQ ID NO: 129. In some embodiments, the masking moiety comprises an amino acid sequence having at least about 93% sequence identity to SEQ ID NO: 129. In some embodiments, the masking moiety comprises an amino acid sequence having at least about 94% sequence identity to SEQ ID NO: 129. In some embodiments, the masking moiety comprises an amino acid sequence having at least about 95% sequence identity to SEQ ID NO: 129. In some embodiments, the masking moiety comprises an amino acid sequence having at least about 96% sequence identity to SEQ ID NO: 129. In some embodiments, the masking moiety comprises an amino acid sequence having at least about 97% sequence identity to SEQ ID NO: 129. In some embodiments, the masking moiety comprises an amino acid sequence having at least about 98% sequence identity to SEQ ID NO: 129. In some embodiments, the masking moiety comprises an amino acid sequence having at least about 99% sequence identity to SEQ ID NO: 129.

In some embodiments, the masking moiety comprises an amino acid sequence having the amino acid sequence of SEQ ID NO: 129 with one to four amino acid substitutions. In some embodiments, the masking moiety comprises an amino acid sequence having the amino acid sequence of SEQ ID NO: 129 with one or two amino acid substitutions.

In some embodiments, the masking moiety comprises residues 24 to 319 of human IL-12Rβ2, namely a sequence having SEQ ID NO: 130.

(SEQ ID NO: 130)
KIDACKRGDVTVKPSHVILLGSTVNITCSLKPRQGCFHYSRENKLILYK
FDRRINFHHGHSLNSQVTGLPLGTTLFVCKLACINSDEIQICGAEIFVG
VAPEQPQNLSCIQKGEQGTVACTWERGRDTHLYTEYTLQLSGPKNLTWQ
KQCKDIYCDYLDFGINLTPESPESNFTAKVTAVNSLGSSSSLPSTFTFL
DIVRPLPPWDIRIKFQKASVSRCTLYWRDEGLVLLNRLRYRPSNSRLWN
MVNVTKAKGRHDLLDLKPFTEYEFQISSKLHLYKGSWSDWSESLRAQTP
EE

In some embodiments, the masking moiety comprises IL-12Rβ1 having SEQ ID NO: 130. In some embodiments, the masking moiety comprises an amino acid sequence having about or at least about 85%, 86%, 87%, 88%, 89%, 90%, 91%, 92%, 93%, 94%, 95%, 96%, 97%, 98%, or 99% sequence identity to any one of the amino acid sequence of SEQ ID NO: 130. In some embodiments, the masking moiety comprises an amino acid sequence having at least about 85% sequence identity to SEQ ID NO: 130. In some embodiments, the masking moiety comprises an amino acid sequence having at least about 90% sequence identity to SEQ ID NO: 130. In some embodiments, the masking moiety comprises an amino acid sequence having at least about 92% sequence identity to SEQ ID NO: 130. In some embodiments, the masking moiety comprises an amino acid sequence having at least about 93% sequence identity to SEQ ID NO: 130. In some embodiments, the masking moiety comprises an amino acid sequence having at least about 94% sequence identity to SEQ ID NO: 130. In some embodiments, the masking moiety comprises an amino acid sequence having at least about 95% sequence identity to SEQ ID NO: 130. In some embodiments, the masking moiety comprises an amino acid sequence having at least about 96% sequence identity to SEQ ID NO: 130. In some embodiments, the masking moiety comprises an amino acid sequence having at least about 97% sequence identity to SEQ ID NO: 130. In some embodiments, the masking moiety comprises an amino acid sequence having at least about 98% sequence identity to SEQ ID NO: 130. In some embodiments, the masking moiety comprises an amino acid sequence having at least about 99% sequence identity to SEQ ID NO: 130.

In some embodiments, the masking moiety comprises an amino acid sequence having the amino acid sequence of SEQ ID NO: 130 with one to four amino acid substitutions. In some embodiments, the masking moiety comprises an amino acid sequence having the amino acid sequence of SEQ ID NO: 130 with one or two amino acid substitutions.

In some embodiments, the masking moiety comprises residues 24 to 319 of human IL-12Rβ2, namely a sequence having SEQ ID NO: 130, with one or more cysteine substitutions. In some embodiments, the masking moiety comprises residues 24 to 319 of human IL-12Rβ2, namely a sequence having SEQ ID NO: 130, with an amino acid substitution at position C242. In some embodiments, the amino acid substitution is at position C242 is C242S. In some embodiments, the masking moiety comprises an amino acid sequence of SEQ ID NO: 131.

(SEQ ID NO: 131)
KIDACKRGDVTVKPSHVILLGSTVNITCSLKPRQGCFHYSRENKLILYK
FDRRINFHHGHSLNSQVTGLPLGTTLFVCKLACINSDEIQICGAEIFVG
VAPEQPQNLSCIQKGEQGTVACTWERGRDTHLYTEYTLQLSGPKNLTWQ
KQCKDIYCDYLDFGINLTPESPESNFTAKVTAVNSLGSSSSLPSTFTFL
DIVRPLPPWDIRIKFQKASVSRSTLYWRDEGLVLLNRLRYRPSNSRLWN
MVNVTKAKGRHDLLDLKPFTEYEFQISSKLHLYKGSWSDWSESLRAQTP
EE

In some embodiments, the masking moiety comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 131. In some embodiments, the masking moiety consists of an amino acid sequence of SEQ ID NO: 131.

In some embodiments, the masking moiety comprises residues 24 to 622 of human IL-12Rβ2, namely a sequence having SEQ ID NO: 132.

(SEQ ID NO: 132)
KIDACKRGDVTVKPSHVILLGSTVNITCSLKPRQGCFHYSRENKLILYK
FDRRINFHHGHSLNSQVTGLPLGTTLFVCKLACINSDEIQICGAEIFVG
VAPEQPQNLSCIQKGEQGTVACTWERGRDTHLYTEYTLQLSGPKNLTWQ
KQCKDIYCDYLDFGINLTPESPESNFTAKVTAVNSLGSSSSLPSTFTFL
DIVRPLPPWDIRIKFQKASVSRCTLYWRDEGLVLLNRLRYRPSNSRLWN
MVNVTKAKGRHDLLDLKPFTEYEFQISSKLHLYKGSWSDWSESLRAQTP
EEEPTGMLDVWYMKRHIDYSRQQISLFWKNLSVSEARGKILHYQVTLQE
LTGGKAMTQNITGHTSWTTVIPRTGNWAVAVSAANSKGSSLPTRINIMN
LCEAGLLAPRQVSANSEGMDNILVTWQPPRKDPSAVQEYVVEWRELHPG
GDTQVPLNWLRSRPYNVSALISENIKSYICYEIRVYALSGDQGGCSSIL
GNSKHKAPLSGPHINAITEEKGSILISWNSIPVQEQMGCLLHYRIYWKE
R

In some embodiments, the masking moiety comprises IL-12Rβ1 having SEQ ID NO: 132. In some embodiments, the masking moiety comprises an amino acid sequence having about or at least about 85%, 86%, 87%, 88%, 89%, 90%, 91%, 92%, 93%, 94%, 95%, 96%, 97%, 98%, or 99% sequence identity to amino acid sequence SEQ ID NO: 132. In some embodiments, the masking moiety comprises an amino acid sequence having at least about 85% sequence identity to SEQ ID NO: 132. In some embodiments, the masking moiety comprises an amino acid sequence having at least about 90% sequence identity to SEQ ID NO: 132. In some embodiments, the masking moiety comprises an amino acid sequence having at least about 92% sequence identity to SEQ ID NO: 132. In some embodiments, the masking moiety comprises an amino acid sequence having at least about 93% sequence identity to SEQ ID NO: 132. In some embodiments, the masking moiety comprises an amino acid sequence having at least about 94% sequence identity to SEQ ID NO: 132. In some embodiments, the masking moiety comprises an amino acid sequence having at least about 95% sequence identity to SEQ ID NO: 132. In some embodiments, the masking moiety comprises an amino acid sequence having at least about 96% sequence identity to SEQ ID NO: 132. In some embodiments, the masking moiety comprises an amino acid sequence having at least about 97% sequence identity to SEQ ID NO: 132. In some embodiments, the masking moiety comprises an amino acid sequence having at least about 98% sequence identity to SEQ ID NO: 132. In some embodiments, the masking moiety comprises an amino acid sequence having at least about 99% sequence identity to SEQ ID NO: 132.

In some embodiments, the masking moiety comprises an amino acid sequence having the amino acid sequence of SEQ ID NO: 132 with one to four amino acid substitutions. In some embodiments, the masking moiety comprises an amino acid sequence having the amino acid sequence of SEQ ID NO: 132 with one or two amino acid substitutions.

In some embodiments, the masking moiety comprises residues 24 to 227 of human IL-12Rβ2, namely a sequence having SEQ ID NO: 133.

(SEQ ID NO: 133)
KIDACKRGDVTVKPSHVILLGSTVNITCSLKPRQGCFHYSRENKLILYK
FDRRINFHHGHSLNSQVTGLPLGTTLFVCKLACINSDEIQICGAEIFVG
VAPEQPQNLSCIQKGEQGTVACTWERGRDTHLYTEYTLQLSGPKNLTWQ
KQCKDIYCDYLDFGINLTPESPESNFTAKVTAVNSLGSSSSLPSTFTFL
DIVRPLPP

In some embodiments, the masking moiety comprises IL-12Rβ1 having SEQ ID NO: 133. In some embodiments, the masking moiety comprises an amino acid sequence having about or at least about 85%, 86%, 87%, 88%, 89%, 90%, 91%, 92%, 93%, 94%, 95%, 96%, 97%, 98%, or 99% sequence identity to amino acid sequence SEQ ID NO: 133. In some embodiments, the masking moiety comprises an amino acid sequence having at least about 85% sequence identity to SEQ ID NO: 133. In some embodiments, the masking moiety comprises an amino acid sequence having at least about 90% sequence identity to SEQ ID NO: 133. In some embodiments, the masking moiety comprises an amino acid sequence having at least about 92% sequence identity to SEQ ID NO: 133. In some embodiments, the masking moiety comprises an amino acid sequence having at least about 93% sequence identity to SEQ ID NO: 133. In some embodiments, the masking moiety comprises an amino acid sequence having at least about 94% sequence identity to SEQ ID NO: 133. In some embodiments, the masking moiety comprises an amino acid sequence having at least about 95% sequence identity to SEQ ID NO: 133. In some embodiments, the masking moiety comprises an amino acid sequence having at least about 96% sequence identity to SEQ ID NO: 133. In some embodiments, the masking moiety comprises an amino acid sequence having at least about 97% sequence identity to SEQ ID NO: 133. In some embodiments, the masking moiety comprises an amino acid sequence having at least about 98% sequence identity to SEQ ID NO: 133. In some embodiments, the masking moiety comprises an amino acid sequence having at least about 99% sequence identity to SEQ ID NO: 133.

In some embodiments, the masking moiety comprises an amino acid sequence having the amino acid sequence of SEQ ID NO: 133 with one to four amino acid substitutions. In some embodiments, the masking moiety comprises an amino acid sequence having the amino acid sequence of SEQ ID NO: 133 with one or two amino acid substitutions.

Antibody-Based Masking Moieties

The masking moiety that masks a cytokine or functional fragment thereof in the targeted cytokine can be an antibody or an antigen binding fragment thereof. In some embodiments, the antibody or an antigen binding fragment binds to a cytokine. The cytokine and anti-cytokine antibody or binding fragment complex in the targeted cytokine thereby reduces or prevents binding of the cytokine or functional fragment thereof to its cognate receptor or ligand (e.g., masking the activity of the cytokine).

The cytokine specific antibody can be an intact antibody, or a binding fragment thereof. In some embodiments, the binding fragments derived from an antibody include, but are not limited to, Fab, Fab′, F(ab′)2, scFv, and single-chain antibodies. In some embodiments, the antibody-based masking moiety is an scFv. In some embodiments, the antibody-based masking moiety is an Fab fragment.

Single-Chain Variable Fragments (scFv)

In some embodiments, a masking moiety comprises an anti-IL-2 scFv having a variable heavy chain (VH) of SEQ ID NO: 172. In some embodiments, a masking moiety comprises an anti-IL-2 scFv having a variable light chain (VL) of SEQ ID NO: 173. In some embodiments, a masking moiety comprises an anti-IL-2 scFv having a VH of SEQ ID NO: 172 and a VL of SEQ ID NO: 173.

In some embodiments, a masking moiety comprises an anti-IL-2 scFv having a hCDR1 of SEQ ID NO: 174, a hCDR2 of SEQ ID NO: 175, and hCDR3 of SEQ ID NO: 176. In some embodiments, a masking moiety comprises an anti-IL-2 scFv having a lCDR1 of SEQ ID NO: 177, a lCDR2 of SEQ ID NO: 178, and lCDR3 of SEQ ID NO: 179. In some embodiments, a masking moiety comprises an anti-IL-2 scFv having a hCDR1 of SEQ ID NO: 174, a hCDR2 of SEQ ID NO: 175, hCDR3 of SEQ ID NO: 176, a lCDR1 of SEQ ID NO: 177, a lCDR2 of SEQ ID NO: 178, and lCDR3 of SEQ ID NO: 179.

An Exemplary Anti-IL-2 scFv Sequence

| | | |
|---|---|---|
| VH | QLQLQESGPGLVKPSQTLSLTCTVSGGSI | SEQ ID |
| | SSGGYYWSWIRQHPGKGLEWIGYIYKSGS | NO: 172 |
| | AYYSPSLKSRVTISVDTSKNQFSLKLSSV | |
| | TAADTAVYYCARTPTVTGDWFDPWGRGTL | |
| | VTVSS | |
| VL | NFMLTQPHSVSESPGKTVTISCTRSSGSI | SEQ ID |
| | ASNYVQWYQQRPGSSPTTVIYEDNQRPSG | NO: 173 |
| | VPDRFSGSIDSSSNSASLTISGLKTEDEA | |
| | DYYCQTYDSIDVYFGGGTKLTVL | |
| hCDR1 | SGGYYWS | SEQ ID |
| | | NO: 174 |
| hCDR2 | GYIYKSGSAY YSPSLKSRV | SEQ ID |
| | | NO: 175 |
| hCDR3 | TPTVTGDWFDP | SEQ ID |
| | | NO: 176 |
| lCDR1 | TRSSGSIASNYVQ | SEQ ID |
| | | NO: 177 |
| lCDR2 | EDNQRPS | SEQ ID |
| | | NO: 178 |
| lCDR3 | QTYDSIDVY | SEQ ID |
| | | NO: 179 |
| scFv1 | NFMLTQPHSVSESPGKTVTISCTRSSGSI | SEQ ID |
| | ASNYVQWYQQRPGSSPTTVIYEDNQRPSG | NO: 180 |
| | VPDRFSGSIDSSSNSASLTISGLKTEDEA | |
| | DYYCQTYDSIDVYFGGGTKLTVLGGGGGS | |
| | GGGGSGGGGSGGGGSQLQLQESGPGLVKP | |
| | SQTLSLTCTVSGGSISSGGYYWSWIRQHP | |
| | GKGLEWIGYIYKSGSAYYSPSLKSRVTIS | |
| | VDTSKNQFSLKLSSVTAADTAVYYCARTP | |
| | TVTGDWFDPWGRGTLVTVSS | |

In some embodiments, a masking moiety comprises a VL linked to a VH via a non-cleavable linker. In some embodiments, a masking moiety comprises a VH linked to a VL via a non-cleavable linker. In some embodiments, a non-cleavable linker is GGGGGSGGGGSGGGGSGGGGS (SEQ ID NO: 182).

In some embodiments, the scFv masking moiety is linked to the Fc domain by a cleavable linker. In some embodiments, the scFv masking moiety is linked to the Fc domain by a cleavable linker described herein (e.g., a linker comprising a cleavable peptide described in Table 1).

In some embodiments, the masking moiety comprises a scFv having an amino acid sequence having about or at least about 85%, 86%, 87%, 88%, 89%, 90%, 91%, 92%, 93%, 94%, 95%, 96%, 97%, 98%, or 99% sequence identity to any one of the amino acid sequence of SEQ ID NO: 180. In some embodiments, the masking moiety comprises an amino acid sequence having at least about 85% sequence identity to SEQ ID NO: 180. In some embodiments, the masking moiety comprises an amino acid sequence having at least about 90% sequence identity to SEQ ID NO: 180. In some embodiments, the masking moiety comprises an amino acid sequence having at least about 92% sequence identity to SEQ ID NO: 180. In some embodiments, the masking moiety comprises an amino acid sequence having at least about 93% sequence identity to SEQ ID NO: 180. In some embodiments, the masking moiety comprises an amino acid sequence having at least about 94% sequence identity to SEQ ID NO: 180. In some embodiments, the masking moiety comprises an amino acid sequence having at least about 95% sequence identity to SEQ ID NO: 180. In some embodiments, the masking moiety comprises an amino acid sequence having at least about 96% sequence identity to SEQ ID NO: 180. In some embodiments, the masking moiety comprises an amino acid sequence having at least about 97% sequence identity to SEQ ID NO: 180. In some embodiments, the masking moiety comprises an amino acid sequence having at least about 98% sequence identity to SEQ ID NO: 180. In some embodiments, the masking moiety comprises an amino acid sequence having at least about 99% sequence identity to SEQ ID NO: 180.

VHH Antibody

VHH antibody (or nanobody) is the antigen binding fragment of heavy chain only antibodies. In some embodiments, the masking moiety is a heavy-chain-only antibody (VHH). In some embodiments, the masking moiety is a heavy-chain-only antibody (VHH), that binds to a cytokine. The VHH may be derived from, for example, an organism that produces VHH antibody such as a camelid, and a shark. In some cases, the VHH may be a recombinant VHH. VHH technology is based on fully functional antibodies from camelids that lack light chains. These heavy-chain antibodies contain a single variable domain (VHH) and two constant domains (CH2 and CH3). A VHH comprises a single chain polypeptide having three CDRs and four framework regions (Flus 1-4). As used herein, “framework region” or “FR” refers to a region in the variable domain which is located between the CDRs. As used herein, “complementary determining region” or “CDR” refers to variable regions in VHHs that contains the amino acid sequences capable of specifically binding to antigenic targets (e.g., cytokines). !

In some embodiments, the VHH comprises three CDRs and four framework regions, designated FR1, CDR1, FR2, CDR2, FR3, CDR3, and FR4. In some embodiments, a VHH may be truncated at the N-terminus or C-terminus such that it comprises only a partial FR1 and/or FR4, or lacks one or both of those framework regions, so tong as the VHH substantially maintains cytokine binding and specificity.

The VHH is not limited to a specific biological source or to a specific method of preparation. For example, the VHH can generally be obtained: (1) by isolating the V_{H}H domain of a naturally occurring heavy chain antibody; (2) by expression of a nucleotide sequence encoding a naturally occurring V_{H}H domain; (3) by “humanization” of a naturally occurring V_{H}H domain or by expression of a nucleic acid encoding a such humanized V_{H}H domain; (4) by “camelization” of a naturally occurring VH domain from any animal species, such as from a mammalian species, such as from a human being, or by expression of a nucleic acid encoding such a camelized VH domain; (5) by “camelization” of a “domain antibody” or “Dab” as described in the art, or by expression of a nucleic acid encoding such a camelized VH domain; (6) by using synthetic or semi-synthetic techniques for preparing proteins, polypeptides or other amino acid sequences known in the art; (7) by preparing a nucleic acid encoding a VHH using techniques for nucleic acid synthesis known in the art, followed by expression of the nucleic acid thus obtained; and/or (8) by any combination of one or more of the foregoing.

In some embodiments, the IL-2 masking moiety is anti-IL-2 VHH antibody. The IL-2 masking moiety comprises a VHH that corresponds to the V_{H}H domains of naturally occurring heavy chain antibodies directed against IL-2. Such anti-IL-2 V_{H}H sequences can generally be generated or obtained by suitably immunizing a species of Camelid with an IL-2 molecule. In some embodiments, the V_{H}H sequences directed against an IL-2 molecule involves suitably immunizing a transgenic mammal that is capable of expressing heavy chain antibodies.

In some embodiments, a masking moiety comprises an anti-IL-2 VHH having a amino acid sequence of SEQ ID NO: 168. In some embodiments, a masking moiety comprises an anti-IL-2 VHH having a hCDR1 of SEQ ID NO: 169, a hCDR2 of SEQ ID NO: 170, and hCDR3 of SEQ ID NO: 171.

An Exemplary Anti-IL-2 scFv Sequence

| | | |
|---|---|---|
| VHH | EVQLVESGGGLVQPGGSLRLSCAASGSIF | SEQ ID |
| | SINVMGWYRQAPGKQRELVAAISSGGSTN | NO: 168 |
| | YADSVKGRFTISRDNAKNTVYLQMNSLKP | |
| | EDTAVYYCMYASSWYEDETDYWGQGTQVT | |
| | VSS | |
| hCDR1 | GSIFSINVMG | SEQ ID |
| | | NO: 169 |
| hCDR2 | AISSGGSTNYADSVKG | SEQ ID |
| | | NO: 170 |
| hCDR3 | ASSWYEDETDY | SEQ ID |
| | | NO: 171 |

In some embodiments, the masking moiety comprises a VHH having an amino acid sequence having about or at least about 85%, 86%, 87%, 88%, 89%, 90%, 91%, 92%, 93%, 94%, 95%, 96%, 97%, 98%, or 99% sequence identity to any one of the amino acid sequence of SEQ ID NO: 168. In some embodiments, the masking moiety comprises an amino acid sequence having at least about 85% sequence identity to SEQ ID NO: 168. In some embodiments, the masking moiety comprises an amino acid sequence having at least about 90% sequence identity to SEQ ID NO: 168. In some embodiments, the masking moiety comprises an amino acid sequence having at least about 92% sequence identity to SEQ ID NO: 168. In some embodiments, the masking moiety comprises an amino acid sequence having at least about 93% sequence identity to SEQ ID NO: 168. In some embodiments, the masking moiety comprises an amino acid sequence having at least about 94% sequence identity to SEQ ID NO: 168. In some embodiments, the masking moiety comprises an amino acid sequence having at least about 95% sequence identity to SEQ ID NO: 168. In some embodiments, the masking moiety comprises an amino acid sequence having at least about 96% sequence identity to SEQ ID NO: 168. In some embodiments, the masking moiety comprises an amino acid sequence having at least about 97% sequence identity to SEQ ID NO: 168. In some embodiments, the masking moiety comprises an amino acid sequence having at least about 98% sequence identity to SEQ ID NO: 168. In some embodiments, the masking moiety comprises an amino acid sequence having at least about 99% sequence identity to SEQ ID NO: 168.

In some embodiments, the VHH masking moiety is linked to the Fc domain by a cleavable linker. In some embodiments, the VHH masking moiety is linked to the Fc domain by a cleavable linker described herein (e.g., a linker comprising a cleavable peptide described in Table 1).

### Linkers

Provided herein are linkers for use in a targeted cytokine or cleavage product thereof. A linker as provided herein refers to a peptide of two more amino acids that is used to link two functional components together in the targeted cytokines described herein.

The targeted cytokine comprises a first linker and a second linker, where at least the first linker or the second linker comprises a proteolytically cleavable peptide.

In some embodiments, a first Fc polypeptide is linked to a cytokine or a variant thereof through a first linker. In some embodiments, a second Fc polypeptide is linked to a masking moiety through a second linker. In some embodiments, a first Fc polypeptide is linked to a cytokine or a variant thereof through a cleavable linker. In some embodiments, a second Fc polypeptide is linked to a masking moiety through a cleavable linker. In some embodiments, a first Fc polypeptide is linked to a cytokine or a variant thereof through a cleavable linker and a second Fc polypeptide is linked to a masking moiety through a non-cleavable linker. In some embodiments, a first Fc polypeptide is linked to a cytokine or a variant thereof through a non-cleavable linker and a second Fc polypeptide is linked to a masking moiety through a cleavable linker. In some embodiments, a first Fc polypeptide is linked to a cytokine or a variant thereof through a cleavable linker and a second Fc polypeptide is linked to a masking moiety through a cleavable linker. In some embodiments, a first Fc polypeptide is linked to a cytokine or a variant thereof through a non-cleavable linker and a second Fc polypeptide is linked to a masking moiety through a non-cleavable linker.

Non-Cleavable Linker

In some embodiments, the non-cleavable linker is between 2 and 25 amino acids in length. In some embodiments, the non-cleavable linker is between 3 and 21 amino acids in length. In some embodiments, the non-cleavable linker is between 3 and 18 amino acids in length. In some embodiments, the non-cleavable linker is between 5 and 18 amino acids in length. In some embodiments, the non-cleavable linker is between 3 and 8 amino acids in length. In some embodiments, the non-cleavable linker is between 4 and 6 amino acids in length.

In some embodiments, the non-cleavable linker is 15 amino acids in length. In some embodiments, the non-cleavable linker is 16 amino acids in length. In some embodiments, the non-cleavable linker is 17 amino acids in length. In some embodiments, the non-cleavable linker is 18 amino acids in length. In some embodiments, the non-cleavable linker is 19 amino acids in length. In some embodiments, the non-cleavable linker is 20 amino acids in length.

In some embodiments, the non-cleavable linker is rich in amino acid residues G, S and P. In some embodiments, the non-cleavable linker only includes amino acid residue types selected from the group consisting of G, S and P. In some embodiments, the non-cleavable linker includes a ‘GS’ repeat. In some embodiments, the non-cleavable linker includes an N′ terminal ‘P’ residue.

In some embodiments, the non-cleavable linker comprises an amino acid sequence as shown in SEQ ID NO: 32 (PGSGS).

In some embodiments, the non-cleavable linker comprises an amino acid sequence as shown in SEQ ID NO: 33 (GGSSPPGGGSSGGGSGP).

In some embodiments, the non-cleavable linker comprises an amino acid sequence GGS.

In some embodiments, the non-cleavable linker includes [(G)nS], where n=4 or 5.

In some embodiments, the non-cleavable linker comprises an amino acid sequence as shown in SEQ ID NO: 134 (GGGGS).

In some embodiments, the non-cleavable linker comprises an amino acid sequence as shown in SEQ ID NO: 135 (GGGGSGGGGS).

In some embodiments, the non-cleavable linker comprises an amino acid sequence as shown in SEQ ID NO: 136 (GGSGGGSGGGGGS).

In some embodiments, the non-cleavable linker comprises an amino acid sequence as shown in SEQ ID NO: 137 (GGSGGSGGSGGSGGSSGP).

In some embodiments, the non-cleavable linker comprises an amino acid sequence as shown in SEQ ID NO: 138 (PGGSGP).

In some embodiments, the non-cleavable linker comprises an amino acid sequence as shown in SEQ ID NO: 56 (GGSPG).

In some embodiments, the linker the non-cleavable linker comprises an amino acid sequence as shown in SEQ ID NO: 181 (GGSSGSGGSGGGSGSGGG).

In some embodiments, wherein the second linker comprises a proteolytically cleavable peptide such that the second linker is a proteolytically cleavable linker and the first linker does not comprise a proteolytically cleavable peptide such that the first linker is a non-proteolytically cleavable linker, the non-cleavable linker is between 3 and 18 amino acids in length. In some embodiments, the non-cleavable linker comprises an amino acid sequence as shown in SEQ ID NO: 134 (GGGGS). In some embodiments, the non-cleavable linker comprises an amino acid sequence as shown in SEQ ID NO: 135 (GGGGSGGGGS). In some embodiments, the non-cleavable linker comprises an amino acid sequence as shown in SEQ ID NO: 136 (GGSGGGSGGGGGS). In some embodiments, the non-cleavable linker comprises an amino acid sequence as shown in SEQ ID NO: 137 (GGSGGSGGSGGSGGSSGP). In some embodiments, the non-cleavable linker comprises an amino acid sequence as shown in SEQ ID NO: 138 (PGGSGP). In some embodiments, the non-cleavable linker comprises an amino acid sequence as shown in SEQ ID NO: 139 (GGSPG).

In some embodiments, it is desirable for the first and second polypeptide chains to be of the same or a similar length to facilitate the first half life extension domain associating with the second half life extension domain and the masking moiety masking the cytokine or functional fragment thereof in the assembled construct. As such where the masking moiety is a shorter amino acid sequence than the cytokine or functional fragment thereof, the difference in length may be compensated fully or in part by using a longer linker L1.

Cleavable Linker

In some embodiments, the cleavable linker is between 2 and 25 amino acids in length. In some embodiments, the cleavable linker is between 3 and 21 amino acids in length. In some embodiments, the cleavable linker is between 3 and 18 amino acids in length. In some embodiments, the cleavable linker is between 5 and 18 amino acids in length. In some embodiments, the cleavable linker is between 3 and 8 amino acids in length. In some embodiments, the cleavable linker is between 4 and 6 amino acids in length.

In some embodiments, the cleavable linker is 15 amino acids in length. In some embodiments, the cleavable linker is 16 amino acids in length. In some embodiments, the cleavable linker is 17 amino acids in length. In some embodiments, the cleavable linker is 18 amino acids in length. In some embodiments, the cleavable linker is 19 amino acids in length. In some embodiments, the cleavable linker is 20 amino acids in length.

In some embodiments, the cleavable linker comprises a proteolytically cleavable peptide (CP) flanked on both sides by a spacer domain (SD) as shown in below formula:

SD-CP-SD

Cleavable Peptides

The cleavable linker comprises a cleavable peptide.

A cleavable peptide is a polypeptide that includes a protease cleavage site, such that the cleavable peptide is proteolytically cleavable. Proteases are enzymes that cleave and hydrolyse the peptide bonds between two specific amino acid residues of target substrate proteins. A “cleavage site” as used herein refers to a recognizable site for cleavage of a portion of the cleavable peptide found in any of the linkers that comprise a cleavable peptide described herein. Thus, a cleavage site may be found in the sequence of a cleavable peptide as described herein. In some embodiments, the cleavage site is an amino acid sequence that is recognized and cleaved by a cleaving agent.

In some embodiments, the protease cleavage site is a tumor-associated protease cleavage site. A “tumor-associated protease cleavage site” as provided herein is an amino acid sequence recognized by a protease whose expression is specific or upregulated for a tumor cell or tumor cell environment thereof.

The tumor cell environment is complex and can comprise multiple different proteases. As such, the precise site at which a given cleavable peptide will be cleaved in the tumor cell environment may vary between tumor types, between patients with the same tumor type and even between cleavage products formed in the same tumor dependent on the specific tumor cell environment. Moreover, even after cleavage, further modification of the initial cleavage product, e.g., by removal of one or two terminal amino acids, may occur by the further action of proteases in the tumor cell environment. A distribution of cleavage products can thus be expected to form in the tumor cell environment of a patient following administration of a single structure of a targeted cytokine as described herein.

It will be understood that a cleavage site as referred to herein refers to a site between two specific amino acid residues within the cleavable peptide that are a target for a protease known to be associated with a tumor cell environment. In this sense, there may be more than one cleavage site present in a cleavable peptide as described herein where different proteases cleave the cleavable peptide at different cleavage sites. It is also possible that more than one protease may act on the same cleavage site within a cleavable peptide. Discussion of protease cleavage sites can be found in the art.

Thus, the cleavable peptides disclosed herein may be cleaved by one or more proteases. In some embodiments, the protease cleavage site is a tumor-associated protease cleavage site. The tumor-associated protease cleavage site can be recognized by a tumor-associated protease. As non-limiting examples, the tumor-associated protease is a matrix metalloproteinase (MMP), selected from the group consisting of MMP1, MMP2, MMP3, MMP7, MMP8, MMP9, MMP10, MMP11, MMP12, MMP 13, MMP14, MMP15, MMP16, MMP17, MMP19, MMP20, MMP21, MMP23A, MMP23B, MMP24, MMP25, MMP27, and MMP28. In one embodiment, the tumor-associated protease is MMP2. In another embodiment, the tumor-associated protease is MMP3. In some embodiments, the tumor-associated protease is MMP7. In yet another embodiment, the tumor-associated protease is MMP9. In yet another embodiment, the tumor-associated protease is MMP10. Other disease associated and tissue selective proteases include but are not limited to Cathepsins (Cathepsin B (catB), cathepsin D, cathepsin F, cathepsin K, cathepsin L, cathepsin V, cathepsin S, cathepsin W), ADAM, ADAMTS, Kallikreins 1 to 15, HTRA1-2-3, HGFAc, PRSS, TMPRSS, elastase, PR-3, granzymes (granzyme A, B, M, H and K), fibroblast activation proteins (FAP), plasmin, urokinase plasminogen activator (uPA), Tryptase, Caspase, Thrombin, Legumain, Chymase, Collagenase, napsin A, and matripatse1-2. In some embodiments, the disease associated and/or tissue selective protease is Cathepsins (Cathepsin B (catB)).

In some embodiments, the cleavable peptide is a substrate for a protease that is co-localized in a region or a tissue expressing the cytokine receptor.

In some embodiments, the cleavable peptide is a 5-mer (i.e. peptide 5 amino acids in length), 6-mer (i.e. peptide 6 amino acids in length), 7-mer (i.e. peptide 7 amino acids in length), 8-mer (i.e. peptide 8 amino acids in length), 9-mer (i.e. peptide 9 amino acids in length), 10-mer (i.e. peptide 10 amino acids in length), 11-mer (i.e. peptide 11 amino acids in length), 12-mer (i.e. peptide 12 amino acids in length), 13-mer (i.e. peptide 13 amino acids in length), 14-mer (i.e. peptide 14 amino acids in length), 15-mer (i.e. peptide 15 amino acids in length), 16-mer (i.e. peptide 16 amino acids in length), 17-mer (i.e. peptide 17 amino acids in length), or 18-mer (i.e. peptide 18 amino acids in length).

In some embodiments, the cleavable peptide is from 5 to 18 amino acids in length. In some embodiments, the cleavable peptide is from 6 to 10 amino acids in length.

In some embodiments, the cleavable peptide within the cleavable linker comprises an amino acid sequence selected from the group consisting of SEQ ID NOs: 34, 35, 36, 37, 38, 39, and 40. In some embodiments, the cleavable peptide within the cleavable linker comprises an amino acid sequence selected from the group consisting of SEQ ID NOs: 34, 35, 36, 37, 38, 39, and 40.

In some embodiments, the cleavable peptide within the cleavable linker comprises SEQ ID NO: 34. In some embodiments, the cleavable peptide within the cleavable linker comprises SEQ ID NO: 35. In some embodiments, the cleavable peptide within the cleavable linker comprises SEQ ID NO: 36. In some embodiments, the cleavable peptide within the cleavable linker comprises SEQ ID NO: 37. In some embodiments, the cleavable peptide within the cleavable linker comprises SEQ ID NO: 38. In some embodiments, the cleavable peptide within the cleavable linker comprises SEQ ID NO: 39. In some embodiments, the cleavable peptide within the cleavable linker comprises SEQ ID NO: 40. In some embodiments, the cleavable peptide within the cleavable linker comprises SEQ ID NO: 183 (PANLVAPDP). In some embodiments, the cleavable peptide within the cleavable linker comprises SEQ ID NO: 184 (IHVTLKSL). In some embodiments, the cleavable peptide within the cleavable linker comprises SEQ ID NO: 185 (NSYTIKGL). In some embodiments, the cleavable peptide within the cleavable linker comprises SEQ ID NO: 186 (SNESLSLS). In some embodiments, the cleavable peptide within the cleavable linker comprises SEQ ID NO: 187 (SQESLSLS). In some embodiments, the cleavable peptide within the cleavable linker comprises SEQ ID NO: 188 (QVSSSLSP). In some embodiments, the cleavable peptide within the cleavable linker comprises SEQ ID NO: 189 (PTSTSLSP). In some embodiments, the cleavable peptide within the cleavable linker comprises SEQ ID NO: 190 (ESLSLSEE). In some embodiments, the cleavable peptide within the cleavable linker comprises SEQ ID NO: 191 (ASLSLAPV). In some embodiments, the cleavable peptide within the cleavable linker comprises SEQ ID NO: 192 (PLGL). In some embodiments, the cleavable peptide within the cleavable linker comprises SEQ ID NO: 193 (RPLALWRS). In some embodiments, the cleavable peptide within the cleavable linker comprises SEQ ID NO: 194 (TQKPLGLS). In some embodiments, the cleavable peptide within the cleavable linker comprises SEQ ID NO: 195 (APAGLIVPYN). In some embodiments, the cleavable peptide within the cleavable linker comprises SEQ ID NO: 196 (PVSLRSGS). In some embodiments, the cleavable peptide within the cleavable linker comprises SEQ ID NO: 197 (GMPKDLYHAS). In some embodiments, the cleavable peptide within the cleavable linker comprises SEQ ID NO: 198 (RSKYLATA). In some embodiments, the cleavable peptide within the cleavable linker comprises SEQ ID NO: 199 (IVGRPRHQGV).

TABLE 1
Exemplary Cleavage Peptides

| SEQ | Sequence (* indicates a cleavage |
|---|---|
| ID NO: | site within the cleavable peptide) |
| 34 | MPYD*LYHP |
| 35 | DSGG*FMLT |
| 36 | HEQ*LTV |
| 37 | RAAA*VKSP |
| 38 | VPLS*LY |
| 39 | DLLA*VVAAS |
| 40 | ISSGLL*SG*RS |
| 183 | PANLVAPDP |
| 184 | IHVTLKSL |
| 185 | NSYTIKGL |
| 186 | SNESLSLS |
| 187 | SQESLSLS |
| 188 | QVSSSLSP |
| 189 | PTSTSLSP |
| 190 | ESLSLSEE |
| 191 | ASLSLAPV |
| 192 | PLGL |
| 193 | RPLALWRS |
| 194 | TQKPLGLS |
| 195 | APAGLIVPYN |
| 196 | PVSLRSGS |
| 197 | GMPKDLYHAS |
| 198 | RSKYLATA |
| 199 | IVGRPRHQGV |
| 200 | GFLG |
| 201 | GLFG |
| 202 | AGRRAAK |
| 203 | FRLWA |
| 204 | FRLWS |
| 205 | NFFGVGGE |
| 206 | PMKRLTLA |
| 207 | FPLATYAP |
| 208 | FLVGGASL |
| 209 | KPMQFLGD |
| 210 | GIVRAKGV |
| 211 | ALFKSSFP |
| 212 | SGRRSPGG |
| 213 | SLGRRPGG |
| 214 | SLSGRRGG |
| 215 | SLSLGRRG |
| 216 | SLSLSGRR |
| 217 | GGPRRL |
| 218 | GGPLRL |
| 219 | GGPKLL |
| 220 | GGPRNL |
| 221 | GGPRML |
| 222 | EHLRSPGG |
| 223 | FRSGVPGG |
| 224 | SLLLRTGN |
| 225 | AGLRSPGG |
| 226 | SLFRSAGP |
| 227 | SLFRAPGP |
| 228 | WLFRSPLG |
| 229 | SRLRSPQG |
| 230 | SLVLSGRR |
| 231 | KQLRHMRG |
| 232 | LSGRSDNH |
| 233 | LSGK |
| 234 | LSGR |
| 235 | RQARVVGG |
| 236 | RQRRVVGG |
| 237 | RQYRVVGG |
| 238 | SKGRSLIG |
| 239 | PRFKIIGG |
| 240 | KQLRVVNG |
| 241 | IQPRITGG |
| 242 | KQSRKFVP |
| 243 | GRQSRAGG |
| 244 | SGRSSPGG |
| 245 | SSGRSPGG |
| 246 | SLSGRSGG |
| 247 | SLSSGRSG |
| 248 | KLSLSGRS |
| 249 | PLRLSRA |
| 250 | PLKLSRA |
| 251 | PLGLSGRS |
| 252 | PLGLRSRA |
| 253 | PLGLKSRA |
| 254 | RGSRAG |
| 255 | RLSRGK |
| 256 | RGSRGG |
| 257 | KGSRAG |
| 258 | KLSRGK |
| 259 | GRSRAG |
| 260 | LRSRGK |
| 261 | GRSRGG |
| 262 | GKSRAG |
| 263 | LKSRGK |

Purely by way of example, in the above table, * indicates a known or observed protease cleavage site within the cleavable peptide.

In some embodiments, the cleavable peptide comprises an amino acid sequence selected from Table 1.

In some embodiments, the cleavable peptide comprises an amino acid sequence PVSLRSGS (SEQ ID NO: 196), or GMPKDLYHAS (SEQ ID NO. 197), or RPLALWRS (SEQ ID NO: 193), or TQKPLGLS (SEQ ID NO:194), or APAGLIVPYN (SEQ ID NO:195), or PANLVAPDP (SEQ ID NO: 183), or IVGRPRHQGV (SEQ ID NO:199), or RSKYLATA (SEQ ID NO:198).

In some embodiments, the cleavable peptide comprises an amino acid sequence of SEQ ID NO: 41 (MPYDLYHPS). In some embodiments, the cleavable peptide may comprise an amino acid sequence of SEQ ID NO: 42 (VPLSLYSG). In some embodiments, the cleavable peptide may comprise an amino acid sequence of SEQ ID NO: 43 (ISSGLLSGRSDQP).

In some embodiments, the cleavable linker comprises an amino acid sequence of SEQ ID NO: 44. In some embodiments, the cleavable linker comprises an amino acid sequence of SEQ ID NO: 45. In some embodiments, the cleavable linker comprises an amino acid sequence of SEQ ID NO: 46. In some embodiments, the cleavable linker comprises an amino acid sequence of SEQ ID NO: 47. In some embodiments, the cleavable linker comprises an amino acid sequence of SEQ ID NO: 48. In some embodiments, the cleavable linker comprises an amino acid sequence of SEQ ID NO: 49. In some embodiments, the cleavable linker comprises an amino acid sequence of SEQ ID NO: 50. In some embodiments, the cleavable linker comprises an amino acid sequence of SEQ ID NO: 51. In some embodiments, the cleavable linker comprises an amino acid sequence of SEQ ID NO: 52. In some embodiments, the cleavable linker comprises an amino acid sequence of SEQ ID NO: 53. In some embodiments, the cleavable linker comprises an amino acid sequence of SEQ ID NO: 54. In some embodiments, the cleavable linker comprises an amino acid sequence of SEQ ID NO: 55. In some embodiments, the cleavable linker comprises an amino acid sequence of SEQ ID NO: 56. In some embodiments, the cleavable linker comprises an amino acid sequence of SEQ ID NO: 57. In some embodiments, the cleavable linker comprises an amino acid sequence of SEQ ID NO: 58. In some embodiments, the cleavable linker comprises an amino acid sequence of SEQ ID NO: 59.

| SEQ | | Sequence |
|---|---|---|
| ID NO | Code | (cleavable peptide underlined) |
| 44 | MPY_00 | GPPSGSSPMPYDLYHPSGGG |
| 45 | MPY_02 | GPPSGSSPGMPYDLYHPSGGG |
| 46 | MPY_03 | GGPMPYDLYHPSGGG |
| 47 | MPY_04 | GSPMPYDLYHPSGGG |
| 48 | MPY_05 | SSGGPMPYDLYHPSGGG |
| 49 | MPY_06 | SGSSPGMPYDLYHPSGGG |
| 50 | MPY_07 | GSSGPSGMPYDLYHPSGGG |
| 51 | MPY_08 | GSSSGPGSMPYDLYHPSGGG |
| 52 | VPL_00 | GPPSGSSPVPLSLYSGSGGG |
| 53 | VPL_02 | GGSPVPLSLYSGSGGG |
| 54 | VPL_03 | GSGGVPLSLYSGSGGG |
| 55 | VPL_04 | GGSPGVPLSLYSGSGGG |
| 56 | VPL_05 | GGSSGPVPLSLYSGSGGG |
| 57 | VPL_06 | GGSSGSGVPLSLYSGSGGG |
| 58 | VPL_07 | GGSPGSPVPLSLYSGSGGG |
| 59 | VPL_08 | GSPGVPLSLYSGSSPMPYDLYHPSGG |
| | (MPY) | |

In some embodiments, the cleavable linker comprises SEQ ID NO: 140. (GGSGGSVPLSLYSGP)

In some embodiments, the cleavable linker comprises SEQ ID NO: 141. (GGSGGSGGSVPLSLYSGP)

In some embodiments, the cleavable linker comprises SEQ ID NO: 142. (GGSGGSMPYDLYHPSGP)

In some embodiments, the cleavable linker comprises SEQ ID NO: 143. (GGSGGSGGSMPYDLYHPSGP)

In some embodiments, the cleavable linker comprises SEQ ID NO: 144. (GGSGGSDSGGFMLTSGP)

In some embodiments, the cleavable linker comprises SEQ ID NO: 145. (GGSGGSGGSDSGGFMLTSGP)

In some embodiments, the cleavable linker comprises SEQ ID NO: 146. (GGSGGSRAAAVKSPSGP)

In some embodiments, the cleavable linker comprises SEQ ID NO: 147. (GGSGGSGGSRAAAVKSPSGP)

In some embodiments, the cleavable linker comprises SEQ ID NO: 148. (GGSGGSISSGLLSGRSSGP)

In some embodiments, the cleavable linker comprises SEQ ID NO: 149. (GGSGGSGGSISSGLLSGRSSGP).

In some embodiments, the cleavable linker comprises an amino acid sequence SPGGGGPMPYDLYHPSGGG (SEQ ID NO: 265).

In some embodiments, the non-cleavable linker comprises an amino acid sequence as shown in SEQ ID NO: 134 (GGGGS) and the cleavable linker comprises SEQ ID NO: 140 (GGSGGSVPLSLYSGP).

In some embodiments, the non-cleavable linker comprises an amino acid sequence as shown in SEQ ID NO: 135 (GGGGSGGGGS) and the cleavable linker comprises SEQ ID NO: 140 (GGSGGSVPLSLYSGP).

In some embodiments, the non-cleavable linker comprises an amino acid sequence as shown in SEQ ID NO: 136 (GGSGGGSGGGGGS) and the cleavable linker comprises SEQ ID NO: 140 (GGSGGSVPLSLYSGP).

In some embodiments, the non-cleavable linker comprises an amino acid sequence as shown in SEQ ID NO: 134 (GGGGS) and the cleavable linker comprises SEQ ID NO: 141 (GGSGGSGGSVPLSLYSGP).

In some embodiments, the non-cleavable linker comprises an amino acid sequence as shown in SEQ ID NO: 135 (GGGGSGGGGS) and the cleavable linker comprises SEQ ID NO: 141 (GGSGGSGGSVPLSLYSGP).

In some embodiments, the non-cleavable linker comprises an amino acid sequence as shown in SEQ ID NO: 136 (GGSGGGSGGGGGS) and the cleavable linker comprises SEQ ID NO: 141 (GGSGGSGGSVPLSLYSGP).

Spacer Domains

A spacer domain may consist of one or more amino acids. The function of the spacer domains, where present, is to link the proteolytically cleavable peptide (CP) to the other functional components in the constructs described herein.

It will be understood that spacer domains do not alter the biological interaction of the proteolytically cleavable peptide with proteases in the tumor-cell environment or in non-tumor cell environment. In other words, even in the presence of spacer domains the inventive proteolytically cleavable peptides disclosed herein retain their advantageous tumor specificity.

In some embodiments, the spacer domains flanking the proteolytically cleavable peptide are different.

In some embodiments, the spacer domains are rich in amino acid residues G, S and P.

In some embodiments, the spacer domains only includes amino acid residue types selected from the group consisting of G, S and P.

In some embodiments, the cleavable linker comprises formula 12:

N′SD1-CP-SD2C′ (12)

where SD1 is a first spacer domain and SD2 is a second spacer domain.

In some embodiments, the cleavable linker comprises formula 12:

N′SD1-CP-SD2C′ (12)

In some embodiments, the first polypeptide chain comprises formula 7 and the second polypeptide chain comprises formula 13 below:

N′HL1-non-cleavable L1-MM C′ (7)

N′HL2-SD1-CP-SD2-C C′ (13)

In some embodiments, the first polypeptide chain comprises formula 14 and the second polypeptide chain comprises formula 10 below:

N′ HL1-SD1-CP-SD2-MM C′ (14) N′ HL2-non-cleavable L2-C C′ (10)

In some embodiments, SD1 consists of a glycine (G).

In some embodiments, the N-terminus of SD1 is a glycine (G).

In some embodiments, the first spacer domain (SD1) is between 3 and 10 amino acids in length. In some embodiments, the first spacer domain (SD1) is between 4 and 9 amino acids in length. In some embodiments, the first spacer domain (SD1) is between 3 and 6 amino acids in length.

In some embodiments, SD1 comprises SEQ ID NO: 60, 61, 62, 63, 64, 65, 66, 67, 68, 69, 70, 71, or 72. In some embodiments, SD2 comprises SEQ ID NO: 60, 61, 62, 63, 64, 65, 66, 67, 68, 69, 70, 71 or 72. In some embodiments, SD1 comprises SEQ ID NO: 266. In some embodiments, SD2 comprises SEQ ID NO: 266.

| SEQ ID NO of SD1 | Sequence |
|---|---|
| 60 | GGSSPP |
| 61 | GSGP |
| 62 | GSPG |
| 63 | GGSG |
| 64 | GPPSGSSPG |
| 65 | GPPSGSSP |
| 66 | GGPS |
| 67 | GSGPS |
| 68 | GSSGGP |
| 69 | GSP |
| 70 | GSGSPS |
| 71 | PSGSSPG |
| 72 | SGSPS |
| 266 | SPGGGGP |

In some embodiments, the SD2 consists of GP.

In some embodiments, the C-terminus sequence of SD2 is -GP C′.

In some embodiments, the second spacer domain (SD2) is between 3 and 6 amino acids in length.

In some embodiments, SD2 comprises SEQ ID NO: 73, 74, or 75.

In some embodiments, SD2 consists of SEQ ID NO: 73, 74, or 75.

| SEQ ID NO of SD2 | Sequence |
|---|---|
| 73 | SGP |
| 74 | SGGG |
| 75 | GSGGG |

Exemplary combinations of SD1 and SD2 in a cleavable linker are shown below:

| Linker structure | SD1 sequence | SD2 sequence |
|---|---|---|
| SD1-CP-SD2 | GPPSGSSPG | SGGG |
| SD1-CP-SD2 | GPPSGSSPG | GSGGG |
| SD1-CP-SD2 | GPPSGSSP | SGGG |
| SD1-CP-SD2 | GGSSPP | SGP |
| SD1-CP-SD2 | GSPG | SGP |
| SD1-CP-SD2 | GGSG | SGP |
| SD1-CP-SD2 | GSGP | SGP |
| SD1-CP-SD2 | GGPS | SGP |
| SD1-CP-SD2 | GSGPS | SGP |
| SD1-CP-SD2 | GSSGGP | SGP |
| SD1-CP-SD2 | GSP | SGP |
| SD1-CP-SD2 | GSGSPS | SGP |
| SD1-CP-SD2 | G | SGP |

### Minimal Cleavage Assay

The cleavage efficiencies may be detected using any protease assay known in the art. In some embodiments, the cleavage efficiency of a cleavable linker is measured using minimal cleavage assay (MCA). The assay is developed and optimized for testing and screening engineered linker variants for their cleavage efficiencies. In particular, the amount of protease and incubation time to yield about 10-20% calculated active cytokine from un-engineered linkers are identified per MMP. The conditions identified are then used to test the cleavage efficiencies of the engineered linker variants described herein. The % calculated active cytokine levels may be used for ranking and selecting specific cleavable linkers.

### Fc Domain

Provided herein are Fc domains for use in a targeted cytokine or cleavage product thereof. A long half-life in vivo is important for therapeutic proteins. Unfortunately, cytokines that are administered to a subject generally have a short half-life since they are normally cleared rapidly from the subject by mechanisms including clearance by the kidney and endocytic degradation. Thus, in the targeted cytokine provided herein, an Fc domain is linked to the cytokine or a masking moiety for the purpose of extending the half-life of the cytokine in vivo, among other things.

In some embodiments, an Fc domain comprises a first Fc polypeptide and a second Fc polypeptide.

An Fc domain or a fragment thereof that is capable of FcRn-mediated recycling, can be reduce or otherwise delay clearance of the targeted cytokine from a subject, thereby prolonging the half-life of the administered targeted cytokine. In some embodiments, the Fc domain or a fragment thereof is any antibody or fragment thereof that is capable of FcRn-mediated recycling, such as any heavy chain polypeptide or portion thereof (e.g., Fc domain or fragment thereof) that is capable of FcRn-mediated recycling.

The Fc domain or a fragment thereof can be any antibody or fragment thereof. However, in some embodiments, either a first Fc polypeptide or a second Fc polypeptide may does not bind to the FcRn receptor, such as a light chain polypeptide. For example, in some embodiments, a first Fc polypeptide does not directly interact with the FcRn receptor, but the targeted cytokine nonetheless has an extended half-life due to comprising a second Fc polypeptide that is capable of interacting with the FcRn receptor, such as by comprising a heavy chain polypeptide. It is recognized in the art that FcRn-mediated recycling requires binding of the FcRn receptor to the Fc region of the antibody or fragment thereof. For instance, studies have shown that residues I253, S254, H435, and Y436 (numbering according to the Kabat EU index numbering system) are important for the interaction between the human Fc region and the human FcRn complex. See, e.g., Firan, M., et al., Int. Immunol. 13 (2001) 993-1002; Shields, R. L., et al, J. Biol. Chem. 276 (2001) 6591-6604). Various mutants of residues 248-259, 301-317, 376-382, and 424-437 (numbering according to the Kabat EU index numbering system) have also been examined and reported. Yeung, Y. A., et al. (J. Immunol. 182 (2009) 7667-7671.

In some embodiments, the antibody or fragment thereof comprises either a heavy chain polypeptide or a light chain polypeptide. In some embodiments, the antibody or fragment thereof comprises a portion of either a heavy chain polypeptide or a light chain polypeptide. In some embodiments, the antibody or fragment thereof comprises an Fc domain or fragment thereof. In some embodiments, the antibody or fragment thereof comprises a CH2 and CH3 domain or a fragment thereof. In some embodiments, the antibody or fragment thereof comprises the constant domain of the heavy chain polypeptide. In some embodiments, the antibody or fragment thereof comprises the constant domain of the light chain polypeptide. In some embodiments, the antibody or fragment thereof comprises a heavy chain polypeptide or fragment thereof (e.g., an Fc domain or fragment thereof). In some embodiments, the antibody or fragment thereof comprises a light chain polypeptide.

In some embodiments, the first and/or second Fc domains each contain one or more modifications that promote the non-covalent association of the first and the second Fc polypeptides. In some embodiments, the first Fc polypeptide comprises an IgG1 Fc domain or fragment thereof including the mutations Y349C; T366S; L368A; and Y407V to form a ‘hole’ in the first half-life extension domain and the second Fc polypeptide comprises an IgG1 Fc domain or fragment thereof including the mutations S354C and T366W to form the ‘knob’ in the second half-life extension domain.

In some embodiments, the first and second Fc polypeptides are each an IgG1, IgG2 or IgG4 Fc domain or fragment thereof. In some embodiments, the first and second Fc polypeptides are each an IgG1 Fc domain or fragment thereof. Human IgG1 Immunoglobulin heavy constant gamma 1 has the sequence:

(SEQ ID NO: 76)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPG

In some embodiments, the first and second Fc polypeptides are derived from the sequence for human IgG1 Immunoglobulin heavy constant gamma 1 having SEQ ID NO: 76 (the ‘parent sequence’), such that the first and second Fc polypeptides each comprise SEQ ID NO: 76 or fragment thereof, with one or more amino acid modifications.

In some embodiments, the first and Fc polypeptides each comprise the portion of SEQ ID NO: 76 shown in bold above, optionally with one or more amino acid modifications, i.e.:

(SEQ ID NO: 77)
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPG

In some embodiments, the first and second Fc polypeptides comprise SEQ ID NO: 77 with amino substitutions to promote association of the first and second Fc polypeptides according to the ‘knob into holes’ approach. In some embodiments, the sequence SEQ ID NO: 77 contains mutations Y349C; T366S; L368A; and Y407V (numbered according to the Kabat EU numbering system) to form the ‘hole’ in the first Fc polypeptide and mutations S354C and T366W (numbered according to the Kabat EU numbering system) to form the ‘knob’ in the second Fc polypeptide. These modified sequences have SEQ ID NOs 78 and 79 shown below:

First Fc polypeptide (Y349C; T366S; L368A; and
Y407V) SEQ ID NO 78:
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVCTLPPSRDELTKNQVSLSCAVK
GFYPSDIAVEWESNGQPENNYKTTPPVLDSDGSFFLVSKLTVDKSRWQQG
NVFSCSVMHEALHNHYTQKSLSLSPG
Second Fc polypeptide (S354C and T366W)
SEQ ID NO 79:
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPCRDELTKNQVSLWCLVK
GFYPSDIAVEWESNGQPENNYKTTPPVLDSDGSFFLYSKLTVDKSRWQQG
NVFSCSVMHEALHNHYTQKSLSLSPG

In some embodiments, the first and second half-life extension domains each further comprise amino substitution N297A, numbered according to the Kabat EU numbering system:

First Fc polypeptide (Y349C; T366S; L368A; Y407V
and N297A) SEQ ID NO 80:
DKTHTCPPCPAPELLGGPSVFLFPPKPKDTLMISRTPEVTCVVVDVSHED
PEVKFNWYVDGVEVHNAKTKPREEQYASTYRVVSVLTVLHQDWLNGKEYK
CKVSNKALPAPIEKTISKAKGQPREPQVCTLPPSRDELTKNQVSLSCAVK
GFYPSDIAVEWESNGQPENNYKTTPPVLDSDGSFFLVSKLTVDKSRWQQG
NVFSCSVMHEALHNHYTQKSLSLSPG
Second Fc polypeptide (S354C, T366W and N297A)
SEQ ID NO 81:
DKTHTCPPCPAPELLGGPSVFLFPPKPKDTLMISRTPEVTCVVVDVSHED
PEVKFNWYVDGVEVHNAKTKPREEQYASTYRVVSVLTVLHQDWLNGKEYK
CKVSNKALPAPIEKTISKAKGQPREPQVYTLPPCRDELTKNQVSLWCLVK
GFYPSDIAVEWESNGQPENNYKTTPPVLDSDGSFFLYSKLTVDKSRWQQG
NVFSCSVMHEALHNHYTQKSLSLSPG

In some embodiments, the first and second Fc polypeptides each further comprise the amino substitution I253A, numbered according to the Kabat EU numbering system.

In some embodiments, the first and second Fc polypeptides each further comprise both the amino substitutions N297A and I253A, numbered according to the Kabat EU numbering system.

First Fc polypeptide (Y349C; T366S; L368A; Y407V,
N297A and I253A) SEQ ID NO 82:
DKTHTCPPCPAPELLGGPSVFLFPPKPKDTLMASRTPEVTCVVVDVSHED
PEVKFNWYVDGVEVHNAKTKPREEQYASTYRVVSVLTVLHQDWLNGKEYK
CKVSNKALPAPIEKTISKAKGQPREPQVCTLPPSRDELTKNQVSLSCAVK
GFYPSDIAVEWESNGQPENNYKTTPPVLDSDGSFFLVSKLTVDKSRWQQG
NVFSCSVMHEALHNHYTQKSLSLSPG
Second Fc polypeptide (S354C, T366W, N297A and
I253A) SEQ ID NO 83:
DKTHTCPPCPAPELLGGPSVFLFPPKPKDTLMASRTPEVTCVVVDVSHED
PEVKFNWYVDGVEVHNAKTKPREEQYASTYRVVSVLTVLHQDWLNGKEYK
CKVSNKALPAPIEKTISKAKGQPREPQVYTLPPCRDELTKNQVSLWCLVK
GFYPSDIAVEWESNGQPENNYKTTPPVLDSDGSFFLYSKLTVDKSRWQQG
NVFSCSVMHEALHNHYTQKSLSLSPG

In some embodiments, the first Fc polypeptide comprises an amino acid sequence having about or at least about 85%, 86%, 87%, 88%, 89%, 90%, 91%, 92%, 93%, 94%, 95%, 96%, 97%, 98%, or 99% sequence identity to any one of the amino acid sequence of any one of SEQ ID NOs: 77-83.

In some embodiments, the second Fc polypeptide comprises an amino acid sequence having about or at least about 85%, 86%, 87%, 88%, 89%, 90%, 91%, 92%, 93%, 94%, 95%, 96%, 97%, 98%, or 99% sequence identity to any one of the amino acid sequence of any one of SEQ ID NOs: 77-83.

In some embodiments, the first Fc polypeptide comprises an amino acid sequence having one or more modifications, such as one or more amino acid substitutions, additions, or deletions, as compared to the amino acid sequence of any one of SEQ ID NOs: 77-83. In some embodiments, the second Fc polypeptide comprises an amino acid sequence having one or more modifications, such as one or more amino acid substitutions, additions, or deletions, as compared to the amino acid sequence of any one of SEQ ID NOs: 77-83. The one or more modifications can be any modifications or alterations described herein, including, in some embodiments, any modifications or alterations disclosed herein that promote heterodimerization of polypeptide chains and/or suppresses homodimerization of polypeptide chains, alter effector function, or enhance effector function.

In some embodiments, the Fc domain or fragment thereof comprises one or more amino acid substitutions altering effector function. In some embodiments, the half-life extension domain is an IgG1 Fc domain or fragment thereof and comprises one or more amino acid substitutions selected from the group consisting of N297A, N297G, N297Q, L234A, L235A, C220S, C226S, C229S, P238S, E233P, L234V, L234F, L235E, P331S, S267E, L328F, D265A, and P329G, numbered according to the Kabat EU numbering system. In some embodiments, the half-life extension domain is an IgG2 Fc domain or fragment thereof and comprises the amino substitution(s): V234A and G237A; H268Q, V309L, A330S, and A331S; and/or V234A, G237A, P238S, H268A, V309L, and A330S, numbered according to the Kabat EU numbering system. In some embodiments, the half-life extension domain is an IgG2 Fc domain or fragment thereof and comprises one or more amino acid substitutions selected from the group consisting of V234A, G237A, H268Q, V309L, A330S, A331S, P238S, H268A, and V309L, numbered according to the Kabat EU numbering system. In some embodiments, the half-life extension domain is an IgG4 Fc domain or fragment thereof and comprises the amino substitution(s): L235A, G237A, and E318A; S228P, L234A, and L235A; H268Q, V309L, A330S, and P331S; and/or S228P and L235A, numbered according to the Kabat EU numbering system. In some embodiments, the half-life extension domain is an IgG2 Fc domain or fragment thereof and comprises one or more amino acid substitutions selected from the group consisting of L235A, G237A, E318A, S228P, L234A, H268Q, V309L, A330S, and P331S, numbered according to the Kabat EU numbering system.

In some embodiments, the Fc domain or fragment thereof that comprises one or more amino acid substitutions enhancing effector function. In some embodiments, the half-life extension domain is an IgG1 Fc domain or fragment thereof and comprises the amino acid substitution(s): S298A, E333A, and K334A; S239D and I332E; S239D, A330L, and I332E; P247I and A339D or A339Q; D280H and K290S; D280H, K290S, and either S298D or S298V; F243L, R292P, and Y300L; F243L, R292P, Y300L, and P396L; F243L, R292P, Y300L, V305I, and P396L; G236A, S239D, and I332E; K326A and E333A; K326W and E333S; K290E, S298G, and T299A; K290E, S298G, T299A, and K326E; K290N, S298G, and T299A; K290N, 5298G, T299A, and K326E; K334V; L235S, S239D, and K334V; K334V and Q331M, S239D, F243V, E294L, or S298T; E233L, Q311M, and K334V; L234I, Q311M, and K334V; K334V and S298T, A330M, or A330F; K334V, Q311M, and either A330M or A330F; K334V, S298T, and either A330M or A330F; K334V, S239D, and either A330M or S298T; L234Y, Y296W, and K290Y, F243V, or E294L; Y296W and either L234Y or K290Y; S239D, A330S, and I332E, V264I; F243L and V264I; L328M; I332E; L328M and I332E; V264I and I332E; S239E and I332E; S239Q and I332E; S239E; A330Y; I332D; L328I and I332E; L328Q and I332E; V264T; V240I; V266I; S239D; S239D and I332D; S239D and I332N; S239D and I332Q; S239E and I332D; S239E and I332N; S239E and I332Q; S239N and I332D; S239N and I332E; S239Q and I332D; A330Y and I332E; V264I, A330Y, and I332E; A330L and I332E; V264I, A330L, and I332E; L234E, L234Y, or L234I; L235D, L235S, L235Y, or L235I; S239T; V240M; V264Y; A330I; N325T; I332E and L328D, L328V, L328T, or L328I; V264I, I332E, and either S239E or S239Q; S239E, V264I, A330Y, and I332E; A330Y, I332E, and either S239D or S239N; A330L, I332E, and either S239D or S239N; V264I, S298A, and I332E; S298A, I332E, and either S239D or S239N; S239D, V264I, and I332E; S239D, V264I, S298A, and I332E; S239D, V264I, A330L, and I332E; S239D, I332E, and A330I; P230A; P230A, E233D, and I332E; E272Y; K274T, K274E, K274R, K274L, or K274Y; F275W; N276L; Y278T; V302I; E318R; S324D, S324I or S324V; K326I or K326T; T335D, T335R, or T335Y; V240I and V266I; S239D, A330Y, I332E, and L234I; S239D, A330Y, I332E, and L235D; S239D, A330Y, I332E, and V240I; S239D, A330Y, I332E, and V264T; and/or S239D, A330Y, I332E, and either K326E or K326T, numbered according to the Kabat EU numbering system. In some embodiments, the Fc domain is an IgG1 Fc domain or fragment thereof and comprises one or more amino acid substitution(s) selected from the group consisting of: P230A, E233D, L234E, L234Y, L234I, L235D, L235S, L235Y, L235I, S239D, S239E, S239N, S239Q, S239T, V240I, V240M, F243L, V264I, V264T, V264Y, V266I, E272Y, K274T, K274E, K274R, K274L, K274Y, F275W, N276L, Y278T, V302I, E318R, S324D, S324I, S324V, N325T, K326I, K326T, L328M, L328I, L328Q, L328D, L328V, L328T, A330Y, A330L, A330I, I332D, I332E, I332N, I332Q, T335D, T335R, and T335Y.

In some embodiments, the Fc domain comprises one or more amino acid substitution(s) that enhance binding of the half-life extension domain to FcRn. In some embodiments, the one or more amino acid substitution(s) increase binding affinity of an Fc-containing polypeptide (e.g., a heavy chain polypeptide or an Fc domain or fragment thereof) to FcRn at acidic pH. In some embodiments, the half-life extension domain comprises one or more amino acid substitution(s) selected from the group consisting of M428F; T250Q and M428F; M252Y, S254T, and T256E; P257I and N434H; D376V and N434H; P257I and Q311I; N434A; N434W; M428F and N434S; V259I and V308F; M252Y, S254T, and T256E; V259I, V308F and M428F; T307Q and N434A; T307Q and N434S; T307Q, E380A, and N434A; V308P and N434A; N434H; and V308P.

Knobs-into-Holes Approach

One strategy for promoting heterodimerization of two Fc polypeptides is an approach termed the “knobs-into-holes”.

In some embodiments, the targeted cytokine comprises a first Fc polypeptide and a second Fc polypeptide, each of which comprises a CH3 domain. In some embodiments, the Fc polypeptide comprising a CH3 domain is a heavy chain polypeptide or a fragment thereof (e.g., an Fc domain or fragment thereof). The CH3 domains of the two Fc polypeptides can be altered by the “knobs-into-holes” technology, which is described in detail with several examples in, e.g., WO 1996/027011; Ridgway, J. B. et al, Protein Eng. (1996) 9(7): 617-621; Merchant, A. M., et al, Nat. Biotechnol. (1998) 16(7): 677-681. See also Klein et al. (2012), MAbs, 4(6): 653-663. Using the knob-into-holes method, the interaction surfaces of the two CH3 domains are altered to increase the heterodimerization of the two half-life extension domains containing the two altered CH3 domains. This occurs by introducing a bulky residue into the CH3 domain of one of the half-life extension domains, which acts as the “knob.” Then, in order to accommodate the bulky residue, a “hole” is formed in the other half-life extension domain that can accommodate the knob. Either of the altered CH3 domains can be the “knob” while the other can be the “hole.” The introduction of a disulfide bridge further stabilizes the heterodimers (Merchant, A. M., et al, Nat. Biotechnol. (1998) 16(7); Atwell, S., et al, J. Mol. Biol. (1997) 270(1): 26-35) as well as increases yield.

It has been reported that heterodimerization yields above 97% can be achieved by introducing the S354C and T366W mutations in a heavy chain to create the “knob” and by introducing the Y349C, T366S, L368A, and Y407V mutations in a heavy chain to create the “hole” (numbering of the residues according to the Kabat EU numbering system). Carter et al. (2001), J. Immunol. Methods, 248: 7-15; Klein et al. (2012), MAbs, 4(6): 653-663.

In some embodiments comprising a first Fc polypeptide and a second Fc polypeptide, the first half-life Fc polypeptide comprises a heavy chain polypeptide or portion thereof (e.g., an Fc domain or fragment thereof) that comprises the amino acid mutations S354C and T366W (numbered according to the Kabat EU numbering system), and the second Fc polypeptide comprises a heavy chain polypeptide or portion thereof (e.g., an Fc domain or fragment thereof) that comprises the amino acid mutations Y349C, T366S, L368A, and Y407V (numbered according to the Kabat EU numbering system). In some embodiments comprising a first Fc polypeptide and a second Fc polypeptide, the first Fc polypeptide comprises a heavy chain polypeptide or portion thereof (e.g., an Fc domain or fragment thereof) that comprises the amino acid mutations Y349C, T366S, L368A, and Y407V (numbered according to the Kabat EU numbering system), and the second Fc polypeptide comprises a heavy chain polypeptide or portion thereof (e.g., an Fc domain or fragment thereof) that comprises the amino acid mutations S354C and T366W (numbered according to the Kabat EU numbering system).

Additional examples of substitutions that can be made to form knobs and holes include those described in US20140302037A1, the contents of which are herein incorporated by reference. For example, in some embodiments, any of the following amino acid substitutions can be made to a first Fc polypeptide (“first domain”) and a paired second Fc polypeptide (“second domain”) that each contain an Fc domain: (a) Y407T in the first domain and T366Y in the second domain; (b) Y407A in the first domain and T366W in the second domain; (c) F405A in the first domain and T394W in the second domain; (d) F405W in the first domain and T394S in the second domain; (e) Y407T in the first domain and T366Y in the second domain; (f) T366Y and F405A in the first domain and T394W and Y407T in the second domain; (g) T366W and F405W in the first domain and T394S and Y407A in the second domain; (h) F405W and Y407A in the first domain and T366W and T394S in the second domain; or (i) T366W in the first domain and T366S, L368A, and Y407V in the second domain, numbered according to the Kabat EU numbering system.

In some embodiments, any of the following amino acid substitutions can be made to a first Fc polypeptide (“first domain”) and a paired second Fc polypeptide (“second domain”) that each contain an Fc domain: (a) Y407T in the second domain and T366Y in the first domain; (b) Y407A in the second domain and T366W in the first domain; (c) F405A in the second domain and T394W in the first domain; (d) F405W in the second domain and T394S in the first domain; (e) Y407T in the second domain and T366Y in the first domain; (f) T366Y and F405A in the second domain and T394W and Y407T in the first domain; (g) T366W and F405W in the second domain and T394S and Y407A in the first domain; (h) F405W and Y407A in the second domain and T366W and T394S in the first domain; or (i) T366W in the second domain and T366S, L368A, and Y407V in the first domain, numbered according to the Kabat EU numbering system.

In embodiments comprising a first Fc polypeptide and a second Fc polypeptide that each comprise an Fc domain, any of the heterodimerizing alterations described herein can be used in the Fc domains to promote heterodimerization of any of the targeted cytokines described herein.

RF Mutation or CH3 Domain Swap for Heterodimeric Protein Purification

Two immunoglobulin heavy chains that differ by at least one amino acid allows isolation of the antigen-binding protein based on a differential affinity of an immunoglobulin heavy chain and a modified or mutated immunoglobulin heavy chain toward an affinity reagent. The antigen-binding proteins that have IgG CH2 and CH3 regions with different affinities with respect to Protein A allow rapid isolation by differential binding of the IgG regions to Protein A.

In one embodiment, a second Fc polypeptide comprises a 95R modification (by IMGT exon numbering; 435R by EU numbering) in the CH3 region. In another embodiment, a second Fc polypeptide further comprises a 96F modification (IMGT; 436F by EU). In some embodiments, a first Fc polypeptide comprises wild-type CH2 and CH3 domains derived from IgG1 or IgG4, and a second Fc polypeptide comprises 95R/96F modifications by IMGT exon numbering. In some embodiments, a first Fc polypeptide comprises wild-type CH2 and CH3 domains derived from IgG1 or IgG4, and a second Fc polypeptide comprises 435R/436F modifications by EU numbering.

In some embodiments, a first Fc polypeptide comprises wild-type CH2 and CH3 domains derived from IgG1 or IgG4, and a second Fc polypeptide comprises CH3 domain derived from IgG3.

In some embodiments, a first Fc polypeptide or a second Fc polypeptide comprises amino acid sequence of SEQ ID NO: 84. SEQ ID NO: 84 comprises “knob mutations” with CH3 domain from IgG3.

(SEQ ID NO: 84)
DKTHTCPPCPAPELLGGPSVFLFPPKPKDTLMISRTPEVTCVVVDVSHED
PEVKFNWYVDGVEVHNAKTKPREEQYASTYRVVSVLTVLHQDWLNGKEYK
CKVSNKALPAPIEKTISKAKGQPREPQVYTLPPCREELTKNQVSLWCLVK
GFYPSDIAVEWESSGQPENNYKTTPPVLDSDGSFFLYSKLTVDKSRWQQG
NIFSCSVMHEALHNRFTQKSLSLSPGGSPG

In some embodiments, a first Fc polypeptide or a second Fc polypeptide comprises amino acid sequence of SEQ ID NO: 85. SEQ ID NO: 85 comprises “knob mutations” with “RF mutations (435R/436F).

(SEQ ID NO: 85)
DKTHTCPPCPAPELLGGPSVFLFPPKPKDTLMISRTPEVTCVVVDVSHED
PEVKFNWYVDGVEVHNAKTKPREEQYASTYRVVSVLTVLHQDWLNGKEYK
CKVSNKALPAPIEKTISKAKGQPREPQVYTLPPCRDELTKNQVSLWCLVK
GFYPSDIAVEWESNGQPENNYKTTPPVLDSDGSFFLYSKLTVDKSRWQQG
NVFSCSVMHEALHNRFTQKSLSLSPGGSPG

In some embodiments, a first Fc polypeptide or a second Fc polypeptide comprises amino acid sequence of SEQ ID NO: 86.

(SEQ ID NO: 86)
DKTHTCPPCPAPELLGGPSVFLFPPKPKDTLMISRTPEVTCVVVDVSHED
PEVKFNWYVDGVEVHNAKTKPREEQYASTYRVVSVLTVLHQDWLNGKEYK
CKVSNKALPAPIEKTISKAKGQPREPQVYTLPPCRDELTKNQVSLWCLVK
GFYPSDIAVEWESNGQPENNYKTTPPVLDSDGSFFLYSKLTVDKSRWQQG
NVFSCSVMHEALHNRFTQKSLSLSPG

In some embodiments, a first Fc polypeptide or a second Fc polypeptide comprises amino acid sequence having at least about 85% identity to SEQ ID NO: 86. In some embodiments, a first Fc polypeptide or a second Fc polypeptide comprises amino acid sequence having at least about 90% identity to SEQ ID NO: 86. In some embodiments, a first Fc polypeptide or a second Fc polypeptide comprises amino acid sequence having at least about 91% identity to SEQ ID NO: 86. In some embodiments, a first Fc polypeptide or a second Fc polypeptide comprises amino acid sequence having at least about 92% identity to SEQ ID NO: 86. In some embodiments, a first Fc polypeptide or a second Fc polypeptide comprises amino acid sequence having at least about 93% identity to SEQ ID NO: 86. In some embodiments, a first Fc polypeptide or a second Fc polypeptide comprises amino acid sequence having at least about 94% identity to SEQ ID NO: 86. In some embodiments, a first Fc polypeptide or a second Fc polypeptide comprises amino acid sequence having at least about 95% identity to SEQ ID NO: 86. In some embodiments, a first Fc polypeptide or a second Fc polypeptide comprises amino acid sequence having at least about 96% identity to SEQ ID NO: 86. In some embodiments, a first Fc polypeptide or a second Fc polypeptide comprises amino acid sequence having at least about 97% identity to SEQ ID NO: 86. In some embodiments, a first Fc polypeptide or a second Fc polypeptide comprises amino acid sequence having at least about 98% identity to SEQ ID NO: 86. In some embodiments, a first Fc polypeptide or a second Fc polypeptide comprises amino acid sequence having at least about 99% identity to SEQ ID NO: 86.

In some embodiments, a first Fc polypeptide or a second Fc polypeptide comprises amino acid sequence having at least about 85% identity to SEQ ID NO: 80. In some embodiments, a first Fc polypeptide or a second Fc polypeptide comprises amino acid sequence having at least about 90% identity to SEQ ID NO: 80. In some embodiments, a first Fc polypeptide or a second Fc polypeptide comprises amino acid sequence having at least about 91% identity to SEQ ID NO: 80. In some embodiments, a first Fc polypeptide or a second Fc polypeptide comprises amino acid sequence having at least about 92% identity to SEQ ID NO: 80. In some embodiments, a first Fc polypeptide or a second Fc polypeptide comprises amino acid sequence having at least about 93% identity to SEQ ID NO: 80. In some embodiments, a first Fc polypeptide or a second Fc polypeptide comprises amino acid sequence having at least about 94% identity to SEQ ID NO: 80. In some embodiments, a first Fc polypeptide or a second Fc polypeptide comprises amino acid sequence having at least about 95% identity to SEQ ID NO: 0. In some embodiments, a first Fc polypeptide or a second Fc polypeptide comprises amino acid sequence having at least about 96% identity to SEQ ID NO: 80. In some embodiments, a first Fc polypeptide or a second Fc polypeptide comprises amino acid sequence having at least about 97% identity to SEQ ID NO: 80. In some embodiments, a first Fc polypeptide or a second Fc polypeptide comprises amino acid sequence having at least about 98% identity to SEQ ID NO: 80. In some embodiments, a first Fc polypeptide or a second Fc polypeptide comprises amino acid sequence having at least about 99% identity to SEQ ID NO: 80.

### Exemplary Targeted Cytokines

In some embodiments, a targeted IL-2 cytokine of the present invention comprises a first chain comprising SEQ ID NO: 87, a second chain comprising SEQ ID NO: 88, and a third chain comprising SEQ ID NO: 8.

(SEQ ID NO: 87)
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYA
STYRVVSVLTVLHQDWLNGKEYKCKVSNKALPAPIEKTISKAKGQPREPQ
VYTLPPCRDELTKNQVSLWCLVKGFYPSDIAVEWESNGQPENNYKTTPPV
LDSDGSFFLYSKLTVDKSRWQQGNVFSCSVMHEALHNRFTQKSLSLSPGG
GSSPPGGGSSGGGSGPAPTSSSTKKTQLQLEHLLLDLQMILNGINNYKNP
KLTAMLTAKFAMPKKATELKHLQCLEEALKPLEEVLNLAQSKNFHLRPRD
LISNINVIVLELKGSETTFMCEYADETATIVEFLNRWITFAQSIISTLT
(SEQ ID NO: 88)
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYA
STYRVVSVLTVLHQDWLNGKEYKCKVSNKALPAPIEKTISKAKGQPREPQ
VCTLPPSRDELTKNQVSLSCAVKGFYPSDIAVEWESNGQPENNYKTTPPV
LDSDGSFFLVSKLTVDKSRWQQGNVFSCSVMHEALHNHYTQKSLSLSPGG
PPSGSSPMPYDLYHPSGGGAVNGTSQFTCFYNSRANISCVWSQDGALQDT
SCQVHAWPDRRRWNQTCELLPVSQASWACNLILGAPDSQKLTTVDIVTLR
VLCREGVRWRVMAIQDFKPFENLRLMAPISLQVVHVETHRSNISWEISQA
SHYFERHLEFEARTLSPGHTWEEAPLLTLKQKQEWISLETLTPDTQYEFQ
VRVKPLQGEFTTWSPWSQPLAFRTKPAALGKD

In some embodiments, a targeted IL-2 cytokine of the present invention comprises a first chain comprising SEQ ID NO: 87, a second chain comprising SEQ ID NO: 89, and a third chain comprising SEQ ID NO: 8.

(SEQ ID NO: 89)
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYA
STYRVVSVLTVLHQDWLNGKEYKCKVSNKALPAPIEKTISKAKGQPREPQ
VCTLPPSRDELTKNQVSLSCAVKGFYPSDIAVEWESNGQPENNYKTTPPV
LDSDGSFFLVSKLTVDKSRWQQGNVFSCSVMHEALHNHYTQKSLSLSPGG
PPSGSSPMPYDLYHPSGGGAVNGTSQFTCFYNSRANISCVWSQDGALQDT
SCQVHAWPDRRRWNQTCELLPVSQASWACNLILGAPDSQKLTTVDIVTLR
VLCREGVRWRVMAIQDFKPFENLRLMAPISLQVVHVETHRACISWEISQA
SHYFERHLEFEARTLSPGHTWEEAPLLTLKQKQEWICLETLTPDTQYEFQ
VRVKPLQGEFTTWSPWSQPLAFRTKPAALGKD

In some embodiments, a targeted IL-2 cytokine of the present invention comprises a first chain comprising SEQ ID NO: 87, a second chain comprising SEQ ID NO: 90, and a third chain comprising SEQ ID NO: 8.

(SEQ ID NO: 90)
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYA
STYRVVSVLTVLHQDWLNGKEYKCKVSNKALPAPIEKTISKAKGQPREPQ
VCTLPPSRDELTKNQVSLSCAVKGFYPSDIAVEWESNGQPENNYKTTPPV
LDSDGSFFLVSKLTVDKSRWQQGNVFSCSVMHEALHNHYTQKSLSLSPGG
GSSGPVPLSLYSGSGGGAVNGTSQFTCFYNSRANISCVWSQDGALQDTSC
QVHAWPDRRRWNQTCELLPVSQASWACNLILGAPDSQKLTTVDIVTLRVL
CREGVRWRVMAIQDFKPFENLRLMAPISLQVVHVETHRSNISWEISQASH
YFERHLEFEARTLSPGHTWEEAPLLTLKQKQEWISLETLTPDTQYEFQVR
VKPLQGEFTTWSPWSQPLAFRTKPAALGKD

In some embodiments, a targeted IL-2 cytokine of the present invention comprises a first chain comprising SEQ ID NO: 87, a second chain comprising SEQ ID NO: 91, and a third chain comprising SEQ ID NO: 8.

(SEQ ID NO: 91)
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYA
STYRVVSVLTVLHQDWLNGKEYKCKVSNKALPAPIEKTISKAKGQPREPQ
VCTLPPSRDELTKNQVSLSCAVKGFYPSDIAVEWESNGQPENNYKTTPPV
LDSDGSFFLVSKLTVDKSRWQQGNVFSCSVMHEALHNHYTQKSLSLSPGG
GSSGPVPLSLYSGSGGGAVNGTSQFTCFYNSRANISCVWSQDGALQDTSC
QVHAWPDRRRWNQTCELLPVSQASWACNLILGAPDSQKLTTVDIVTLRVL
CREGVRWRVMAIQDFKPFENLRLMAPISLQVVHVETHRACISWEISQASH
YFERHLEFEARTLSPGHTWEEAPLLTLKQKQEWICLETLTPDTQYEFQVR
VKPLQGEFTTWSPWSQPLAFRTKPAALGKD

In some embodiments, a targeted cytokine comprises a first polypeptide comprising a heavy chain of a targeting moiety and a first Fc polypeptide linked to a cytokine via a non-cleavable linker in N-to-C terminus orientation, a second polypeptide comprising a heavy chain of the targeting moiety, a second Fc polypeptide linked to a masking moiety via a cleavable linker in N-to-C terminus orientation, and a third polypeptide comprising a light chain of the targeting moiety that associates with the heavy chain of the targeting moiety.

In some embodiments, a targeted cytokine comprises a first polypeptide comprising a heavy chain of a targeting moiety and a first Fc polypeptide linked to a cytokine via a cleavable linker in N-to-C terminus orientation, a second polypeptide comprising a heavy chain of the targeting moiety, a second Fc polypeptide linked to a masking moiety via a non-cleavable linker in N-to-C terminus orientation, and a third polypeptide comprising a light chain of the targeting moiety that associates with the heavy chain of the targeting moiety.

In some embodiments, a targeted cytokine comprises a first polypeptide comprising a scFv targeting moiety (e.g., heavy chain variable region fused to a light chain variable region (VH-VL or VL-VH)), a first Fc polypeptide linked to a cytokine via a non-cleavable linker in N-to-C terminus orientation, and a second polypeptide comprising a scFv targeting moiety (e.g., heavy chain variable region fused to a light chain variable region (VH-VL or VL-VH)), a second Fc polypeptide linked to a masking moiety via a cleavable linker in N-to-C terminus orientation.

In some embodiments, a targeted cytokine comprises a first polypeptide comprising a scFv targeting moiety (e.g., heavy chain variable region fused to a light chain variable region (VH-VL or VL-VH)), a first Fc polypeptide linked to a cytokine via a cleavable linker in N-to-C terminus orientation, and a second polypeptide comprising a scFv targeting moiety (e.g., heavy chain variable region fused to a light chain variable region (VH-VL or VL-VH)), a second Fc polypeptide linked to a masking moiety via a non-cleavable linker in N-to-C terminus orientation.

### Other Therapeutically Active Domains

In various embodiments, the cytokine or functional fragment thereof within a targeted masked cytokine is replaced by any therapeutical active domain to generate a targeted masked therapeutical active domain. The targeted masked therapeutically active domain comprises a targeting moiety, a masking moiety, a Fc domain and a therapeutically active domain.

In some embodiments, a therapeutically active domain is a cell engager. In some embodiments, a therapeutically active domain is a T cell engager. In some embodiments, a therapeutically active domain is a bispecific T cell engager (BiTE®). In some embodiments, a therapeutically active domain is a NK cell engager. In some embodiments, a cell engager is an anti-CD3 antibody or antigen-binding fragment thereof. In some embodiments, a cell engager is an anti-CD16 antibody or antigen-binding fragment thereof. In some embodiments, a cell engager is an anti-NKG2D antibody or antigen-binding fragment thereof. In some embodiments, a cell engager is an anti-CD16/NKG2D bispecific antibody or antigen-binding fragment thereof. In some embodiments, a cell engager is an anti-CD33 antibody or antigen-binding fragment thereof. In some embodiments, a cell engager is an anti-CD3/CD33 bispecific antibody or antigen-binding fragment thereof. In some embodiments, a cell engager is an anti-B-cell maturation antigen (BCMA) antibody or antigen-binding fragment thereof. In some embodiments, a cell engager is an anti-CD19 antibody or antigen-binding fragment thereof. In some embodiments, a cell engager is an anti-CD3/CD19 bispecific antibody or antigen-binding fragment thereof. In some embodiments, a cell engager is an anti-CD56 antibody or antigen-binding fragment thereof. In some embodiments, a cell engager is an anti-CD4 antibody or antigen-binding fragment thereof. In some embodiments, a cell engager is an anti-CD8 antibody or antigen-binding fragment thereof. In some embodiments, a cell engager is an anti-CD25 antibody or antigen-binding fragment thereof. In some embodiments, a cell engager is an anti-CD127 antibody or antigen-binding fragment thereof. In some embodiments, a cell engager is an anti-FoxP3 antibody or antigen-binding fragment thereof. In some embodiments, a cell engager is an anti-CD161 antibody or antigen-binding fragment thereof. In some embodiments, a cell engager is an anti-CD94 antibody or antigen-binding fragment thereof. In some embodiments, a cell engager is an anti-CD57 antibody or antigen-binding fragment thereof.

In some embodiments, a therapeutically active domain is co-stimulatory domain. In some embodiments, a therapeutically active domain is an agonistic antibody. In some embodiments, a therapeutically active domain is an antagonistic antibody. In some embodiments, a therapeutically active domain is a CD28. In some embodiments, a therapeutically active domain is B7. In some embodiments, a therapeutically active domain is ICOS, In some embodiments, a therapeutically active domain is CD226. In some embodiments, a therapeutically active domain is 41BB. In some embodiments, a therapeutically active domain is OX40. In some embodiments, a therapeutically active domain is CD27. In some embodiments, a therapeutically active domain is GITR. In some embodiments, a therapeutically active domain is HVEM. In some embodiments, a therapeutically active domain is CD40. In some embodiments, a therapeutically active domain is BAFFR. In some embodiments, a therapeutically active domain is BAFF. In some embodiments, a therapeutically active domain is TNF. In some embodiments, a therapeutically active domain is TNF receptor. In some embodiments, a therapeutically active domain is CTLA-4. In some embodiments, a therapeutically active domain is PD-1. In some embodiments, a therapeutically active domain is CD30. In some embodiments, a therapeutically active domain is CD40L. In some embodiments, a therapeutically active domain is TIM-1. In some embodiments, a therapeutically active domain is TIM-2. In some embodiments, a therapeutically active domain is TIM-3. In some embodiments, a therapeutically active domain is CD2. In some embodiments, a therapeutically active domain is CD137. In some embodiments, a therapeutically active domain is 2B4.

In some embodiments, a therapeutically active domain is an anti-CD28 antibody. In some embodiments, a therapeutically active domain is an anti-B7 antibody. In some embodiments, a therapeutically active domain is an anti-ICOS antibody. In some embodiments, a therapeutically active domain is an anti-CD226 antibody. In some embodiments, a therapeutically active domain is an anti-41BB antibody. In some embodiments, a therapeutically active domain is an anti-OX40 antibody. In some embodiments, a therapeutically active domain is an anti-CD27 antibody. In some embodiments, a therapeutically active domain is an anti-GITR antibody. In some embodiments, a therapeutically active domain is an anti-HVEM antibody. In some embodiments, a therapeutically active domain is an anti-CD40 antibody. In some embodiments, a therapeutically active domain is an anti-BAFFR antibody. In some embodiments, a therapeutically active domain is an anti-BAFF antibody. In some embodiments, a therapeutically active domain is an anti-TNF antibody. In some embodiments, a therapeutically active domain is an anti-TNFR antibody. In some embodiments, a therapeutically active domain is an anti-CTLA-4 antibody. In some embodiments, a therapeutically active domain is an anti-PD-1 antibody. In some embodiments, a therapeutically active domain is an anti-CD30 antibody. In some embodiments, a therapeutically active domain is an anti-CD40L antibody. In some embodiments, a therapeutically active domain is an anti-TIM-1 antibody. In some embodiments, a therapeutically active domain is an anti-TIM-2 antibody. In some embodiments, a therapeutically active domain is an anti-TIM-3 antibody. In some embodiments, a therapeutically active domain is an anti-CD2 antibody. In some embodiments, a therapeutically active domain is an anti-CD137 antibody. In some embodiments, a therapeutically active domain is an anti-2B4 antibody.

### Additional Masked Constructs

Different aspects of the present invention described in this application can be used for additional masked Constructs. In some embodiments, additional masked constructs are referred to as therapeutically activatable constructs or prodrugs or masked cytokines. Such constructs are recombinant fusion protein constructs described herein are adaptable and applicable beyond targeted or masked cytokines. For example, in accordance with the present disclosure, an Fc domain can be linked cleavable or non-cleavable linker and fused to a masked therapeutically active domain comprising a therapeutically active domain and a masking moiety. Alternatively, an Fc domain can be fused to a targeted therapeutically active domain can be by a cleavable or non-cleavable linker wherein the recombinant fusion protein comprises a therapeutically active domain, a masking moiety, and a targeting moiety. Upon the cleavage by a tumor associated protease, e.g., at the protease cleavage site, the masked and targeted therapeutically active molecules are released and become active at the site of disease, and are able to specifically target a cell of interest for effective treatment of various diseases without causing undesired side effects.

In some embodiments, a therapeutically active domain is linked to the Fe domain using a linker a non-cleavable linker. In some embodiments, a therapeutically active domain is linked to the Fc domain using a linker a non-cleavable linker.

It will be understood by a skilled person that the various moieties of the targeted masked cytokine described herein may be replaced or removed to generate other types of recombinant polypeptides for a particular purpose.

In some embodiments, the recombinant fusion protein does not comprise a targeting moiety. In some embodiments, the fusion protein is a masked cytokine polypeptide only. In some embodiments, the masked cytokine comprises a masking moiety, a linker, a Fc domain and a cytokine. The linker may be a non-cleavable linker or a cleavable linker.

### Methods for Producing the Masked Constructs

Some embodiments of the methods and compositions provided herein relate to methods for producing a targeted masked agent such as a targeted masked cytokine. The targeted masked cytokines as described herein can be produced and manufactured using any known technology in the art. The targeted masked cytokines and/or constructs may be produced using any mammalian expression system.

Mammalian cells are commonly used for producing recombinant proteins. The cells are be derived from human, rat, mouse and other mammals. Commonly used mammalian cell lines include but are not limited to, HEK cells, CHO cells, recombinant CHO cells, BHK cells, NS0 cells, SP2/O-Ag14 cells, HT-1080 cells, PER.C6 cells, CAP (CEVEC's Amniocyte Production), Hela cells, and HuH-7 (Human hepatoma) cells. The cell line can be used for transient gene expression or as stable cell line for stable expression.

In some embodiments, the cells are Chinese Hamster ovary (CHO) cells. The CHO cells can be recombinant CHO cell lines, e.g., CHO-K1, CHO DUX and CHO DG44.

In some embodiments, the cells are human cells. In some embodiments, the cells are Human Embryo Kidney (HEK) cells or variants thereof (e.g., HKB11).

The host cells are transformed to express a nucleic acid or vector encoding a targeted masked cytokine described herein. Where appropriate, the engineered host cells can be cultured in conventional nutrient media.

In other embodiments, the targeted masked cytokines may be produced using non-mammalian expression systems, such as baculovirus expression systems, bacterial systems, yeast cells, insect cell lines and plant cells. The insect cell lines include but are not limited to Sf9, Sf21 and BTI 5B1-4. E. coli cells are commonly used bacteria to express a recombinant protein.

In some embodiments, the masked cytokines and/or constructs may be produced using a cell-free protein express system. Cell-free expression systems can use mRNA transcribed from a DNA construct comprising a promoter operably linked to a nucleic acid encoding the polypeptide or fragment thereof.

### Methods of Treatment

Provided herein are methods for treating or preventing a disease in a subject comprising administering to the subject an effective amount of any targeted cytokine described herein or compositions thereof. In some embodiments, methods are provided for treating or preventing a disease in a subject comprising administering to the subject any composition described herein. In some embodiments, the subject (e.g., a human patient) has been diagnosed with cancer or is at risk of developing such a disorder. In some embodiments, methods are provided for treating or preventing disease in a subject comprising administering to the subject an effective amount of any targeted cytokine described herein or compositions thereof, wherein the targeted cytokine is activated upon cleavage by an enzyme. In some embodiments, the targeted cytokine is activated at a tumor microenvironment. The targeted cytokine is therapeutically active after it has cleaved. Thus, in some embodiments, the active agent is the cleavage product.

For the prevention or treatment of disease, the appropriate dosage of an active agent will depend on the type of disease to be treated, as defined herein, the severity and course of the disease, whether the agent is administered for preventive or therapeutic purposes, previous therapy, the subject's clinical history and response to the agent, and the discretion of the attending physician. The agent is suitably administered to the subject at one time or over a series of treatments.

In some embodiments of the methods described herein, an interval between administrations of a targeted cytokine described herein is about one week or longer. In some embodiments of the methods described herein, an interval between administrations of a targeted cytokine described herein is about two days or longer, about three days or longer, about four days or longer, about five days or longer, or about six days or longer. In some embodiments of the methods described herein, an interval between administrations of a targeted cytokine described herein is about one week or longer, about two weeks or longer, about three weeks or longer, or about four weeks or longer. In some embodiments of the methods described herein, an interval between administrations of a targeted cytokine described herein is about one month or longer, about two months or longer, or about three months or longer. As used herein, an interval between administrations refers to the time period between one administration of the targeted cytokine and the next administration of the targeted cytokine. As used herein, an interval of about one month includes four weeks. In some embodiments, the treatment includes multiple administrations of the targeted cytokine, wherein the interval between administrations may vary. For example, in some embodiments, the interval between the first administration and the second administration is about one week, and the intervals between the subsequent administrations are about two weeks. In some embodiments, the interval between the first administration and the second administration is about two days, three days, four days, or five days, or six days, and the intervals between the subsequent administrations are about one week.

In some embodiments, the targeted cytokine is administered on multiple occasions over a period of time. The dosage that is administered to the subject on multiple occasions can, in some embodiments, be the same dosage for each administration, or, in some embodiments, the targeted cytokine can be administered to the subject at two or more different dosages. For example, in some embodiments, a targeted cytokine is initially administered at one dosage on one or more occasions and is later administered at a second dosage on one or more occasions beginning at a later time point.

In some embodiments, a targeted polypeptide described herein is administered at a flat dose. In some embodiments, a targeted polypeptide described herein is administered to a subject at a dosage from about 25 mg to about 500 mg per dose. In some embodiments, the targeted polypeptide is administered to a subject at a dosage of about 25 mg to about 50 mg, about 50 mg to about 75 mg, about 75 mg to about 100 mg, about 100 mg to about 125 mg, about 125 mg to about 150 mg, about 150 mg to about 175 mg, about 175 mg to about 200 mg, about 200 mg to about 225 mg, about 225 mg to about 250 mg, about 250 mg to about 275 mg, about 275 mg to about 300 mg, about 300 mg to about 325 mg, about 325 mg to about 350 mg, about 350 mg to about 375 mg, about 375 mg to about 400 mg, about 400 mg to about 425 mg, about 425 mt to about 450 mg, about 450 mg, to about 475 mg, or about 475 mg to about 500 mg per dose.

In some embodiments, a targeted polypeptide described herein is administered to a subject at a dosage based on the subject's weight or body surface area (BSA). Depending on the type and severity of the disease, about 1 μg/kg to 15 mg/kg (e.g., 0.1 mg/kg-10 mg/kg) of targeted polypeptide can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the targeted polypeptide would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. In some embodiments, a targeted polypeptide described herein is administered to a subject at a dosage from about 0.1 mg/kg to about 10 mg/kg or about 1.0 mg/kg to about 10 mg/kg. In some embodiments, a targeted polypeptide described herein is administered to a subject at a dosage of about any of 0.1 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 9.5 mg/kg, or 10.0 mg/kg. In some embodiments, a targeted polypeptide described herein is administered to a subject at a dosage of about or at least about 0.1 mg/kg, about or at least about 0.5 mg/kg, about or at least about 1.0 mg/kg, about or at least about 1.5 mg/kg, about or at least about 2.0 mg/kg, about or at least about 2.5 mg/kg, about or at least about 3.0 mg/kg, about or at least about 3.5 mg/kg, about or at least about 4.0 mg/kg, about or at least about 4.5 mg/kg, about or at least about 5.0 mg/kg, about or at least about 5.5 mg/kg, about or at least about 6.0 mg/kg, about or at least about 6.5 mg/kg, about or at least about 7.0 mg/kg, about or at least about 7.5 mg/kg, about or at least about 8.0 mg/kg, about or at least about 8.5 mg/kg, about or at least about 9.0 mg/kg, about or at least about 9.5 mg/kg, about or at least about 10.0 mg/kg, about or at least about 15.0 mg/kg, about or at least about 20 mg/kg, about or at least about 30 mg/kg, about or at least about 40 mg/kg, about or at least about 50 mg/kg, about or at least about 60 mg/kg, about or at least about 70 mg/kg, about or at least about 80 mg/kg, about or at least about 90 mg/kg, or about or at least about 100 mg/kg. Any of the dosing frequencies described above may be used.

A method of treatment contemplated herein is the treatment of a disorder or disease such as cancer with any of the targeted cytokines or compositions described herein. Disorders or diseases that are treatable with the formulations of this present invention include leukemia, lymphoma, head and neck cancer, colorectal cancer, prostate cancer, pancreatic cancer, melanoma, breast cancer, neuroblastoma, lung cancer, ovarian cancer, osteosarcoma, bladder cancer, cervical cancer, liver cancer, kidney cancer, skin cancer (e.g., Merkel cell carcinoma) or testicular cancer.

In some embodiments, provided herein is a method of treatment or prevention of a cancer by administration of any targeted cytokines or compositions described herein. In some embodiments, provided herein is a method of treatment or prevention of a cancer by administration of any targeted cytokine or composition described herein in combination with an anti-cancer agent. The anti-cancer agent can be any agent capable of reducing cancer growth, interfering with cancer cell replication, directly or indirectly killing cancer cells, reducing metastasis, reducing tumor blood supply, or reducing cell survival. In some embodiments, the anti-cancer agent is selected from the group consisting of a PD-1 inhibitor, an EGFR inhibitor, a HER2 inhibitor, a VEGFR inhibitor, a CTLA-4 inhibitor, a BTLA inhibitor, a B7H4 inhibitor, a B7H3 inhibitor, a CSFIR inhibitor, an HVEM inhibitor, a CD27 inhibitor, a KIR inhibitor, an NKG2A inhibitor, an NKG2D agonist, a TWEAK inhibitor, an ALK inhibitor, a CD52 targeting antibody, a CCR4 targeting antibody, a PD-L1 inhibitor, a MT inhibitor, a PDGFR inhibitor, a BAFF inhibitor, an HD AC inhibitor, a VEGF ligand inhibitor, a CD19 targeting molecule, a FOFR1 targeting molecule, a DFF3 targeting molecule, a DKK1 targeting molecule, a MUC1 targeting molecule, a MUG 16 targeting molecule, a PSMA targeting molecule, an MSFN targeting molecule, an NY-ES0-1 targeting molecule, a B7H3 targeting molecule, a B7H4 targeting molecule, a BCMA targeting molecule, a CD29 targeting molecule, a CD151 targeting molecule, a CD 123 targeting molecule, a CD33 targeting molecule, a CD37 targeting molecule, a CDH19 targeting molecule, a CEA targeting molecule, a Claudin 18.2 targeting molecule, a CFEC12A targeting molecule, an EGFRVIII targeting molecule, an EPCAM targeting molecule, an EPHA2 targeting molecule, an FCRH5 targeting molecule, an FLT3 targeting molecule, a GD2 targeting molecule, a glypican 3 targeting molecule, a gpA33 targeting molecule, a GPRC5D targeting molecule, an IL-23R targeting molecule, an IL-1RAP targeting molecule, a MCSP targeting molecule, a RON targeting molecule, a ROR1 targeting molecule, a STEAP2 targeting molecule, a TfR targeting molecule, a CD166 targeting molecule, a TPBG targeting molecule, a TROP2 targeting molecule, a proteasome inhibitor, an ABE inhibitor, a CD30 inhibitor, a FLT3 inhibitor, a MET inhibitor, a RET inhibitor, an IL-1(3 inhibitor, a MEK inhibitor, a ROS1 inhibitor, a BRAE inhibitor, a CD38 inhibitor, a RANKE inhibitor, a B4GALNT1 inhibitor, a SLAMF7 inhibitor, an IDH2 inhibitor, an mTOR inhibitor, a CD20 targeting antibody, a BTK inhibitor, a PI3K inhibitor, a FLT3 inhibitor, a PARP inhibitor, a CDK4 inhibitor, a CDK6 inhibitor, an EGFR inhibitor, a RAF inhibitor, a JAK1 inhibitor, a JAK2 inhibitor, a JAK3 inhibitor, an IL-6 inhibitor, a IL-17 inhibitor, a Smoothened inhibitor, an IL-6R inhibitor, a BCL2 inhibitor, a PTCH inhibitor, a PIGF inhibitor, a TGFB inhibitor, a CD28 agonist, a CD3 agonist, CD40 agonist, a GITR agonist, a 0X40 agonist, a VISTA agonist, a CD137 agonist, a LAG3 inhibitor, a TIM3 inhibitor, a TIGIT inhibitor, and an IL-2R inhibitor.

In some embodiments, provided herein is a method of treatment or prevention of a cancer by administration of any targeted cytokine described herein in combination with an anti-inflammatory agent. The anti-inflammatory agent can be any agent capable of preventing, counteracting, inhibiting, or otherwise reducing inflammation.

In some embodiments, the anti-inflammatory agent is a cyclooxygenase (COX) inhibitor. The COX inhibitor can be any agent that inhibits the activity of COX-1 and/or COX-2. In some embodiments, the COX inhibitor selectively inhibits COX-1 (i.e., the COX inhibitor inhibits the activity of COX-1 more than it inhibits the activity of COX-2). In some embodiments, the COX inhibitor selectively inhibits COX-2 (i.e., the COX inhibitor inhibits the activity of COX-2 more than it inhibits the activity of COX-1). In some embodiments, the COX inhibitor inhibits both COX-1 and COX-2.

In some embodiments, the COX inhibitor is a selective COX-1 inhibitor and is selected from the group consisting of SC-560, FR122047, P6, mofezolac, TFAP, flurbiprofen, and ketoprofen. In some embodiments, the COX inhibitor is a selective COX-2 inhibitor and is selected from the group consisting of celecoxib, rofecoxib, meloxicam, piroxicam, deracoxib, parecoxib, valdecoxib, etoricoxib, a chromene derivative, a chroman derivative, N-(2-cyclohexyloxynitrophenyl) methane sulfonamide, parecoxib, lumiracoxib, RS 57067, T-614, BMS-347070, JTE-522, S-2474, SVT-2016, CT-3, ABT-963, SC-58125, nimesulide, flosulide, NS-398, L-745337, RWJ-63556, L-784512, darbufelone, CS-502, LAS-34475, LAS-34555, S-33516, diclofenac, mefenamic acid, and SD-8381. In some embodiments, the COX inhibitor is selected from the group consisting of ibuprofen, naproxen, ketorolac, indomethacin, aspirin, naproxen, tolmetin, piroxicam, and meclofenamate. In some embodiments, the COX inhibitor is selected from the group consisting of SC-560, FR122047, P6, mofezolac, TFAP, flurbiprofen, ketoprofen, celecoxib, rofecoxib, meloxicam, piroxicam, deracoxib, parecoxib, valdecoxib, etoricoxib, a chromene derivative, a chroman derivative, N-(2-cyclohexyloxynitrophenyl) methane sulfonamide, parecoxib, lumiracoxib, RS 57067, T-614, BMS-347070, JTE-522, S-2474, SVT-2016, CT-3, ABT-963, SC-58125, nimesulide, flosulide, NS-398, L-745337, RWJ-63556, L-784512, darbufelone, CS-502, LAS-34475, LAS-34555, S-33516, diclofenac, mefenamic acid, SD-8381, ibuprofen, naproxen, ketorolac, indomethacin, aspirin, naproxen, tolmetin, piroxicam, and meclofenamate.

In some embodiments, the anti-inflammatory agent is an NF-KB inhibitor. The NF-KB inhibitor can be any agent that inhibits the activity of the NF-KB pathway. In some embodiments, the NF-KB inhibitor is selected from the group consisting of an IKK complex inhibitor, an IKB degradation inhibitor, an NF-KB nuclear translocation inhibitor, a p65 acetylation inhibitor, an NF-KB DNA binding inhibitor, an NF-KB transactivation inhibitor, and a p53 induction inhibitor.

In some embodiments, the IKK complex inhibitor is selected from the group consisting of TPCA-1, NF-KB Activation Inhibitor VI (BOT-64), BMS-345541, amlexanox, SC-514 (GK-01140), IMD-0354, and IKK-16. In some embodiments, the IKB degradation inhibitor is selected from the group consisting of BAY-11-7082, MG-115, MG-132, lactacystin, epoxomicin, parthenolide, carfilzomib, and MLN-4924 (pevonedistat). In some embodiments, the NF-KB nuclear translocation inhibitor is selected from the group consisting of JSH-23 and rolipram. In some embodiments, the p65 acetylation inhibitor is selected from the group consisting of gallic acid and anacardic acid. In some embodiments, the NF-KB DNA binding inhibitor is selected from the group consisting of GYY-4137, p-XSC, CV-3988, and prostaglandin E2 (PGE2). In some embodiments, the NF-KB transactivation inhibitor is selected from the group consisting of LY-294002, wortmannin, and mesalamine. In some embodiments, the p53 induction inhibitor is selected from the group consisting of quinacrine and flavopiridol. In some embodiments, the NF-KB inhibitor is selected from the group consisting of TPCA-1, NF-KB Activation Inhibitor VI (BOT-64), BMS-345541, amlexanox, SC-514 (GK-01140), IMD-0354, IKK-16, BAY-11-7082, MG-115, MG-132, lactacystin, epoxomicin, parthenolide, carfilzomib, MLN-4924 (pevonedistat), JSH-23 rolipram, gallic acid, anacardic acid, GYY-4137, p-XSC, CV-3988, prostaglandin E2 (PGE2), LY-294002, wortmannin, mesalamine, quinacrine, and flavopiridol.

In some embodiments, provided herein is a method of treatment or prevention of a cancer by administration of any targeted cytokine or composition described herein in combination with an anti-cancer therapeutic protein. The anti-cancer therapeutic protein can be any therapeutic protein capable of reducing cancer growth, interfering with cancer cell replication, directly or indirectly killing cancer cells, reducing metastasis, reducing tumor blood supply, or reducing cell survival. Exemplary anti-cancer therapeutic proteins may come in the form of an antibody or fragment thereof, an antibody derivative, a bispecific antibody, a chimeric antigen receptor (CAR) T cell, a fusion protein, or a bispecific T-cell engager (BiTE®). In some embodiments, provided herein is a method of treatment or prevention of a cancer by administration of any targeted cytokine or composition described herein in combination with CAR-NK (Natural Killer) cells.

### EXAMPLES

While certain compounds, compositions and methods of the present invention have been described with specificity in accordance with certain embodiments, the following examples serve only to illustrate the compounds of the invention and are not intended to limit the same.

### Example 1. Construction of a Targeted IL-2 Cytokine

In this example, various targeted IL-2 cytokines were designed and constructed. An exemplary targeted IL-2 cytokine comprises an IL-2 cytokine, a masking moiety that binds to IL-2, an Fc domain, and a targeting moiety.

### Fc Domain Engineering

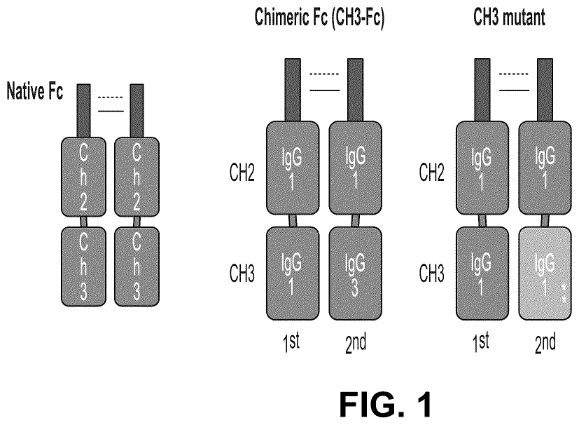

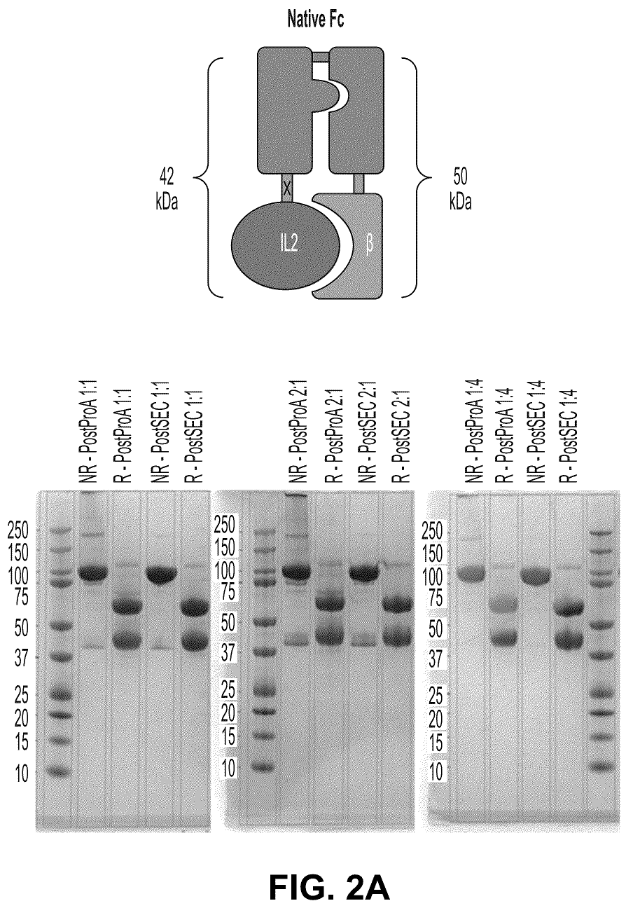

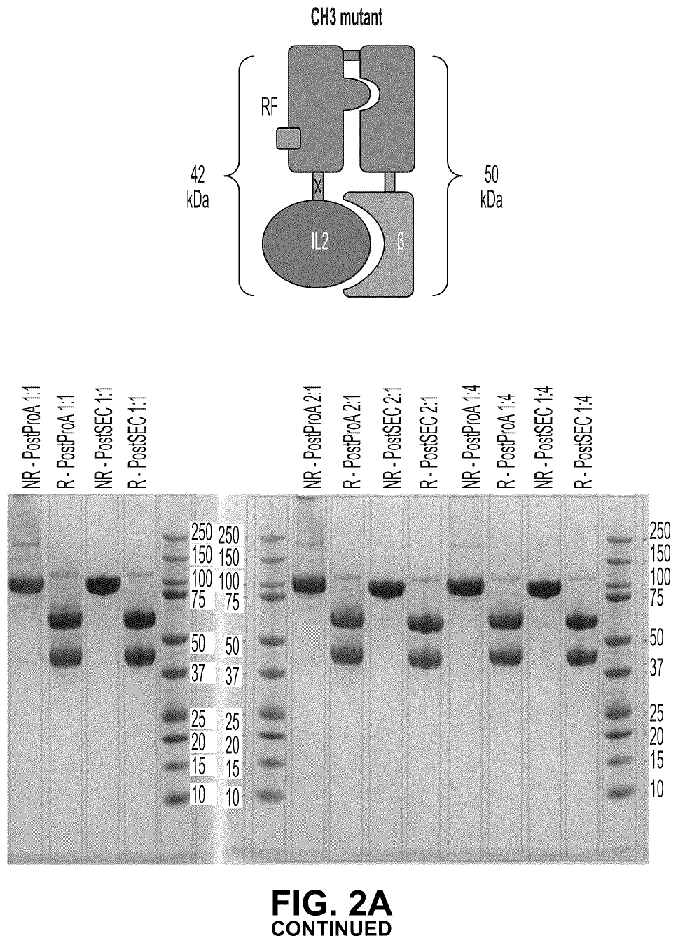

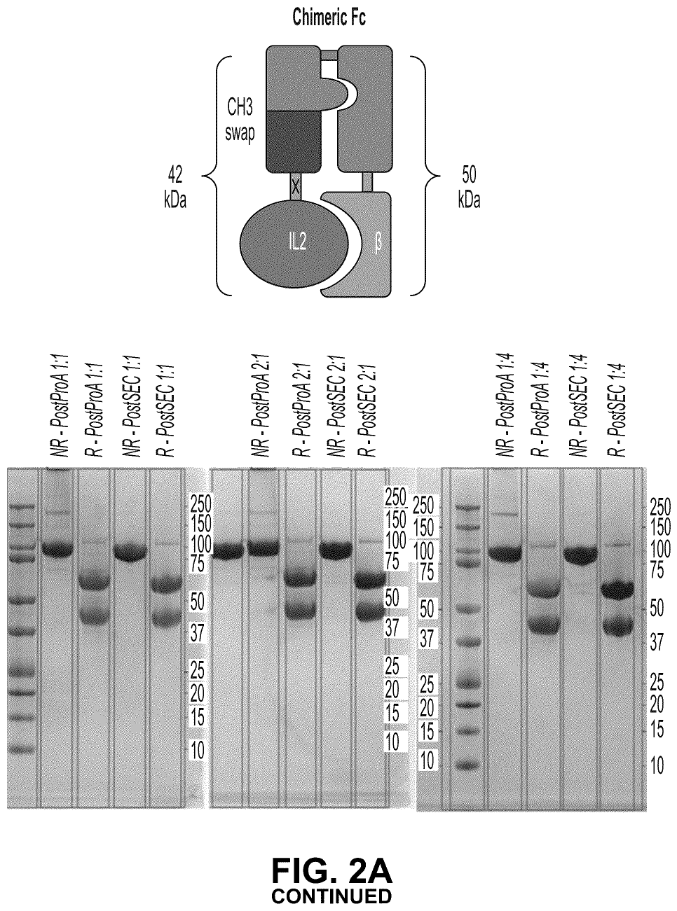

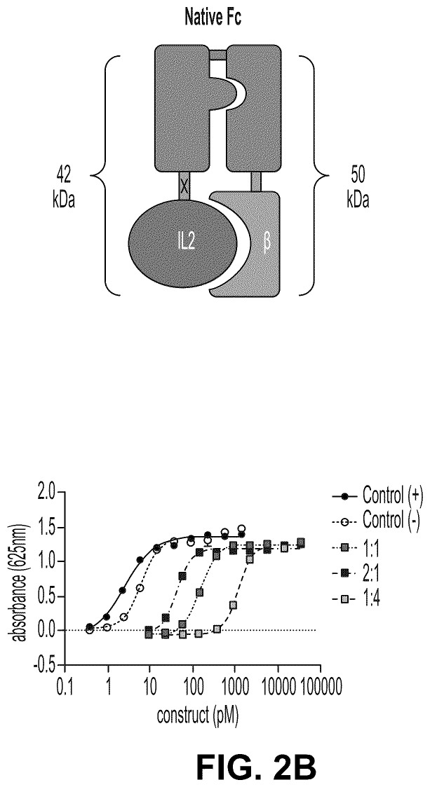

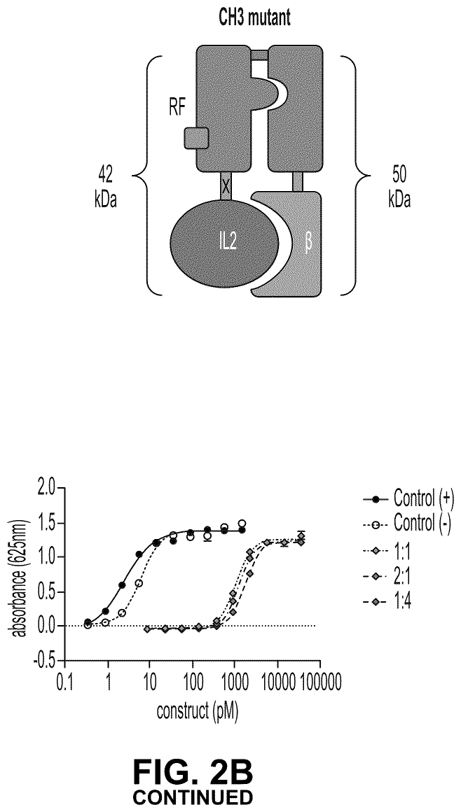

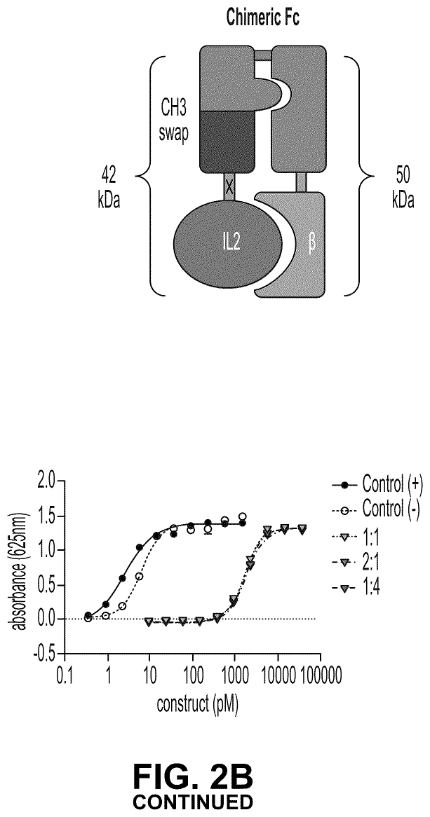

For efficient purification of heterodimeric targeted IL-2 cytokine, Fc domain was engineered. CH2 and CH3 domains of the first chain (“knob chain”) and the CH2 domain of the second chain (“hole chain”) were derived from either IgG1 or IgG4. CH3 of the second chain was derived from either wild-type IgG3, or IgG1 with mutations. The exemplary schematics are shown in FIG. 1. To assess if the heterodimeric Fc polypeptide can be purified without impurities, IL-2 cytokine and an IL-2 receptor β (IL-2Rβ; also known as CD122) was each fused to Fc chain as illustrated in FIG. 2A. The constructs shown in FIG. 2A were generated, purified, and characterized.

Plasmids encoding the constructs (e.g., native Fc, CH3 mutant, and chimeric Fc) were transfected into cells at different ratios of knob and hole chain (K:H of 1:1, 2:1, and 1:4). The Fc polypeptide chain was purified using protein A chromatography or by size exclusion chromatography (SEC). The samples after each protein A chromatography (“PostProA”) and SEC (“PostSEC”) were analysed by both non-reducing (NR) and reducing (R) gel electrophoresis. The impurities of different constructs were also assessed by measuring EC50 of active IL-2. As shown in FIG. 2A, the gel electrophoresis results show that residual “free knob” protein are present (˜40 KDa band) in both post Protein A and SEC purifications when cells were transfected with the native Fc format. However, both the CH3 mutant and the chimeric Fc formats successfully eliminated knob impurities. These findings were confirmed in a cell-based reporter assay (HEK Blue IL-2 assay) which determines % calculated active cytokine (FIG. 2B). As for the native Fc format, EC50 value shifts depending on the amount of impurity (free Knob). On the other hand, EC50 remains unchanged for both CH3 mutant and the chimeric Fc formats due to lack of active impurity.

To further examine whether these formats work in the context of the targeted cytokine, PD-1 targeting moiety fused to each knob and hole chain. In this example, four different targeted cytokine molecules (TC) were constructed and purity was determined by SEC, Non-reducing capillary electrophoresis sodium dodecyl sulfate (NR CE-SDS) and reducing capillary electrophoresis sodium dodecyl sulfate (Red CE-SDS), as shown in FIG. 3. The results show that the CH3 “RF” knob mutations effectively eliminated free knob in all targeted cytokine molecules tested.

### Targeting Moiety Engineering

Targeted IL-2 cytokine constructs were generated that include an IL-2 polypeptide or functional fragment thereof, a masking moiety, and an Fc domain, and a targeting moiety. A targeting moiety can be engineered to be in different formats. For example, for bivalent targeting, a targeted IL-2 cytokine comprises 1) chain 1 with variable heavy and IgG1 or IgG4 heavy constant region with hole mutations; 2) chain 2 with variable heavy and IgG1 or IgG4 heavy constant with knob mutations and either includes CH3 of IgG1 with “RF mutations” or IgG3 Ch3 domain, with IL-2 cytokine (or its variants) fused to the C-terminal; and 3) chain 3 with variable light and IgG kappa or lambda constant (FIG. 4A; Format A). For monovalent targeting, format B1 or B2 can be constructed, which comprises a target-specific Fab fused to either hole chain (B1) or to knob chain (B2) through CH1 domain. In this format, “hole chain” comprises Y349C/T366S/L368A/Y407V mutations, and “knob chain” has S354C/T366W mutations and either includes CH3 of IgG1 with “RF mutations” or IgG3 Ch3 domain, with IL-2 cytokine (or its variants) fused to the C-terminal. Fab light chain is comprised of variable light and immunoglobulin kappa or lambda constant regions. Format C as shown in FIG. 4A can also be used for monovalent targeting, which comprises 1) chain 1 with a variable light and IgG kappa or lambda constant region fused to IgG1 or 4 constant heavy region from upper hinge with “hole mutations” (Y349C/T366S/L368A/Y407V); 2) chain 2 with a variable light and IgG kappa or lambda constant region fused to variable heavy and IgG1 or IgG4 heavy constant with knob mutations (S354C/T366W) and either includes CH3 of IgG1 with “RF mutations” (H435R and Y436F) IgG3 Ch3 domain, with IL-2 cytokine (or its variants) fused to the C-terminal. EC50 value of each construct was measured using the cell-based reporter assay (HEK Blue IL-2 assay), and the results show that IL-2 of all formats tested were active (FIG. 4B).

### Example 2. In Vitro Assessment of Targeted Cytokines

In this example, various PD-1 targeting IL-2 cytokines were designed and constructed and tested for their abilities to target PD-1.

### On-Cell Binding

Various PD-1 targeting IL-2 cytokines were constructed, including both bivalent targeting and monovalent targeting formats. TC0 (negative control) does not comprise a masking moiety nor a targeting moiety that binds PD-1. TC1, a bivalent targeted cytokine, does not comprise a masking moiety. TC5, a bivalent targeted cytokine comprises a masking moiety. TC6, TC7, and TC8 are monovalent targeted cytokines without a masking moiety. The constructs used in this example comprise “knob and hole” mutations in the Fc region.

To assess whether these targeted cytokines bind to the PD-1, cell binding assays were performed. Specifically, mixed donor lots of human peripheral blood mononuclear cells (PBMCs) were thawed and seeded in medium (RPMI1640, 10% fetal bovine serum (FBS), 2 mM 1-Glutamine, 50 U/mL penicillin, 50 μg/mL streptomycin, 1 μM each of non-essential amino acids, 10 mM HEPES, 1 mM sodium pyruvate, 55 μM B-mercaptoethanol, 10 μg/mL gentamycin) into tissue culture treated plates that were coated for 1 h at 37° C. with 1 μg/ml anti-CD3. Anti-CD28 was added in solution at a concentration of 5 μg/ml. Two days later the PBMCs were collected and transferred into a 96 well v-bottom plate at 100,000 cells per well in FACS Buffer (2% FBS in phosphate buffered saline (PBS)). Raji-PD-1 cells were thawed in warm media (IMDM, 10% FBS, 2 mM L-Glutamine, 50 U/mL penicillin, 50 μg/mL streptomycin) and resuspended in FACS Buffer then transferred to a 96 well v-bottom plate at 100,000 cells per well. Test articles were diluted to twice the indicated concentration in FACS buffer. Test articles were added to cells at a 1:1 ratio to the final concentration indicated in the figure and incubated for 1 h at 4° C. The cells were washed three times with FACS buffer and fixed in 4% paraformaldehyde (PFA). Cells were then washed once in FACS buffer. All cells were subsequently stained with anti-human IgG PE for 30 minutes on ice. Cells were washed twice with FACS Buffer. Then to detect CD8+ T cells, PBMCs were stained in 1004 with anti-CD3 PE/Dazzle 594, anti-CD45 PerCP Cy5.5, anti-CD8 BV510, anti-CD4 BV421, and FC Block for 30 minutes. Cells were washed twice to remove unbound antibody and analyzed on the BD Celesta by gating on CD3+CD8+ T cells. Data were fit and EC50s generated using a nonlinear regression [Agonist] vs. response—Variable slope (four parameter) in GraphPad Prism except for TC0 and the isotype control which did produce full sigmoidal curves.

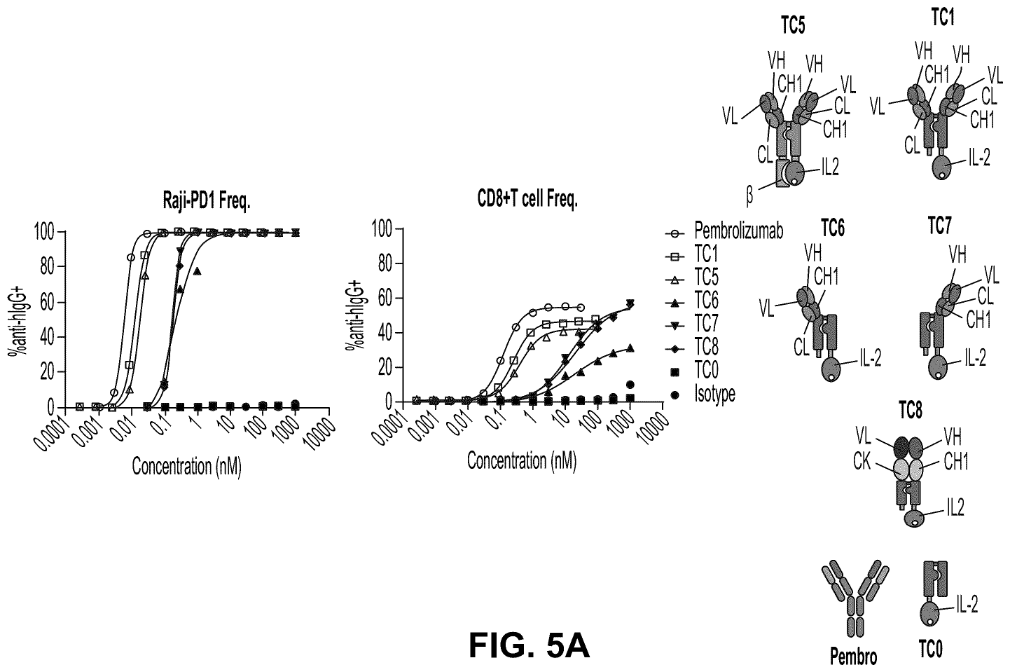

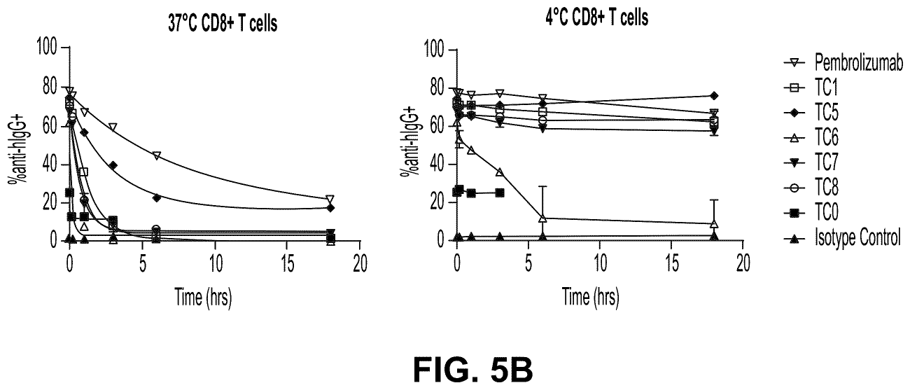

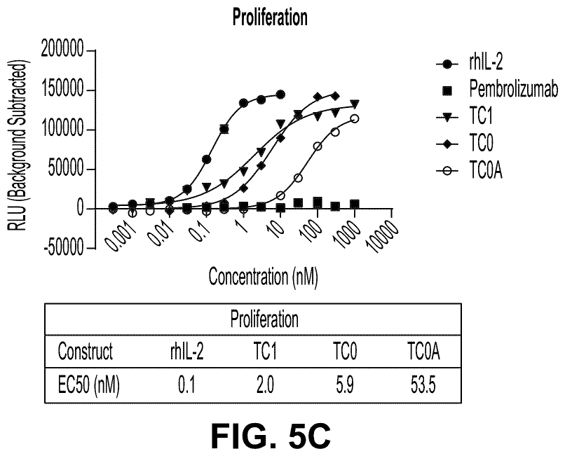

Results in FIG. 5A, shows that on Raji PD-1 cells and CD3/CD28 activated CD8+ T cells the bivalent TC1 and TC5 are able to bind PD-1 with low and similar EC50 values to Pembrolizumab. The monovalent formats (TC6, TC7, and TC8) also bind to PD-1 tightly, albeit with slightly higher EC50 values. TC0, which expresses IL-2 but is not targeted to PD-1 did not bind CD8+ T cells or Raji-PD-1 cells. The EC50 values are shown in Table 2.

TABLE 2
EC values for various PD-1 targeting IL-2 cytokine against PD-1

| | | | | | | |
|---|---|---|---|---|---|---|
| Construct | Pembrolizumab | TC6 | TC7 | TC8 | TC1 | TC5 |

CD8+ T cells

| | | | | | | |
|---|---|---|---|---|---|---|
| EC50 (nM) | 0.1 | 19.3 | 12.8 | 20.9 | 0.3 | 0.4 |

Raji PD-1 Cells

| | | | | | | |
|---|---|---|---|---|---|---|
| EC50 (nM) | 0.006 | 0.2 | 0.2 | 0.2 | 0.01 | 0.02 |

### Surface Dwell Time

Next, surface dwell time of PD-1 targeting cytokines on CD8+ Tcells were measured.

Mixed donor lots of human peripheral blood mononuclear cells (PBMCs) were thawed and seeded in medium (RPMI1640, 10% fetal bovine serum (FBS), 2 mM 1-Glutamine, 50U/mL penicillin, 50 μg/mL streptomycin, 1 μM each of non-essential amino acids, 10 mM HEPES, 1 mM sodium pyruvate, 55 μM B-mercaptoethanol, 10 μg/mL gentamycin) into tissue culture treated plates that were coated for 1 h at 37° C. with 1 μg/ml anti-CD3. Anti-CD28 was added in solution at a concentration of 5 μg/ml. Two days later the PBMCs were collected and transferred into a 96 well v-bottom plate at 100,000 cells per well in media. All cells were stained in 100 μL of saturating concentrations (as determined by binding assays) of indicated molecules in media for 1 h at 4° C. The cells were washed three times with cold FACS buffer (2% FBS in phosphate buffered saline (PBS)). Cells were then split into two plates, and one was kept at 37° C. while one was kept at 4° C. on ice to prevent internalization. At each of the indicated time points, the cells were harvested and fixed with 4% paraformaldehyde (PFA). Cells were washed and stored at 4° C. until staining. All cells were subsequently stained with anti-human IgG PE for 30 minutes on ice. Cells were washed twice with FACS Buffer. Then, to detect CD8+ T cells, PBMCs were stained in 100 μL with anti-CD3 PE/Dazzle 594, anti-CD45 PerCP Cy5.5, anti-CD8 BV510, anti-CD4 BV421, and FC Block for 30 minutes. Cells were washed twice to remove unbound antibody and analyzed on the BD Celesta by gating on CD3+CD8+ T cells. Data from the 37° C. plate were fit and EC50s generated using a nonlinear regression One Phase Decay in GraphPad Prism except for the isotype control which did not bind to cells.

As shown in FIG. 5B, the half-life of bivalent masked construct TC5 is 2.1 h while bivalent unmasked TC1 exhibits a 0.9 h half-life. The monovalent constructs exhibit reduced half lives ranging from 0.1-0.6 h. All constructs except TC6 are stable on the surface at 4° C.

TABLE 3
Half Life of Targeted Cytokines on CD8+ T cells at 37° C.

| | | | | | | | |
|---|---|---|---|---|---|---|---|
| Construct | TC1 | TC6 | TC7 | TC5 | TC8 | Ipi | Pembrolizumab |

37° C. CD8+ T cells

| | | | | | | | |
|---|---|---|---|---|---|---|---|
| Half Life (h) | 0.9 | 0.1 | 0.5 | 2.1 | 0.6 | 0.05 | 5.7 |

### Proliferation of CD3 and CD28 Primed Human PBMCs

Proliferation of CD3 and CD28 primed human PBMCs in response to the targeted cytokines was assessed.

Mixed donor lots of human peripheral blood mononuclear cells (PBMCs) were thawed and seeded in medium (RPMI1640, 10% fetal bovine serum (FBS), 2 mM 1-Glutamine, 50 U/mL penicillin, 50 μg/mL streptomycin, 1 μM each of non-essential amino acids, 10 mM HEPES, 1 mM sodium pyruvate, 55 μM B-mercaptoethanol, 10 μg/mL gentamycin) into tissue culture treated plates that were coated for 1 h at 37° C. with 1 μg/ml anti-CD3. Anti-CD28 was added in solution at a concentration of 5 μg/ml. Two days later the PBMCs were collected and transferred into a 96 well round-bottom plate at 12,500 cells per well. Test articles were diluted to twice the indicated concentration in FACS buffer. Test articles were added to cells at a 1:1 ratio to the final concentration indicated in the figure. Cells were incubated at 37° C. for four additional days. Cell Titer-Glo and the plate of cells were brought to room temperature. Cells were mixed and 100 μLs were transferred to a white, opaque, flat-bottom plate. An equivalent volume of Cell Titer-Glo was added to the plate. It was shaken at 300 RPM for two minutes and then incubated at RT in the dark for 10 minutes. The plate was read for luminescence on an Agilent BioTek plate reader. Data were fit and EC50s generated using a nonlinear regression [Agonist] vs. response—Variable slope (four parameter) in GraphPad Prism except for Pembrolizumab which did produce a full sigmoidal curve.

As shown in FIG. 5C, targeted and unmasked TC1 exhibits an EC50 20-fold higher than rhIL-2.

### Example 3. In Vivo Efficacy of PD-1 Targeting IL-2 Cytokine

In this example, the efficacy of various PD-1 targeting IL-2 cytokines in vivo was assessed by measuring anti-tumor activity in mice. The targeted cytokines tested in this example include TC7 (monovalent format B2), TC9 (same construct as TC7, prepared in different cycle), TC6 (monovalent format B1), TC1 (bivalent), TC8 (monovalent format C). Pembrolizumab and vehicle were used as negative controls.

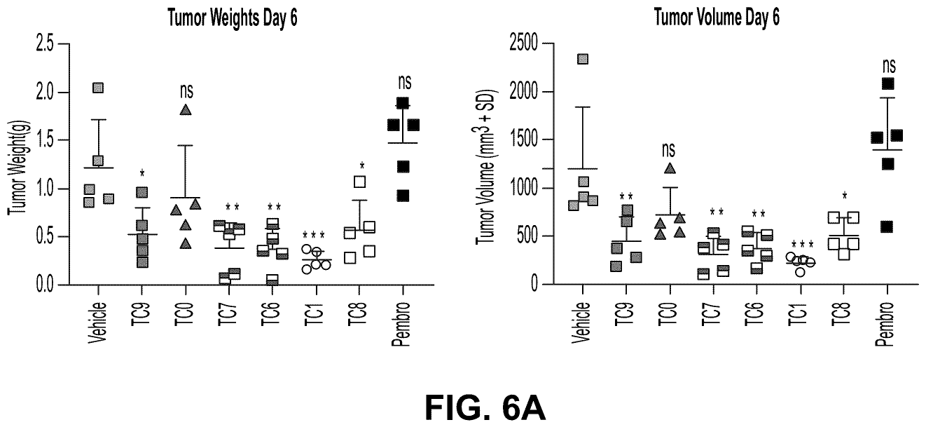

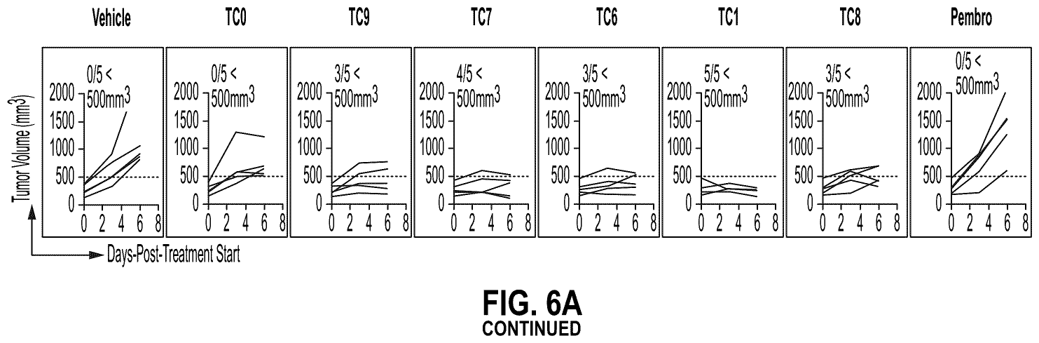

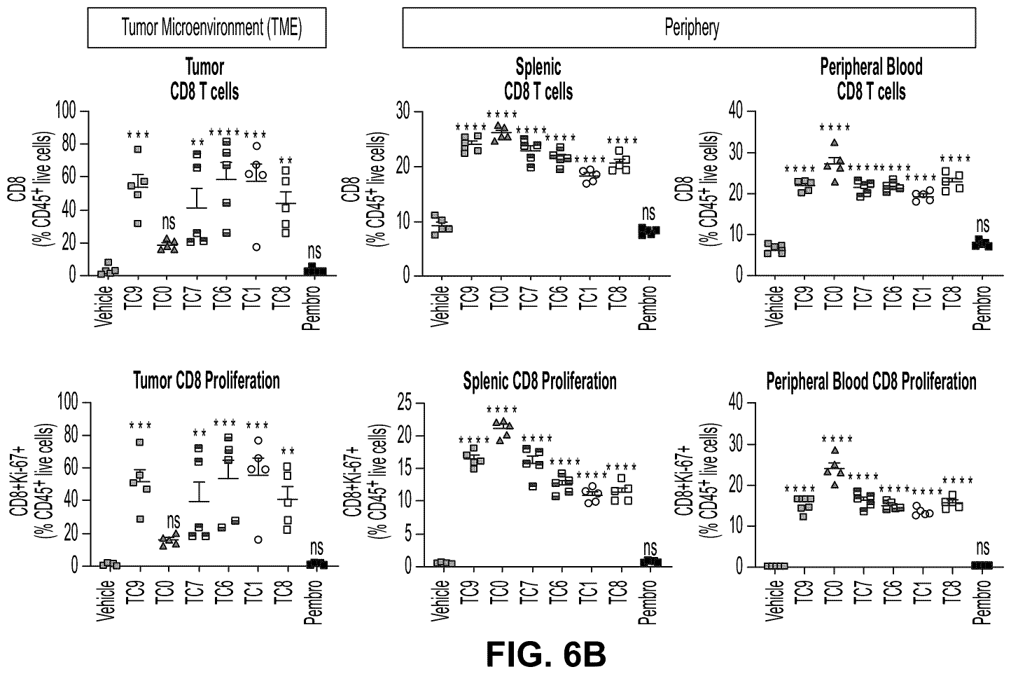

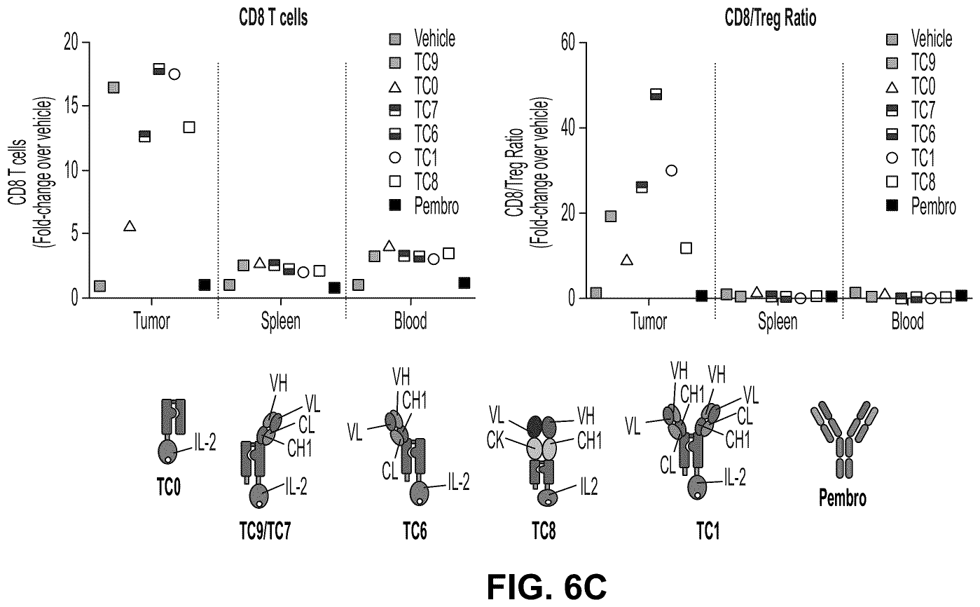

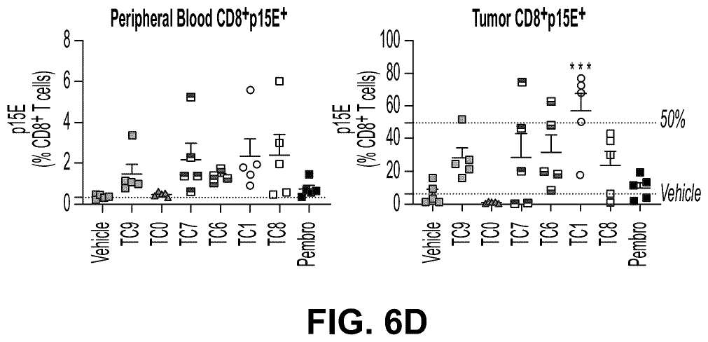

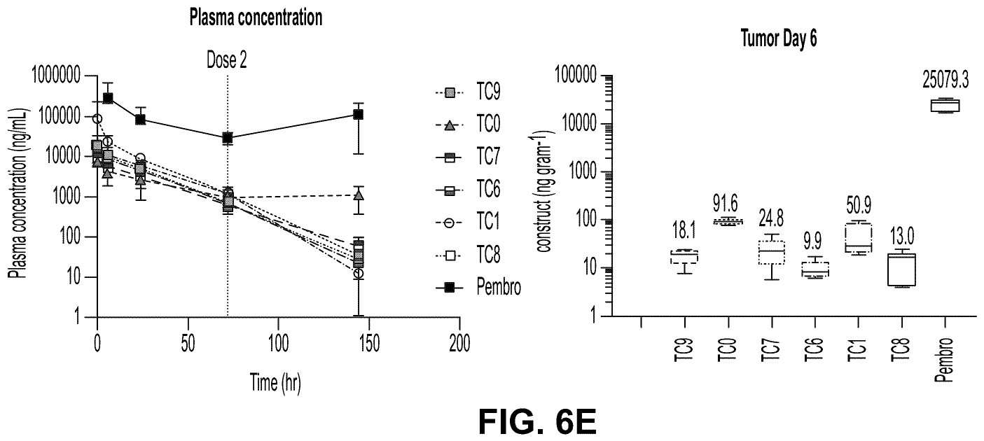

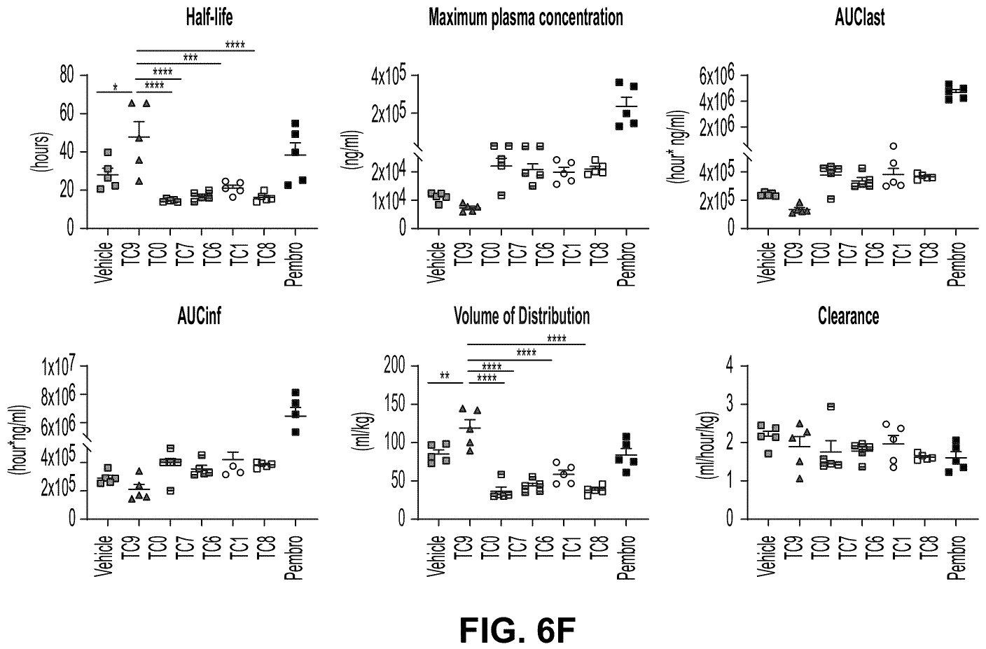

MC38 tumor cells were injected subcutaneously into the right flank of each mouse. Upon reaching ˜100 mm3 sized tumors (day 0), the mice received an equimolar intravenous dosing of the construct according to dose listed in Table 4. Specifically, all targeted cytokines were dosed at 5.4 nMoles/kg, and pembrolizumab was dosed at 71.43 nMoles/kg. Samples were collected from various tissues (including tumor, spleen, blood) at various time points after dosing. FIG. 6A shows that treatment with targeted, unmasked molecules show anti-tumor activity as early as day 6 against MC38 tumors.

TABLE 4
Dosing schedule

| Molecule | Dose (mg/kg) | nMoles/kg |
|---|---|---|
| TC9 | 0.62 | 5.4 |
| TC0 | 0.36 | 5.4 |
| TC7 | 0.63 | 5.4 |
| TC6 | 0.62 | 5.4 |
| TC1 | 0.78 | 5.4 |
| TC8 | 0.62 | 5.4 |
| Pembrolizumab | 10 | 71.43 |

The proliferation of immune cells following treatment with the targeted IL-2 cytokine constructs generated is also assessed. Samples taken from tumor, spleen, and peripheral blood were assessed for the proliferation of CD8+ T cells. FIG. 6B shows that all targeted, unmasked IL-2 cytokines significantly increase CD8 T cells in tumor environment and periphery. FIG. 6C shows that targeted, unmasked IL-2 cytokines induced higher fold-change in CD8 T cell expansion in the tumor microenvironment compared to periphery. FIG. 6D shows that the targeted, unmasked IL-2 cytokines increase antigen-specific CD8 T cell. FIG. 6E illustrates that all the targeted IL-2 cytokines tested accumulated in tumor. Particularly, the bivalent TC1 molecule showed an increase in drug accumulation in the tumor as compared to the monovalent molecules. Additionally, FIG. 6F illustrates that the targeted IL-2 cytokine molecules showed increase in drug exposure.

### Example 4. In Vitro and In Vivo Assessment of Targeted, Masked Cytokines

This example illustrates that the targeted, masked IL-2 cytokines of the present invention bind to PD-1 in vitro and are activated by proteases.

In this experiment, various PD-1 targeting masked IL-2 cytokines in bivalent format were constructed, as shown in FIG. 3. TC3 molecule comprises a first Fc polypeptide with “hole mutations” (Y349C; T366S; L368A; and Y407V) and a second Fc polypeptide with “knob mutations” (S354C and T366W) RF mutation (H435R and Y436F). CD122 was fused to the first Fc polypeptide via a cleavable linker comprising MPYDLYHP (SEQ ID NO: 34) sequence, and the IL-2 cytokine is fused to the second Fc polypeptide via a non-cleavable linker. Bivalent Fab are fused to each Fc polypeptide. TC4 molecule comprises a first Fc polypeptide with “hole mutations” (Y349C; T366S; L368A; and Y407V) and a second Fc polypeptide with “knob mutations” (S354C and T366W) RF mutation (H435R and Y436F). CD122 was fused to the first Fc polypeptide via a cleavable linker comprising VPLSLYSG (SEQ ID NO: 42) sequence, and the IL-2 cytokine is fused to the second Fc polypeptide via a non-cleavable linker. Bivalent Fab are fused to each Fc polypeptide. TC2 molecule comprises a first Fc polypeptide with “hole mutations” (Y349C; T366S; L368A; and Y407V) and a second Fc polypeptide with “knob mutations” (S354C and T366W) RF mutation (H435R and Y436F). CD122 was fused to the first Fc polypeptide via a non-cleavable linker, and the IL-2 cytokine is fused to the second Fc polypeptide via a non-cleavable linker. Bivalent Fab are fused to each Fc polypeptide. TC1 molecule comprises a first Fc polypeptide with “hole mutations” (Y349C; T366S; L368A; and Y407V) and a second Fc polypeptide with “knob mutations” (S354C and T366W). An IL-2 cytokine is fused to the second Fc polypeptide via a non-cleavable linker. Bivalent Fab are fused to each Fc polypeptide.

### In Vitro Assessment

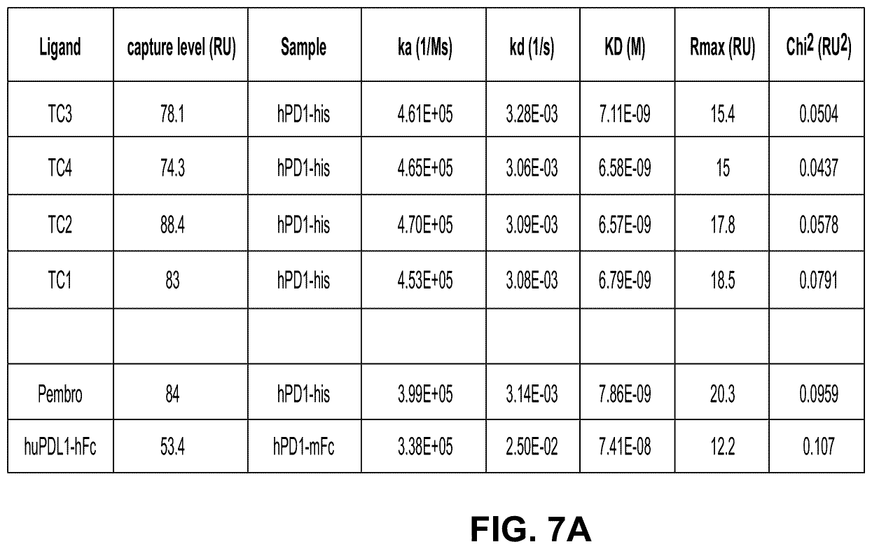

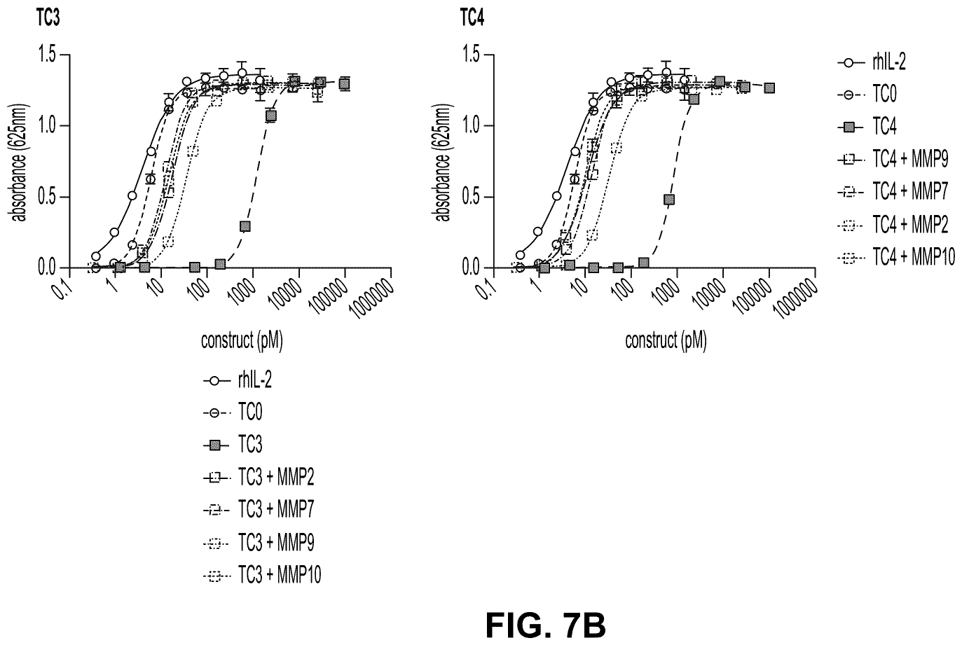

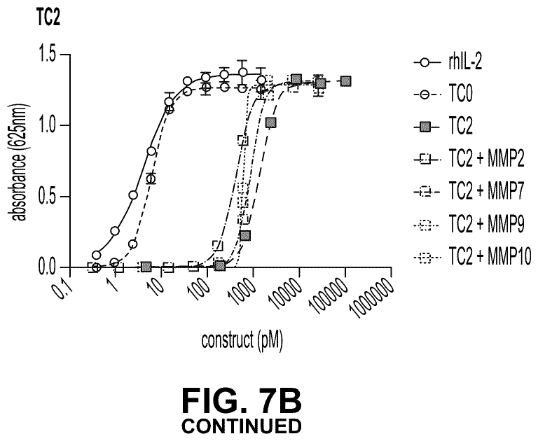

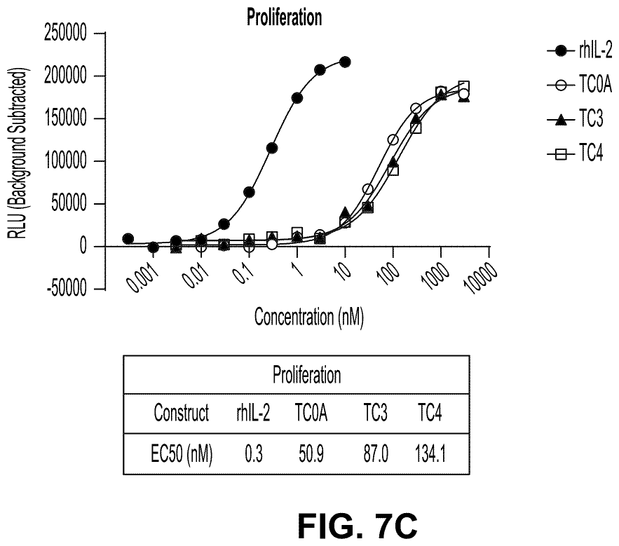

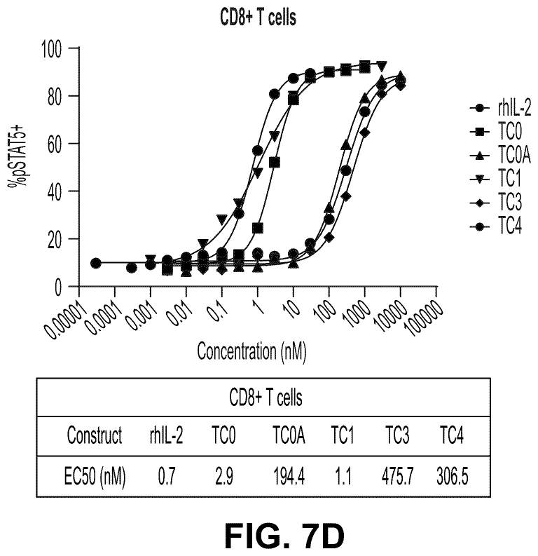

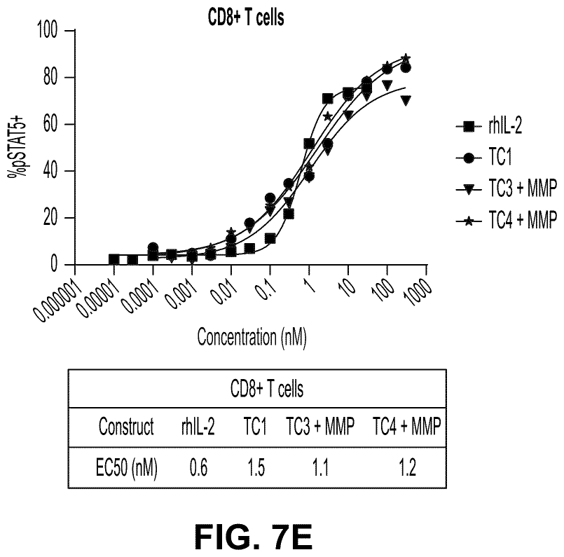

The constructed targeted IL-2 cytokines were tested for its binding affinity to PD-1 in vitro. The results show that the all four targeted IL-2 cytokines (TC3, TC4, TC2, and TC1) bound PD-1 with high affinity, similar to Pembrolizumab (FIG. 7A). Next, cleavage assay was performed to examine whether the masked the PD-1 targeting IL-2 cytokines are activated in the presence of protease. Recombinant human IL-2 (rhIL-2) and TC0 were used as positive controls, and the TC2, which does not comprise a cleavable linker, was used as a negative control. After incubation of different matrix metalloproteases (MMPs), the samples were tested in cell-based reporter assay (HEK Blue IL-2 assay) to determine % calculated active cytokine. FIG. 7B shows that TC3 and TC4, which comprises cleavable linker between the Fc polypeptide and the CD122 (masking moiety) were successfully activated in the presence of proteases.

### Proliferation

The constructed targeted IL-2 cytokines were tested for its proliferation according to methods described in Example 2. As shown in FIG. 7C, targeted and masked molecules all display similar EC50s that are over 100x higher than rhIL-2.

Signaling (pSTAT5)

The activation of Stat5 proteins is one of the earliest signaling events mediated by IL-2 family cytokines, allowing the rapid delivery of signals from the membrane to the nucleus. The efficacy of the targeted cytokines was tested in terms of pSTAT5 signaling.

TC3 and TC4 were cleaved by diluting to 1 μM in MMP Buffer ((150 mM NaCl; 50 mM Tris, pH 7.5; 10 mM CaCl2), filtered, and stored at room temperature) with 381 nM MMP10 (910-MP-010) for 48 h at 37° C. Mixed donor lots human peripheral blood mononuclear cells (PBMCs) were thawed in complete media (RPMI1640, 10% fetal bovine serum (FBS), 2 mM 1-Glutamine, 50 U/mL penicillin, 50 μg/mL streptomycin, 1 μM each of non-essential amino acids, 10 mM HEPES, 1 mM sodium pyruvate, 55 μM B-mercaptoethanol, 10 μg/mL gentamycin) and seeded into non-tissue culture treated, round-bottom 96-well plates in 100 μL with 500,000 cells/well. Cells were rested overnight at 37° C. On the following day, test articles were diluted to twice the indicated concentration in media. In FIG. 7E, 50 uls were removed from the plates to reduce the volume prior to addition of the test articles. Test articles were added to cells at a 1:1 ratio to the final concentration indicated in the figure and placed at 37° C. for 20 minutes. Cells were immediately fixed with 4% paraformaldehyde (PFA) at 37° C. for 15 minutes. Cells were transferred to a v-bottom plate to prevent cell loss. Cells were washed twice with FACS Buffer (2% FBS in phosphate buffered saline (PBS)) and then resuspended in 200 μL Perm III buffer on ice for 1 h. Cells were washed three times with FACS buffer and then stained in 100 μL with anti-pSTAT5, anti-CD3 PE/Dazzle 594, anti-CD45 PerCP Cy5.5, anti-CD8 BV510, anti-CD4 PE, and FC Block for 30 minutes. Cells were washed twice to remove unbound antibody. Cells were analyzed on the BD Celesta by gating on CD3+CD8+ T cells. Data were fit and EC50s generated using a nonlinear regression [Agonist] vs. response—Variable slope (four parameter) in GraphPad Prism. Note that FIG. 7D and FIG. 7E were performed on different lots of human PBMCs.

As shown in FIG. 7D, masked constructs TC3 and TC4 exhibit 280-436 fold higher EC50s than the unmasked TC1. In FIG. 7E, cleaved constructs exhibit EC50s equivalent to parental unmasked construct.

### Tumor Specific Cleavage In Vivo

This experiment illustrates that the PD-1 targeting cleavable-masked IL-2 cytokines of the present invention are indeed cleaved at a target site of tumor to minimize toxicity.

Mice (C57Black6/hPD-1) were inoculated with MC38 (murine colon adenocarcinoma cell line). When tumor volume reached ˜300-450 mm³, animals were dosed intravenously with 8 mg/kg TC2, TC4 or TC3 at d0 and d3. Blood was collected (using heparin-coated tubes) at 5 min, 6 h, 1 day, 3 days, and 6 days, and tumors were collected at takedown of 6 days. Plasma and tumor lysates were prepared using standard protocols. Targeted cytokines drugs were immunoprecipitated in both biological matrix, using biotinylated anti-human IgG and Streptavidin magnetic beads. The percentage of cleaved drug was determined using fluorescent triplex Western Blot. The lower limit of detection is 2.5% of cleaved drug.

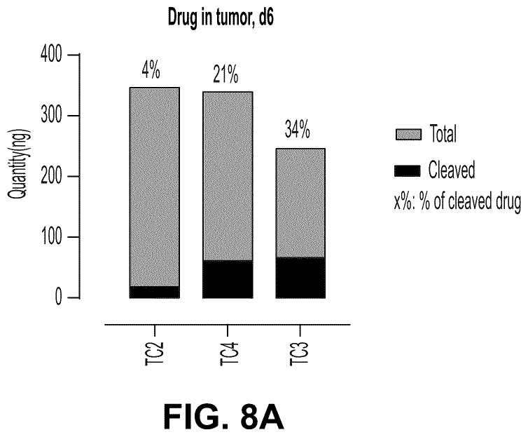

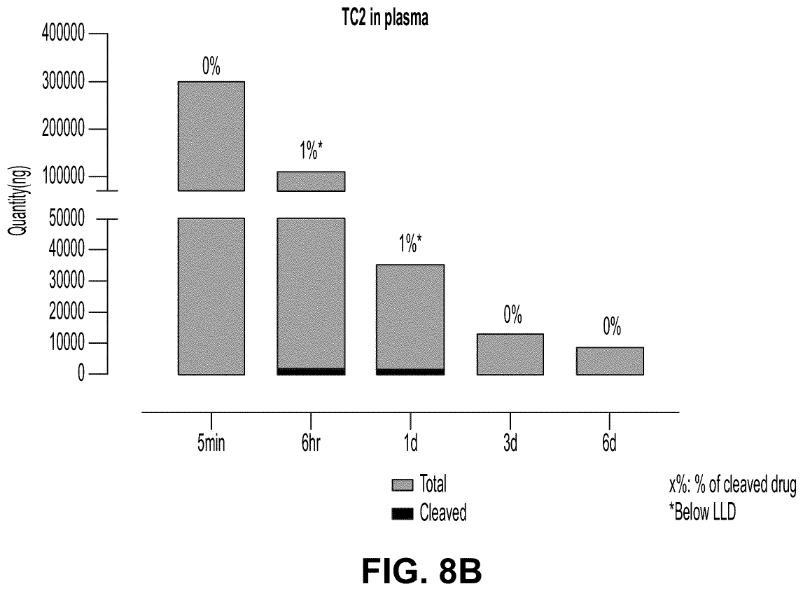

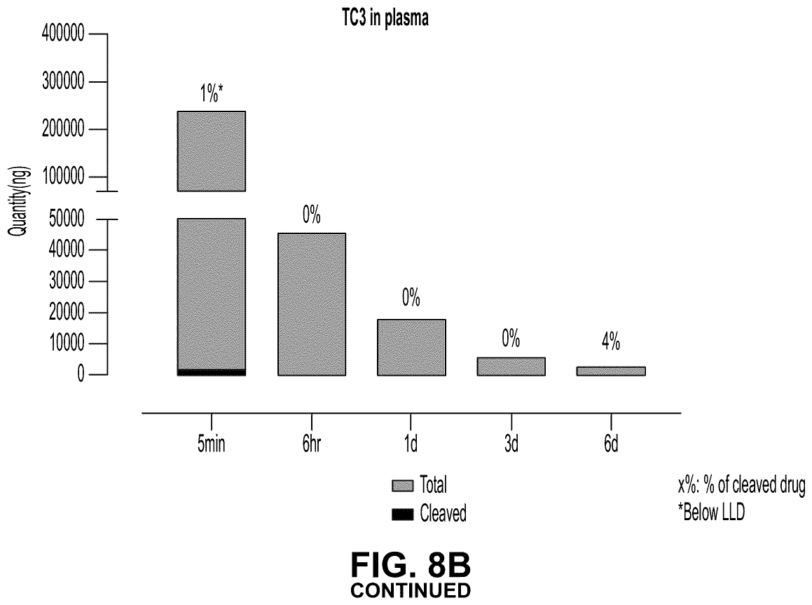

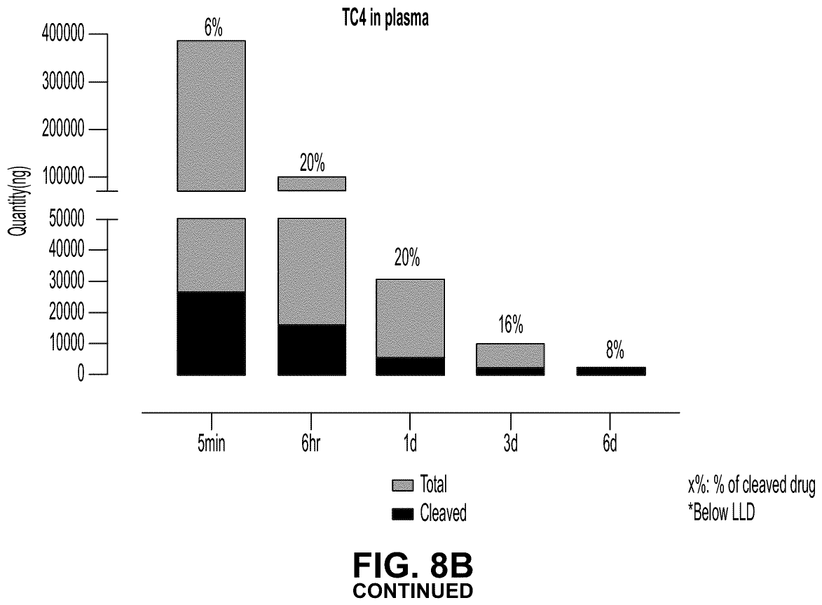

FIG. 8A shows that the same quantity of cleaved TC3 and TC4 were measured in tumor at d6. Additionally, minimal cleaved TC4 was observed in plasma, and substantially no cleavage was detected for TC3 (FIG. 8B).

### In Vivo Efficacy

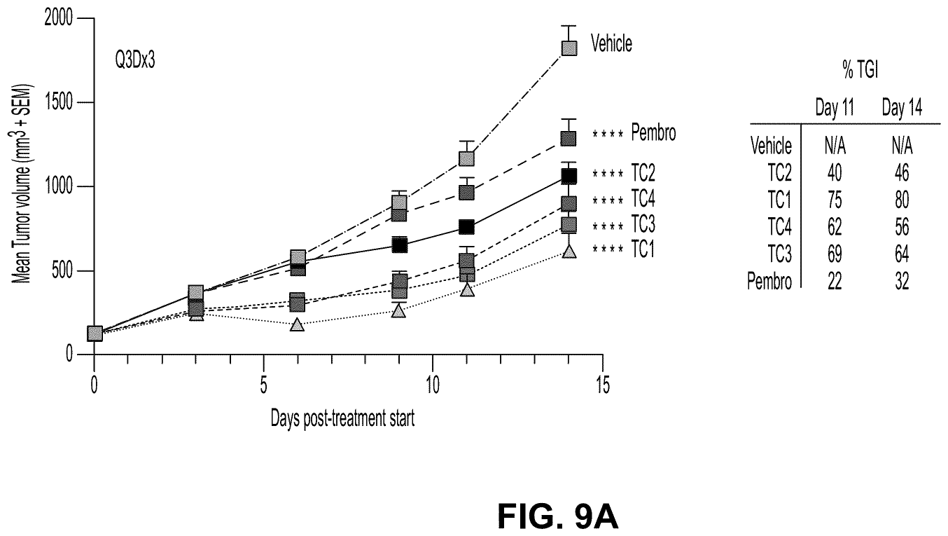

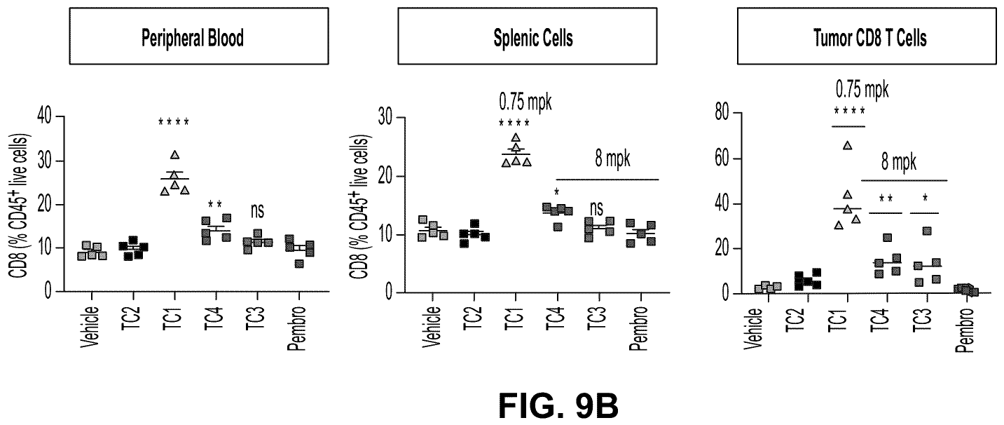

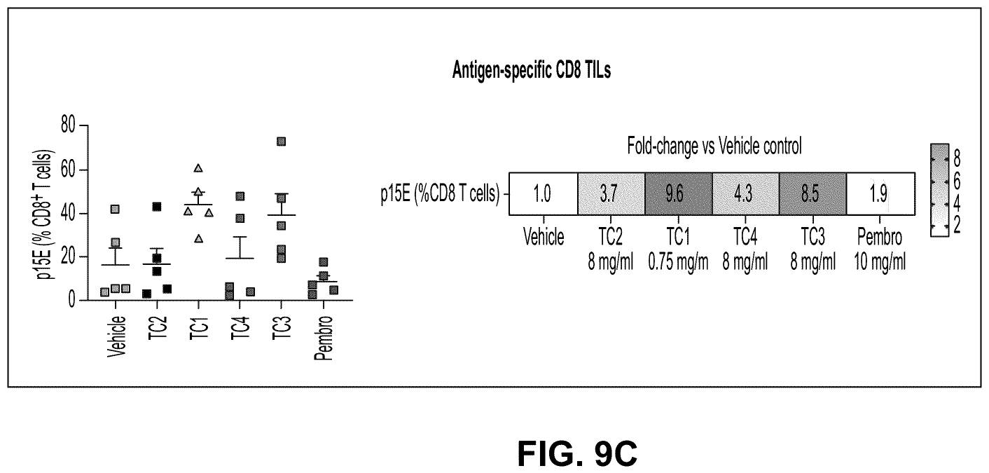

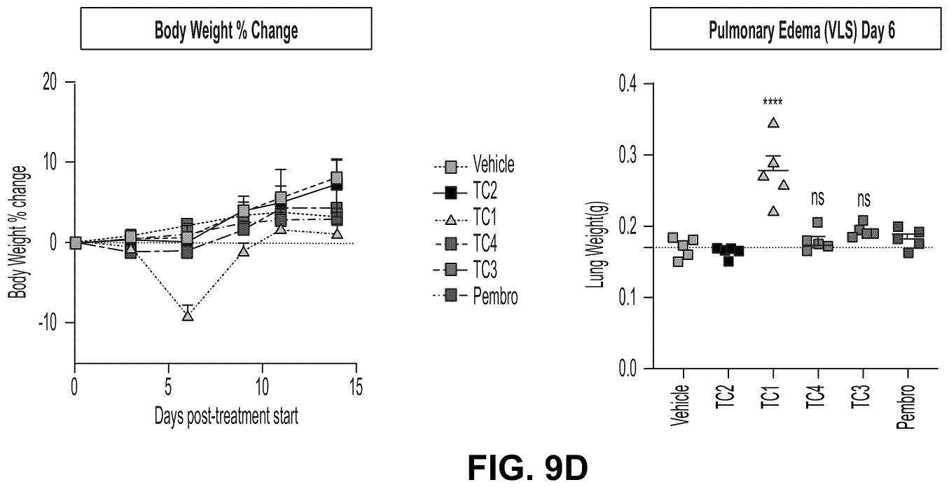

MC38 tumor cells were injected subcutaneously into the right flank of each mouse. Upon reaching ˜100 mm3 sized tumors (day 0), the mice received a 8 mg/kg of pembrolizumab, TC3, TC4, or TC2, or 0.75 mg/kg of TC1. Samples were collected from various tissues (including tumor, spleen, and blood) and tumor size was measured at various time points after dosing. FIG. 9A shows that treatment with targeted, masked-cleavable IL-2 cytokines show anti-tumor activity (TC3 and TC4) and exhibit significant tumor growth inhibition (TGI). Furthermore, the treatment with targeted, masked-cleavable IL-2 cytokines did not change the percentage of immune cells in the periphery but increased the percentage of CD8+ T cells in the tumor microenvironment (FIG. 9B). Additionally, FIG. 9C shows that the treatment with targeted, unmasked PD-1 targeting IL-2 cytokine may increase antigen specific T cells. In vivo tolerability of the targeted cytokine molecules was examined. FIG. 9D shows that the targeted, masked-cleavable IL-2 cytokines (TC3 and TC4) tested in this example were well tolerated in vivo and overcome toxicity observed with targeted unmasked IL-2 cytokine (TC1).

### Example 5. Engineering of Cleavable Linker for Cleavage Efficiency

In this example, cleavable linkers in the targeted IL-2 cytokines were further engineered to increase cleavage efficiency.

16 unique cleavable linkers were designed, 8 each with VPLSLYSG (SEQ ID NO: 42) or MPYDLYHP (SEQ ID NO: 34) cleavage peptide (CP). The cleavage peptide sequences were flanked on the N-terminus by a spacer domain 1 (SD1), and on the C-terminus by a spacer domain 2 (SD2), as illustrate by the formula: SD1-CP-SD2. Each spacer domain consisted of glycine, serine, and/or proline residues, with a total length between 14-20 amino acid residues. SD1 domain contained about 2-8 residues, whereas the CP+SD2 contained about 12 residues total. Proline residues were tested in various positions within the linkers.

The cleavable linker sequences shown in Table 5 were generated and incorporated into bivalent PD-1 targeting IL-2 cytokines. The various linker designs were tested based on energy/strain, solvent accessibility of substrate, and secondary structure.

TABLE 5
Engineered Cleavable Linkers (Cleavage peptide
underlined)

| Targeted | | | SEQ |
|---|---|---|---|
| Cytokine | Linker | | ID |
| Molecule | code | Linker Sequence | NO |
| TC3 | MPY_00 | GPPSGSSPMPYDLYHPSGGG | 44 |
| TC13 | MPY_02 | GPPSGSSPGMPYDLYHPSGGG | 45 |
| TC14 | MPY_03 | GGPMPYDLYHPSGGG | 46 |
| TC15 | MPY_04 | GSPMPYDLYHPSGGG | 47 |
| TC16 | MPY_05 | SSGGPMPYDLYHPSGGG | 48 |
| TC17 | MPY_06 | SGSSPGMPYDLYHPSGGG | 49 |
| TC18 | MPY_07 | GSSGPSGMPYDLYHPSGGG | 50 |
| TC19 | MPY_08 | GSSSGPGSMPYDLYHPSGGG | 51 |
| TC4 | VPL_00 | GPPSGSSPVPLSLYSGSGGG | 52 |
| TC21 | VPL_02 | GGSPVPLSLYSGSGGG | 53 |
| TC22 | VPL_03 | GSGGVPLSLYSGSGGG | 54 |
| TC23 | VPL_04 | GGSPGVPLSLYSGSGGG | 55 |
| TC24 | VPL_05 | GGSSGPVPLSLYSGSGGG | 56 |
| TC25 | VPL_06 | GGSSGSGVPLSLYSGSGGG | 57 |
| TC26 | VPL_07 | GGSPGSPVPLSLYSGSGGG | 58 |
| TC27 | VPL_08 | GSPGVPLSLYSGSSPMPYDLYHPSGG | 59 |
| | (MPY) | | |
| TC28 | Non- | PGSGS | 32 |
| | cleavable | | |
| TC29 | Parental, | | |
| | no mask | | |

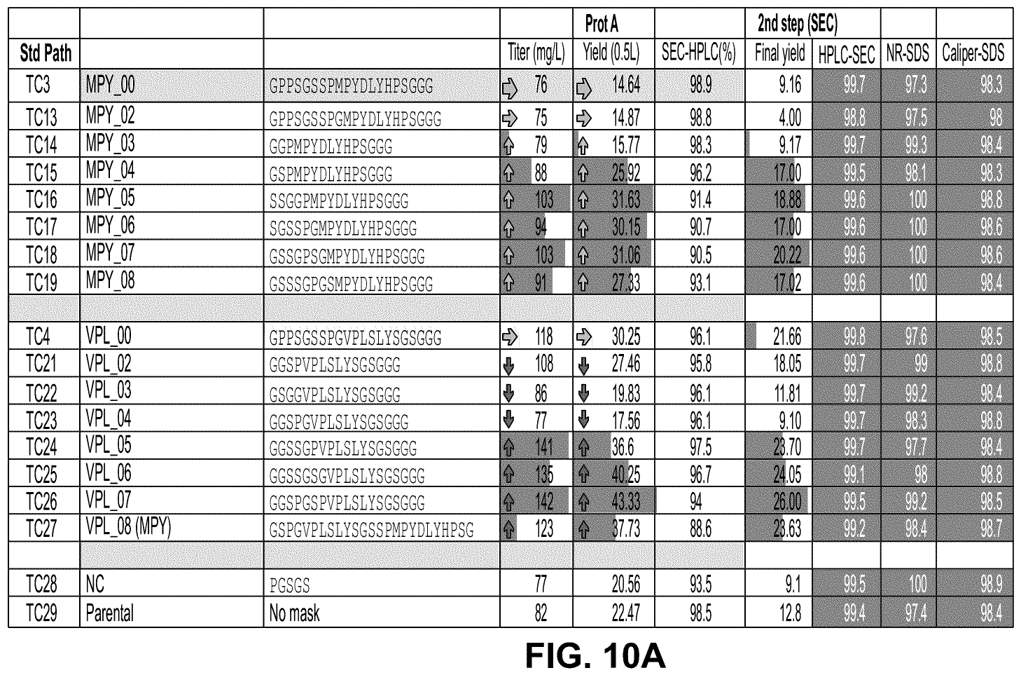

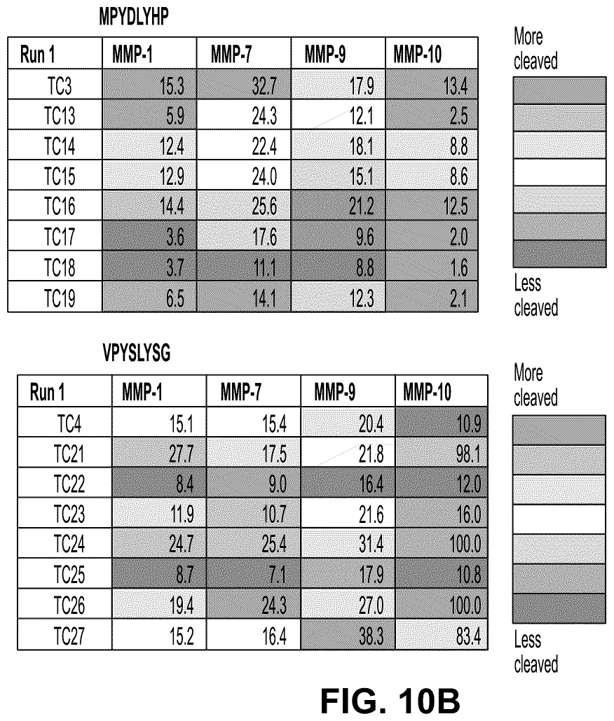

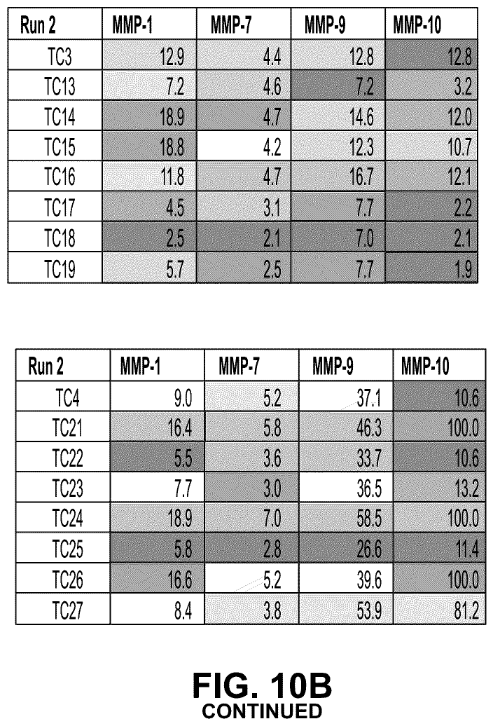

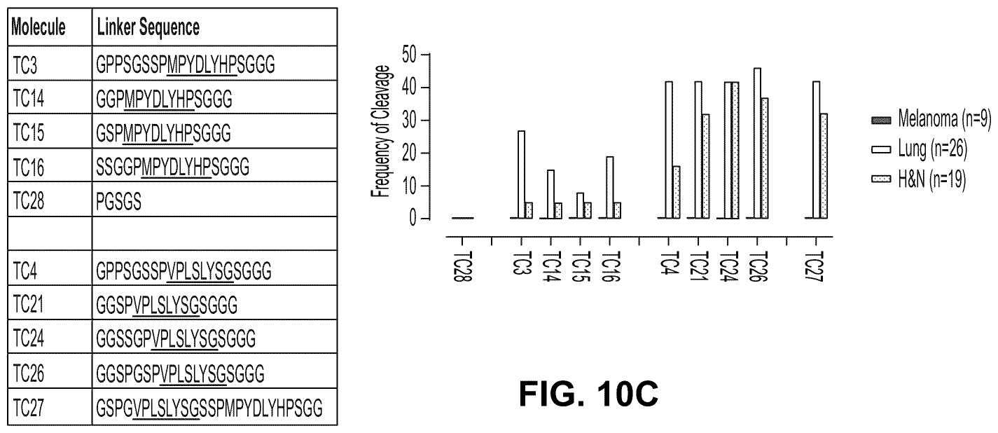

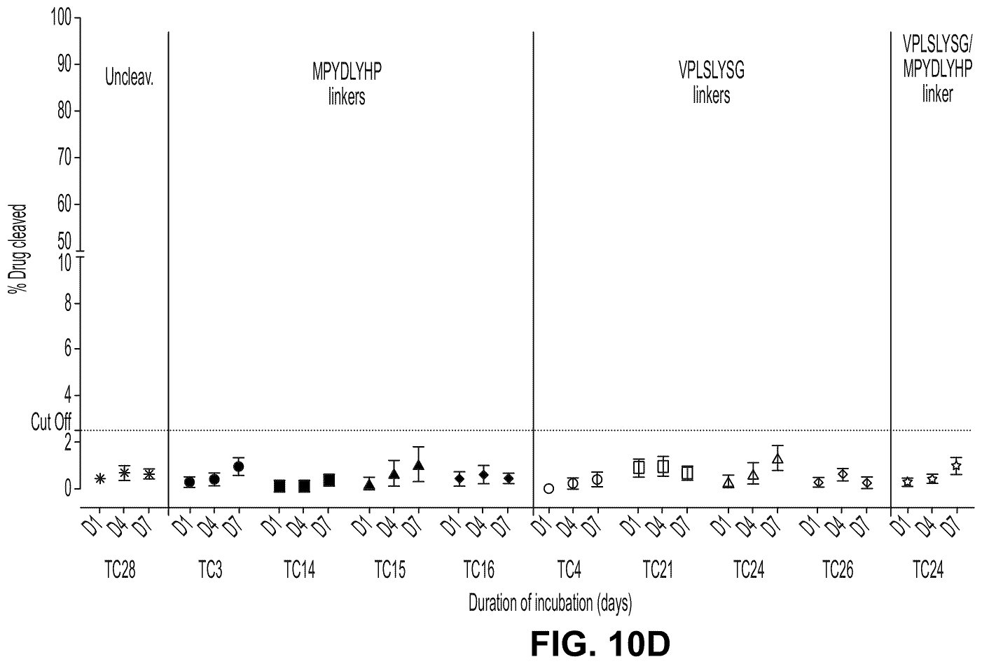

The targeted cytokines were expressed, purified by Protein A chromatography followed by SEC. The protein yield for each molecule is shown in FIG. 10A. The results show that the engineered protease substrate variants impacted titer and final yields. Furthermore, engineered substrate linker have an increased cleavage efficiency (FIG. 10B). For example, TC21, TC24, TC26, and TC27 have cleavage efficiency of greater than 80% when MMP-10 protease is used.

Next, to assess whether the cleavage is specific in tumor, but not in plasma, ex vivo experiments were performed.

Human primary tumor tissues (from Melanoma, Lung and HNC tumor patients) were gently dissociated and culture for 1-3 days in RPMI media. Conditioned media containing proteases secreted by the tumor and its microenvironment, was collected for incubation with targeted cytokines (1 uM) for overnight, at 37 C. The percentage of cleaved drug was determined using fluorescent triplex Western Blot.

FIG. 10C shows that targeted cytokines were activated by culture supernatants derived from human primary tumors from lung (n=26) and H&N (n=19) cancer patients.

Ex vivo cleavage by human plasma was measured. Specifically, targeted cytokines (1 uM final concentration) were incubated in human plasma from 8 lung tumor patients (prepared from blood collected using heparin-coated tubes) at 37° C. Samples were collected on days 1, 4 and 7 of the incubation periods. The percentage of cleaved drug was determined using fluorescent triplex Western. As shown in FIG. 10D, no substantial ex vivo cleavage of targeted cytokines, was observed in human plasma from lung tumor patients over 7 days of incubation.

Overall, these results show that the targeted IL-2 cytokine molecules have tumor specific cleavage with up to −50% cleavage efficiency (FIG. 10C), while having substantially no cleavage (below −2%) in plasma (FIG. 10D).

### Example 6. Minimal Cleavage Assay of Targeted IL-2 Cytokine with Engineered Cleavable Linkers

Minimal cleavage assay (MCA) was developed to rank in vitro cleavage efficiencies of engineered protease linkers with matrix metalloproteases (MMPs).

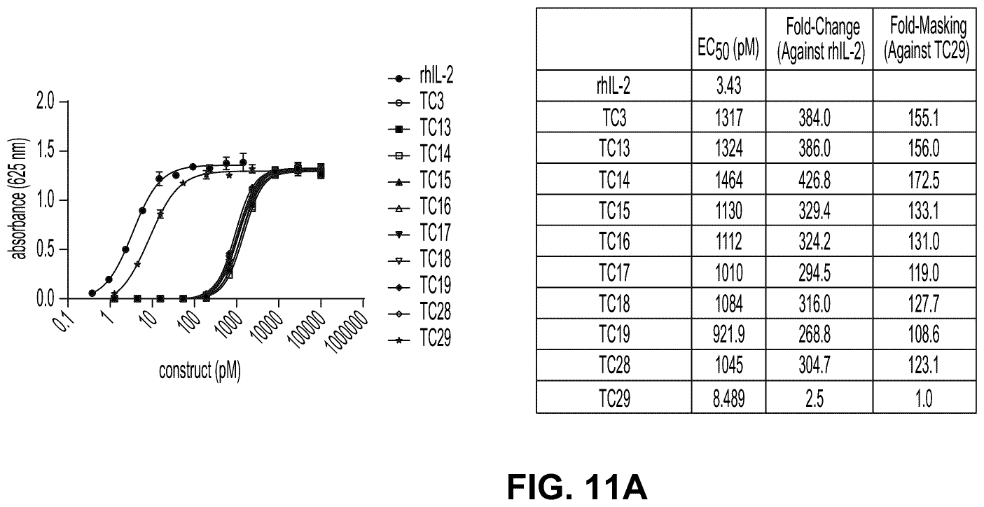

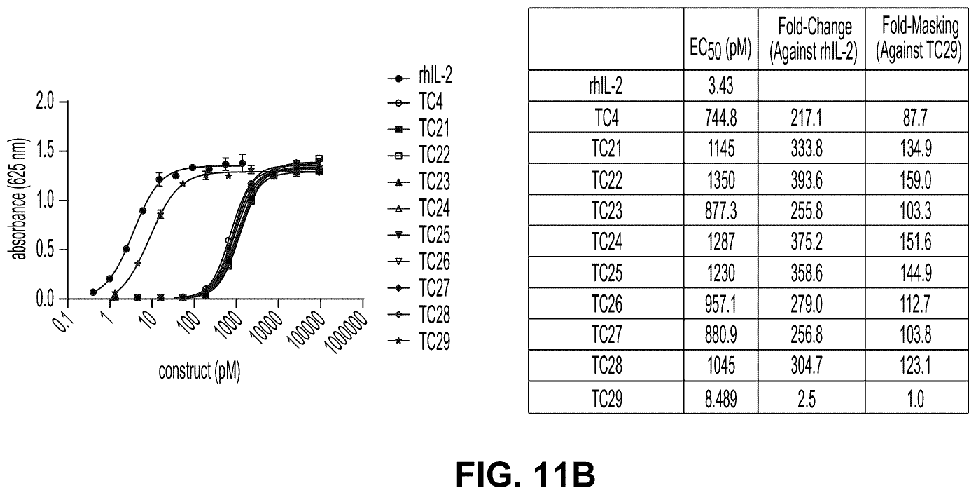

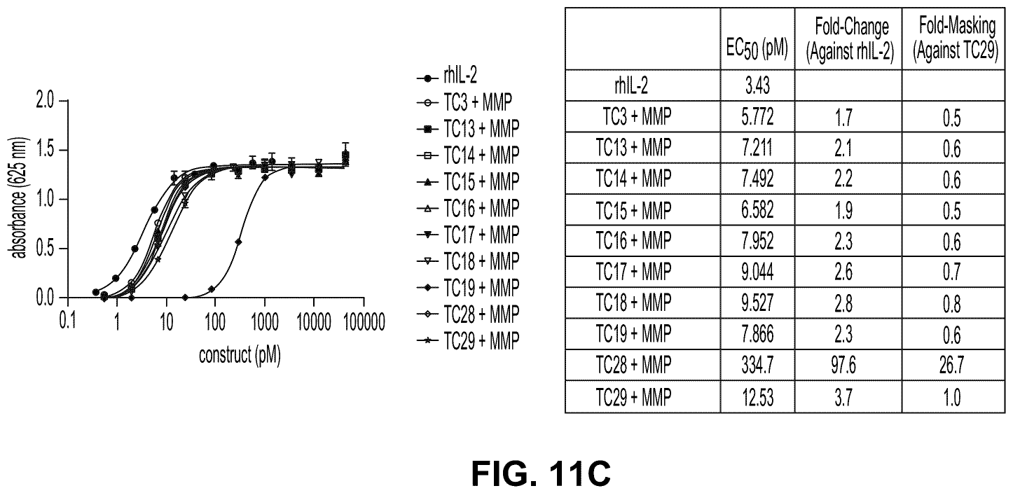

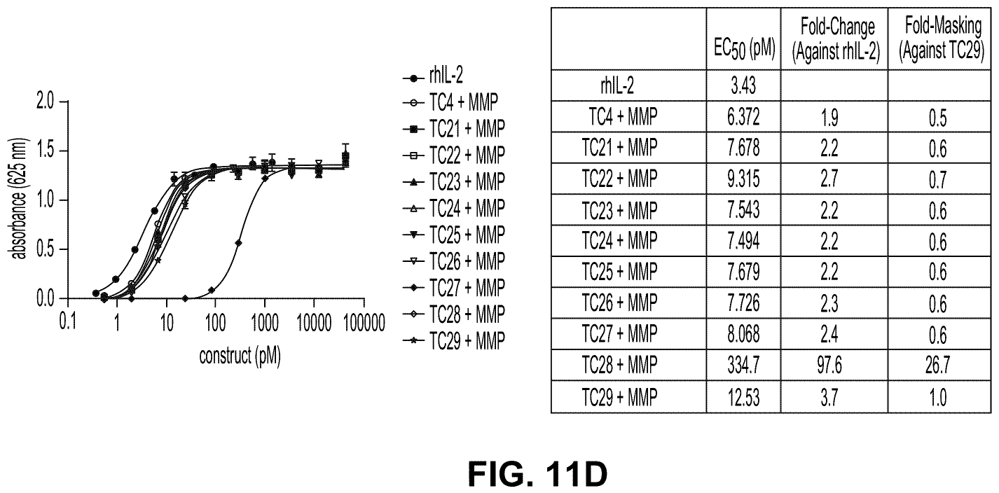

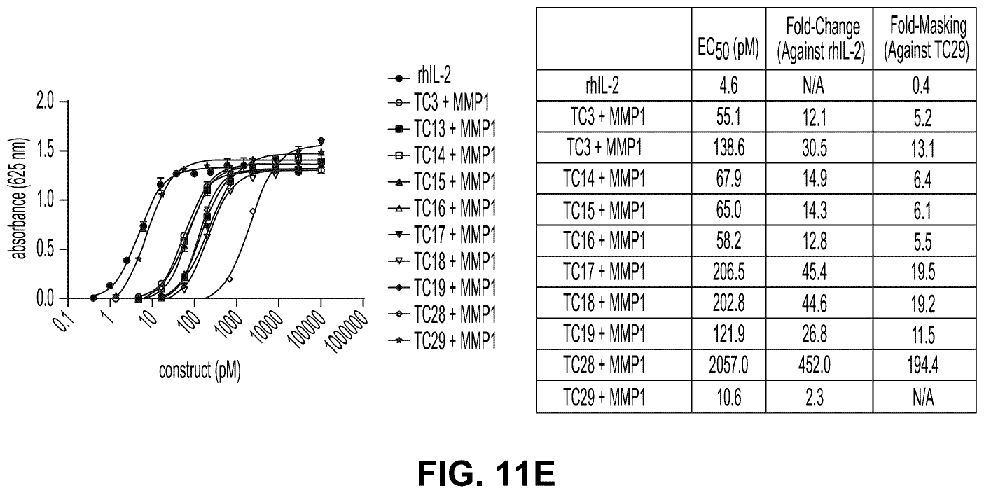

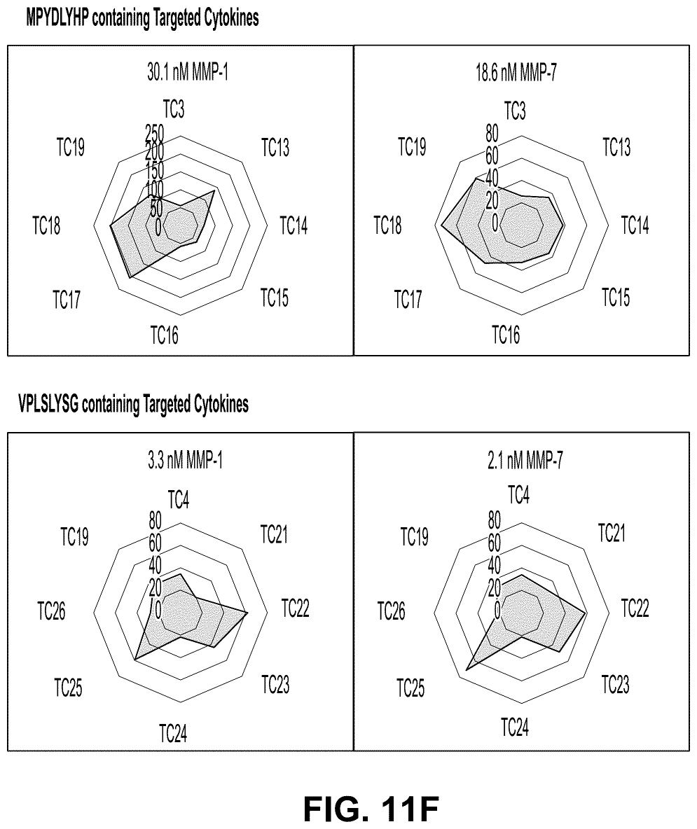

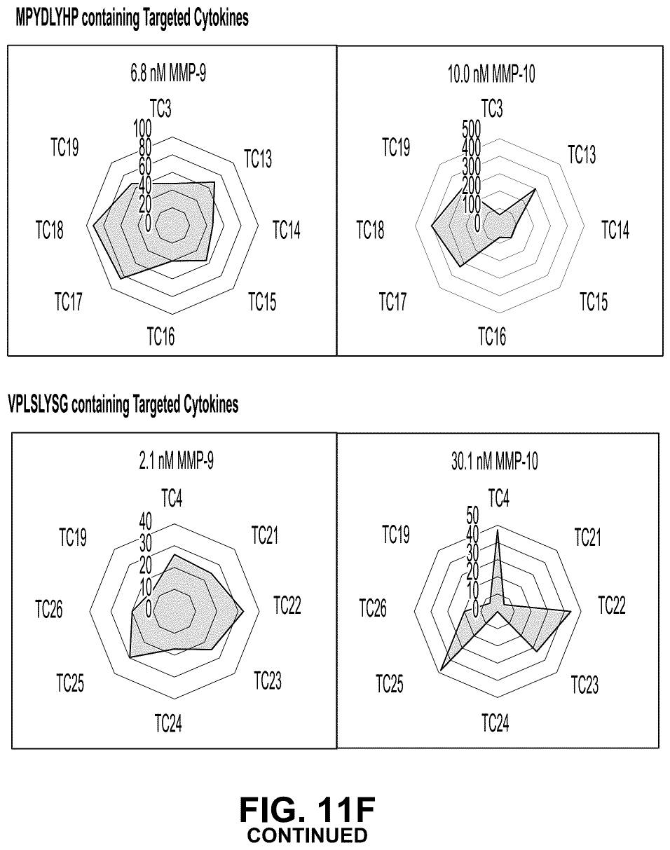

First, the targeted cytokines described in Example 5, Table 4 were assessed to verify that the masking moiety indeed “masks” the IL-2 cytokine. Recombinant human IL-2 (rhIL-2) and TC29 (targeted, unmasked cytokine) were used as positive controls for active IL-2, and TC28 (targeted, masked cytokine with non-cleavable linker) was used as a negative control. FIG. 11A and FIG. 11B show that the targeted cytokines containing either the MPYDLYHP (SEQ ID NO: 34) or VPLSLYSG (SEQ ID NO: 42) sequence as the cleavable substrate demonstrate comparable masking of IL-2.

To demonstrate that the targeted masked cytokines can regain IL-2 function by cleavage of the mask, full cleavage by MMP10 was performed. FIG. 11C and FIG. 11D illustrate that the targeted cytokines containing either the MPYDLYHP (SEQ ID NO: 34) or VPLSLYSG (SEQ ID NO: 42) sequence regained IL-2 function upon cleavage by the MMP10 protease.

For minimal cleavage assay, the amount of protease and incubation time to yield about 10-20% calculated active cytokine from un-engineered linkers were identified per MMP in method development. The conditions identified were used to test engineered linker variants and the % calculated active cytokine levels were used for ranking. Specifically, the MMPs were activated overnight with APMA. Constructs bearing engineered substrate linkers were prepped at 2 uM concentration. Reactions quenched with EDTA at hour 2. Samples were tested in cell-based reporter assay (HEK Blue IL-2 assay) to determine % calculated active cytokine.

EC50 values (FIG. 11E) from the experiment were obtained, and these values were used to populate the spider plots shown in FIG. 11F. The results show that cleavage with limited amounts of recombinant human proteases and subsequent analysis was capable or rank-ordering individual targeted cytokines containing the same protease substrate sequence. The numbers in the spider plot represent EC50 values in HEK Blue IL-2 assay after 2 hour cleave reactions with indicated amount of recombinant human protease. Therefore, the closer to the center of the spider plot, the more cleavage.

### Example 7. On-Cell Binding Assay of Targeted Cytokines with Engineered Masking Moiety

In this example, binding of the targeted masked cytokines on CD3/CD28 activated CD8+ T cells was assessed.

The targeted masked cytokines shown in Table 6 were constructed. TC46 and TC47 comprises a modified CD122 that comprises stabilizing mutations.

TABLE 6
Features of Targeted Cytokines used in Example 7

| | | | Comprises SEQ |
|---|---|---|---|
| | Masking Moiety | Cleavable Linker | ID NOs |
| TC3 | CD122 (C122S, | GPPSGSSPMPYDLYHPSGGG | SEQ ID NO: 87, |
| | C168S) | | SEQ ID NO: 88, |
| | | | SEQ ID NO: 8 |
| TC24 | CD122 (C122S, | GGSSGPVPLSLYSGSGGG | SEQ ID NO: 87, |
| | C168S) | | SEQ ID NO: 90, |
| | | | SEQ ID NO: 8 |
| TC46 | CD122 (C122A, | GPPSGSSPMPYDLYHPSGGG | SEQ ID NO: 87, |
| | N123C) | | SEQ ID NO: 89, |
| | | | SEQ ID NO: 8 |
| TC47 | CD122 (C122A, | GGSSGPVPLSLYSGSGGG | SEQ ID NO: 87, |
| | N123C) | | SEQ ID NO: 91, |
| | | | SEQ ID NO: 8 |
| TC29 | No Masking moiety | | |
| (Negative | | | |
| Control) | | | |

Mixed donor lots of human peripheral blood mononuclear cells (PBMCs) were thawed and seeded in medium (RPMI1640, 10% fetal bovine serum (FBS), 2 mM 1-Glutamine, 50 U/mL penicillin, 50 μg/mL streptomycin, 1 μM each of non-essential amino acids, 10 mM HEPES, 1 mM sodium pyruvate, 55 μM B-mercaptoethanol, 10 μg/mL gentamycin) into tissue culture treated plates that were coated for 1 h at 37° C. with 1 μg/ml anti-CD3. Anti-CD28 was added in solution at a concentration of 5 μg/ml. Two days later the PBMCs were collected and transferred into a 96 well v-bottom plate at 100,000 cells per well in FACS Buffer (2% FBS in phosphate buffered saline (PBS)). Test articles were diluted to twice the indicated concentration in FACS buffer. Test articles were added to cells at a 1:1 ratio to the final concentration indicated in the figure and incubated for 1 h at 4° C. The cells were washed three times with FACS buffer and fixed in 4% PFA. Cells were then washed once in FACS buffer. All cells were stained in 100 μL of indicated molecules in FACS buffer for 1 h at 4° C. The cells were washed three times with FACS buffer and fixed in 4% paraformaldehyde (PFA). All cells were subsequently stained in 100 μL with anti-human IgG PE, anti-CD3 PE/Dazzle 594, anti-CD45 PerCP Cy5.5, anti-CD8 BV510, and anti-CD4 BV421 for 30 minutes. Cells were washed twice to remove unbound antibody and analyzed on the BD Celesta by gating on CD3+CD8+ T cells. Data were fit and EC50s generated using a nonlinear regression [Agonist] vs. response—Variable slope (four parameter) in GraphPad Prism except for TKO and the isotype control which did produce full sigmoidal curves.

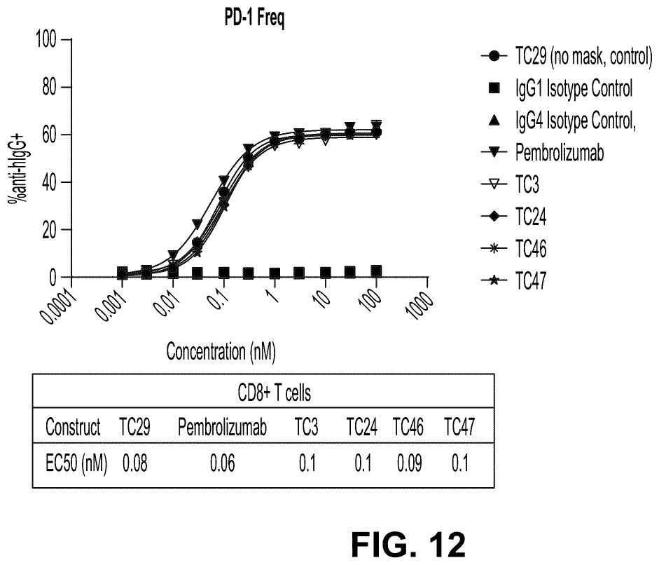

As shown in FIG. 12, all constructs bind CD3/CD28 activated CD8+ T cells with equivalent EC50 comparable to Pembrolizumab. Isotype controls exhibited limited to no binding at all concentrations tested.

### Example 8. Efficacy and Peripheral Activation of PD-1 Targeting Cytokines in hPD-1 Mice Bearing MC38 Tumors in Comparison to Pembrolizumab or the Combination of Pembrolizumab and Non-Targeted Cytokine

In this example, efficacy of PD-1 targeted cytokines was compared to Pembrolizumab (PD-1 antibody) or the combination of Pembrolizumab+non-targeted cytokine.

TC3, TC29, and TC0 was produced in house with aglycosylation hIgG1 isoform. Clinical grade Pembrolizumab was sourced from Myonex and has a human IgG4 domain. Female C57BL/6 hPD-1 mice (n=8 in each treatment group) were inoculated with 5×10⁵ MC38 tumor cells subcutaneously in the right flank. Mice with established MC38 tumors were treated on day 0, 3, 6 with 0.2 mL of vehicle control (closed circle); 0.31 mg/kg of TC0 (Fc-IL2, schematic represented in FIG. 5A) (open square, dash line; 26-30% TGI); 0.75 mg/kg of TC29 (open triangle, dash line: 54-75% TGI); TC0+Pembrolizumab (open square; 56-61% TGI); 8 mg/kg of TC3 (close square; 63-66% TGI) or 6 mg/kg of Pembrolizumab (close triangle; 22-47% TGI). A Two-way ANOVA Dunnet's with mixed-effect multiple comparison post-test was performed to determine the statistical significance of treatment vs vehicle (*P<0.05; **P<0.01; ***P<0.001; ****P<0.0001).

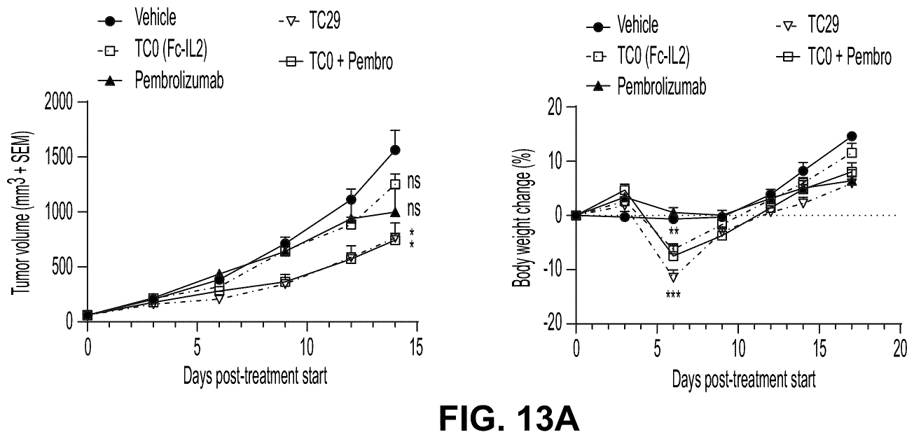

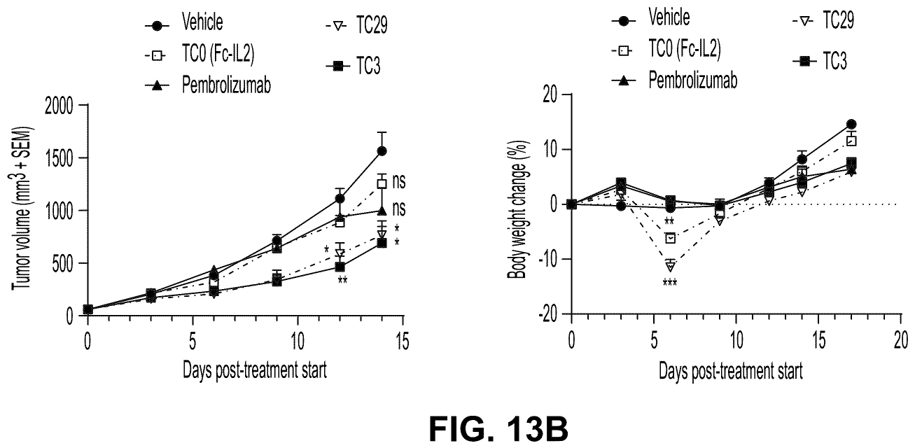

FIG. 13A and FIG. 13B show that all mice treated with the control group developed tumors that rapidly grew whereas treatment with PD-1 targeted cytokine resulted in day tumor growth.

Female C57BL/6 hPD-1 mice (n=2 in each treatment group) were inoculated with 5×10⁵ MC38 tumor cells subcutaneously in the right flank. Mice with established MC38 tumors were treated on day 0, and 3 with vehicle control or PD-1 targeted (Pembro Fab₂), unmasked TC29. TC29 comprises an IL2 variant (R38A, F42A, Y45A, E62A, C125A) a first Fc polypeptide with “hole mutations” (Y349C; T366S; L368A; and Y407V) and a second Fc polypeptide with “knob mutations” (S354C and T366W) and RF mutations (H435R and Y436F). Tumors were harvested tumors and single cell suspension samples were prepared for CD45 enrichment. Samples were analyzed by scRNA-seq. TC29 treated samples showed an enrichment of T and NK cells upon treatment, while vehicle-treated samples had higher proportions of DCs, macrophages and monocytes (data not shown). different Macrophage subtypes were found in MC38 tumor samples (infiltrating vs. resident; inflammatory vs. M2). Relative frequency of Monocytes and Macrophages (among Cd45+ cells) was higher in vehicle-treated samples, with the exception of Arg1+ and Mrc1+M2 Macrophages, which were the most abundant M cell subtype found in TC29-treated samples. Most NK and T cell subtypes were dramatically increased in TC29-treated MC38 tumors as compared to vehicle-treated controls. CD4+ T cells (which were mainly Foxp3+ and Il2ra+) were not significantly changed in TC29- vs. vehicle-treated samples. Stem-like, effector as well as exhausted CD8 cells were highly abundant in all TC29-treated samples.

### Example 9. In Vivo Pharmacodynamics and Efficacy of VaPD-1 Targeting Cytokines in Human PD-1 Expressing Mice Bearing MC38 Tumors

In this example, efficacy of various PD-1 targeted cytokines was assessed.

Female C57BL/6 hPD-1 mice (n=8 in each treatment group) were inoculated with 5×10⁵ MC38 tumor cells subcutaneously in the right flank. Mice with established MC38 tumors were treated on day 0, 3 and day 6 with 0.2 mL of vehicle control (closed circle); 8 mg/kg of masked, uncleavable TC2 (open square, dash line; 40-46% TGI); 0.75 mg/kg of unmasked PD-1 targeting TC1 (open triangle, dash line: 75-80% TGI); TC4 (open diamond, dotted line; 56-62% TGI); 8 mg/kg of TC3 (close square; 64-69% TGI) or 8 mg/kg of Pembrolizumab (close triangle; 22-32% TGI). Tumor size and body weight change were measured throughout the study. Plasma pharmacokinetic levels of total PD-1 multifunctional were measured at 10 minutes, 6 hours, 1 day, 3 days (prior second dose) and day 6 after treatment initiation using a Mesoscale Discovery (MSD) assay.

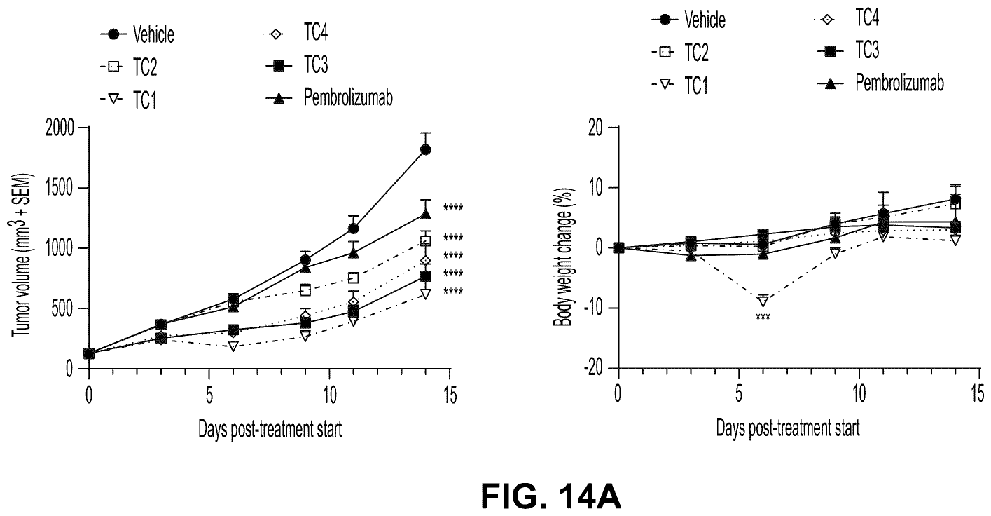

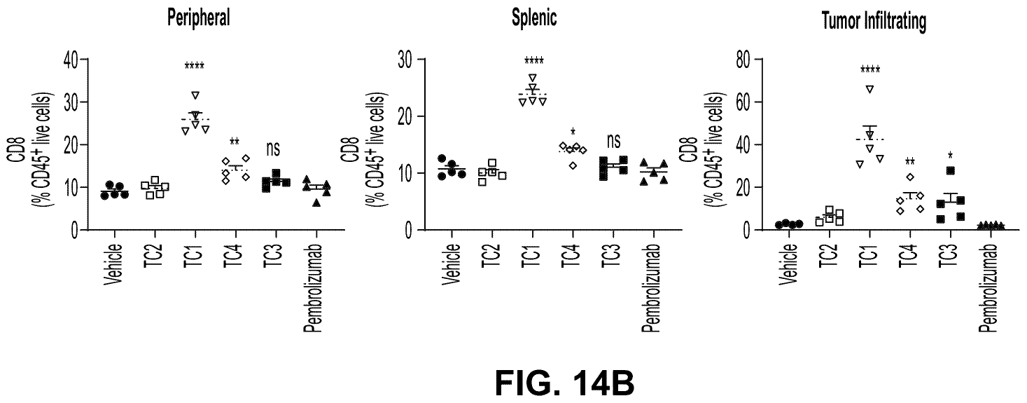

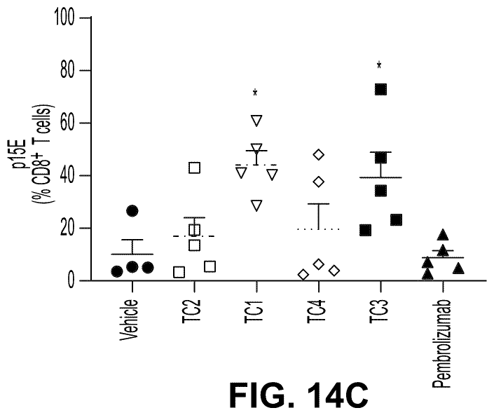

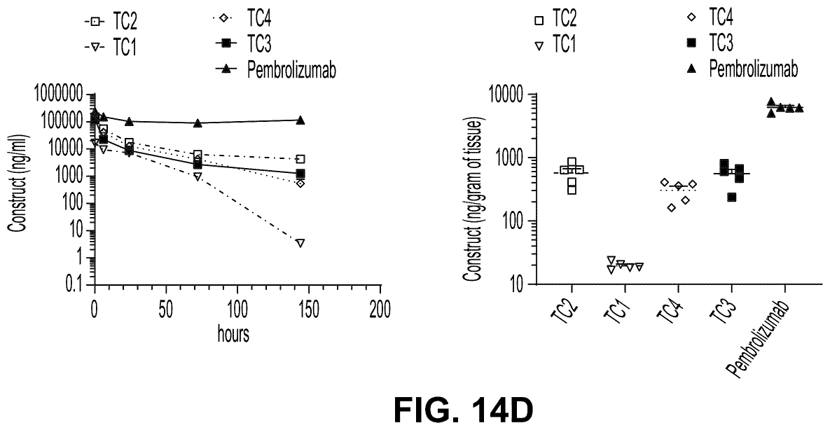

FIG. 14A shows that all mice treated with the control group developed tumors that rapidly grew whereas treatment with PD-1 targeted cytokines resulted in day tumor growth. FIG. 14B shows that treatment with PD-1 targeted masked cytokines induced activation/expansion of CD T cells in MC38 tumors. Additionally, treatment with PD-1 targeted masked molecule increased tumor antigen specific CD8 T cells (FIG. 14C).

Pharmacokinetic parameters of total PD-1 targeted cytokines and pembrolizumab were measured and are shown in FIG. 14D and Table 7.

TABLE 7
Pharmacokinetic Parameters of total PD-1 multifunctional and Pembrolizumab

| | Dose | | | | | |
|---|---|---|---|---|---|---|
| Test | Level | Half-life | Cmax | Cmax_D | AUClast | AUClast_D |
| article | (mg/kg) | (hr) | (ug/mL) | (kg*ug/mL/mg | (hr*ug/mL) | (hr*kg*ug/mL/mg) |
| TC2 | 8 | 22.7 ± 4.02 | 150 ± 12 | 18.7 ± 1.5 | 1800 ± 239 | 229 ± 29.9 |
| TC1 | 0.75 | 18.7 ± 1.22 | 16.4 ± 2.48 | 21.9 ± 3.3 | 422 ± 47.4 | 562 ± 63.2 |
| TC4 | 8 | 21.2 ± 2.14 | 180 ± 74 | 22.4 ± 9.28 | 1560 ± 524 | 195 ± 65.5 |
| TC3 | 8 | 22.6 ± 1.09 | 119 ± 14.5 | 14.9 ± 1.81 | 989 ± 80.4 | 124 ± 10.1 |
| Pembro | 10 | 66.5 ± 8.0 | 237 ± 23.4 | 23.7 ± 2.3 | 8060 ± 2040 | 806 ± 204.0 |

### Example 10. Production and Characterization of Engineered Cytokine Fusion Proteins

In this example, various targeted cytokines were designed and produced. Exemplary targeted IL-2 cytokine constructs comprising an IL-2 cytokine, a masking moiety that binds to IL-2 (e.g., CD122, anti-IL-2 (e.g., anti-IL2 VHH, scFv)), an Fc domain comprising, and a targeting moiety (e.g., anti-PD1) are summarized in Table 8. The exemplary constructs described below comprise a first Fc polypeptide with “hole mutations” (Y349C; T366S; L368A; and Y407V) and a second Fc polypeptide with “knob mutations” (S354C and T366W) and RF mutations (H435R and Y436F). As shown below, exemplary PD1 targeting moieties were derived from pembrolizumab (pembro) or nivolumab (nivo).

TABLE 8
Exemplary Engineered Cytokine Fusion Proteins

| | Cleavage | | | | Half-life |
|---|---|---|---|---|---|
| Construct | Peptide | IL-2 Mutations | Mask | Targeting | extension |
| TC48 | MPYDLYHP | R38A, F42A, Y45A, | CD122* | Pembro | YTE |
| | | E62A, C125A | | (Fab × 2) | |
| TC49 | MPYDLYHP | R38A, F42A, Y45A, | SCFV1 | Pembro | YTE |
| | | E62A, C125A | | (Fab × 2) | |
| TC50 | MPYDLYHP | R38A, F42A, Y45A, | CD122* | Pembro | YTE |
| | | E62A, C125A | | scFv | |
| | | | | VH/VL | |
| TC51 | MPYDLYHP | R38A, F42A, Y45A, | SCFV1 | Pembro | YTE |
| | | E62A, C125A | | scFv | |
| | | | | VH/VL | |
| TC52 | MPYDLYHP | R38A, F42A, Y45A, | CD122* (xG) | Pembro (Fab × 2) | |
| | | E62A, C125A | | | |
| TC53 | MPYDLYHP | R38A, F42A, Y45A, | SCFV1 | Pembro (Fab × 2) | |
| | | E62A, C125A | | | |
| TC54 | MPYDLYHP | R38A, F42A, Y45A, | CD122* (xMan) | Pembro (Fab × 2) | |
| | | E62A, C125A | | | |
| TC55 | MPYDLYHP | R38A, F42A, Y45A, | CD122* (xMan, | Pembro (Fab × 2) | |
| | | E62A, C125A | xG) | | |
| TC56 | MPYDLYHP | R38A, F42A, Y45A, | QVQ_VHHv1 | Pembro (Fab × 2) | |
| | | E62A, C125A | | | |
| TC57 | MPYDLYHP | R38A, F42A, Y45A, | QVQ_VHHv2 | Pembro (Fab × 2) | |
| | | E62A, C125A | | | |
| TC58 | MPYDLYHP | R38A, F42A, Y45A, | CD122* | Pembro scFv VH/VL | |
| | | E62A, C125A | | | |
| TC59 | MPYDLYHP | R38A, F42A, Y45A, | SCFV1 | Pembro | YTE |
| | | E62A, C125A | | scFv | |
| | | | | VH/VL | |
| TC60 | MPYDLYHP | R38A, F42A, Y45A, | CD122* | Pembro scFv VH/VL | |
| | | E62A, C125A | | | |
| TC61 | MPYDLYHP | R38A, F42A, Y45A, | SCFV1 | Pembro scFv VH/VL | |
| | | E62A, C125A | | | |
| TC62 | MPYDLYHP | R38A, F42A, Y45A, | CD122* | Nivo scFv | |
| | | E62A, C125A | | | |
| TC63 | MPYDLYHP | R38A, F42A, Y45A, | SCFV1 | Nivo scFv | |
| | | E62A, C125A | | | |
| TC64 | MPYDLYHP | R38A, F42A, Y45A, | CD122* | Pembro (Fab × 2) | |
| | | E62A, C125A | | | |
| TC65 | MPYDLYHP | R38A, F42A, Y45A, | VHHv2 | Pembro | YTE |
| | | E62A, C125A | | (Fab × 2) | |
| TC66 | MPYDLYHP | R38A, F42A, Y45A, | SCFV1 | Nivo (Fab × | |
| | | E62A, C125A | | 2) | |
| TC67 | MPYDLYHP | R38A, F42A, Y45A, | VHHv2 | Nivo (Fab × | |
| | | E62A, C125A | | 2) | |

| | | | | |
|---|---|---|---|---|
| TC68 | MPYDLYHP | wtIL-2 (C125A) | VHH | Pembro (Fab × 2) |
| TC69 | VPLSLYSG | wtIL-2 (C125A) | VHH | Pembro (Fab × 2) |
| TC70 | RAAAVKSP | wtIL-2 (C125A) | VHH | Pembro (Fab × 2) |
| TC71 | PANLVAPDP | wtIL-2 (C125A) | VHH | Pembro (Fab × 2) |
| TC72 | MPYDLYHP | R38A, F42A, Y45A, | VHH | Pembro (Fab × 2) |
| | | E62A, C125A | | |
| TC73 | VPLSLYSG | R38A, F42A, Y45A, | VHH | Pembro (Fab × 2) |
| | | E62A, C125A | | |
| TC74 | RAAAVKSP | R38A, F42A, Y45A, | VHH | Pembro (Fab × 2) |
| | | E62A, C125A | | |
| TC75 | PANLVAPDP | R38A, F42A, Y45A, | VHH | Pembro (Fab × 2) |
| | | E62A, C125A | | |
| TC80 | MPYDLYHP | R38A, F42A, Y45A, | SCFV1 | Pembro (Fab × 2) |
| | | E62A, C125A | | |
| TC82 | — | wtIL-2 (C125A) | | Pembro (Fab × 2) |
| TC83 | non-cleavable | R38A, F42A, Y45A, | SCFV1 | Pembro (Fab × 2) |
| | | E62A, C125A | | |
| TC84 | non-cleavable | R38A, F42A, Y45A, | VHH | Pembro (Fab × 2) |
| | | E62A, C125A | | |
| TC85 | MPYDLYHP | N88D, C125A | SCFV1 | — |

Targeted cytokines in Table 7 were generated and purified from HEK or CHO cells as described in Example 1. All molecules were purified with greater than 95% purity as measured by capillary electrophoresis sodium dodecyl sulfate (CE-SDS). 3 chain molecules have non-reduced purity range of >95%, 2 chain molecules have non-reduced purity range of >97% and both 3 chain and 2 chain molecules have reduced purity range of >95%. Moreover, all molecules were purified with <0.3% knob impurity and there was noticeable heterogeneity in all samples under reducing conditions.

Pharmacokinetic parameters of exemplary constructs are shown in Table 9, below.

TABLE 9
Exemplary Pharmacokinetic Parameters

| | | | | | AUCₗₐₛₜ | | AUC_{0-INF} | | | |
|---|---|---|---|---|---|---|---|---|---|---|
| | | | Cmax (ug/mL) | | (hr*ug/mL) | | (hr*ug/mL) | | Clearance | |
| | T_{1/2} (hr) (±SD) | | (±SD) | | (±SD) | | (±SD) | | (mL/hr/kg) | |
| Dose (mg/kg) | 2 | 8 | 2 | 8 | 2 | 8 | 2 | 8 | 2 | 8 |
| MC86 | 58 ± | 58 ± | 70 ± | 394 ± | 380 ± | 2259 ± | 394 ± | 2383 ± | 5.09 ± | 3.37 ± |
| Control | 5.96 | 4.52 | 6.59 | 67.7 | 31.5 | 178.7 | 31.5 | 173.3 | 0.407 | 0.407 |
| (untargeted) | | | | | | | | | | |
| MC87 | 45 ± | 55 ± | 100 ± | 244 ± | 392 ± | 1380 ± | 398 ± | 1434 ± | 5.14 ± | 5.63 ± |
| Control | 2.79 | 11.8 | 33.2 | 49.6 | 70.7 | 164.1 | 71.4 | 155.9 | 0.823 | 0.654 |
| (untargeted) | | | | | | | | | | |
| TC85 | 190 ± | 152.6 ± | 152 ± | 696 ± | 3956 ± | 12453 ± | 5757 ± | 15393 ± | 0.349 ± | 0.522 ± |
| | 20.79 | 26.07 | 1.8 | 135.9 | 143 | 280 | 486 | 1036 | 0.03 | 0.04 |
| MC88 | 62 ± | 73 ± | 131 ± | 636 ± | 1656 ± | 5023 ± | 1748 ± | 5485 ± | 1.15 ± | 1.48 ± |
| (untargeted) | 4.84 | 10.53 | 10.77 | 99.0 | 169 | 768 | 158 | 719 | 0.107 | 0.175 |
| Dose (mg/kg) | 4 | 16 | 4 | 16 | 4 | 16 | 4 | 16 | 4 | 16 |
| TC46 | 53 ± | 62 ± | 254 ± | 1047 ± | 1283 ± | 5217 ± | 1312 ± | 5458 ± | 3.09 ± | 2.98 ± |
| | 9.06 | 5.67 | 46.78 | 204 | 181 | 844 | 857 | 857 | 0.401 | 0.427 |

### Example 11. Masking and Proteolytic Cleavage

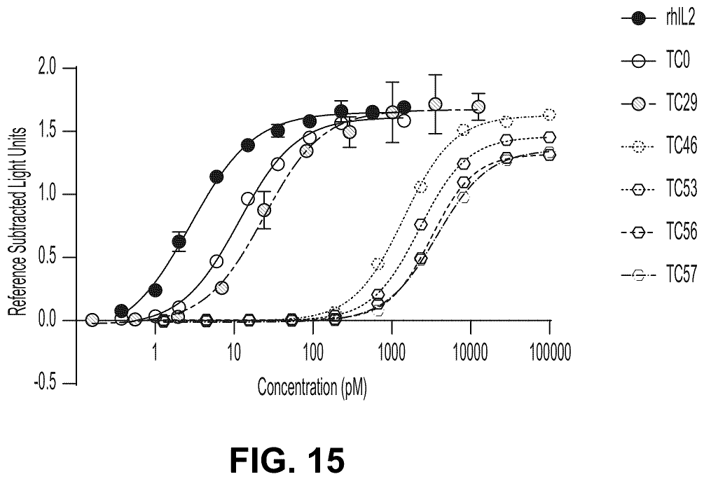

This example demonstrates proteolytic cleavage and masking activity of targeted cytokines described in Example 10. EC50 value of each construct was measured using a cell-based reporter assay (HEK Blue IL-2 assay). As shown in Table 9 and FIG. 15, all constructs were efficiently masked (Intact Fold Masking) and their activity restored following incubation with MMPs (+MMP) compared to TC0. Non-reduced gels show that all constructs were fully cleaved and additional bands were observed after incubation with 990 ng MMP10.

TABLE 9
Proteolytic cleavage and masking activity of targeted cytokines

| | | | | | | Intact | +MMP |
|---|---|---|---|---|---|---|---|
| Molecule | PD1 Targeting | | | Average n = 2 | | Fold Masking | Fold Masking |
| Name | Moiety | HLE | Mask | EC₅₀ (pM) | STD | Against TC0 | Against TC0 |
| TC29 | Pembro (Fab) | MST | N/A | 27.08 | 4.0 | 1.2 | 2.1 |
| TC46 | Pembro (Fab) | MST | CD122 | 1661.5 | 365.6 | 76.1 | 1.8 |
| TC54 | Pembro (Fab) | MST | CD122(xMan) | 2112.5 | 123.7 | 96.8 | 1.9 |
| TC52 | Pembro (Fab) | MST | CD122(xG) | 1493 | 87.7 | 68.4 | 1.4 |
| TC55 | Pembro (Fab) | MST | CD122(xMan, | 1837.5 | 460.3 | 84.2 | 1.8 |
| | | | xG) | | | | |
| TC53 | Pembro (Fab) | MST | SCFV1 | 3193.5 | 434.9 | 146.4 | 2.0 |
| TC56 | Pembro (Fab) | MST | VHH v1 | 6090 | 943.3 | 279.1 | 1.5 |
| TC57 | Pembro (Fab) | MST | VHH v2 | 5235.5 | 133.6 | 239.9 | 1.1 |
| TC48 | Pembro (Fab) | YTE | CD122 | 2985.5 | 471.6 | 136.8 | 2.0 |
| TC49 | Pembro (Fab) | YTE | SCFV1 | 3995.5 | 528.2 | 183.1 | 1.3 |
| TC58 | Pembro (scFv) | MST | CD122 | 3576.5 | 323.1 | 163.9 | 1.8 |
| | VHVL | | | | | | |
| TC59 | Pembro (scFv) | MST | SCFV1 | 5716 | 77.8 | 262.0 | 1.6 |
| | VHVL | | | | | | |
| TC50 | Pembro (scFv) | YTE | CD122 | 2564.5 | 246.8 | 117.5 | 1.6 |
| | VHVL | | | | | | |
| TC51 | Pembro (scFv) | YTE | SCFV1 | 4986.5 | 27.6 | 228.5 | 1.2 |
| | VHVL | | | | | | |
| TC60 | Pembro (scFv) | MST | CD122 | 2678 | 90.5 | 122.7 | 1.5 |
| | VLVH | | | | | | |
| TC61 | Pembro (scFv) | MST | SCFV1 | 4867.5 | 234.1 | 223.1 | 2.3 |
| | VLVH | | | | | | |
| TC62 | Nivo (scFv) | MST | CD122 | 2014.5 | 320.3 | 92.3 | 1.9 |
| TC63 | Nivo (scFv) | MST | SCFV1 | 3939.5 | 478.7 | 180.5 | 1.5 |

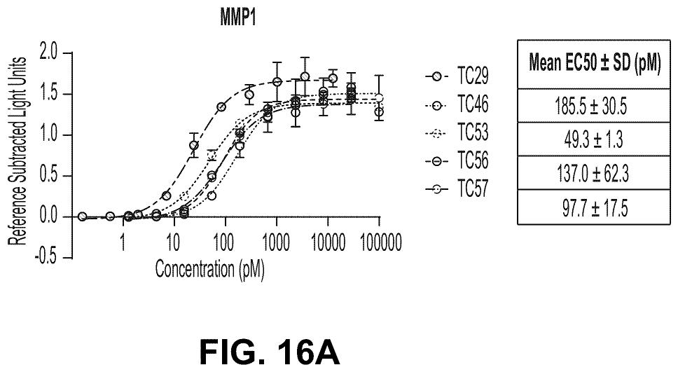

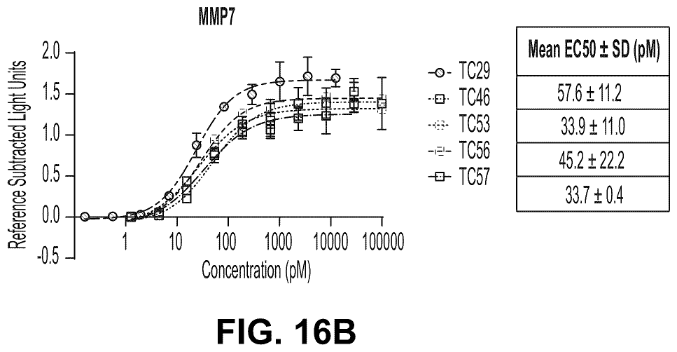

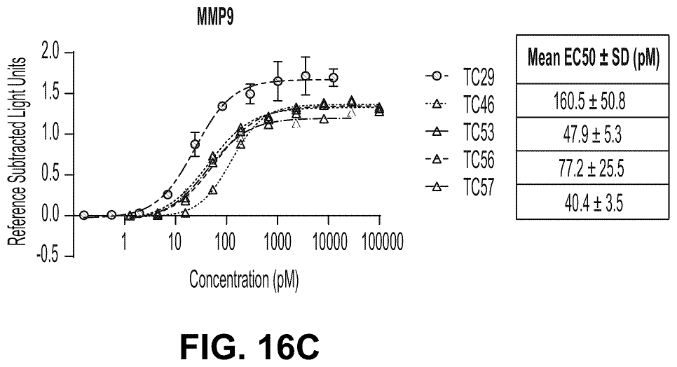

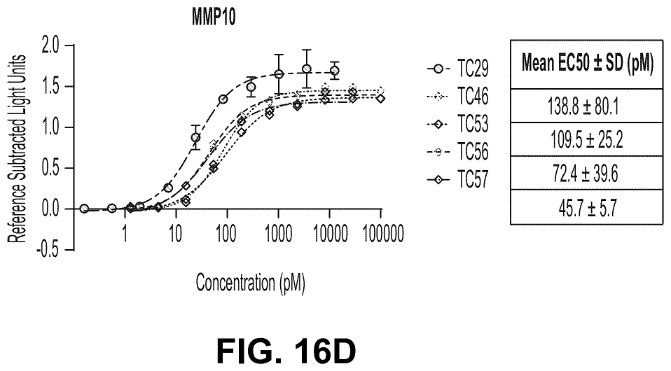

Masking ability of CD122, anti-IL2 scFv and anti-IL2 VHH in targeted cytokine constructs was further evaluated in a limited protease assay. Briefly, 2 uM of each construct was prepared and incubated with pre-determined amounts of protease to ensure minimal cleavage (30.1 nM MMP-1, 18.6 nM MMP-7, 6.8 nM MMP-9, 10.0 nM MMP-10) for 2 hours at 37° C. Reactions were quenched with EDTA and frozen. Samples were thawed and analyzed using RGA, SDS-PAGE. As shown in Table 10 and FIG. 16A-16D, all constructs were efficiently masked and cleaved. VHH masked constructs (TC56 and TC57) and scFv masked construct (TC53) demonstrated even greater masking than CD122 masked construct (TC46).

TABLE 10
Limited Protease Cleavage Assay

| No | EC50 | Fold Change | Fold Masking | Fold Masking |
|---|---|---|---|---|
| MMP | (pM) | against rhIL-2 | against TC0 | against TC29 |
| rhIL2 | 2.8 | | | |
| TC0 | 11.5 | | | |
| TC29 | 23.9 | | | |
| TC46 | 1415.0 | 506.1 | 122.9 | 59.3 |
| TC53 | 2249.0 | 804.4 | 195.4 | 94.2 |
| TC56 | 3189.0 | 1140.6 | 277.1 | 133.5 |

Masking and cleavage efficiency of additional constructs was measured by incubating cytokine constructs comprising CD122, scFv, or VHH masks with MMP 10. As demonstrated above, all constructs were efficiently masked and cleaved (Table 12).

TABLE 12
EC50 of masked constructs

| | EC50 | Fold change | Fold masking |
|---|---|---|---|
| Molecule | (pM) | against IL2 | against TC29 |
| rhIL2 | 3.2 | | |
| TC0 | 18.9 | 5.9 | |
| TC29 | 38.2 | 11.9 | |
| TC29 + MMP10 | 23.4 | 7.3 | |
| TC46 | 1912 | 596.8 | 50.0 |
| TC46 + MMP10 | 25.1 | 7.8 | 0.7 |
| TC64 | 1787 | 557.7 | 46.8 |
| TC64 + MMP10 | 17 | 5.3 | 0.4 |
| TC49 | 2592 | 809.0 | 67.8 |
| TC49 + MMP10 | 17.5 | 5.5 | 0.5 |
| TC57 | 2775 | 866.1 | 72.6 |
| TC57 + MMP10 | 19.7 | 6.1 | 0.5 |
| TC65 | 3120 | 973.8 | 81.6 |
| TC65 + MMP10 | 19.3 | 6.0 | 0.5 |
| TC66 | 1830 | 1586 | 1708 |
| TC66 + MMP10 | 15.5 | 8.2 | 11.8 |
| TC67 | 2473 | 1911 | 2192 |
| TC67 + MMP10 | 18 | 10.2 | 14.1 |

Additional IL-2 variants were tested for masking activity and PD-1 blocking activity in the targeted cytokine formats described herein. As shown in Table 13, masked cytokine constructs are >100-fold masked compared to a parental benchmark molecule, TC29. All molecules showed comparable PD-1 blockade potency to pembrolizumab, via PD-(L)1 blockade RGA assay.

TABLE 13
Masking Efficiency of Exemplary Constructs

| Type of IL-2 | Construct | EC50 (pM) |
|---|---|---|
| WT (C125A) | TC82 (not masked) | 1.7 |
| | TC68 | 165.3 |
| | TC68 + MMP | 0.5 |
| | TC69 | 37.84 |
| | TC69 + MMP | 0.7 |
| | TC70 | 57.92 |
| | TC70 + MMP | 0.4 |
| | TC71 | 28.79 |
| | TC71 + MMP | 0.9 |
| Not-alpha (R38A, F42A, | TC29 | 8.4 |
| Y45A, E62A, C125A) | TC53 | 2314 |
| | TC53 + MMP | 9.6 |
| | TC57 | 1554 |
| | TC57 + MMP | 8.1 |
| | TC73 | 1417 |
| | TC73 + MMP | 5.8 |
| | TC74 | 442.7 |
| | TC74 + MMP | 5.3 |
| | TC75 | 1469 |
| | TC75 + MMP | 8.9 |
| | TC91 | 1380 |
| | TC91 + MMP | 22.2 |
| | TC92 | 1160 |
| | TC92 + MMP | 19.7 |
| | TC93 | 1646 |
| | TC93 + MMP | 6.4 |
| | TC83 | 1713 |
| | TC83 + MMP | 41.2 |
| | TC84 | 201.8 |
| | TC84 + MMP | 122.1 |

Constructs were tested to determine the catalytic efficiency of recombinant human MMP-2, MMP-7, MMP-9, and MMP-14. The catalytic efficiency (kcat/Km) of MMPs (R&D Systems) to cleave each drug molecule was assessed by cleavage time-course assay. All the MMPs were activated at 37° C. before cleavage reactions, following the manufacturer's instruction. Briefly, MMP-2 and MMP-7 were activated by 1 mM 4-aminophenyl-mercuric acetate (APMA) (Sigma-Aldrich) for 1 hr; MMP-9 was incubated with 1 mM APMA for 20 hrs; and MMP-14 was activated by 0.1 μg/mL Trypsin 3 (R&D Systems) for 1 hr. The activation and cleavage reaction were conducted in the same buffer containing 50 mM Tris-HCl pH7.5, 150 mM NaCl, 10 mM CaCl₂, 0.05% Brij35 for MMP-2, MMP-7, and MMP-9; while 5 μM ZnCl₂ was added to the buffers for MMP-14.

To initiate a cleavage reaction, 20 nM active MMP was added to 2 μM drug molecule (targeted cytokine constructs) in a 904 solution, and incubated at 37° C. Aliquots (164) were removed and quenched by 4 μL 250 mM EDTA at 0, 30, 60, 150, 1360 minutes. The percentage of un-cleaved drug in the samples was determined by non-reducing CE-SDS technology (PerkinElmer), and used to calculate for % product=100−(un-cleaved drug %). Cleavage time course was fitted in GraphPad Prism using one-phase association model:

% Product=(Plateau−% Product 0 min)*(1−exp(−K*time))

The derived first-order constant K was then divided by MMP concentration to yield catalytic efficiency. Results are shown in Table 14.

TABLE 14
Cleavage Efficiency of Exemplary Targeted Cytokines

| | | Est. k_{cat}/K_{M}(M⁻¹s⁻¹) | | | | | | | |
|---|---|---|---|---|---|---|---|---|---|
| | | MMP-2 | | MMP-7 | | MMP-9 | | MMP-14 | |
| TC53 | MPYDLYHP | 4.9E+03 | 4.7E+03 | 2.4E+04 | 2.3E+04 | 1.9E+04 | 1.8E+04 | n/a | n/a |
| TC68 | | 3.8E+03 | 3.8E+03 | 1.3E+04 | 1.3E+04 | 1.6E+04 | 1.5E+04 | n/a | n/a |
| TC57 | | 4.0E+03 | 4.4E+03 | 1.3E+04 | 1.5E+04 | 1.3E+04 | 1.4E+04 | n/a | n/a |
| TC3 | | no exp. | | 1.3E+04 | | 3.5E+03 | | n/a | |
| TC46 | | no exp. | | 1.3E+04 | | 3.5E+03 | | n/a | |
| TC69 | VPLSLY | 5.1E+04 | 5.0E+04 | 7.8E+04 | 7.8E+04 | 3.5E+04 | 3.6E+04 | 4.1E+03 | 4.5E+03 |
| TC73 | | 4.3E+04 | 4.1E+04 | 7.1E+04 | 6.7E+04 | 3.0E+04 | 2.9E+04 | 4.0E+03 | 4.3E+03 |
| TC24 | | no exp. | | 1.4E+05 | | 4.2E+04 | | n/a | |
| TC47 | | no exp. | | 1.3E+05 | | 3.8E+04 | | n/a | |
| TC70 | RAAAVKSP | 1.1E+04 | 1.1E+04 | n/a | n/a | 3.9E+02 | 3.7E+02 | n/a | n/a |
| TC74 | | 9.6E+03 | 9.8E+03 | n/a | n/a | 8.5E+02 | 8.6E+02 | n/a | n/a |
| TC71 | PANLVAPDP | 1.3E+05 | 1.4E+05 | 1.9E+04 | 2.2E+04 | 2.8E+04 | 3.0E+04 | 5.0E+03 | 5.0E+03 |
| TC75 | | 1.2E+05 | 1.1E+05 | 1.8E+04 | 1.9E+04 | 2.5E+04 | 2.6E+04 | 4.8E+03 | 4.7E+03 |

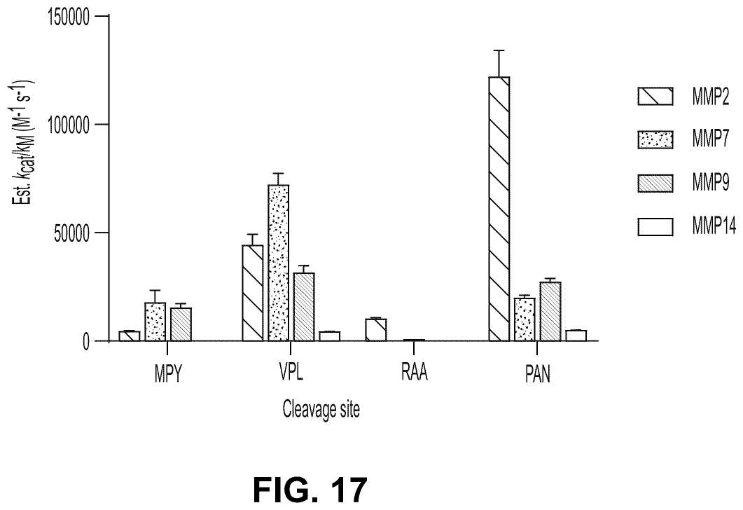

Average catalytic efficiency shown in FIG. 17, demonstrated that constructs with the same protease cleavage site had similar cleavage efficiencies. Cleavage sites engineered into the targeted cytokines did not affect stability of the molecules or binding to FcRn. (data not shown).

### Example 12. In Vitro Assessment of Targeting Capacity by PD-1 Binding

In this example, PD-1 targeted cytokines were evaluated for targeting capacity by PD-1 binding.

### ELISA and PD-1 Blockade Assay

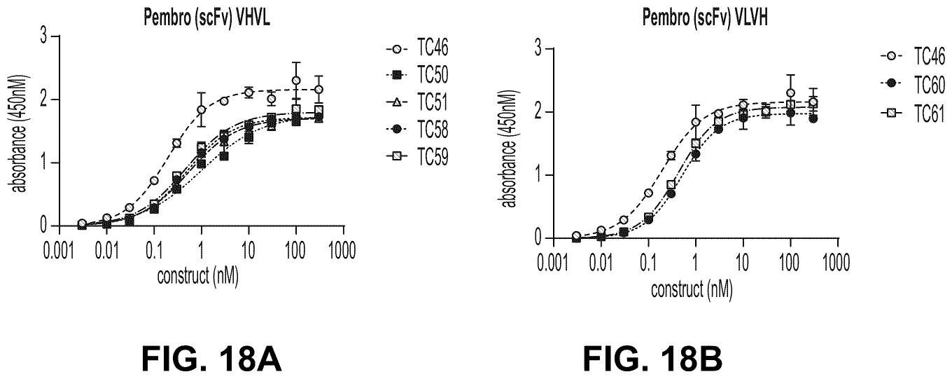

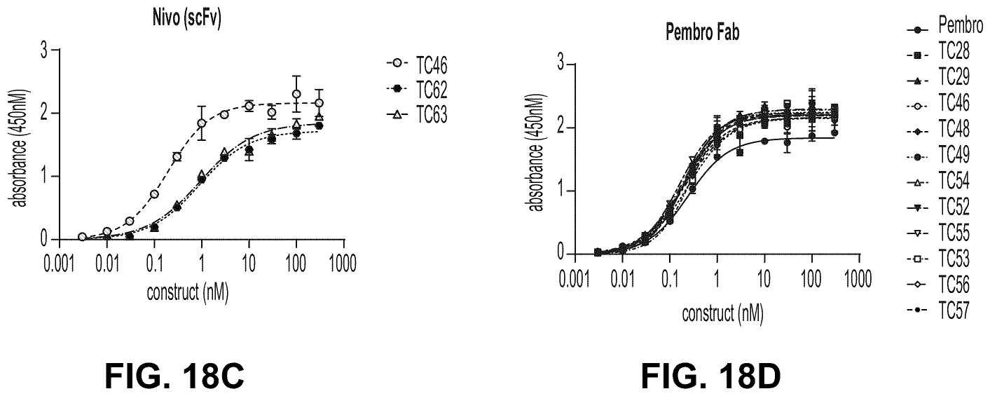

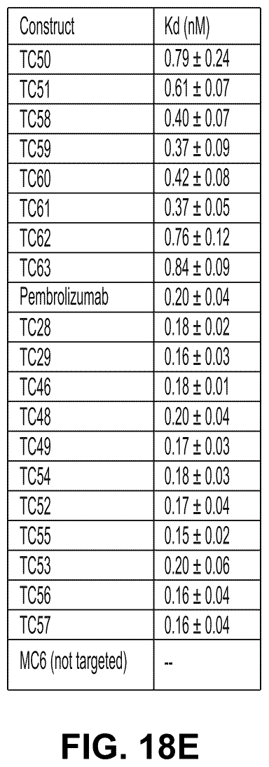

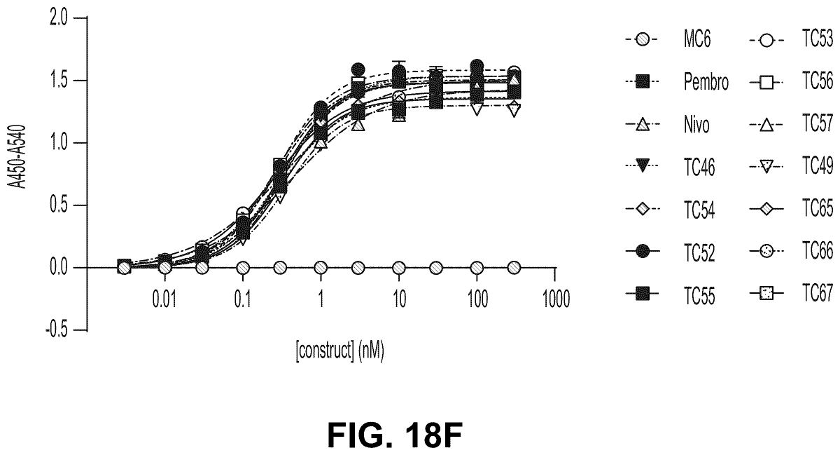

As shown in FIGS. 18A-18F, and Table 15, PD-1 targeted cytokines have high affinity for PD-1 as measured by ELISA.

TABLE 15
PD-1 binding affinity measured by ELISA

| | | | | | | Kd |
|---|---|---|---|---|---|---|
| | Targeting | Fc | Mask | Bmax | Hill Slope | (nM) |
| XR034 (Pembro IgG) | | | | 1.37 | 1.07 | 0.32 |
| Nivolumab | | | | 1.43 | 0.80 | 0.43 |
| TC46 | Pembro | — | CD122* | 1.54 | 1.14 | 0.33 |
| TC54 | Pembro | — | CD122*xG | 1.49 | 1.20 | 0.33 |
| TC52 | Pembro | — | CD122*xMan | 1.48 | 1.25 | 0.31 |
| TC55 | Pembro | — | CD122*xGxMan | 1.49 | 1.27 | 0.34 |
| TC53 | Pembro | — | SCFV1 | 1.59 | 1.21 | 0.28 |
| TC56 | Pembro | — | VHHv1 | 1.51 | 1.15 | 0.27 |
| TC57 | Pembro | — | VHHv2 | 1.54 | 1.14 | 0.26 |
| TC49 | Pembro | YTE | SCFV1 | 1.30 | 1.21 | 0.37 |
| TC65 | Pembro | YTE | VHHv2 | 1.35 | 1.23 | 0.33 |
| TC66 | Nivo | — | SCFV1 | 1.51 | 0.78 | 0.34 |
| TC67 | Nivo | — | VHHv2 | 1.42 | 0.96 | 0.24 |

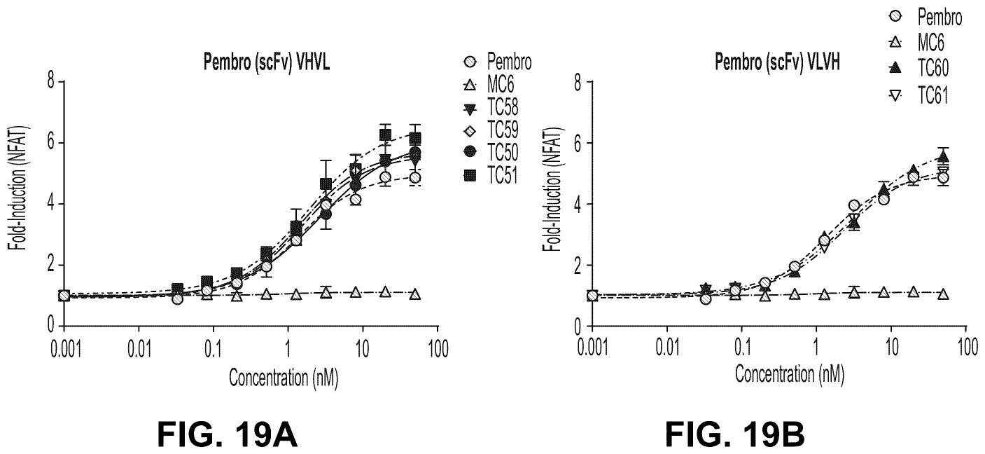

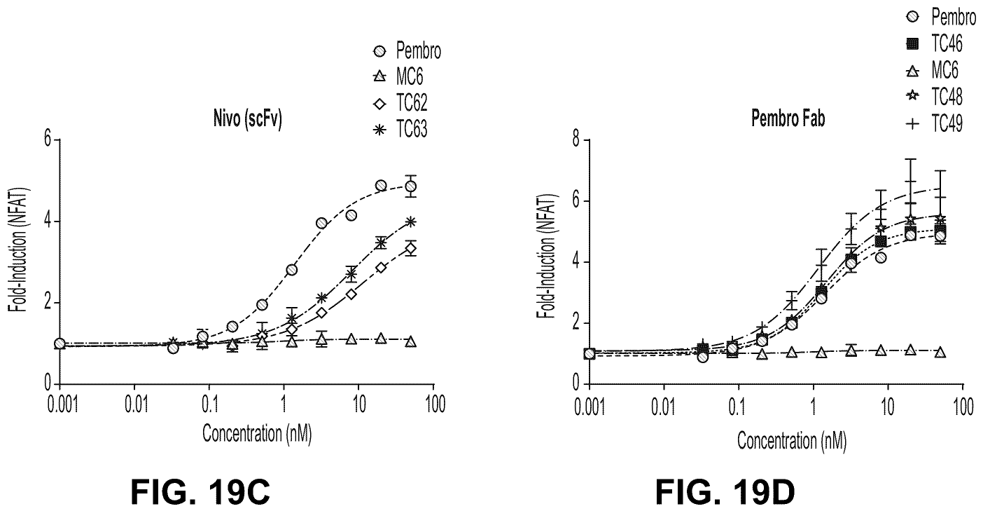

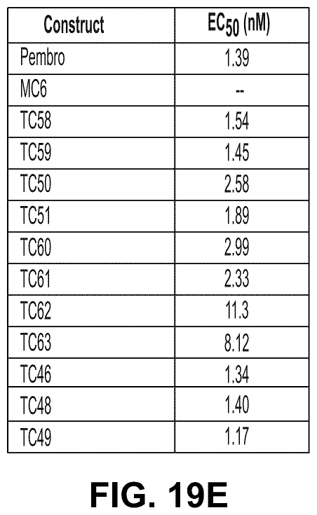

All molecules effectively block the PD1/PD-L1 checkpoint. Exemplary PD-1 RGA assay results are shown in FIGS. 19A-19F and Table 16. Briefly, the PD-1/PD-L1 blockade bioassay is a biologically relevant MOA-based assay used to measure the potency and stability of antibodies and other biologics designed to block the PD-1/PD-L1 interaction. When a PD-L1 expressing cell and a PD-1 effector cell are co-cultured, the PD-1/PD-L1 interaction inhibits TCR signaling and NFAT-mediated luciferase activity. Addition of an antibody that blocks either PD-1 or PD-L1 releases the inhibitory signal and results in TCR signaling and NFAT-mediated luciferase activity. Fold Induction can be calculated as shown in below formula:

$$Fold Induction = \frac{RLU ( Antibody - Background )}{RLU ( No antibody contorl - Background )}$$

In this blockade assay, pembro Fab and scFv molecules block PD-1/PD-L1 interactions in the same capacity as pembrolizumab. Human IgG isotype was used as a control (data not shown).

TABLE 16
EC50 of Exemplary Constructs
Measured by PD-1 Blockade RGA Assay

| Molecules | EC50 (nM) |
|---|---|
| pembrolizumab | 0.700 |
| TC53 | 0.751 |
| TC57 | 0.952 |
| TC68 | 0.686 |
| TC69 | 0.677 |
| TC70 | 0.956 |
| TC71 | 0.837 |
| TC73 | 0.993 |
| TC74 | 0.764 |
| TC75 | 0.992 |
| TC92 | 0.987 |
| TC93 | 0.991 |
| TC83 | 0.935 |
| TC84 | 0.914 |
| TC29 | 0.779 |

### On Cell Binding

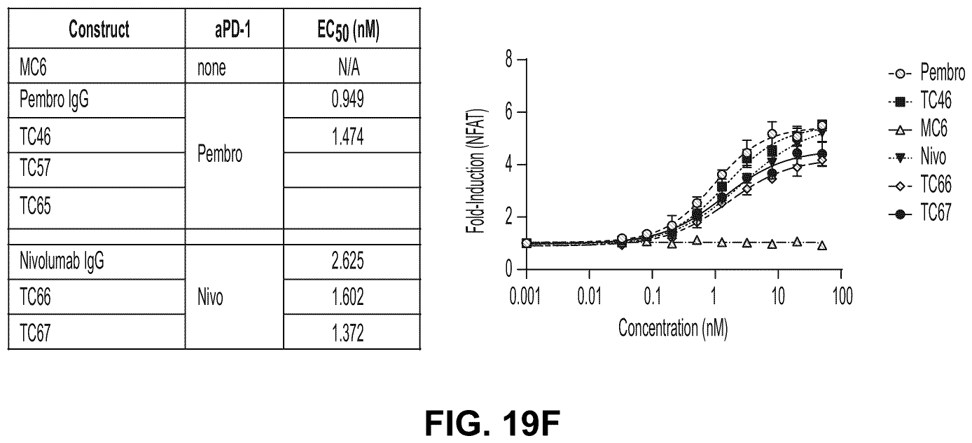

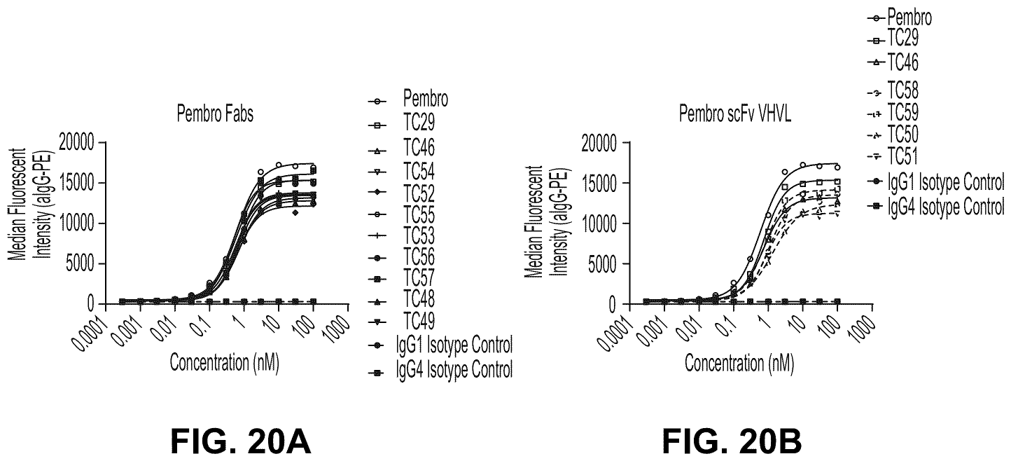

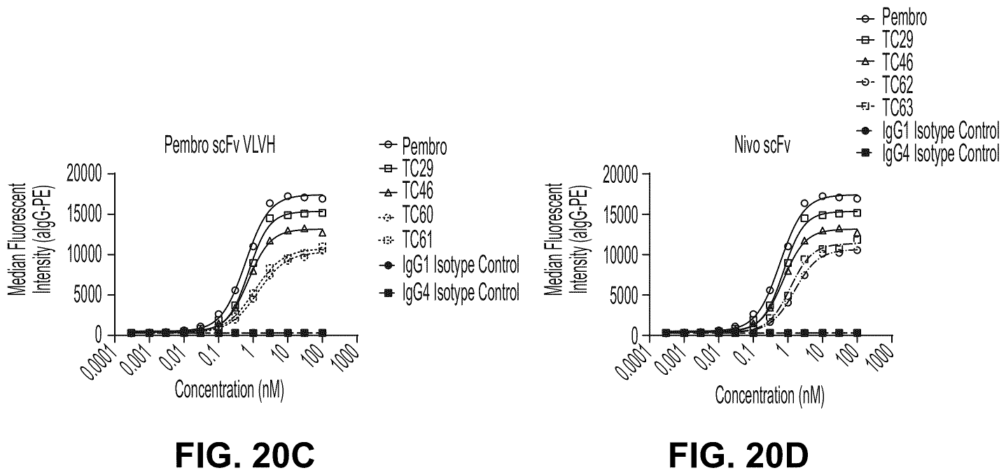

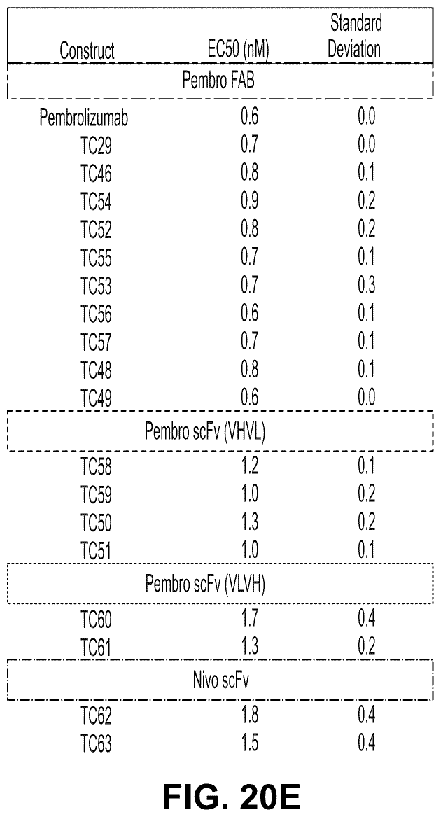

To assess whether these targeted cytokines bind to the PD-1, cell binding assays were performed as described in Example 2. As shown in FIG. 20A-FIG. 20E, all constructs bind PD-1 on Raji cells with similar EC50.

### Example 13: In Vivo Efficacy of Targeted Masked Cytokines in hPD-1 Mice

This example demonstrates in vivo efficacy of targeted cytokines. Pharmacokinetic and pharmacodynamic studies were performed in mice bearing human FcRn and hPD1. Tumor cell lines were grown and maintained at 37° C. and 5% CO2 upon reaching 50-70% confluence, cells were passaged for a total of two passages, prior to in vivo implantation. Cells were harvested using TrypLE Express, re-suspended in PBS, and 0.5-1×10⁶ cells in 100 μL PBS were subcutaneously implanted into the right flank of female B-hPD1 mice.

In vivo mouse assessments of targeted cytokine constructs were performed in C57BL/6-Pdcd1tm1(PDCD1) Bcgen/Bcgen transgenic mice purchased from Biocytogen (Beijing, China) and were 8-12 weeks old at the start of the study. C57BL/6 mice were engineered to express human PD-1 exon 2 (encoding the extracellular domain of PD-1) in place of the mouse counterpart (term B-hPD-1). Tumor cell were injected subcutaneously into the right flank of each mouse, and tumor volume was calculated twice weekly (Length*(Width\hat{ }2)/2) using dial calipers. For the efficacy studies, mice were randomized into treatment groups (N=8 mice each) when the mean tumor volume reached approximately 100-150 mm³. For the tumor pharmacodynamics (PD) studies, mice were randomized into treatment groups (N=5 mice each) when tumor volume reached approximately 200-400 mm³. Dosing for Efficacy or Pharmacodynamics (PD) was initiated following randomization on the same day, Day 0. Tumor volume and body weights were recorded two times per week for the duration of the study.

Immunophenotyping was performed using flow cytometry. In the pharmacodynamics (PD) studies, mice were euthanized by CO₂ asphyxiation and peripheral blood, spleens and tumors were harvested. Cell suspensions were prepared from spleens by mechanical disruption followed by lysis of red blood cells. Tumor cell suspensions were prepared by combining mechanical dissociation with enzymatic degradation of the extracellular matrix to achieve single-cell suspensions. The tumor tissues were enzymatically digested using Miltenyi Tumor Dissociation Kit reagents and the gentleMACS Dissociator was used for the mechanical dissociation steps. Following tumor digestion, debris was separated by sedimentation, and suspensions were passed through a 40 pin nylon cell strainer. Single cell suspensions were incubated with antibody cocktails for cell surface staining. Intercellular staining was performed using anti-mouse Foxp3-PE and following the fixation/permeabilization protocol from eBioscience. The optimal concentration for each antibody was pre-determined by titration.

The gating strategy applied for the analysis of peripheral blood was as follows: a parental gate was created around the lymphocyte population as identified by low forward scatter (FSC) and low side scatter (SSC) characteristics. From the lymphocyte gate, subpopulations of immune cells were identified on dot plots: leukocytes (CD45+) helper T cells (CD4+), cytotoxic T lymphocytes (CD8+), Natural Killer (NK) cells and regulatory T cells (CD4+FOXP3+CD25+).

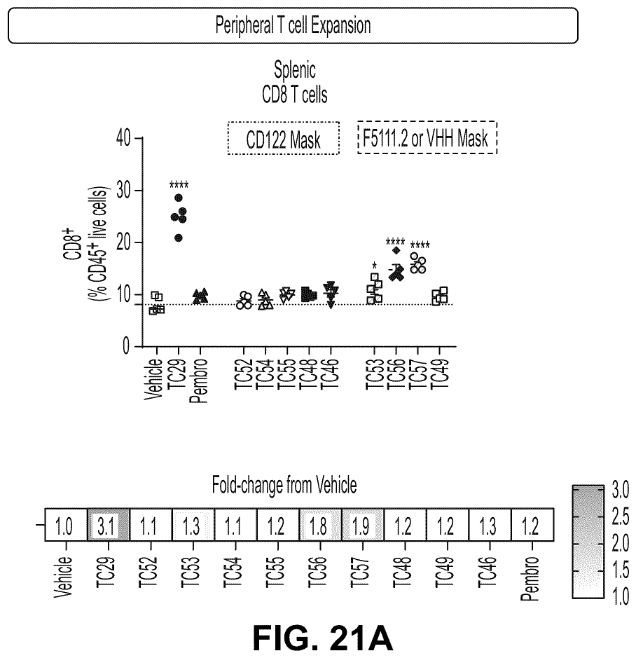

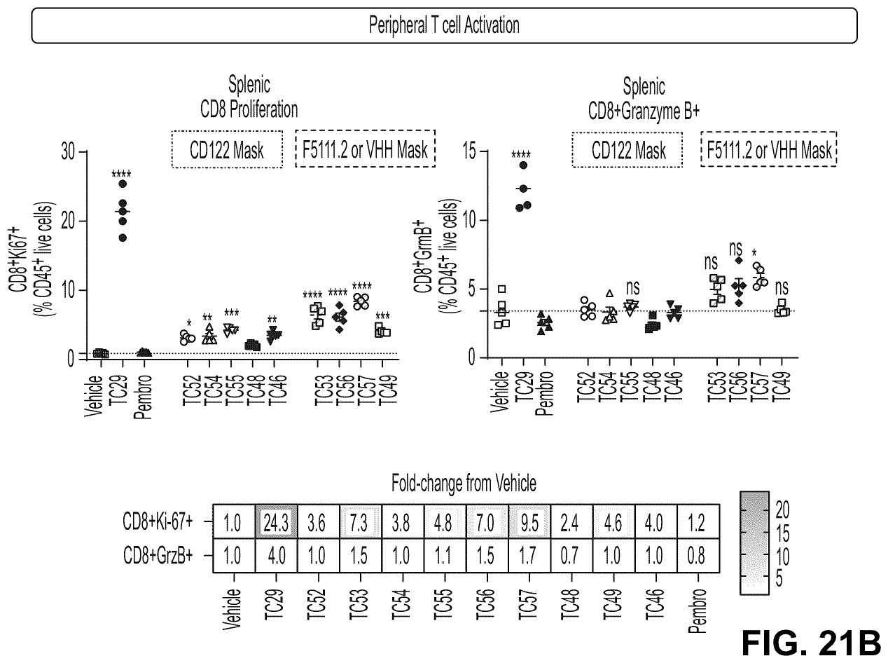

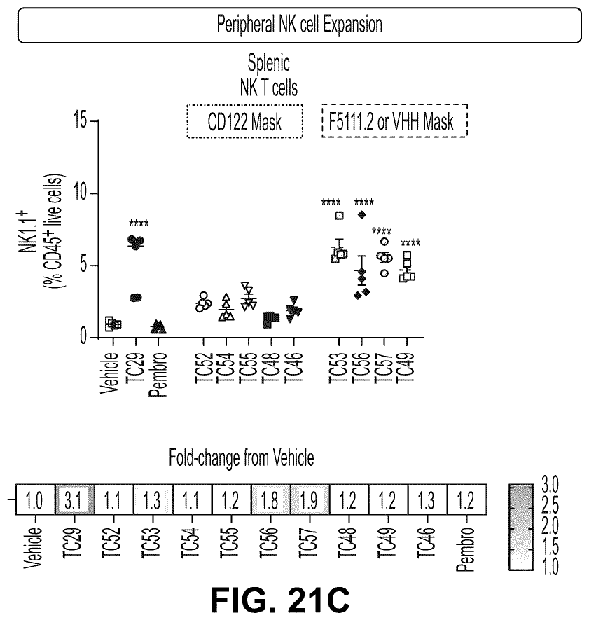

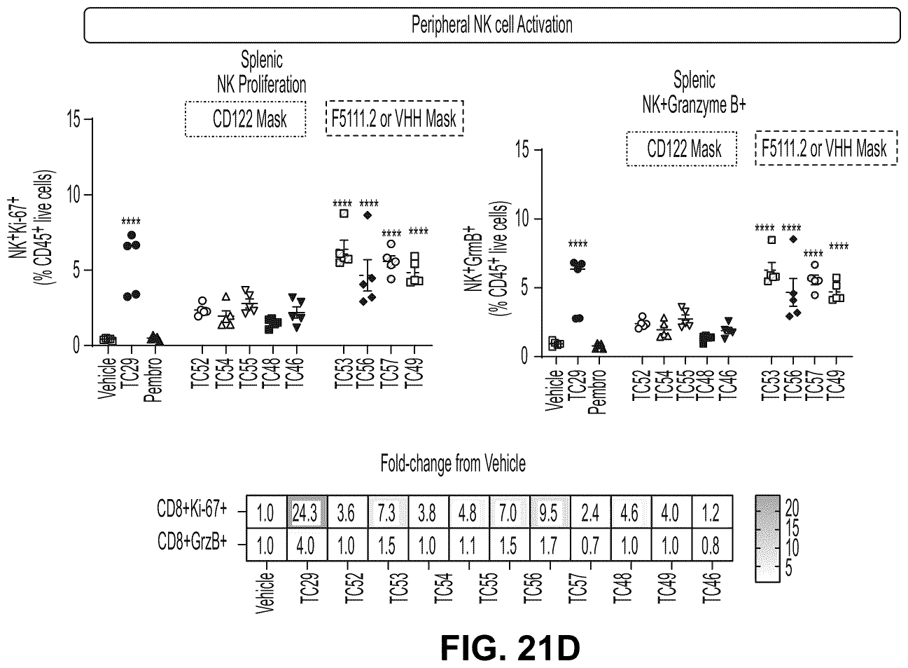

PD1 targeted masked molecules reduced peripheral CD8+ T cell expansion and activation induced by the parental unmasked TC29 and increase CD8+ T cell expansion in MC38 TME (FIG. 21A-FIG. 21B). At 10-fold higher dose PD1-multifunctional CD122 containing mask molecule reduces peripheral NK cell expansion and activation induced by the parental unmasked TC29 (FIG. 21C-FIG. 21D).

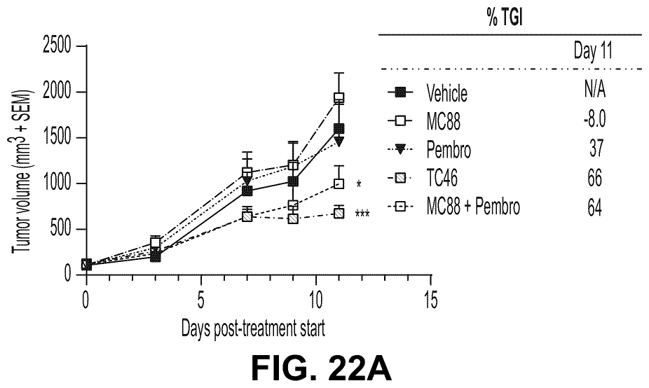

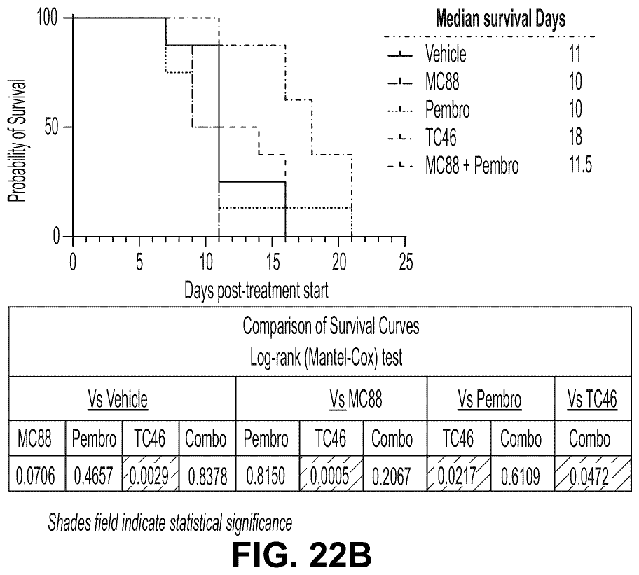

0.5×10⁶ MC38 tumor cells were injected subcutaneously into the right flank of hPD1 mice. Upon reaching ˜150 mm³ sized tumors (day 0), the mice received an intravenous (IV) dosing of the construct or intraperitoneal (I.P.) dosing of pembrolizumab as shown in Table 17. FIG. 22A-22B shows that treatment with an exemplary targeted cytokine showed improved anti-tumor activity and overall survival compared to monotherapy or combination masked cytokine and pembrolizumab.

TABLE 17
Dosing groups for MC38 Mouse Studies

| | | Mouse | Half-life | M.W. | | mg/kg | Dosing |
|---|---|---|---|---|---|---|---|
| | Test Molecule | # | extension | (kDa) | ROA | dose | Schedule |
| 1 | Vehicle Control | 8 | — | — | I.V | — | Q3D |
| 2 | MC88 (not targeted) | 8 | agly- | 93 | I.V | 4 | Q3D |
| | | | hIgG1 | | | | |
| 3 | Pembrolizumab | 8 | IgG4 | 146 | I.P | 6 | Q3D |
| 4 | TC46 | 8 | agly- | 189 | I.V | 8 | Q3D |
| | | | hIgG1 | | | | |
| 5 | MC88 (not targeted) | 8 | agly- | 93 | I.V | 4 | Q3D |
| | | | hIgG1 | | | | |
| | Pembrolizumab | | IgG4 | 146 | I.P | 6 | Q3D |

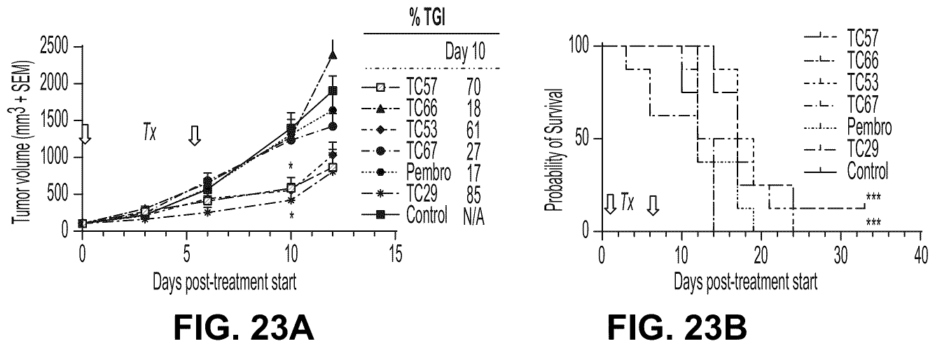

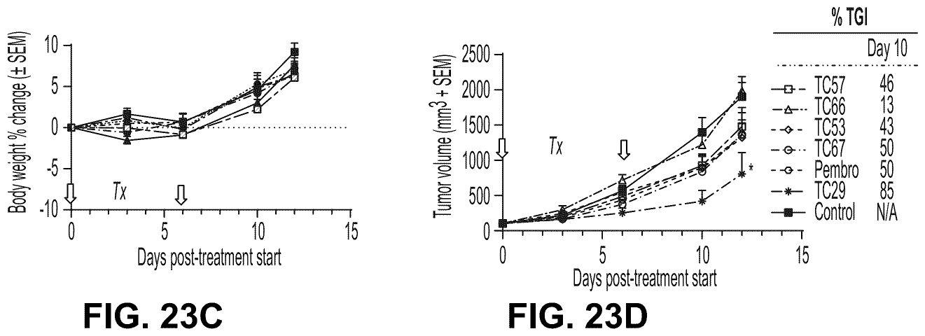

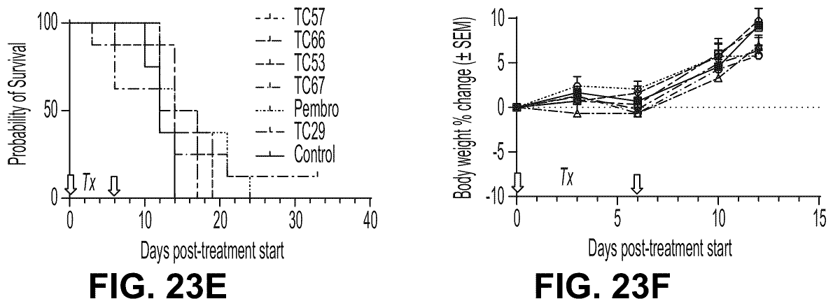

Additional targeted cytokines were evaluated for efficacy in MC38 mice. As described above, B-hPD-1 C57BL/6 mice were inoculated with MC38 tumor cells. When tumors reached ˜100-150 mm³ treatment was initiated on Day 0, 6 as summarized in Table 18. Tumor volume, body weights and overall survival were measured. Results of animals receiving approximately 8 mg/kg are shown in FIG. 23A-FIG. 23C. Animals who received approximately 2 mg/kg are shown in FIG. 23D-FIG. 23F. Plasma collection occurred pre-dose, Day 6 and Day 14. Anti-drug antibodies (ADAs) were measured from collected plasma. Targeted cytokine constructs demonstrated efficacy (e.g., significant tumor growth inhibition and survival) against the MC38 tumors in vivo.

TABLE 18
Dosing and test groups for MC38 studies

| | | | | | | Apprx. | Equal | |
|---|---|---|---|---|---|---|---|---|
| | | | Test | Mouse | M.W. | Dosing | molar Dose | Dosing |
| αPD1 | Mask | | Molecule | # | (kDa) | (mg/kg) | (nM) | Schedule |
| Pembro | VHH | 1 | TC57 | 8 | 177 | 7.50 | 4230 | D0, D6 |
| αPD1 | | 2 | TC57 | 8 | 177 | 2.50 | 1410 | D0, D6 |
| Nivo | | 3 | TC66 | 8 | 174 | 7.36 | 4230 | D0, D6 |
| αPD1 | | 4 | TC66 | 8 | 174 | 2.45 | 1410 | D0, D6 |
| Pembro | SCFV1 | 5 | TC53 | 8 | 190 | 8.00 | 4230 | D0, D6 |
| αPD1 | | 6 | TC53 | 8 | 190 | 2.67 | 1410 | D0, D6 |
| Nivo | | 7 | TC67 | 8 | 188 | 8.00 | 4230 | D0, D6 |
| αPD1 | | 8 | TC67 | 8 | 188 | 2.67 | 1410 | D0, D6 |
| Benchmark | Control | 9 | Pembro | 8 | 145 | 6.00 | 4230 | D0, D6 |
| | | 10 | Pembro | 8 | 145 | 2.00 | 4230 | D0, D6 |
| Unmask | Control | 11 | TC29 | 8 | 162 | 0.75 | 463 | D0, D6 |
| Benchmark | | | | | | | | |
| | | 12 | Vehicle | 8 | — | — | — | D0, D6 |

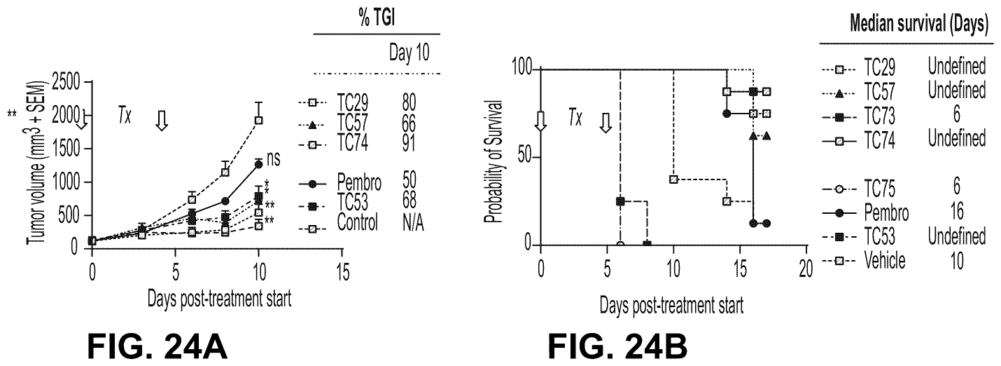

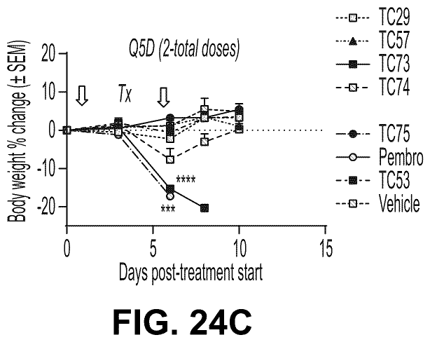

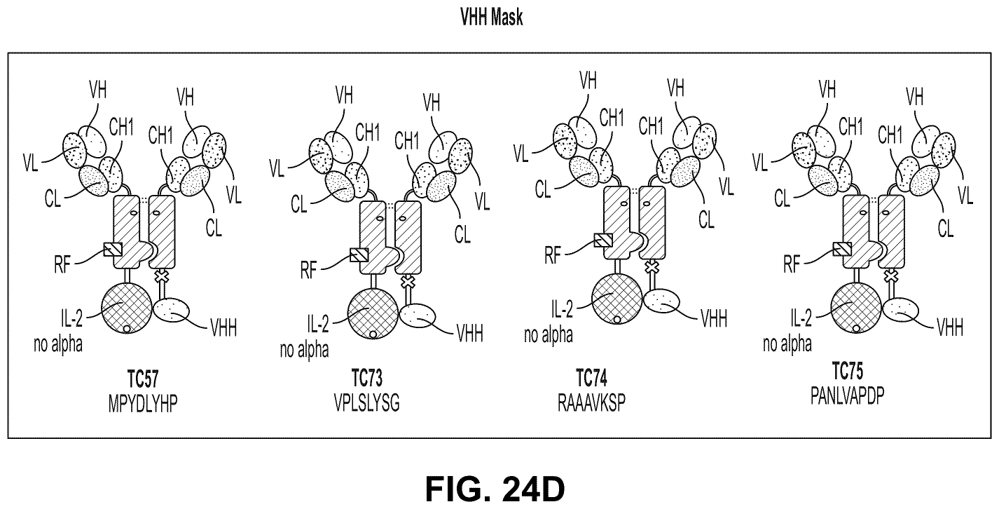

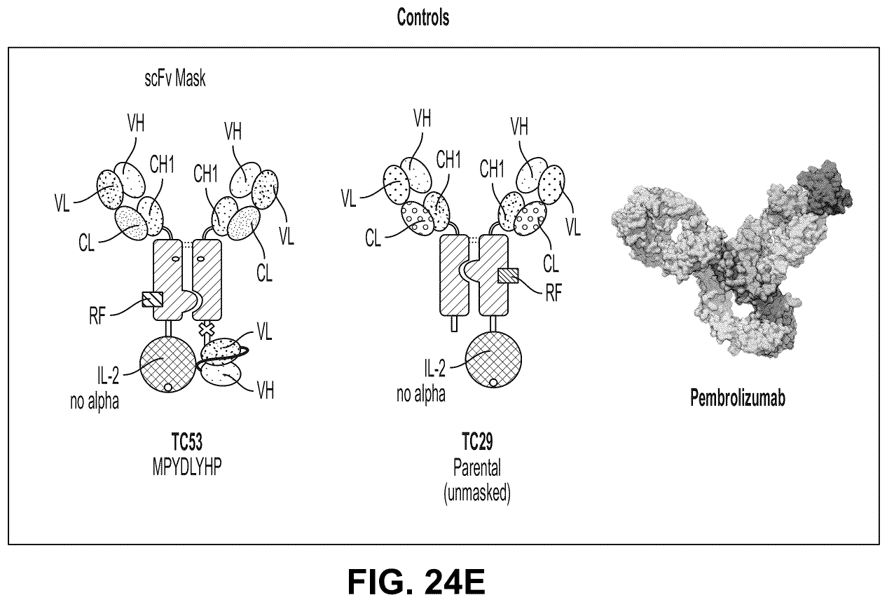

Efficacy (% TGI and survival) was also measured in mice treated with exemplary constructs according to Table 19 to evaluate different cleavage substrates. Quantitative measurement of cleaved molecules in MC38 tumor bearing mice demonstrated a higher quantity of cleaved product in tumor than spleens in all molecules (FIG. 24A-24D).

TABLE 19
Dosing in hPD1 transgenic mice to evaluate cleavage substrates

| | | | | | | | Equal | |
|---|---|---|---|---|---|---|---|---|
| | | | | | | Apprx. | molar | |
| | | | | Mouse | M.W. | Dosing | Dose | Dosing |
| Test Molecule | IL2 | Mask | Substrate | # | (kDa) | (mg/kg) | (nM) | Schedule |
| TC29 | IL2(not | Non- | — | N = 8 | 162 | 0.75 | 463 | Q5D |
| | alpha) | masked/P | | | | | | |
| TC57 | | VHH | MPY | N = 8 | 177 | 7.50 | 4230 | Q5D |
| TC73 | | | VPL | N = 8 | 177 | 7.50 | 4230 | Q5D |
| TC74 | | | RAA | N = 8 | 177 | 7.50 | 4230 | Q5D |
| TC75 | | | PAN | N = 8 | 177 | 7.50 | 4230 | Q5D |
| Pembrolizumab | — | — | — | N = 8 | 146 | 6 | 4230 | Q5D |
| TC53 | IL2(not | SCFV1 | MPY | N = 8 | 190 | 8 | 4230 | Q5D |
| | alpha) | | | | | | | |
| Vehicle | — | — | — | N = 8 | — | — | — | Q5D |

### Example 14: In Vivo NHP PK/PD of Targeted Masked Cytokines

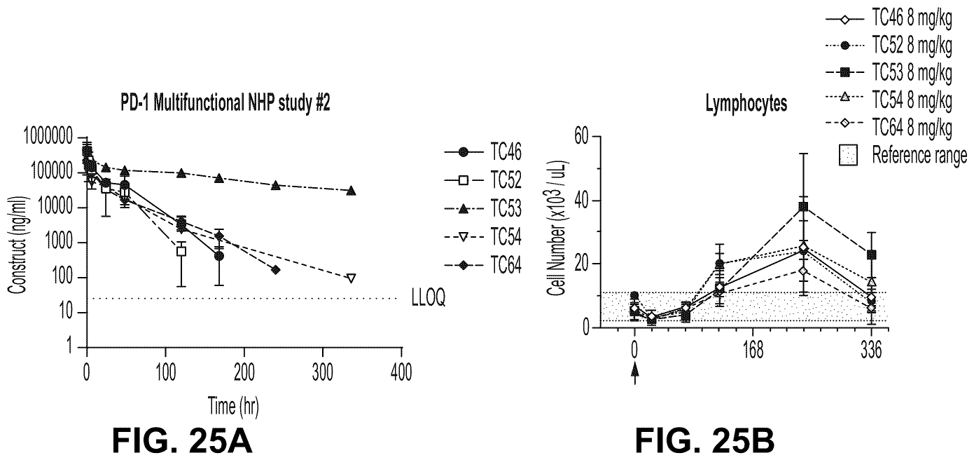

This example demonstrates PK/PD of the targeted cytokine constructs in non-human primates (NHP) according to Table 20. Briefly, naïve NHP (Cambodian) animals were given a single 30-minute intravenous infusion and observed for 14 days. Samples were taken Day 1—pre-dose, 5 min, 2 hr, 6 hr, 24 hr, 48 hr, 120 hr, 168 hr, 240 hr, 336 hr for PK studies, Day 1—pre dose, 336 hr to assess anti-drug antibodies (ADA) and pre-dose, 24 hr, 72 hr, 120 hr, 240 hr for clinical pathology studies. All animals tolerated single 8 mg/kg dose with no significant adverse effects observed. Anti-IL2 scFv SCFV1 mask construct did not develop ADAs and showed substantially increased exposure compared to CD122-masked molecules (Table 21 and FIG. 25A). All molecules demonstrated changes in lymphocytes as shown in FIG. 25B.

TABLE 20
Dosing in NHP Animals

| | | Dose | Dose | Dose | |
|---|---|---|---|---|---|
| | | Level | Volume | Concen- | No. of |
| Group | | (mg/ | (mL/ | tration | Animals |
| No. | Test Material | kg) | kg) | (mg/mL) | Females |
| 1 | TC46: PembroFab—IgG1 | 8 | 5 | 1.6 | 3 |
| | (N297A, MST, RF)— | | | | |
| | CD122* | | | | |
| 2 | TC52: PembroFab—IgG1 | 8 | | | 3 |
| | (N297A, MST, RF)— | | | | |
| | CD122(xG) | | | | |
| 3 | TC53: PembroFab—IgG1 | 8 | | | 3 |
| | (N297A, MST, | | | | |
| | RF)—SCFV1 | | | | |
| 4 | TC54: PembroFab—IgG1 | 8 | | | 3 |
| | (N297A, MST, RF)— | | | | |
| | CD 122*(xMan) | | | | |
| 5 | TC64: PembroFab—IgG1 | 8 | | | 3 |
| | (LALAPG, MST, RF)— | (or | | | |
| | CD122* | molar | | | |
| | | equiv. | | | |
| | | dose | | | |
| | | level) | | | |

TABLE 21
PK parameters in NHP Studies

| | | | | AUCₗₐₛₜ | AUC_{0-INF} | |
|---|---|---|---|---|---|---|
| | | | Cmax (ug/mL) | (hr*ug/mL) | (hr*ug/mL) | Clearance |
| Construct | Dose | T_{1/2} (hr) (±SD) | (±SD) | (±SD) | (±SD) | (mL/hr/kg) |
| TC46 | 8 mg/kg | 23.3 ± 3.72 | 438 ± 214 | 5130 ± 710 | 5140 ± 708 | 1.58 ± 0.213 |
| TC52 | 30-min IV infusion | 14.9 ± 4.06 | 419 ± 331 | 3820 ± 2560 | 3820 ± 2560 | 2.71 ± 1.41 |
| TC53 | | 143 ± 24.2 | 261 ± 19.8 | 19700 ± 1610 | 34000 ± 636 | 0.236 ± 0.004 |
| TC54 | | 21.6 ± 9.94 | 173 ± 25.9 | 2730 ± 988 | 2760 ± 1040 | 3.18 ± 1.17 |
| TC64 | | 29.5 ± 1.98 | 232 ± 27.8 | 3300 ± 331 | 3360 ± 350 | 2.40 ± 0.265 |

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A targeted cytokine comprising:
b) a targeting moiety;
c) a cytokine or a fragment thereof;
d) a masking moiety; and
e) an Fc domain comprising a first Fc polypeptide linked to the cytokine or a fragment thereof through a first linker and a second Fc polypeptide linked to the masking moiety through a second linker,
wherein the masking moiety binds to the cytokine or a fragment thereof,
wherein the first or the second linker is a cleavable linker such that the masking moiety releases the cytokine or a fragment thereof upon cleavage,
wherein the cleavable linker comprises MPYDLYHP (SEQ ID NO: 34) or VPLSLYSG (SEQ ID NO: 42) and
wherein the targeting moiety is linked to the Fc domain through one or both of the first and second Fc polypeptides.

2. A targeted cytokine comprising:
a) a targeting moiety;
b) a cytokine or a fragment thereof;
c) a masking moiety; and
d) an Fc domain comprising a first Fc polypeptide linked to the cytokine or a fragment thereof through a first linker and a second Fc polypeptide linked to the masking moiety through a second linker,
wherein the masking moiety binds to the cytokine or a fragment thereof,
wherein the first or the second linker is a cleavable linker such that the masking moiety releases the cytokine or a fragment thereof upon cleavage,
wherein the cleavable linker comprises an amino acid sequence selected from Table 1 and
wherein the targeting moiety is linked to the Fc domain through one or both of the first and second Fc polypeptides.

3-4. (canceled)

5. A targeted cytokine comprising:
a) a targeting moiety;
b) a cytokine or a fragment thereof;
c) a masking moiety; and
d) an Fc domain comprising a first Fc polypeptide linked to the cytokine or a fragment thereof through a first linker and a second Fc polypeptide linked to the masking moiety through a second linker,
wherein the masking moiety binds to the cytokine or a fragment thereof,
wherein the first or the second linker is a tumor specific cleavable linker such that the masking moiety releases the cytokine or a fragment thereof upon cleavage by a tumor specific protease,
wherein the tumor specific cleavable linker comprises between 8-50 amino acid residues, and
wherein the targeting moiety is linked to the Fc domain through one or both of the first and second Fc polypeptides.

6. (canceled)

7. A targeted cytokine comprising:
a) a targeting moiety;
b) a cytokine or a fragment thereof;
c) a masking moiety; and
d) an Fc domain comprising a first Fc polypeptide linked to the cytokine or a fragment thereof through a first linker and a second Fc polypeptide linked to the masking moiety through a second linker;
wherein the masking moiety binds to the cytokine or a fragment thereof,
wherein the first or the second linker is a cleavable linker such that the masking moiety releases the cytokine or a fragment thereof upon cleavage to yield active cytokine,
wherein the cleavable linker has an in vitro cleavage efficiency yielding at least 10% active cytokine and
wherein the targeting moiety is linked to the Fc domain through one or both of the first and second Fc polypeptides.

8. A targeted cytokine comprising:
a. a targeting moiety;
b. a cytokine or a fragment thereof;
c. a masking moiety comprising CD122 or a fragment thereof comprising one or more mutations selected from the group consisting of F8C, A94C, L106C, C122S, C122V, C122A, N123C, N123Q, C168V, C168A, C168S, L169C, Q177C, V184C, S195C, R204C; and
d. an Fc domain comprising a first Fc polypeptide linked to the cytokine or a fragment thereof through a first linker and a second Fc polypeptide linked to the masking moiety through a second linker,
wherein the masking moiety binds to the cytokine or a fragment thereof,
wherein the first or the second linker is a cleavable linker such that the masking moiety releases the cytokine or a fragment thereof upon cleavage, and
wherein the targeting moiety is linked to the Fc domain through one or both of the first and second Fc polypeptides.

9. A targeted cytokine comprising:
a) a targeting moiety,
b) a masking moiety,
c) a cytokine or fragment thereof, and
d) a Fc domain comprising
1) a first Fc polypeptide comprising a CH3 domain comprising a modification that reduces or eliminates binding to Protein A, and
2) a second Fc polypeptide comprising a CH3 domain that binds to Protein A.

10-13. (canceled)

14. The targeted cytokine of claim 2, wherein the cleavable linker comprises MPYDLYHP (SEQ ID NO: 34), RAAAVKSP (SEQ ID NO: 37), VPLSLYSG (SEQ ID NO: 42), PVSLRSGS (SEQ ID NO: 196), GMPKDLYHAS (SEQ ID NO. 197), RPLALWRS (SEQ ID NO: 193), TQKPLGLS (SEQ ID NO:194), APAGLIVPYN (SEQ ID NO:195), PANLVAPDP (SEQ ID NO: 183), IVGRPRHQGV (SEQ ID NO:199), or RSKYLATA (SEQ ID NO:198).

15-18. (canceled)

19. The targeted cytokine of claim 2, wherein the targeting moiety specifically binds PD-1, PD-L1, PD-L2, CTLA-4, TIGIT, TIM-3, LAG-3, CD25, CD16a, CD16b, NKG2D, NKP44, NKP30, CD19, CD20, CD30, CD38, BCMA, human epidermal growth factor receptor 2 (HER2), human epidermal growth factor receptor 3 (HERS), delta-like protein 3 (DLL3), delta-like protein 4 (DLL4), epidermal growth factor receptor (EGFR), glypican-3 (GPC3), c-MET, vascular endothelial growth factor receptor 1 (VEGF R1), vascular endothelial growth factor receptor 2 (VEGF R2), Nectin-4, Liv-1, glycoprotein NMB (GPNMB), prostate specific membrane antigen (PSMA), Trop-2, carbonic anhydrase IX (CA9), endothelin B receptor (ETBR), six transmembrane epithelial antigen of the prostate 1 (STEAP1), folate receptor alpha (FR-a), SLIT and NTRK-like protein 6 (SLITRK6), carbonic anhydrase VI (CA6), ectonucleotide pyrophosphatase/phosphodiesterase family member 3 (ENPP3), mesothelin, trophoblast glycoprotein (TPBG), CD19, CD20, CD22, CD33, CD40, CD56, CD66e, CD70, CD74, CD79b, CD98, CD 123, CD 138, CD352, CD47, signal-regulatory protein alpha (SIRPα), Claudin 18.2, Claudin 6, 5T4, fibroblast activation protein alpha (FAPα), the melanoma-associated chondroitin sulfate proteoglycan (MCSP), epithelial cellular adhesion molecule (EPCAM), or combinations thereof.

20-22. (canceled)

23. A targeted cytokine comprising:
a) a targeting moiety that specifically binds to PD-1 comprising
i) HCDR1 of SEQ ID NO: 4 (GYTFTNYY), HCDR2 of SEQ ID NO: 5 (INPSNGGT), HCDR3 SEQ ID NO: 6 (ARRDYRFDMGFDY), LCDR1 of SEQ ID NO: 9 (KGVSTSGYSY), LCDR2 of SEQ ID NO: 10 (LAS), and LCDR3 of SEQ ID NO: 11 (QHSRDLPLT); or
ii) HCDR1 of GITFSNSG (SEQ ID NO: 161), HCDR2 of VIWYDGSKRYYADSVKG (SEQ ID NO: 162), HCDR3 of ATNDDY (SEQ ID NO: 163), LCDR1 of QSVSSY (SEQ ID NO: 164), LCDR2 of DAS(SEQ ID NO: 165), and LCDR3 of QQSSNWPRT (SEQ ID NO: 166);
b) a cytokine or fragment thereof, and
c) an Fc domain.

24-44. (canceled)

45. The targeted cytokine of claim 2, wherein the targeting moiety comprises one or more antigen binding domains, a peptide, a polypeptide, a protein, a ligand, or an agent that specifically binds to the cytokine or the fragment thereof.

46-51. (canceled)

52. The targeted cytokine of claim 2,
wherein the C-terminus of the first antigen binding domain is linked to the N-terminus of the first Fc polypeptide;
wherein the C-terminus of the first Fc polypeptide is linked to the N-terminus of the cytokine or a fragment thereof;
wherein the C-terminus of the second antigen binding domain is linked to the N-terminus of the second Fc polypeptide; and
wherein the C-terminus of the second Fc polypeptide is linked to the N-terminus of the masking moiety.

53-54. (canceled)

55. The targeted cytokine of claim 2, wherein the first and/or the second Fc domains each contain one or more modifications that promote the non-covalent association of the first and the second Fc polypeptide.

56-64. (canceled)

65. The targeted cytokine of claim 2, wherein the cytokine or a fragment thereof is IL-1, IL-2, IL-3, IL-4, IL-5, IL-6, IL-7, IL-9, IL-10, IL-11, IL-12, IL-13, IL-14, IL-15, IL-16, IL-17, IL-20, TNF-α, TNF-β, CXCL8 (IL-18), G-CSF, GM-CSF, LIF, OSM, IFN-α, IFN-β, IFN-γ, CD154, LT-β, 4-1BBL, APRIL, CD70, CD153, CD178, GITRL, LIGHT, OX40L, TALL-1, TRAIL, TWEAK, TRANCE, TGF-β, M-CSF, or MSP or a fragment thereof.

66-67. (canceled)

68. The targeted cytokine of claim 72, wherein the masking moiety is CD121, IL-18Rα, IL-18Rβ, CD25, CD122, CD132, CD124, CD213a13, CD132, CD127, IL-9R, CD213a1, CD213a2, CD1243, CD132, IL-15Ra, CDw131, CDw125, CD131, CD116, CD126, CD130, IL-11Ra, CD114, CD212, LIFR, OSMR, IL-20Rα, IL-20Rβ, IL-14R, CD4, CDw127, CD118, CDw119, CD40, LTβR, CD120a, CD120b, CDw137, BCMA, TACI, CD27, CD30, CD95, GITR, LTbR, HVEM, OX40, TRAILR1-4, Apo3, RANK, OPG, TGF-13R1, TGF-βR2, TGF-βR3, CD115, or CDw136.

69-79. (canceled)

80. The targeted cytokine of claim 2, wherein the masking moiety comprises a Fab, a single chain Fv (scFv), a single domain antibody (VHH), one or more CDRs, a variable heavy chain (VH), a variable light chain (VL), a Fab-like bispecific antibodies (bsFab), a single-domain antibody-linked Fab (s-Fab), an antibody, or a combination thereof.

81-125. (canceled)

126. A nucleic acid encoding the targeted cytokine of claim 2.

127. (canceled)

128. A host cell comprising the nucleic acid of claim 126.

129. A method of producing a targeted cytokine comprising culturing the host cell of claim 128 under a condition that produces the targeted cytokine.

130-132. (canceled)

133. A method of treating or preventing a neoplastic disease in a subject, the method comprising administering to the subject an effective amount of the targeted cytokine of claim 2.

134. A method of treating or preventing an inflammatory or autoimmune disease in a subject, the method comprising administering to the subject an effective amount of the targeted cytokine of claim 2.

135-138. (canceled)


SEQUENCE LISTING (266 sequences)

SEQ ID NO: 1 - protein, 120 aa, synthetic construct [sequence omitted]

SEQ ID NO: 2 - protein, 223 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 3 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 8 aa, synthetic construct
GYTFTNYY

SEQ ID NO: 5 - protein, 8 aa, synthetic construct
INPSNGGT

SEQ ID NO: 6 - protein, 13 aa, synthetic construct
ARRDYRFDMGFDY

SEQ ID NO: 7 - protein, 119 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKTSENLYFQ

SEQ ID NO: 8 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 10 aa, synthetic construct
KGVSTSGYSY

SEQ ID NO: 10 - skipped

SEQ ID NO: 11 - protein, 9 aa, synthetic construct
QHSRDLPLT

SEQ ID NO: 12 - protein, 153 aa, synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 13 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 14 - protein, 133 aa, synthetic construct
APTSSSTKKTQLQLEHLLLDLQMILNGINNYKNPKLTAMLTAKFAMPKKATELKHLQCLEEALKPLEEVLNLAQSKNFHLRPRDLISNINVIVLELKGSETTFMCEYADETATIVEFLNRWITFAQSIISTLT

SEQ ID NO: 15 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKD

SEQ ID NO: 16 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRSNISWEISQASHYFERHLEFEARTLSPGHTWEEAPLLTLKQKQEWISLETLTPDTQYEFQVRVKPLQGEFTTWSPWSQPLAFRTKPAALGKD

SEQ ID NO: 17 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRVNISWEISQASHYFERHLEFEARTLSPGHTWEEAPLLTLKQKQEWIVLETLTPDTQYEFQVRVKPLQGEFTTWSPWSQPLAFRTKPAALGKD

SEQ ID NO: 18 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWIVLETLTPDTQYEFQVRVKPLQGEFTTWSPWSQPLAFRTKPAALGKD

SEQ ID NO: 19 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWIVLETLTPDTQYEFQVRVKPLQGEFTTWSPWSQPLAFRTKPAALGKD

SEQ ID NO: 20 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRVNISWEISQASHYFERHLEFEARTLSPGHTWEEAPLLTLKQKQEWIALETLTPDTQYEFQVRVKPLQGEFTTWSPWSQPLAFRTKPAALGKD

SEQ ID NO: 21 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRACISWEISQASHYFERHLEFEARTLSPGHTWEEAPLLTLKQKQEWICLETLTPDTQYEFQVRVKPLQGEFTTWSPWSQPLAFRTKPAALGKD

SEQ ID NO: 22 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRVCISWEISQASHYFERHLEFEARTLSPGHTWEEAPLLTLKQKQEWICLETLTPDTQYEFQVRVKPLQGEFTTWSPWSQPLAFRTKPAALGKD

SEQ ID NO: 23 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWIALETLTPDTQYEFQVRVKPLQGEFTTWSPWSQPLAFRTKPAALGKD

SEQ ID NO: 24 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHCETHRCQISWEISQASHYFERHLEFEARTLSPGHTWEEAPLLTLKQKQEWIALETLTPDTQYEFQVRVKPLQGEFTTWSPWSQPLAFRTKPAALGKD

SEQ ID NO: 25 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCQISWEISQASHYFERHLEFEARTLSPGHTWEEAPLLTLKQKQEWIACETLTPDTQYEFQVRVKPLQGEFTTWSPWSQPLAFRTKPAALGKD

SEQ ID NO: 26 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRCMAPISLQVVHVETHRANISWEISQASHYFERHLEFEARTLSPGHTWEEAPLLTLKQKQEWIALETLTPDTQYEFQVRVKPLQGEFTTWCPWSQPLAFRTKPAALGKD

SEQ ID NO: 27 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRCMAPISLQVVHVETHRANISWEISQASHYFERHLEFEARTLSPGHTWEEAPLLTLKQKQEWIALETLTPDTQYEFQVRCKPLQGEFTTWSPWSQPLAFRTKPAALGKD

SEQ ID NO: 28 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWIALETLTPDTQYEFQVRCKPLQGEFTTWCPWSQPLAFRTKPAALGKD

SEQ ID NO: 29 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRANISWEISQASHYFERHLEFEARTLSPGHTWEEAPLLTLKQKQEWIALETLTPDTCYEFQVRVKPLQGEFTTWSPWSQPLAFCTKPAALGKD

SEQ ID NO: 30 - protein, 213 aa, synthetic construct
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRCMAPISLQVVHVETHRVNISWEISQASHYFERHLEFEARTLSPGHTWEEAPLLTLKQKQEWIVLETLTPDTQYEFQVRVKPLQGEFTTWCPWSQPLAFRTKPAALGKD

SEQ ID NO: 31 - protein, 213 aa, synthetic construct
AVNGTSQCTCFYNSRANISCVWSQDGALQDTSCQVHAWPDRRRWNQTCELLPVSQASWACNLILGAPDSQKLTTVDIVTLRVLCREGVRWRVMCIQDFKPFENLRLMAPISLQVVHVETHRVNISWEISQASHYFERHLEFEARTLSPGHTWEEAPLLTLKQKQEWIVLETLTPDTQYEFQVRVKPLQGEFTTWSPWSQPLAFRTKPAALGKD

SEQ ID NO: 32 - protein, 5 aa, synthetic construct
PGSGS

SEQ ID NO: 33 - protein, 17 aa, synthetic construct
GGSSPPGGGSSGGGSGP

SEQ ID NO: 34 - protein, 8 aa, synthetic construct
MPYDLYHP

SEQ ID NO: 35 - protein, 8 aa, synthetic construct
DSGGFMLT

SEQ ID NO: 36 - protein, 6 aa, synthetic construct
HEQLTV

SEQ ID NO: 37 - protein, 8 aa, synthetic construct
RAAAVKSP

SEQ ID NO: 38 - protein, 6 aa, synthetic construct
VPLSLY

SEQ ID NO: 39 - protein, 9 aa, synthetic construct
DLLAVVAAS

SEQ ID NO: 40 - protein, 10 aa, synthetic construct
ISSGLLSGRS

SEQ ID NO: 41 - protein, 9 aa, synthetic construct
MPYDLYHPS

SEQ ID NO: 42 - protein, 8 aa, synthetic construct
VPLSLYSG

SEQ ID NO: 43 - protein, 13 aa, synthetic construct
ISSGLLSGRSDQP

SEQ ID NO: 44 - protein, 20 aa, synthetic construct
GPPSGSSPMPYDLYHPSGGG

SEQ ID NO: 45 - protein, 21 aa, synthetic construct
GPPSGSSPGMPYDLYHPSGGG

SEQ ID NO: 46 - protein, 15 aa, synthetic construct
GGPMPYDLYHPSGGG

SEQ ID NO: 47 - protein, 15 aa, synthetic construct
GSPMPYDLYHPSGGG

SEQ ID NO: 48 - protein, 17 aa, synthetic construct
SSGGPMPYDLYHPSGGG

SEQ ID NO: 49 - protein, 18 aa, synthetic construct
SGSSPGMPYDLYHPSGGG

SEQ ID NO: 50 - protein, 19 aa, synthetic construct
GSSGPSGMPYDLYHPSGGG

SEQ ID NO: 51 - protein, 20 aa, synthetic construct
GSSSGPGSMPYDLYHPSGGG

SEQ ID NO: 52 - protein, 20 aa, synthetic construct
GPPSGSSPVPLSLYSGSGGG

SEQ ID NO: 53 - protein, 16 aa, synthetic construct
GGSPVPLSLYSGSGGG

SEQ ID NO: 54 - protein, 16 aa, synthetic construct
GSGGVPLSLYSGSGGG

SEQ ID NO: 55 - protein, 17 aa, synthetic construct
GGSPGVPLSLYSGSGGG

SEQ ID NO: 56 - protein, 18 aa, synthetic construct
GGSSGPVPLSLYSGSGGG

SEQ ID NO: 57 - protein, 19 aa, synthetic construct
GGSSGSGVPLSLYSGSGGG

SEQ ID NO: 58 - protein, 19 aa, synthetic construct
GGSPGSPVPLSLYSGSGGG

SEQ ID NO: 59 - protein, 26 aa, synthetic construct
GSPGVPLSLYSGSSPMPYDLYHPSGG

SEQ ID NO: 60 - protein, 6 aa, synthetic construct
GGSSPP

SEQ ID NO: 61 - protein, 4 aa, synthetic construct
GSGP

SEQ ID NO: 62 - protein, 4 aa, unidentified
GSPG

SEQ ID NO: 63 - protein, 4 aa, synthetic construct
GGSG

SEQ ID NO: 64 - protein, 9 aa, synthetic construct
GPPSGSSPG

SEQ ID NO: 65 - protein, 8 aa, synthetic construct
GPPSGSSP

SEQ ID NO: 66 - protein, 4 aa, synthetic construct
GGPS

SEQ ID NO: 67 - protein, 5 aa, synthetic construct
GSGPS

SEQ ID NO: 68 - protein, 6 aa, synthetic construct
GSSGGP

SEQ ID NO: 69 - skipped

SEQ ID NO: 70 - protein, 6 aa, synthetic construct
GSGSPS

SEQ ID NO: 71 - protein, 7 aa, synthetic construct
PSGSSPG

SEQ ID NO: 72 - protein, 5 aa, synthetic construct
SGSPS

SEQ ID NO: 73 - skipped

SEQ ID NO: 74 - protein, 4 aa, synthetic construct
SGGG

SEQ ID NO: 75 - protein, 5 aa, synthetic construct
GSGGG

SEQ ID NO: 76 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 77 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 78 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 79 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 80 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 81 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 82 - protein, 226 aa, synthetic construct
DKTHTCPPCPAPELLGGPSVFLFPPKPKDTLMASRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 83 - protein, 226 aa, synthetic construct
DKTHTCPPCPAPELLGGPSVFLFPPKPKDTLMASRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 84 - protein, 230 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREELTKNQVSLWCLVKGFYPSDIAVEWESSGQPENNYKTTPPVLDSDGSFFLYSKLTVDKSRWQQGNIFSCSVMHEALHNRFTQKSLSLSPGGSPG

SEQ ID NO: 85 - protein, 230 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPGGSPG

SEQ ID NO: 86 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 87 - protein, 599 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPGGGSSPPGGGSSGGGSGPAPTSSSTKKTQLQLEHLLLDLQMILNGINNYKNPKLTAMLTAKFAMPKKATELKHLQCLEEALKPLEEVLNLAQSKNFHLRPRDLISNINVIVLELKGSETTFMCEYADETATIVEFLNRWITFAQSIISTLT

SEQ ID NO: 88 - protein, 682 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGPPSGSSPMPYDLYHPSGGGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRSNISWEISQASHYFERHLEFEARTLSPGHTWEEAPLLTLKQKQEWISLETLTPDTQYEFQVRVKPLQGEFTTWSPWSQPLAFRTKPAALGKD

SEQ ID NO: 89 - protein, 682 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGPPSGSSPMPYDLYHPSGGGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRACISWEISQASHYFERHLEFEARTLSPGHTWEEAPLLTLKQKQEWICLETLTPDTQYEFQVRVKPLQGEFTTWSPWSQPLAFRTKPAALGKD

SEQ ID NO: 90 - protein, 680 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSSGPVPLSLYSGSGGGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRSNISWEISQASHYFERHLEFEARTLSPGHTWEEAPLLTLKQKQEWISLETLTPDTQYEFQVRVKPLQGEFTTWSPWSQPLAFRTKPAALGKD

SEQ ID NO: 91 - protein, 680 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGSSGPVPLSLYSGSGGGAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRACISWEISQASHYFERHLEFEARTLSPGHTWEEAPLLTLKQKQEWICLETLTPDTQYEFQVRVKPLQGEFTTWSPWSQPLAFRTKPAALGKD

SEQ ID NO: 92 - protein, 162 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 93 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 94 - protein, 114 aa, synthetic construct
NWVNVISDLKKIEDLIQSMHIAATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 95 - protein, 114 aa, synthetic construct
NWVNVISDLKKIEDLIQSMHIDATLYTESDVHPSCKVTAMKCFLLALQVISLESGDASIHDTVENLIILANNSLSSNGNVTESGCKECEELEEKNIKEFLQSFVHIVQMFINTS

SEQ ID NO: 96 - protein, 114 aa, synthetic construct
NWVNVISDLKKIEDLIQSMHIDATLYTESDVHPSCKVTAMKCFLLALQVISLASGDASIHDTVENLIILANNSLSSNGNVTESGCKECEELEEKNIKEFLQSFVHIVQMFINTS

SEQ ID NO: 97 - protein, 114 aa, synthetic construct
NWVNVISDLKKIEDLIQSMHIDATLYTESDVHPSCKVTAMKCFLLRLQVISLRSGDASIHDTVENLIILANNSLSSNGNVTESGCKECEELEEKNIKEFLQSFVHIVQMFINTS

SEQ ID NO: 98 - protein, 114 aa, synthetic construct
NWVNVISDLKKIEDLIQSMHIDATLYTESDVHPSCKVTAMKCFLLSLQVISLSSGDASIHDTVENLIILANNSLSSNGNVTESGCKECEELEEKNIKEFLQSFVHIVQMFINTS

SEQ ID NO: 99 - protein, 114 aa, synthetic construct
NWVNVISDLKKIEDLIQSMHIDATLYTESDVHPSCKVTAMKCFLLELQVISLASGDASIHDTVENLIILANNSLSSNGNVTESGCKECEELEEKNIKEFLQSFVHIVQMFINTS

SEQ ID NO: 100 - protein, 18 aa, synthetic construct
NWVNVISDLKKIEDLIQS

SEQ ID NO: 101 - protein, 79 aa, synthetic construct
KVTAMKCFLLELQVISLESGDASIHDTVENLIILANNSLSSNGNVTESGCKECEELEEKNIKEFLQSFVHIVQMFINTS

SEQ ID NO: 102 - protein, 114 aa, synthetic construct
NWVNVISDLKKIEDLIQSMHIDATLYTESDVHPSCKVTAMKCFLLELQVISLESGDASIHDTVENLIILAQNSLSSNGNVTESGCKECEELEEKNIKEFLQSFVHIVQMFINTS

SEQ ID NO: 103 - protein, 114 aa, synthetic construct
NWVNVISDLKKIEDLIQSMHIDATLYTESDVHPSCKVTAMKCFLLELQVISLESGDASIHDTVENLIILANNSLSSNGQVTESGCKECEELEEKNIKEFLQSFVHIVQMFINTS

SEQ ID NO: 104 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFIQTS

SEQ ID NO: 105 - protein, 114 aa, synthetic construct
NWVNVISDLKKIEDLIQSMHIDATLYTESDVHPSCKVTAMKCFLLELQVISLESGDASIHDTVENLIILAQNSLSSNGQVTESGCKECEELEEKNIKEFLQSFVHIVQMFINTS

SEQ ID NO: 106 - protein, 114 aa, synthetic construct
NWVNVISDLKKIEDLIQSMHIDATLYTESDVHPSCKVTAMKCFLLELQVISLESGDASIHDTVENLIILAQNSLSSNGNVTESGCKECEELEEKNIKEFLQSFVHIVQMFIQTS

SEQ ID NO: 107 - protein, 114 aa, synthetic construct
NWVNVISDLKKIEDLIQSMHIDATLYTESDVHPSCKVTAMKCFLLELQVISLESGDASIHDTVENLIILANNSLSSNGQVTESGCKECEELEEKNIKEFLQSFVHIVQMFIQTS

SEQ ID NO: 108 - protein, 114 aa, synthetic construct
NWVNVISDLKKIEDLIQSMHIDATLYTESDVHPSCKVTAMKCFLLELQVISLESGDASIHDTVENLIILAQNSLSSNGQVTESGCKECEELEEKNIKEFLQSFVHIVQMFIQTS

SEQ ID NO: 109 - protein, 328 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 110 - protein, 219 aa, synthetic construct
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 111 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 112 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 113 - protein, 306 aa, synthetic construct; identical to SEQ ID NO: 111

SEQ ID NO: 114 - protein, 10 aa, synthetic construct
KSKREKKDRV

SEQ ID NO: 115 - protein, 303 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKDNTERVFTDKTSATVICRKNASISVRAQDRYYSSSWSEWASVPCS

SEQ ID NO: 116 - protein, 304 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKDNTEGRVFTDKTSATVICRKNASISVRAQDRYYSSSWSEWASVPCS

SEQ ID NO: 117 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKSKREKKDRVFTDKTSATVICRKNASISVRAQDRYYSSSWSEWASVPCS

SEQ ID NO: 118 - protein, 304 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKDNTEGRVFTDKTSATVICRKNASISVRAQDRYYSSSWSEWASVPCS

SEQ ID NO: 119 - protein, 15 aa, synthetic construct
GGGGSGGGGSGGGGS

SEQ ID NO: 120 - protein, 518 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 121 - protein, 515 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKDNTERVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 122 - protein, 516 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKDNTEGRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 123 - protein, 518 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 124 - protein, 516 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKDNTEGRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 125 - protein, 662 aa, synthetic construct
MEPLVTWVVPLLFLFLLSRQGAACRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 126 - protein, 213 aa, synthetic construct
CRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENP

SEQ ID NO: 127 - protein, 522 aa, synthetic construct
CRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSD

SEQ ID NO: 128 - protein, 189 aa, synthetic construct
KIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSL

SEQ ID NO: 129 - protein, 199 aa, synthetic construct
KIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIV

SEQ ID NO: 130 - protein, 296 aa, synthetic construct
KIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEE

SEQ ID NO: 131 - protein, 296 aa, synthetic construct
KIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRSTLYWRDEGLVLLNRLRYRPSNSRLWNMVNVTKAKGRHDLLDLKPFTEYEFQISSKLHLYKGSWSDWSESLRAQTPEE

SEQ ID NO: 132 - protein, 599 aa, synthetic construct
KIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKAN

SEQ ID NO: 133 - protein, 204 aa, unidentified
KIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPP

SEQ ID NO: 134 - protein, 5 aa, synthetic construct
GGGGS

SEQ ID NO: 135 - protein, 10 aa, synthetic construct
GGGGSGGGGS

SEQ ID NO: 136 - protein, 13 aa, synthetic construct
GGSGGGSGGGGGS

SEQ ID NO: 137 - protein, 18 aa, synthetic construct
GGSGGSGGSGGSGGSSGP

SEQ ID NO: 138 - protein, 6 aa, unidentified
PGGSGP

SEQ ID NO: 139 - protein, 5 aa, synthetic construct
GGSPG

SEQ ID NO: 140 - protein, 15 aa, unidentified
GGSGGSVPLSLYSGP

SEQ ID NO: 141 - protein, 18 aa, synthetic construct
GGSGGSGGSVPLSLYSGP

SEQ ID NO: 142 - protein, 17 aa, synthetic construct
GGSGGSMPYDLYHPSGP

SEQ ID NO: 143 - protein, 20 aa, synthetic construct
GGSGGSGGSMPYDLYHPSGP

SEQ ID NO: 144 - protein, 17 aa, synthetic construct
GGSGGSDSGGFMLTSGP

SEQ ID NO: 145 - protein, 20 aa, synthetic construct
GGSGGSGGSDSGGFMLTSGP

SEQ ID NO: 146 - protein, 17 aa, synthetic construct
GGSGGSRAAAVKSPSGP

SEQ ID NO: 147 - protein, 20 aa, synthetic construct
GGSGGSGGSRAAAVKSPSGP

SEQ ID NO: 148 - protein, 19 aa, synthetic construct
GGSGGSISSGLLSGRSSGP

SEQ ID NO: 149 - protein, 22 aa, synthetic construct
GGSGGSGGSISSGLLSGRSSGP

SEQ ID NO: 150 - protein, 518 aa, synthetic construct; identical to SEQ ID NO: 120

SEQ ID NO: 151 - protein, 515 aa, synthetic construct; identical to SEQ ID NO: 121

SEQ ID NO: 152 - protein, 516 aa, synthetic construct; identical to SEQ ID NO: 122

SEQ ID NO: 153 - protein, 518 aa, synthetic construct; identical to SEQ ID NO: 123

SEQ ID NO: 154 - protein, 516 aa, synthetic construct; identical to SEQ ID NO: 124

SEQ ID NO: 155 - protein, 862 aa, synthetic construct
MAHTFRGCSLAFMFIITWLLIKAKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKANWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 156 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 157 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 158 - protein, 213 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 159 - protein, 113 aa, synthetic construct [sequence omitted]

SEQ ID NO: 160 - protein, 107 aa, synthetic construct [sequence omitted]

SEQ ID NO: 161 - protein, 8 aa, synthetic construct
GITFSNSG

SEQ ID NO: 162 - protein, 17 aa, synthetic construct
VIWYDGSKRYYADSVKG

SEQ ID NO: 163 - protein, 6 aa, synthetic construct
ATNDDY

SEQ ID NO: 164 - protein, 6 aa, synthetic construct
QSVSSY

SEQ ID NO: 165 - skipped

SEQ ID NO: 166 - protein, 9 aa, synthetic construct
QQSSNWPRT

SEQ ID NO: 167 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFAQSIISTLT

SEQ ID NO: 168 - protein, 119 aa, synthetic construct
EVQLVESGGGLVQPGGSLRLSCAASGSIFSINVMGWYRQAPGKQRELVAAISSGGSTNYADSVKGRFTISRDNAKNTVYLQMNSLKPEDTAVYYCMYASSWYEDETDYWGQGTQVTVSS

SEQ ID NO: 169 - protein, 10 aa, synthetic construct
GSIFSINVMG

SEQ ID NO: 170 - protein, 16 aa, synthetic construct
AISSGGSTNYADSVKG

SEQ ID NO: 171 - protein, 11 aa, synthetic construct
ASSWYEDETDY

SEQ ID NO: 172 - protein, 121 aa, synthetic construct
QLQLQESGPGLVKPSQTLSLTCTVSGGSISSGGYYWSWIRQHPGKGLEWIGYIYKSGSAYYSPSLKSRVTISVDTSKNQFSLKLSSVTAADTAVYYCARTPTVTGDWFDPWGRGTLVTVSS

SEQ ID NO: 173 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQTYDSIDVYFGGGTKLTVL

SEQ ID NO: 174 - protein, 7 aa, synthetic construct
SGGYYWS

SEQ ID NO: 175 - protein, 19 aa, synthetic construct
GYIYKSGSAYYSPSLKSRV

SEQ ID NO: 176 - protein, 11 aa, synthetic construct
TPTVTGDWFDP

SEQ ID NO: 177 - protein, 13 aa, synthetic construct
TRSSGSIASNYVQ

SEQ ID NO: 178 - protein, 7 aa, synthetic construct
EDNQRPS

SEQ ID NO: 179 - protein, 9 aa, synthetic construct
QTYDSIDVY

SEQ ID NO: 180 - protein, 252 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQTYDSIDVYFGGGTKLTVLGGGGGSGGGGSGGGGSGGGGSQLQLQESGPGLVKPSQTLSLTCTVSGGSISSGGYYWSWIRQHPGKGLEWIGYIYKSGSAYYSPSLKSRVTISVDTSKNQFSLKLSSVTAADTAVYYCARTPTVTGDWFDPWGRGTLVTVSS

SEQ ID NO: 181 - protein, 18 aa, synthetic construct
GGSSGSGGSGGGSGSGGG

SEQ ID NO: 182 - protein, 21 aa, synthetic construct
GGGGGSGGGGSGGGGSGGGGS

SEQ ID NO: 183 - protein, 9 aa, synthetic construct
PANLVAPDP

SEQ ID NO: 184 - protein, 8 aa, synthetic construct
IHVTLKSL

SEQ ID NO: 185 - protein, 8 aa, synthetic construct
NSYTIKGL

SEQ ID NO: 186 - protein, 8 aa, synthetic construct
SNESLSLS

SEQ ID NO: 187 - protein, 8 aa, synthetic construct
SQESLSLS

SEQ ID NO: 188 - protein, 8 aa, synthetic construct
QVSSSLSP

SEQ ID NO: 189 - protein, 8 aa, synthetic construct
PTSTSLSP

SEQ ID NO: 190 - protein, 8 aa, synthetic construct
ESLSLSEE

SEQ ID NO: 191 - protein, 8 aa, synthetic construct
ASLSLAPV

SEQ ID NO: 192 - protein, 4 aa, synthetic construct
PLGL

SEQ ID NO: 193 - protein, 8 aa, synthetic construct
RPLALWRS

SEQ ID NO: 194 - protein, 8 aa, synthetic construct
TQKPLGLS

SEQ ID NO: 195 - protein, 10 aa, synthetic construct
APAGLIVPYN

SEQ ID NO: 196 - protein, 8 aa, synthetic construct
PVSLRSGS

SEQ ID NO: 197 - protein, 10 aa, synthetic construct
GMPKDLYHAS

SEQ ID NO: 198 - protein, 8 aa, synthetic construct
RSKYLATA

SEQ ID NO: 199 - protein, 10 aa, unidentified
IVGRPRHQGV

SEQ ID NO: 200 - protein, 4 aa, synthetic construct
GFLG

SEQ ID NO: 201 - protein, 4 aa, synthetic construct
GLFG

SEQ ID NO: 202 - protein, 7 aa, synthetic construct
AGRRAAK

SEQ ID NO: 203 - protein, 5 aa, synthetic construct
FRLWA

SEQ ID NO: 204 - protein, 5 aa, synthetic construct
FRLWS

SEQ ID NO: 205 - protein, 8 aa, synthetic construct
NFFGVGGE

SEQ ID NO: 206 - protein, 8 aa, synthetic construct
PMKRLTLA

SEQ ID NO: 207 - protein, 8 aa, synthetic construct
FPLATYAP

SEQ ID NO: 208 - protein, 8 aa, synthetic construct
FLVGGASL

SEQ ID NO: 209 - protein, 8 aa, synthetic construct
KPMQFLGD

SEQ ID NO: 210 - protein, 8 aa, synthetic construct
GIVRAKGV

SEQ ID NO: 211 - protein, 8 aa, synthetic construct
ALFKSSFP

SEQ ID NO: 212 - protein, 8 aa, synthetic construct
SGRRSPGG

SEQ ID NO: 213 - protein, 8 aa, synthetic construct
SLGRRPGG

SEQ ID NO: 214 - protein, 8 aa, synthetic construct
SLSGRRGG

SEQ ID NO: 215 - protein, 8 aa, synthetic construct
SLSLGRRG

SEQ ID NO: 216 - protein, 8 aa, synthetic construct
SLSLSGRR

SEQ ID NO: 217 - protein, 6 aa, synthetic construct
GGPRRL

SEQ ID NO: 218 - protein, 6 aa, synthetic construct
GGPLRL

SEQ ID NO: 219 - protein, 6 aa, synthetic construct
GGPKLL

SEQ ID NO: 220 - protein, 6 aa, synthetic construct
GGPRNL

SEQ ID NO: 221 - protein, 6 aa, synthetic construct
GGPRML

SEQ ID NO: 222 - protein, 8 aa, synthetic construct
EHLRSPGG

SEQ ID NO: 223 - protein, 8 aa, synthetic construct
FRSGVPGG

SEQ ID NO: 224 - protein, 8 aa, synthetic construct
SLLLRTGN

SEQ ID NO: 225 - protein, 8 aa, synthetic construct
AGLRSPGG

SEQ ID NO: 226 - protein, 8 aa, synthetic construct
SLFRSAGP

SEQ ID NO: 227 - protein, 8 aa, synthetic construct
SLFRAPGP

SEQ ID NO: 228 - protein, 8 aa, synthetic construct
WLFRSPLG

SEQ ID NO: 229 - protein, 8 aa, synthetic construct
SRLRSPQG

SEQ ID NO: 230 - protein, 8 aa, synthetic construct
SLVLSGRR

SEQ ID NO: 231 - protein, 8 aa, synthetic construct
KQLRHMRG

SEQ ID NO: 232 - protein, 8 aa, synthetic construct
LSGRSDNH

SEQ ID NO: 233 - protein, 4 aa, synthetic construct
LSGK

SEQ ID NO: 234 - protein, 4 aa, synthetic construct
LSGR

SEQ ID NO: 235 - protein, 8 aa, synthetic construct
RQARVVGG

SEQ ID NO: 236 - protein, 8 aa, synthetic construct
RQRRVVGG

SEQ ID NO: 237 - protein, 8 aa, synthetic construct
RQYRVVGG

SEQ ID NO: 238 - protein, 8 aa, synthetic construct
SKGRSLIG

SEQ ID NO: 239 - protein, 8 aa, synthetic construct
PRFKIIGG

SEQ ID NO: 240 - protein, 8 aa, synthetic construct
KQLRVVNG

SEQ ID NO: 241 - protein, 8 aa, synthetic construct
IQPRITGG

SEQ ID NO: 242 - protein, 8 aa, synthetic construct
KQSRKFVP

SEQ ID NO: 243 - protein, 8 aa, synthetic construct
GRQSRAGG

SEQ ID NO: 244 - protein, 8 aa, synthetic construct
SGRSSPGG

SEQ ID NO: 245 - protein, 8 aa, synthetic construct
SSGRSPGG

SEQ ID NO: 246 - protein, 8 aa, unidentified
SLSGRSGG

SEQ ID NO: 247 - protein, 8 aa, synthetic construct
SLSSGRSG

SEQ ID NO: 248 - protein, 8 aa, synthetic construct
KLSLSGRS

SEQ ID NO: 249 - protein, 7 aa, synthetic construct
PLRLSRA

SEQ ID NO: 250 - protein, 7 aa, synthetic construct
PLKLSRA

SEQ ID NO: 251 - protein, 8 aa, synthetic construct
PLGLSGRS

SEQ ID NO: 252 - protein, 8 aa, synthetic construct
PLGLRSRA

SEQ ID NO: 253 - protein, 8 aa, synthetic construct
PLGLKSRA

SEQ ID NO: 254 - protein, 6 aa, synthetic construct
RGSRAG

SEQ ID NO: 255 - protein, 6 aa, synthetic construct
RLSRGK

SEQ ID NO: 256 - protein, 6 aa, synthetic construct
RGSRGG

SEQ ID NO: 257 - protein, 6 aa, synthetic construct
KGSRAG

SEQ ID NO: 258 - protein, 6 aa, synthetic construct
KLSRGK

SEQ ID NO: 259 - protein, 6 aa, unidentified
GRSRAG

SEQ ID NO: 260 - protein, 6 aa, synthetic construct
LRSRGK

SEQ ID NO: 261 - protein, 6 aa, synthetic construct
GRSRGG

SEQ ID NO: 262 - protein, 6 aa, synthetic construct
GKSRAG

SEQ ID NO: 263 - protein, 6 aa, synthetic construct
LKSRGK

SEQ ID NO: 264 - protein, 216 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 265 - protein, 19 aa, synthetic construct
SPGGGGPMPYDLYHPSGGG

SEQ ID NO: 266 - protein, 7 aa, synthetic construct
SPGGGGP